(19) United States
(12) Patent Application Publication
(10) Pub. No.: US 2024/0301034 A1
(43) Pub. Date: Sep. 12, 2024

(54) EXTRACELLULAR VESICLES LOADED WITH STRUCTURAL BIOMOLECULES

(71) Applicant: Spot Biosystems Ltd., Palo Alto, CA (US)

(72) Inventors: Ly James LEE, Columbus, OH (US); Junfeng SHI, Dublin, OH (US); Kwang Joo KWAK, Dublin, OH (US); Wen Jing LU, Beijing (CN); Yi YOU, Beijing (CN); Yu TIAN, Beijing (CN); Feng LAN, Beijing (CN); Andrew Stephen LEE, Palo Alto, CA (US)

(21) Appl. No.: 18/491,744

(22) Filed: Oct. 20, 2023

Related U.S. Application Data
(63) Continuation of application No. PCT/US2022/026015, filed on Apr. 22, 2022.
(60) Provisional application No. 63/179,058, filed on Apr. 23, 2021.

(51) Int. Cl. C07K 14/78; A61K 38/00; A61M 5/32; A61P 17/02; C12N 5/077

(52) U.S. Cl. CPC C07K 14/78; A61M 5/3295; A61P 17/02; C12N 5/0656; A61K 38/00

(57) ABSTRACT

Described herein are compositions of therapeutic extracellular vesicles, and methods and systems of producing the therapeutic extracellular vesicles. Also described herein are methods of treating a disease or condition with the therapeutic extracellular vesicles.

### CROSS-REFERENCE

This application is a continuation of International Application No. PCT/US22/26015, filed Apr. 22, 2022, which claims the benefit of U.S. Provisional Application No. 63/179,058, filed on Apr. 23, 2021, each of which is incorporated herein by reference in their entirety for all purposes.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BACKGROUND

Extracellular vesicles are secreted by a wide variety of cell types. In general, extracellular vesicles such as exosomes, microvesicles, and apoptotic bodies are membrane-bound and can be loaded with a therapeutic cargo. Exosomes are a type of extracellular vesicle that are secreted by most eukaryotic cells. Exosome biogenesis may begin when endosomal invaginations pinch off into the multivesicular body, forming intraluminal vesicles. If the multivesicular body fuses with the plasma membrane of the cell, the intraluminal vesicles may be released as exosomes. Microvesicles are another type of extracellular vesicles that are outward budded from cell surface membrane. Apoptotic bodies, on the other hand, are extracellular vesicles that are formed from dead cell debris.

The extracellular matrix (ECM) provides a structural support for many different types of tissue and also influences certain cell processes in the body. The components of ECM in the skin include collagen, elastic fibers, proteoglycans, and glycosaminoglycans. Age-related changes to the proteins of the ECM can have a number of different consequences. Wrinkles and reduced elasticity are typical phenomena of skin aging and are likely a result of a progressive reduction in the amount of extracellular matrix (ECM) (e.g., collagen) in the dermis. The reduction in collagen in aging skin can be due to a reduction in collagen production and/or an increase in the degradation of collagen. Structural changes in collagen and other ECM proteins are also possibly involved in dermal aging.

Vascular endothelial growth factor (VEGF) is a signaling protein that promotes angiogenesis, the growth of new blood vessels. VEGF forms part of the mechanism that restores the blood supply to cells and tissues when they are deprived of oxygenated blood due to compromised blood circulation.

### SUMMARY

Described herein, in some aspects, is a plurality of extracellular vesicles comprising an exogenous extracellular matrix messenger RNA (mRNA). In some cases, the exogenous extracellular matrix mRNA encodes a collagen. In some cases, the collagen is selected from the group consisting of Collagen type I, Collagen type II, Collagen type III, Collagen type IV, Collagen type V, Collagen type VI, Collagen type VII, Collagen type VIII, Collagen type IX, Collagen type X, Collagen type XI, Collagen type XII, Collagen type XIII, Collagen type XIV, Collagen type XV, Collagen type XVI, Collagen type XVII, Collagen type XVIII, Collagen type XIX, Collagen type XX, Collagen type XXI, Collagen type XXII, Collagen type XXIII, Collagen type XXIV, Collagen type XXV, Collagen type XXVI, Collagen type XXVII, Collagen type XXVIII, or any combination thereof. In some cases, the collagen is collagen type I. In some cases, the collagen is collagen type II. In some cases, the collagen is alpha 1 chain of collagen type I (Col1A1). In some cases, the exogenous extracellular matrix mRNA is the mRNA encoding pro-alpha1(I) chain. In some cases, the collagen is Col1A1, and the plurality of extracellular vesicles do not comprise an alpha 2 chain of collagen type I (Col1A2). In some cases, the collagen is Col1A2.

In some cases, the plurality of extracellular vesicles comprises an average of at least one copy of the exogenous extracellular matrix mRNA per 450 EVs. In some cases, the plurality of extracellular vesicles comprises an average of at least one copy of the exogenous extracellular matrix mRNA per 200 EVs. In some cases, the plurality of extracellular vesicles comprises an average of at least one copy of the exogenous extracellular matrix mRNA per about 0.1 to 100 EVs. In some cases, the plurality of extracellular vesicles comprises an average of at least one copy of the exogenous extracellular matrix mRNA per 2000 EVs, per 1000 EVs, per 500 EVs, per 400 EVs, per 300 EVs, per 200 EVs, per 150 EVs, per 100 EVs, per 90 EVs, per 80 EVs, per 70 EVs, 60 EVs, 50 EVs, per 40 EVs, per 30 EVs, per 20 EVs, per 15 EVs, per 10 EVs, per 5 EVs, per 2 EVs, or per 1 EV. In some cases, the plurality of extracellular vesicles comprise an average of at least 1, 2, 5, 10, 50, 100, 200, 300, 500, or 1000 copies of the exogenous extracellular matrix mRNA per EV. In some cases, the plurality of extracellular vesicles comprise an average of about 1 to 10 copies of the exogenous extracellular matrix mRNA per EV. In some cases, the plurality of extracellular vesicles comprise an average of about 1 copy of the exogenous extracellular matrix mRNA per EV. In some cases, the plurality of extracellular vesicles comprise an average of at least one copy of the exogenous extracellular matrix mRNA per 30 EVs.

In some cases, the plurality of extracellular vesicles are produced by transfecting a cell with a plasmid encoding an extracellular matrix protein. In some cases, the transfecting of the cell is performed by cellular nanoporation. In some cases, the transfecting of the cell is performed by electroporation. In some cases, the cell is a human cell. In some cases, the cell is a fibroblast selected from the group consisting of dermal fibroblast, human fibroblast, adult fibroblast, human adult fibroblast, neonatal fibroblast, neonatal human fibroblast, and neonatal human dermal fibroblast. In some cases, the cell is an adherent cell. In some cases, the cell is a human dermal fibroblast (e.g., neonatal human dermal fibroblast).

In some cases, the plurality of extracellular vesicles comprises an extracellular vesicle selected from the group consisting of exosome, microvesicle, apoptotic body, and any combination thereof. In some cases, the plurality of extracellular vesicles comprises at least one exosome. In some cases, the plurality of extracellular vesicles comprises at least one apoptotic body. In some cases, the plurality of extracellular vesicles comprises at least one microvesicle. In some cases, the plurality of extracellular vesicles comprises a mixture of at least any two of the following: exosome, microvesicle or apoptotic body.

In some cases, the plurality of extracellular vesicles are formulated for injection via an intravenous route, an intramuscular route, a subcutaneous route, or any combination thereof. In some cases, the formulation comprises one or more stabilizing agents and/or one or more preservatives. In some cases, the formulation comprises one or more DNAses and/or RNase inhibitors. In some cases, the formulation comprises one or more DNAses, DNAse inhibitors, RNAse, and/or RNAse inhibitors.

In some cases, the plurality of extracellular vesicles comprises at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles. In some cases, the plurality of extracellular vesicles comprises at least 1×10¹³ extracellular vesicles. In some cases, the plurality of extracellular vesicles comprises at 1×10¹⁴ extracellular vesicles. In some cases, the plurality of extracellular vesicles comprises at least 1×10⁹ extracellular vesicles and at most 1×10²⁰ extracellular vesicles. In some cases, the plurality of extracellular vesicles comprises at most 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹, 1×10²⁰, 1×10²⁵, or 1×10³⁰ extracellular vesicles. In some cases, the plurality of extracellular vesicles comprises at least 1×10⁶ extracellular vesicles and at most 1×10³⁰ extracellular vesicles. In some cases, the plurality of extracellular vesicles comprises at least 1 ng, 10 ng, 20 ng, 40 ng, 50 ng, 100 ng, 500 ng, 1 μg, 10 μg, 20 μg, 30 μg, 40 μg, or 50 μg of the extracellular matrix mRNA. In some cases, the plurality of extracellular vesicles comprises at most 1 ng, 10 ng, 20 ng, 40 ng, 50 ng, 100 ng, 500 ng, 1 μg, 10 μg, 20 μg, 30 μg, 40 μg, or 50 μg of the extracellular matrix mRNA. In some cases, the plurality of extracellular vesicles comprises at least 1 ng and at most 20 μg of the extracellular matrix mRNA. In some cases, the plurality of extracellular vesicles comprises at least 5 ng and at most 30 μg of the extracellular matrix mRNA.

In some cases, the plurality of extracellular vesicles comprises an extracellular vesicle comprising a targeting polypeptide. In some cases, the targeting polypeptide targets dermal cells. In some cases, the targeting polypeptide targets aminopeptidase N, CD26/DPP4, fibroblast activation protein α, or any combination thereof. In some cases, the plurality of extracellular vesicles does not comprise one or more of the following miRNAs: hsa-miR-29c-3p, hsa-miR-29a-3p, hsa-miR-378a-3p, hsa-miR-125b-5p, hsa-miR-23a-3p, hsa-miR-449a, hsa-miR-196a-5p, hsa-miR-744-5p, hsa-miR-223-3p, hsa-miR-23a-3p, hsa-miR-133a-3p, hsa-miR-223-3p, hsa-miR-5011-5p, hsa-miR-325, or hsa-miR-199b-5p.

In some cases, the plurality of extracellular vesicles comprises a size distribution with a peak at about 50-200 nm in diameter. In some cases, the plurality of extracellular vesicles comprises a size distribution with a peak at about 100 nm in diameter. In some cases, the plurality of extracellular vesicles comprises a size distribution with a peak at about 75-130 nm in diameter. In some cases, the extracellular vesicles are greater than 20 nm, 30 nm, 50 nm, 75 nm, or 100 nm in diameter. In some cases, at least 90% of the extracellular vesicles are greater than 20 nm, 30 nm, 50 nm, 75 nm, or 100 nm in diameter. In some cases, the extracellular vesicles do not comprise extracellular vesicles less than 50 nm in diameter. In some cases, the extracellular vesicles do not comprise extracellular vesicles less than 30 nm in diameter. In some cases, the extracellular vesicles do not comprise extracellular vesicles less than 20 nm in diameter. In some cases, the extracellular vesicles do not comprise extracellular vesicles less than 10 nm in diameter. In some cases, the extracellular vesicles do not comprise extracellular vesicles less than 5 nm in diameter.

In some cases, the exogenous extracellular matrix mRNA is present at a level that is at least 2-fold higher than a level of the exogenous extracellular matrix mRNA in an identical amount of naturally-occurring extracellular vesicles. In some cases, the exogenous extracellular matrix mRNA is present at a level that is at least 3-fold higher than a level of the exogenous extracellular matrix mRNA in an identical amount of naturally-occurring extracellular vesicles. In some cases, the exogenous extracellular matrix mRNA is present at a level that is at least 1.5 fold, at least 2-fold, at least 3-fold, at least 4-fold, at least 5-fold, at least 6-fold, at least 7-fold, at least 10-fold, at least 15-fold, at least 20-fold, at least 50-fold, at least 75-fold, at least 100-fold, at least 500-fold, at least 1000-fold, at least 1500-fold, or at least 2000-fold higher than a level of the exogenous extracellular matrix mRNA in an identical amount of naturally-occurring extracellular vesicles. In some cases, the exogenous extracellular matrix mRNA is present at a level that is about 3000-fold higher than a level of the exogenous extracellular matrix mRNA in an identical amount of naturally-occurring extracellular vesicles. In some cases, the exogenous extracellular matrix mRNA is present at a level that is about 2000-fold higher than a level of the exogenous extracellular matrix mRNA in an identical amount of naturally-occurring extracellular vesicles. In some cases, the plurality of extracellular vesicles expresses higher levels of at last one of the following markers compared to a comparable number of naturally-occurring extracellular vesicles: CD9, CD63, TSG101, or ARF6.

Described herein, in some aspects, is an extracellular vesicle comprising an exogenous extracellular matrix messenger RNA (mRNA). In some cases, the exogenous extracellular matrix mRNA encodes a collagen. In some cases, the collagen is selected from the group consisting of Collagen type I, Collagen type II, Collagen type III, Collagen type IV, Collagen type V, Collagen type VI, Collagen type VII, Collagen type VIII, Collagen type IX, Collagen type X, Collagen type XI, Collagen type XII, Collagen type XIII, Collagen type XIV, Collagen type XV, Collagen type XVI, Collagen type XVII, Collagen type XVIII, Collagen type XIX, Collagen type XX, Collagen type XXI, Collagen type XXII, Collagen type XXIII, Collagen type XXIV, Collagen type XXV, Collagen type XXVI, Collagen type XXVII, Collagen type XXVIII, or any combination thereof. In some cases, the collagen is collagen type I. In some cases, the collagen is collagen type II. In some cases, the collagen is alpha 1 chain of collagen type I (Col1A1). In some cases, the exogenous extracellular matrix mRNA is the mRNA encoding pro-alpha1(I) chain. In some cases, the collagen is Col1A1, and the extracellular vesicle does not comprise an alpha 2 chain of collagen type I (Col1A2). In some cases, the collagen is Col1A2. In some cases, the extracellular vesicle comprises an extracellular vesicle selected from the group consisting of exosome, microvesicle, apoptotic body, and any combination thereof. In some cases, the extracellular vesicle is an exosome. In some cases, the extracellular vesicle is an apoptotic body. In some cases, the extracellular vesicle is a microvesicle.

Described herein, in some aspect, is a vessel comprising the plurality of extracellular vesicles of any one of the preceding claims, wherein the vessel further comprises a cell. In some cases, the cell comprises a human cell, a human fibroblast cell, a fibroblast cell, a dermal fibroblast, a human fibroblast, an adult fibroblast, a human adult fibroblast, a neonatal fibroblast, a neonatal human fibroblast, a neonatal human dermal fibroblast, or any combination thereof. In some cases, the plurality of extracellular vesicles are present in the vessel at a ratio of at least 1000, 2000, 5000, 10000, or 12000 extracellular vesicles per cell. In some cases, the plurality of extracellular vesicles are present in the vessel at a ratio of no greater than 1000, 2000, 5000, 10000, 12000, 15000, 20000, 25000, 50000, 75000, 90000, or 100000 extracellular vesicles per cell.

Described herein, in some aspects, is a needle comprising the plurality of extracellular vesicles disclosed herein. In some cases, the needle is a microneedle. In some cases, the needle is solid. In some cases, the needle is a hydrogel needle. In some cases, at least 50%, 70%, 75%, 80%, 85%, 90%, 95%, or 99% of the needle comprises hydrogel. In some cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In some cases, the hydrogel comprises hyaluronic acid. In some cases, the hydrogel comprises at least 1%, 5%, 7%, 10%, 12%, 15%, 20%, 25% 50%, 70%, 75%, 80%, 85%, 90%, 95%, or 99% hyaluronic acid. In some cases, the hydrogel comprises at most 1%, 5% 7%, 10%, 12%, 15%, 20%, 25% 50%, 70%, 75%, 80%, 85%, 90%, 95%, or 99% hyaluronic acid. In some cases, the hydrogel comprises at least 5% hyaluronic acid. In some cases, the hydrogel comprises about 15% hyaluronic acid. In some cases, the hydrogel comprises at least 5% hyaluronic acid and at most 30% hyaluronic acid. In some cases, the hydrogel comprises greater than 10% hyaluronic acid and at most 20% hyaluronic acid. In some cases, the hydrogel comprises greater than 10% hyaluronic acid and at most 15% hyaluronic acid. In some cases, the hydrogel comprises greater than 10% hyaluronic acid and at most 18% hyaluronic acid.

Described herein, in some aspects, is a syringe comprising the plurality of extracellular vesicles disclosed herein. In some cases, the plurality of extracellular vesicles are suspended in hyaluronic acid within the syringe.

Described herein, in some aspects, is a hydrogel microneedle, wherein the hydrogel microneedle comprises a plurality of extracellular vesicles. In some cases, the hydrogel comprises at least 1%, 5%, 7%, 10%, 12%, 15%, 20%, 25% 50%, 70%, 75%, 80%, 85%, 90%, 95%, or 99% hyaluronic acid. In some cases, the hydrogel comprises at least 5%, 7%, 10%, 15%, or 20% hyaluronic acid. In some cases, the hydrogel comprises no greater than 1%, 5%, 7%, 10%, 12%, 15%, 20%, 25% 50%, 70%, 75%, 80%, 85%, 90%, 95%, or 99% hyaluronic acid. In some cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In some cases, the hydrogel comprises hyaluronic acid. In some cases, the plurality of extracellular vesicles comprises an extracellular vesicle selected from the group consisting of exosome, microvesicle, apoptotic body, and any combination thereof. In some cases, the plurality of extracellular vesicles comprise at least one exosome. In some cases, the plurality of extracellular vesicles comprise at least one apoptotic body. In some cases, the plurality of extracellular vesicles comprise at least one microvesicle. In some cases, the plurality of extracellular vesicles comprises at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles. In some cases, the plurality of extracellular vesicles comprises no great than 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, 1×10³⁰, 1×10³⁰, 1×10⁴⁰, 1×10⁵⁰, 1×10⁶⁰, 1×10⁷⁰, 1×10⁸⁰, 1×10⁹⁰, or 1×10¹⁰⁰ extracellular vesicles. In some cases, the plurality of extracellular vesicles comprises about 1×10⁶ to 1×10³⁰ extracellular vesicles. In some cases, the plurality of extracellular vesicles comprises about 1×10¹⁰ to 1×10¹⁴ extracellular vesicles. In some cases, the plurality of extracellular vesicles comprises about 1×10¹³ to 1×10¹⁴ extracellular vesicles.

In some cases, the hydrogel microneedle has a length less than 2 mm. In some cases, the hydrogel microneedle has a length less than 1 mm. In some cases, the hydrogel microneedle has a length of at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000 μm. In some cases, the hydrogel microneedle has a length no greater than 100, no greater than 200, no greater than 300, no greater than 400, no greater than 500, no greater than 600, no greater than 700, no greater than 800, no greater than 900, no greater than 1000, no greater than 1100, no greater than 1200, no greater than 1300, no greater than 1400, no greater than 1500, no greater than 1600, no greater than 1700, no greater than 1800, no greater than 1900, or no greater than 2000 μm. In other cases, the hydrogel microneedle has a length from 100 μm to 3000 μm, from 500 μm to 2500 μm, from 1000 μm to 2500 μm, or from 1000 μm to 2000 μm. In specific embodiments, the hydrogel microneedle has a length of about 1000 μm. In specific embodiments, the hydrogel microneedle has a length of about 2000 μm.

In some cases, the hydrogel microneedle has a base with a diameter of no greater than 10000 μm, 9000 μm, 8000 μm, 7000 μm, 6000 μm, 5000 μm, 4000 μm, 3000 μm, 2000 μm, 1800 μm, 1500 μm, 1000 μm, 900 μm, 800 μm 700 μm, 600 μm, 500 μm, 400 μm, 300 μm, 200 μm, or 100 μm. In some cases, the hydrogel microneedle has a base with a diameter of more than 10000 μm, 9000 μm, 8000 μm, 7000 μm, 6000 μm, 5000 μm, 4000 μm, 3000 μm, 2000 μm, 1800 μm, 1500 μm, 1000 μm, 900 μm, 800 μm 700 μm, 600 μm, 500 μm, 400 μm, 300 μm, 200 μm, or 100 μm. In some cases, the hydrogel microneedle comprises extracellular vesicles loaded with one or more mRNA cargos.

Described herein, in some aspects, is a needle comprising a plurality of extracellular vesicles wherein the extracellular vesicles comprise at least one extracellular matrix messenger RNA (mRNA). In some cases, the extracellular matrix mRNA encodes a collagen. In some cases, the collagen is selected from the group consisting of collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or any combination thereof. In some cases, the collagen is collagen type I. In some cases, the collagen is collagen type II. In some cases, the collagen is alpha 1 chain of collagen type I (Col1A1). In some cases, the collagen is Col1A1, and the plurality of extracellular vesicles do not comprise an alpha 2 chain of collagen type I (Col1A2). In some cases, the collagen is Col1A2. In some cases, the extracellular matrix mRNA is the mRNA encoding pro-alpha1(I) chain.

In some cases, the plurality of extracellular vesicles comprises an extracellular vesicle selected from the group consisting of exosome, microvesicle, apoptotic body, and any combination thereof. In some cases, the plurality of extracellular vesicles comprise at least one exosome. In some cases, the plurality of extracellular vesicles comprise at least one apoptotic body. In some cases, the plurality of extracellular vesicles comprise at least one microvesicle. In some cases, the plurality of extracellular vesicles comprise a mixture of any two of the following: exosome, microvesicle or apoptotic body.

In some cases, the needle is a microneedle. In some cases, the needle is solid. In some cases, the needle is a hydrogel needle, and optionally wherein at least 50%, 70%, 75%, 80%, 85%, 90%, 95%, or 99% of the needle comprises hydrogel. In some cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In some cases, the hydrogel comprises hyaluronic acid. In some cases, the hydrogel comprises at least 1%, 5%, 7%, 10%, 12%, 15%, 20%, 25% 50%, 70%, 75%, 80%, 85%, 90%, 95%, or 99% hyaluronic acid. In some cases, the hydrogel comprises at least 5% hyaluronic acid. In some cases, the hydrogel comprises no greater than 1%, 5%, 7%, 10%, 12%, 15%, 20%, 25% 50%, 70%, 75%, 80%, 85%, 90%, 95%, or 99% hyaluronic acid. In some cases, the hydrogel comprises about 15% hyaluronic acid. In some cases, the hydrogel comprises at least 5% hyaluronic acid and at most 30% hyaluronic acid. In some cases, the hydrogel comprises greater than 10% hyaluronic acid and at most 20% hyaluronic acid. In some cases, the hydrogel comprises greater than 10% hyaluronic acid and at most 15% hyaluronic acid. In some cases, the hydrogel comprises greater than 10% hyaluronic acid and at most 18% hyaluronic acid.

In some cases, the needle has a length less than 2 mm. In some cases, the needle has a length less than 1 mm. In some cases, the needle has a length of at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000 μm. In some cases, the needle has a length no greater than 100, no greater than 200, no greater than 300, no greater than 400, no greater than 500, no greater than 600, no greater than 700, no greater than 800, no greater than 900, no greater than 1000, no greater than 1100, no greater than 1200, no greater than 1300, no greater than 1400, no greater than 1500, no greater than 1600, no greater than 1700, no greater than 1800, no greater than 1900, or no greater than 2000 μm. In other cases, the needle has a length from 100 μm to 3000 μm, from 500 μm to 2500 μm, from 1000 μm to 2500 μm, or from 1000 μm to 2000 μm. In specific embodiments, the needle has a length of about 1000 μm. In specific embodiments, the needle has a length of about 2000 μm.

In some cases, the needle has a base with a diameter of no greater than 10000 μm, 9000 μm, 8000 μm, 7000 μm, 6000 μm, 5000 μm, 4000 μm, 3000 μm, 2000 μm, 1800 μm, 1500 μm, 1000 μm, 900 μm, 800 μm, 700 μm, 600 μm, 500 μm, 400 μm, 300 μm, 200 μm, or 100 μm. In some cases, the needle has a base with a diameter of more than 10000 μm, 9000 μm, 8000 μm, 7000 μm, 6000 μm, 5000 μm, 4000 μm, 3000 μm, 2000 μm, 1800 μm, 1500 μm, 1000 μm, 900 μm, 800 μm, 700 μm, 600 μm, 500 μm, 400 μm, 300 μm, 200 μm, or 100 μm.

Described herein, in some aspects, is a method of treating a skin condition in a subject comprising administering at least one extracellular matrix mRNA to a subject in need thereof, thereby treating the skin condition. In some cases, the exogenous extracellular matrix mRNA encodes a collagen. In some cases, the collagen is selected from the group consisting of collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or any combination thereof. In some cases, the collagen is collagen type I. In some cases, the collagen is collagen type II. In some cases, the collagen is alpha 1 chain of collagen type I (Col1A1). In some cases, the collagen is Col1A1, but not an alpha 2 chain of collagen type I (Col1A2). In some cases, the collagen is Col1A2. In some cases, the extracellular matrix mRNA is the mRNA encoding pro-alpha1(I) chain.

In some cases, the skin condition is skin damage. In some cases, the skin damage is caused by aging or sun damage. In some cases, the skin condition is a wound.

In some cases, the administering comprises administering at least 1 ng, 10 ng, 20 ng, 40 ng, 50 ng, 100 ng, 500 ng, 1 μg, 10 μg, 20 μg, 30 μg, 40 μg or 50 μg of the extracellular matrix mRNA to the subject. In some cases, the administering comprises administering no greater than 500 ng, 1 μg, 10 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, or 500 μg of the extracellular matrix mRNA to the subject. In some cases, the administering comprises administering 1 ng-20 μg of the extracellular matrix mRNA to the subject. In some cases, the administering comprises administering 1 ng-10 μg of the extracellular matrix mRNA to the subject. In some cases, the administering comprises administering 10 ng-10 μg, 10 ng-1 μg, 50 ng-1 μg, 100 ng-1 μg, or 500 ng-1 μg of the extracellular matrix mRNA to the subject. In some cases, the administering comprises administering about 50 ng of the extracellular matrix mRNA to the subject.

In some cases, the extracellular matrix mRNA is encapsulated in at least one extracellular vesicle (EV). In some cases, the EV does not comprise one or more of the following miRNAs: hsa-miR-29c-3p, hsa-miR-29a-3p, hsa-miR-378a-3p, hsa-miR-125b-5p, hsa-miR-23a-3p, hsa-miR-449a, hsa-miR-196a-5p, hsa-miR-744-5p, hsa-miR-223-3p, hsa-miR-23a-3p, hsa-miR-133a-3p, hsa-miR-223-3p, hsa-miR-5011-5p, hsa-miR-325, or hsa-miR-199b-5p.

In some cases, the at least one EV is an exosome. In some cases, the at least one EV is an apoptotic body or a microvesicle. In some cases, the at least one EV comprises a mixture of at least two of the following: exosome, microvesicle and/or apoptotic body.

In some cases, the treating the skin damage results in at least a 10% reduction in appearance of wrinkles. In some cases, the at least a 10% reduction in appearance of wrinkles occurs within 7-14 days of the administering of the at least one extracellular vesicle comprising extracellular matrix mRNA to the subject. In some cases, the treating the skin damage results in at least a 20%, 30%, 40%, 50%, 60%, 70%, 80%, 90% reduction in appearance of wrinkles. In some cases, the at least a 20%, 30%, 40%, 50%, 60%, 70%, 80%, 90% reduction in appearance of wrinkles occurs within 7-14 days of the administering of the at least one extracellular vesicle comprising extracellular matrix mRNA to the subject. In some cases, the treating the skin damage results in an at least 5%, 10%, 20%, 30%, 40%, 50%, 60%, 70%, 80%, or 90% reduction in total wrinkle number. In some cases, the treating the skin damage results in about 30%, 40%, 50%, 60%, 70%, 80%, 90%, or 95% reduction in total wrinkle number. In some cases, the treating the skin damage results in an at least 5%, 10%, 20%, 30%, 40%, 50%, 60%, 70%, 80%, or 90% reduction in total wrinkle area. In some cases, the treating the skin damage results in an no greater than 5%, 10%, 20%, 30%, 40%, 50%, 60%, 70%, 80%, or 90% reduction in total wrinkle area. In some cases, the treating the skin damage results in about 70%, 80%, or 90% reduction in total wrinkle area. In some cases, the wrinkle area is measure by microscopic photography of treated portion of the skin and quantified by a photography analysis software.

In some cases, the treating the skin condition comprises treating skin damage and wherein the treating the skin damage results in a reduction of skin damage that lasts for at least 20 days, 30 days, 40 days, 50 days, 60 days, 70 days, 80 days, 90 days, or 100 days following the treating the skin condition. In some cases, the reduction in skin damage is one or more of the following: reduction in total wrinkle number, reduction in total wrinkle area, reduction in appearance of wrinkles, or any combination thereof.

In some cases, the administering is via a subcutaneous injection. In some cases, the subcutaneous injection is performed at or near a site of the skin damage or the wound. In some cases, the subcutaneous injection is performed with a needle with a gauge of at least 23 s, 23, 22 s, 22, 21, 20, 19, 18, 17, 16, 15, 14, 13, 12, 11, or 10. In some cases, the subcutaneous injection is performed with a needle with a gauge of no greater than 34, 33, 32, 31, 30, 29, 28, 27, 26 s, 26, 25 s, 25, 24, 23 s, 23, 22 s, 22, 21, or 20. In some cases, the subcutaneous injection is performed with a needle with a gauge of 34, 33, 32, 31, 30, 29, 28, 27, 26 s, 26, 25 s, 25, 24, 23 s, 23, 22 s, 22, 21, 20, 19, 18, 17, 16, 15, 14, 13, 12, 11, or 10. In some cases, the subcutaneous injection is performed with a needle with a gauge of 28.

In some cases, the administering comprises administering at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles to the subject in need thereof, and at least a portion of the extracellular vesicles comprise the at least one extracellular matrix mRNA. In some cases, the administering comprises administering no greater than 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles to the subject in need thereof, and at least a portion of the extracellular vesicles comprise the at least one extracellular matrix mRNA. In some cases, the administering comprises administering about 1×10⁷ to 1×10¹⁷, 1×10⁸ to 1×10¹⁶, 1×10⁹ to 1×10¹⁵ extracellular vesicles to the subject in need thereof, and at least a portion of the extracellular vesicles comprise the at least one extracellular matrix mRNA. In some cases, the administering comprises administering about 1×10¹⁰ to 1×10¹⁴ extracellular vesicles to the subject in need thereof, and at least a portion of the extracellular vesicles comprise the at least one extracellular matrix mRNA.

In some cases, the extracellular vesicles are administered in multiple doses or as a single dose. In some cases, the extracellular vesicles are administered to the subject in intervals of at least once a day, once every week, once every 2 weeks, once every 4 weeks, once every 5 weeks, once every 6 weeks, once every 7 weeks, once every 8 weeks, once every 10 weeks, once every 12 weeks, or once every 16 weeks. In some cases, the extracellular vesicles are administered to the subject at most once a day, once every week, once every 2 weeks, once every 4 weeks, once every 5 weeks, once every 6 weeks, once every 7 weeks, once every 8 weeks, once every 10 weeks, once every 12 weeks, or once every 16 weeks. In some cases, the administering is performed a single time and administers at most 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10², or 1×10³⁰ extracellular vesicles to the subject. In some cases, the administering is performed a single time and administers at least 10, 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles to the subject. In some cases, the administering is performed multiple times and each time administers at most 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles to the subject. In some cases, the administering is performed multiple time and each time administers at least 10, 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles to the subject. In some cases, the extracellular vesicles are administered in an at least one dose that comprises at least 1,000 extracellular vesicles, wherein at least a portion of the extracellular vesicles comprise the at least one extracellular matrix mRNA. In some cases, the administering comprises administering the at least one extracellular matrix mRNA to a tissue of the subject.

In some cases, the tissue is a subcutis. In some cases, the tissue is a dermis. I In some cases, the tissue is a epidermis. In some cases, the tissue is a gum. In some cases, the tissue is a lip.

In some cases, within 72 hours of the administering of the at least one extracellular vesicle comprising the extracellular matrix mRNA to the subject, the tissue has a concentration of an extracellular matrix protein of at least 6000 μg/ml. In some cases, following the administering of the extracellular matrix mRNA to tissue of the subject, the tissue of the subject has a concentration of an extracellular matrix protein of at least 200, 300, 500, 750, 1000, 2000, 3000, 4000, or 5000 μg/ml. In some cases, following the administering of the extracellular matrix mRNA to tissue of the subject, the tissue of the subject has a concentration of an extracellular matrix protein of no greater than 200, 300, 500, 750, 1000, 2000, 3000, 4000, 5000, 6000, 7000, 8000, 9000, 10000, 11000, 12000, 13000, 14000, 15000, 16000, 17000, or 18000 μg/ml. In some cases, following the administering of extracellular vesicles comprising the extracellular matrix mRNA to tissue of the subject, the tissue of the subject has a concentration of an extracellular matrix protein of at least 200, 300, 500, 750, 1000, 2000, 3000, 4000, or 5000 μg/ml. In some cases, within 72 hours of the administering of the at least one extracellular vesicle comprising the extracellular matrix mRNA to the subject, the tissue has a concentration of an extracellular matrix protein of at least 200, 300, 500, 750, 1000, 2000, 3000, 4000, or 5000 μg/ml. In some cases, within 72 hours of the administering of the at least one extracellular vesicle comprising the extracellular matrix mRNA to the subject, the tissue has a concentration of an extracellular matrix protein of no greater than 200, 300, 500, 750, 1000, 2000, 3000, 4000, 5000, 6000, 7000, 8000, 9000, 10000, 11000, 12000, 13000, 14000, 15000, 16000, 17000, or 18000 μg/ml. In some cases, within about 4 days of the administering of the at least one extracellular vesicle comprising the extracellular matrix mRNA to the subject, the tissue has a peak concentration of an extracellular matrix protein.

In some cases, the administering of the at least one extracellular vesicle comprising the extracellular matrix mRNA to the subject is repeated at least every day, every 3 days, every 5 days, every 7 days, every 10 days, every 20 days, every 30 days, every 40 days, or every 50 days. In some cases, the administering of the at least one extracellular vesicle comprising the extracellular matrix mRNA to the subject is repeated about every 10 days, every 20 days, every 30 days, every 40 days, or every 50 days. In some cases, the administering of the at least one extracellular vesicle comprising the extracellular matrix mRNA to the subject is repeated about every 30 days. In some cases, the administering of the at least one extracellular vesicle comprising the extracellular matrix mRNA to the subject is repeated about every 60 days. In some cases, the administering of the at least one extracellular vesicle comprising the extracellular matrix mRNA to the subject is repeated about every 90 days.

In some cases, the method further comprises producing the extracellular vesicles comprising the at least one extracellular matrix mRNA by: (a) introducing a vector or a plasmid correspond to the extracellular matrix mRNA into a donor cell via transfection; (b) culturing the donor cell for a sufficient amount of time in a culture medium for the production of extracellular vesicles encapsulating the at least one extracellular matrix mRNA transcribed from the vector or the plasmid; and (c) collecting the extracellular vesicles from the culture medium. In some cases, in step (c) the collecting the extracellular vesicles occurs about 8-24 hr after the transfection. In some cases, the extracellular vesicles are present at a ratio of at least 1000, 2000, 5000, 10000, or 12000 extracellular vesicles per donor cell. In some cases, the extracellular vesicles are present at a ratio of no greater than 1000, 2000, 5000, 10000, 12000, 15000, 17000, 20000, 25000, 30000, 35000, 40000, 45000, or 50000 extracellular vesicles per donor cell. In some cases, the donor cell is a human cell, a human fibroblast cell, a fibroblast cell, a dermal fibroblast, a human fibroblast, an adult fibroblast, a human adult fibroblast, a neonatal fibroblast, a neonatal human fibroblast, a neonatal human dermal fibroblast, or any combination thereof.

Described herein, in some aspects, is a method of producing an extracellular vesicle comprising extracellular matrix mRNA comprising: a) introducing a vector or a plasmid correspond to the extracellular matrix mRNA into a donor cell via transfection; (b) culturing the donor cell for a sufficient amount of time in a culture medium for the production of extracellular vesicles encapsulating the at least one extracellular matrix mRNA transcribed from the vector or the plasmid; and (c) collecting the extracellular vesicles from the culture medium. In some cases, in step (c) the collecting the extracellular vesicles occurs about 8-24 hr after the transfection. In some cases, the extracellular vesicles encapsulating the extracellular matrix mRNA transcribed from the vector or plasmid are present at a level of at least 1000, 2000, 5000, 10000, or 12000 extracellular vesicles per donor cell. In some cases, the extracellular vesicles are present at a ratio of no greater than 1000, 2000, 5000, 10000, 12000, 15000, 17000, 20000, 25000, 30000, 35000, 40000, 45000, or 50000 extracellular vesicles per donor cell. In some cases, the donor cell is a human cell, a human fibroblast cell, a fibroblast cell, a dermal fibroblast, a human fibroblast, an adult fibroblast, a human adult fibroblast, a neonatal fibroblast, a neonatal human fibroblast, a neonatal human dermal fibroblast, or any combination thereof.

Described herein, in some aspect, is a microneedle device, the microneedle device comprising a substrate and a plurality of microneedles, wherein the plurality of microneedles protrude from the substrate, and wherein at least one microneedle of the plurality of microneedles comprises at least one extracellular vesicle (EV).

In some cases, a density of the plurality of microneedles on the substrate is 0.1 microneedles to 100 microneedles/mm² of the substrate. In some cases, a density of the plurality of microneedles on the substrate is at least 0.3 microneedles/mm² of the substrate. In some cases, a density of the plurality of microneedles on the substrate is about 0.59 microneedle/mm² of the substrate. In some cases, the plurality of microneedles comprise about 10-1000 microneedles, and wherein the area of the substrate is 20-1000 mm². In some cases, the plurality of microneedles comprise about 100 microneedles, and wherein the areas of the substrate is about 169 mm².

In some cases, the plurality of microneedles are arranged in at least 2 rows and at least 2 microneedles in each row. In some cases, the plurality of microneedles are arranged in 10 rows and 10 microneedles in each row.

In some cases, the at least one microneedle of the plurality of microneedles comprises a hydrogel. In some cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof.

In some cases, the at least one microneedle of the plurality of microneedles is conical in shape. In some cases, the at least one microneedle of the plurality of microneedles has a tapered shape.

In some cases, the at least one microneedle of the plurality of microneedles comprises a base with a diameter of less than 1000 μm. In some cases, the at least one microneedle of the plurality of microneedles comprises a base with a diameter of from about 100 μm to about 800 μm. In some cases, the at least one microneedle of the plurality of microneedles comprises a base with a diameter of about 400 μm.

In some cases, a length of the at least one microneedle of the plurality of microneedles is at least 100 μm. In some cases, a length of the at least one microneedle of the plurality of microneedles is from 100 μm to 3000 μm. In some cases, a length of the at least one microneedle of the plurality of microneedles is about 1000 μm. In some cases, a length of the microneedle of the plurality of microneedles is about 2000 μm.

In some cases, a center-to-center distance between the microneedle of the plurality of microneedles is at least 100 m. In some cases, a center-to-center distance between the microneedle of the plurality of microneedles is about 100 to 2000 μm. In some cases, a center-to-center distance between the microneedle of the plurality of microneedles is about 1000 μm.

In some cases, at least one microneedle of the plurality of microneedles comprises at least 3×10⁵ EVs. In some cases, at least one microneedle of the plurality of microneedles comprises about 3×10⁸ to 3×10¹⁰ EVs.

In some cases, the EVs are suspended in hydrogel. In some cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In some cases, the hydrogel comprises hyaluronic acid.

In some cases, the EVs comprise an exogenous extracellular matrix mRNA. In some cases, the exogenous extracellular matrix mRNA comprises collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or any combination thereof. In some cases, the exogenous extracellular matrix mRNA is the mRNA encoding pro-alpha1(I) chain.

In some cases, the EVs comprise exogenous VEGF mRNA. In some cases, the exogenous VEGF mRNA comprises VEGFA, VEGFB, VEGFC, VEGFD, PIGF, or any combination thereof.

Described herein, in some aspect, is a method of administering extracellular vesicles (EVs) to a tissue of a subject, the method comprising administering the needle, the syringe, the hydrogel needle, the microneedle, or the microneedle device disclosed herein to the tissue of the subject. In some cases, the method comprises administering the microneedle device disclosed herein to the tissue of the subject. In some cases, the microneedle device is removed after at least 5, 10, 15, 20, 25, or 30 minutes. In some cases, the microneedle device is removed after about 10, 15, 20, or 30 minutes.

In some cases, the EVs comprise at least one mRNA. In some cases, the administering comprises administering at least 1 ng, 10 ng, 50 ng, 100 ng, 1 μg, 10 μg, or 20 μg of the at least one mRNA to the tissue of the subject. In some cases, the administering comprises administering 1 ng-10 μg of the extracellular matrix mRNA to the tissue of the subject. In some cases, the administering comprises administering 1 ng-20 μg of the extracellular matrix mRNA to the tissue of the subject

In some cases, the tissue is subcutis. In some cases, the tissue is dermis. In some cases, the tissue is epidermis. In some cases, the tissue is a gum. In some cases, the tissue is a lip. In some cases, the subject is a mammal. In some cases, the subject is a human. In some cases, the subject is a rodent. In some cases, the subject is a monkey. In some cases, the subject is a rabbit.

In some cases, the administering is performed a single time and administers at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, or 1×10¹⁴ extracellular vesicles. In some cases, the administering is performed a single time and administers at most 1×10⁷, 1×10⁸, 1×10⁹, 1×10¹⁰, 1×10¹¹, 1×10¹², 1×10¹³, 1×10¹⁴, 1×10¹⁵, 1×10¹⁷, 1×10¹⁸, 1×10¹⁹, 1×10²⁰, or 1×10³⁰ extracellular vesicles. In some cases, the administering is performed a single time and administers at most 1×10¹³⁻¹⁴ extracellular vesicles. In some cases, the administering is performed multiple times and administers each time at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, or 1×10¹⁴ extracellular vesicles. In some cases, the administering is performed multiple times and administers each time at most 1×10⁷, 1×10⁸, 1×10⁹, 1×10¹⁰, 1×10¹¹, 1×10¹², 1×10¹³, 1×10¹⁴, 1×10¹⁵, 1×10¹⁷, 1×10¹⁸, 1×10¹⁹, 1×10²⁰, or 1×10³⁰ extracellular vesicles. In some cases, the administering is performed multiple times and administers each time at most 1×10¹³-14 extracellular vesicles. In some cases, the administering is performed multiple times and administers at most 1×10¹³⁻¹⁴ extracellular vesicles within 6-8 weeks. In some cases, the administering is performed multiple times over a period of time and administers at most 1×10⁷, 1×10⁸, 1×10⁹, 1×10¹⁰, 1×10¹¹, 1×10¹², 1×10¹³, 1×10¹⁴, 1×10¹⁵, 1×10¹⁷, 1×10¹⁸, 1×10¹⁹, or 1×10²⁰, 1×10³⁰ extracellular vesicles.

In some cases, the method results in less breakage of the EVs than administering the EVs in a conventional microneedle or a syringe. In some cases, the method results in less irritation at an administration site than administering the EVs in a conventional microneedle or a syringe. In some cases, the method results in more even distribution of EVs in the tissue than administering the EVs in a conventional microneedle or a syringe. In some cases, the conventional microneedle is a solid microneedle or a hollow microneedle.

Described herein, in some aspect, is a method of treating a skin condition in a subject in need thereof, the method comprising applying the needle, the syringe, the hydrogel needle, the microneedle, or the microneedle device disclosed herein to the subject. In some cases, the method comprising administering the microneedle disclosed herein to the subject.

In some cases, the skin condition is skin damage. In some cases, the skin damage is caused by aging or sun damage. In some cases, the skin condition is a wound.

In some cases, the administering comprises administering at least 0.1 ng, 1 ng, 5 ng, 10 ng, 20 ng, 30 ng, 40 ng, 50 ng, 100 ng, 200 ng, 300 ng, 400 ng, 500 ng, 600 ng, 700 ng, 800 ng, 900 ng, 1 μg, or 10 μg of the extracellular matrix mRNA. In some cases, the administering comprises administering no greater 0.1 ng, 1 ng, 5 ng, 10 ng, 20 ng, 30 ng, 40 ng, 50 ng, 100 ng, 200 ng, 300 ng, 400 ng, 500 ng, 600 ng, 700 ng, 800 ng, 900 ng, 1 μg, 10 μg, 20 μg, 30 μg, 40 μg, 50 μg, 60 μg, 70 μg, 80 μg, 90 μg, 100 μg, 200 μg, 300 μg, 400 μg, 500 μg, 600 μg, 700 μg, 800 μg, or 900 μg of the extracellular matrix mRNA. In some cases, the administering comprises administering about 0.1 ng-50 μg, 1 ng-50 μg, 1 ng-30 μg, 1 ng-25 μg of the extracellular matrix mRNA. In some cases, the administering comprises administering about 1 ng-20 μg of the extracellular matrix mRNA.

In some cases, the method results in at least a 10%, 20%, 30%, 40%, 50%, 60%, 70%, 80%, or 90% reduction in appearance of wrinkles. In some cases, the method results in at least 10%, 20%, 30%, 40%, 50%, 60%, 70%, 80%, or 90% more collagen fibers. In some cases, the method results in a higher dermal thickness by at least 10%, 20%, 30%, 40%, 50%, 60%, 70%, 80%, or 90%. In some cases, the method results in a prolonged effect for at least 30 days, at least 40 days, at least 50 days, at least 60 days, at least 70 days, at least 80 days, or at least 90 days.

In some cases, at least 50%, 70%, 75%, 80%, 85%, 90%, 95%, or 99% of the needle, the microneedle, or the microneedle of the microneedle device comprises hydrogel. In some case, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In some cases, the hydrogel comprises hyaluronic acid. In some cases, the hydrogel comprises at least 1%, 5%, 7%, 10%, 12%, 15%, 20%, 25% 50%, 70%, 75%, 80%, 85%, 90%, 95%, or 99% hyaluronic acid. In some cases, the hydrogel comprises at least 5% hyaluronic acid. In some cases, the hydrogel comprises about 15% hyaluronic acid. In some cases, the hydrogel comprises at least 5% hyaluronic acid and at most 30% hyaluronic acid. In some cases, the hydrogel comprises greater than 10% hyaluronic acid and at most 20% hyaluronic acid. In some cases, the hydrogel comprises greater than 10% hyaluronic acid and at most 15% hyaluronic acid. In some cases, the hydrogel comprises greater than 10% hyaluronic acid and at most 18% hyaluronic acid.

In some cases, the administering is performed a single time and administers at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, or 1×10¹⁴ extracellular vesicles. In some cases, the administering is performed a single time and administers at most 1×10⁷, 1×10⁸, 1×10⁹, 1×10¹⁰, 1×10¹¹, 1×10¹², 1×10¹³, 1×10¹⁴, 1×10¹⁵, 1×10¹⁷, 1×10¹⁸, 1×10¹⁹, 1×10²⁰, or 1×10³⁰ extracellular vesicles. In some cases, the administering is performed a single time and administers at most 1×10¹³⁻¹⁴ extracellular vesicles. In some cases, the administering is performed multiple times and administers each time at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, or 1×10¹⁴ extracellular vesicles. In some cases, the administering is performed multiple times and administers each time at most 1×10⁷, 1×10⁸, 1×10⁹, 1×10¹⁰, 1×10¹¹, 1×10¹², 1×10¹³, 1×10¹⁴, 1×10¹⁵, 1×10¹⁷, 1×10¹⁸, 1×10¹⁹, 1×10²⁰, or 1×10³⁰ extracellular vesicles. In some cases, the administering is performed multiple times and administers each time at most 1×10¹³-14 extracellular vesicles. In some cases, the administering is performed multiple times and administers at most 1×10¹³⁻¹⁴ extracellular vesicles within 6-8 weeks. In some cases, the administering is performed multiple times over a period of time and administers at most 1×10⁷, 1×10⁸, 1×10⁹, 1×10¹⁰, 1×10¹¹, 1×10¹², 1×10¹³, 1×10¹⁴, 1×10¹⁵, 1×10¹⁷, 1×10¹⁸, 1×10¹⁹, or 1×10²⁰, 1×10³⁰ extracellular vesicles.

Described herein, in some aspect, is a method of manufacturing a microneedle device, wherein the method comprises: (a) mixing extracellular vesicles (EVs) with a first batch of polymerizable solution; and (b) casting the mixture from (a) to a polydimethylsiloxane (PDMS) mold with at least one needle-like shape. In some cases, the mixing in (a) is performed under vacuum.

In some cases, the method further concentrating the EVs in a tip of the at least one needle-like shape of the PDMS mold. In some cases, the concentrating is by maintaining the PDMS mold at a temperature of at most 10° C. In some cases, the concentrating is by maintaining the PDMS mold at about 4° C. In some cases, the concentrating lasts from about 2 to about 6 hours.

In some cases, the method further comprises adding a second batch of the polymerizable solution on top of the PDMS mold. In some cases, the polymerizable solution comprises hydrogel. In some cases, the hydrogel comprises hyaluronic acid, sodium alginate, polylactic acid, polyglycolic acid, and polylactic-glycolic acid, cartilage thioflavin, silk protein, maltose, chitosan, carboxymethyl cellulose, or any combination thereof. In some cases, the hydrogel comprises hyaluronic acid. In some cases, the hydrogel comprises at least 5% hyaluronic acid. In some cases, the hydrogel comprises at least 5% hyaluronic acid. In some cases, the hydrogel comprises about 15% hyaluronic acid. In some cases, the hydrogel comprises at least 5% hyaluronic acid and at most 30% hyaluronic acid. In some cases, the hydrogel comprises greater than 10% hyaluronic acid and at most 20% hyaluronic acid. In some cases, the hydrogel comprises greater than 10% hyaluronic acid and at most 15% hyaluronic acid. In some cases, the hydrogel comprises greater than 10% hyaluronic acid and at most 18% hyaluronic acid.

In some cases, the final ratio between EVs and the polymerizable solution is at least 1:2, 1:3, 1:4, 1:5, 1:10, 1:15, 1:20, 1:25, 1:30, 1:35, 1:40, 1:45, 1:50, 1:55, 1:60, 1:65, 1:70, 1:80, 1:90, or 1:100. In some cases, the final ratio between EVs and the polymerizable solution is no greater than 1:2, 1:3, 1:4, 1:5, 1:10, 1:15, 1:20, 1:25, 1:30, 1:35, 1:40, 1:45, or 1:50. In some cases, the final ratio between EVs and the polymerizable solution is about 1:5, 1:10, 1:15, 1:20, 1:25, 1:30, 1:35, or 1:40.

In some cases, the EVs comprise exogenous extracellular matrix mRNA. In some cases, the exogenous extracellular matrix mRNA comprises collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or any combination thereof. In some cases, the EVs comprise exogenous VEGF mRNA. In some cases, the exogenous VEGF mRNA comprises VEGFA, VEGFB, VEGFC, VEGFD, PIGF, or any combination thereof.

Described herein, in some aspect, is a method of producing a heterodimer or a heterotrimer of collagen type I in a tissue, wherein the method comprises administering to the tissue the plurality of extracellular vesicles disclosed herein. In some cases, the heterodimer or the heterotrimer comprises at least one alpha chain of collagen type I (Col1A1) and at least one alpha chain of collagen type I (Col1A2), and wherein the Col1A1 is exogenously delivered by the plurality of extracellular vesicles disclosed herein. In some cases, the Col1A2 is endogenous to the tissue.

Described herein, in some aspect, is a plurality of extracellular vesicles comprising an average of at least one copy of exogenous VEGF mRNA per 400 extracellular vesicles. In some cases, the exogenous VEGF mRNA is selected from the group consisting of VEGFA, VEGFB, VEGFC, VEGFD, PIGF, and any combination thereof. In some cases, the plurality of extracellular vesicles comprises an extracellular vesicle selected from the group consisting of exosome, microvesicle, apoptotic body, or any combination thereof. In some cases, the plurality of extracellular vesicles comprises an extracellular vesicle comprising a targeting polypeptide.

In some cases, the plurality of extracellular vesicles comprise at least one copy of exogenous VEGF mRNA per at most 1, 5, 10, 15, 20, 25, 30, 40, 45, 50, 60, 70, 80, 90, 100, 150, 200, 250, 300, or 350 extracellular vesicles. In some cases, the plurality of extracellular vesicles comprise at least 1, 2, 5, 10, 20, 25, 30, 35, 30, 50, 60, 70 90 or 100 copies of VEGF mRNA per extracellular vesicle. In some cases, the plurality of extracellular vesicles comprise at least 1, 10, 20, 30, 40, 50, 60, 70, 80, 90, or 100 copies of VEGF mRNA per extracellular vesicle. In some cases, the plurality of extracellular vesicles comprise at most 1, 10, 20, 30, 40, 50, 60, 70, 80, 90, 100, 150, 200, 250, 300, 350, 400, 450, 500, 600, 700, 800, 900, or 1000 copies of VEGF mRNA per extracellular vesicle. In some cases, the plurality of extracellular vesicles comprise at least one copy of exogenous VEGF mRNA per about 0.001 to 100, 0.01 to 100, or 0.01 to 50 extracellular vesicles. In some cases, the plurality of extracellular vesicles comprise at least one copy of exogenous VEGF mRNA per about 0.02 to 50 extracellular vesicles.

In some cases, the plurality of extracellular vesicles comprise at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles. In some cases, the plurality of extracellular vesicles comprise at most 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles.

In some cases, the plurality of extracellular vesicles are formulated for intravenous injection, intramuscular injection, subcutaneous injection, or injection via a coronary artery catheter.

In some cases, the mixture comprises at least two extracellular vesicles that comprise between one and six copies of exogenous VEGF mRNA, and wherein the mixture comprises exosomes, microvesicles, and apoptotic bodies. In some cases, said mixture is formulated for injection via an intravenous, intramuscular, or subcutaneous route. In some cases, said mixture is formulated for injection via a coronary artery catheter. In some cases, VEGF mRNA is present at a level that is at least 2-fold, at least 3-fold, at least 4-fold, at least 5-fold, at least 6-fold, at least 7-fold, at least 10-fold, at least 15-fold, at least 20-fold, at least 50-fold, at least 75-fold, at least 100-fold, at least 500-fold, at least 1000-fold, at least 1500-fold, or at least 2000-fold higher than a level of VEGF mRNA in an identical amount of naturally-occurring extracellular vesicles. In some cases, the plurality of extracellular vesicles comprise at least 10 μg, 50 μg, 100 μg, 200 μg, 300 μg, 400 μg, 500 μg, 600 μg, 700 μg, 800 μg, 900 μg, 1 ng, 100 ng, 500 ng, 1000 ng, 10 μg, 50 μg, 100 μg, 150 μg, or 200 μg of VEGF mRNA. In some cases, the plurality of extracellular vesicles comprise no greater than 10 μg, 50 μg, 100 μg, 200 μg, 300 μg, 400 μg, 500 μg, 600 μg, 700 μg, 800 μg, 900 μg, 1 ng, 100 ng, 500 ng, 1000 ng, 10 μg, 50 μg, 100 μg, 150 μg, 200 μg, 300 μg, 400 μg, 500 μg, 600 μg, 700 μg, 800 μg, 900 μg, or 1000 μg of VEGF mRNA. In some cases, the plurality of extracellular vesicles comprise about 1 ng to 200 μg VEGF mRNA.

Described herein, in some aspect, is a method of treating a blood flow disorder in a subject comprising administering at least one extracellular vesicle comprising VEGF mRNA to the subject, thereby treating the blood flow disorder. In some cases, the treating the blood flow disorder results in at least a 5% increase in revascularization. In some cases, the at least 5% increase in revascularization occurs within 14 days of the administering of the at least one extracellular vesicle comprising VEGF mRNA.

In some cases, the administering of the at least one extracellular vesicle comprising VEGF mRNA comprises administering the at least one extracellular vesicle comprising VEGF mRNA to the subject via an intravenous, intramuscular or subcutaneous injection or via a coronary artery catheter. In some cases, the intravenous, intramuscular or subcutaneous injection is performed with a needle with a gauge of at least 14 gauge.

In some cases, the blood flow disorder is ischemia. In some cases, the at least one extracellular vesicle comprising VEGF mRNA is administered in at least one dose. In some cases, the at least one extracellular vesicle comprising VEGF mRNA comprises at least 1,000 extracellular vesicles comprising VEGF mRNA. In some cases, the at least one extracellular vesicle comprising VEGF mRNA is administered in at least two doses.

In some cases, the administering comprises administering a single time at least 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or 1×10¹⁶ EVs. In some cases, the administering comprises administering a single time no greater than 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶ 1×10¹⁷, 1×10¹⁸, 1×10¹⁹, 1×10²⁰, 1×10²¹, 1×10²², 1×10²³, or 1×10²⁴ EVs. In some cases, the administering comprises administering a single time about 1×10⁵ to 1×10²⁰, 1×10⁶ to 1×10¹⁹, 1×10⁷ to 1×10¹⁸, 1×10⁸ to 1×10¹⁷, or 1×10⁹ to 1×10¹⁶ EVs. In some cases, the administering comprises administering a single time about 1×10¹⁰ to 1×10¹⁶ EVs. In some cases, the administering comprises administering a single time at least 10 μg, 50 μg, 100 μg, 200 μg, 300 μg, 400 μg, 500 μg, 600 μg, 700 μg, 800 μg, 900 μg, 1 ng, 100 ng, 500 ng, 1000 ng, 10 μg, 50 μg, 100 μg, 150 μg, or 200 μg of VEGF mRNA. In some cases, the administering comprises administering a single time no greater than 10 μg, 50 μg, 100 μg, 200 μg, 300 μg, 400 μg, 500 μg, 600 μg, 700 μg, 800 μg, 900 μg, 1 ng, 100 ng, 500 ng, 1000 ng, 10 μg, 50 μg, 100 μg, 150 μg, 200 μg, 300 μg, 400 μg, 500 μg, 600 μg, 700 μg, 800 μg, 900 μg, or 1000 μg of VEGF mRNA.

In some cases, the administering comprises administering multiple times and each time at least 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or 1×10¹⁶ EVs. In some cases, the administering comprises administering multiple times and each time no greater than 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶ 1×10¹⁷, 1×10¹⁸, 1×10¹⁹, 1×10²⁰, 1×10²¹, 1×10²², 1×10²³, or 1×10²⁴ EVs. In some cases, the administering comprises administering multiple times and each time about 1×10⁵ to 1×10²⁰, 1×10⁶ to 1×10¹⁹, 1×10⁷ to 1×10¹⁸, 1×10⁸ to 1×10¹⁷, or 1×10⁹ to 1×10¹⁶ EVs. In some cases, the administering comprises administering multiple times and each time about 1×10¹⁰ to 1×10¹⁶ EVs. In some cases, the administering comprises administering multiple times and each time at least 10 μg, 50 μg, 100 μg, 200 μg, 300 μg, 400 μg, 500 μg, 600 μg, 700 μg, 800 μg, 900 μg, 1 ng, 100 ng, 500 ng, 1000 ng, 10 μg, 50 μg, 100 μg, 150 μg, or 200 μg of VEGF mRNA. In some cases, the administering comprises administering multiple times and each time no greater than 10 μg, 50 μg, 100 μg, 200 μg, 300 μg, 400 μg, 500 μg, 600 μg, 700 μg, 800 μg, 900 μg, 1 ng, 100 ng, 500 ng, 1000 ng, 10 μg, 50 μg, 100 μg, 150 μg, 200 μg, 300 μg, 400 μg, 500 μg, 600 μg, 700 μg, 800 μg, 900 μg, or 1000 μg of VEGF mRNA.

In some cases, wherein administering of the at least one extracellular vesicle comprising VEGF mRNA to the subject comprises administering of the at least one extracellular vesicle comprising VEGF mRNA to a tissue of the subject. In some cases, the administering is performed using microneedles loaded with extracellular vesicles comprising VEGF mRNA.

In some cases, the microneedles comprise hydrogel. In some cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof.

In some cases, the extracellular vesicles are administered to the subject in intervals of at least once a day, once every week, once every 2 weeks, once every 4 weeks, once every 5 weeks, once every 6 weeks, once every 7 weeks, once every 8 weeks, once every 10 weeks, once every 12 weeks, or once every 16 weeks. In some cases, the extracellular vesicles are administered to the subject at most once a day, once every week, once every 2 weeks, once every 4 weeks, once every 5 weeks, once every 6 weeks, once every 7 weeks, once every 8 weeks, once every 10 weeks, once every 12 weeks, or once every 16 weeks.

Described herein, in some aspect, is a method of producing an extracellular vesicle comprising VEGF mRNA comprising: (a) introducing a vector or plasmid encoding VEGF into a donor cell via transfection; (b) incubating the donor cell for a sufficient time for the production of extracellular vesicles encapsulating VEGF mRNA transcribed from the vector or plasmid; and (c) collecting the extracellular vesicles encapsulating the VEGF mRNA transcribed from the vector or plasmid.

Described herein, in some aspect, is a method of treating a blood flow disorder a subject in need thereof, the method comprising administering the extracellular vesicles disclosed herein. In some cases, the blood flow disorder is ischemia.

Described herein, in some aspects, is a plurality of extracellular vesicles comprising an average of at least one copy of exogenous VEGF mRNA. In some embodiments, the exogenous VEGF mRNA is selected from the group consisting of VEGFA, VEGFB, VEGFC, VEGFD, PIGF, and any combination thereof. In some embodiments, the plurality of extracellular vesicles comprises an extracellular vesicle selected from the group consisting of exosome, microvesicle, apoptotic body, or any combination thereof. In some embodiments, the plurality of extracellular vesicles comprises an extracellular vesicle comprising a targeting polypeptide.

In some aspects, described herein are mixtures of extracellular vesicles, wherein the mixture comprises at least two extracellular vesicles that comprise between one and six copies of exogenous VEGF mRNA, and wherein the mixture comprises exosomes, microvesicles, and apoptotic bodies. In some embodiments, the mixture is formulated for injection via an intravenous, intramuscular, or subcutaneous route. In some embodiments, the mixture is formulated for injection via a coronary artery catheter.

In some aspects, described herein are methods of treating a blood flow disorder in a subject comprising administering at least one extracellular vesicle comprising VEGF mRNA to the subject, thereby treating the blood flow disorder. In some embodiments, treating the blood flow disorder results in at least a 5% increase in revascularization. In some embodiments, the at least 5% increase in revascularization occurs within 14 days of the administering of the at least one extracellular vesicle comprising VEGF mRNA. In some embodiments, administering of the at least one extracellular vesicle comprising VEGF mRNA comprises administering the at least one extracellular vesicle comprising VEGF mRNA to the subject via an intravenous, intramuscular or subcutaneous injection or via a coronary artery catheter. In some embodiments, the intravenous, intramuscular or subcutaneous injection is performed with a needle with a gauge of at least 14 gauge. In some embodiments, the blood flow disorder is ischemia. In some embodiments, the at least one extracellular vesicle comprising VEGF mRNA is administered in at least one dose. In some embodiments, the dose comprises at least 1,000 extracellular vesicles comprising VEGF mRNA. In some embodiments, the at least one extracellular vesicle comprising VEGF mRNA is administered in at least two doses. In some embodiments, administering of the at least one extracellular vesicle comprising VEGF mRNA to the subject comprises administering of the at least one extracellular vesicle comprising VEGF mRNA to a tissue of the subject.

In some aspects, described herein is a plurality of extracellular vesicles comprising an average of at least one copy of exogenous extracellular matrix mRNA. In some embodiments, the exogenous extracellular matrix mRNA is selected from the group consisting of collagen, COL1, collagen type I, collagen type II, collagen type III, collagen type V, collagen type XI, collagen type IX, collagen type XII, collagen type XIV, collagen type VIII, collagen type X, collagen type IV, collagen type VI, collagen type VII, collagen type XIII, collagen type XV, collagen type XVII, and collagen type XVIIII. In some embodiments, the plurality of extracellular vesicles comprises an extracellular vesicle selected from the group consisting of exosome, microvesicle, apoptotic body, or any combination thereof. In some embodiments, the plurality of extracellular vesicles comprises an extracellular vesicle comprising a targeting polypeptide.

In some aspects, described herein are mixtures of extracellular vesicles, wherein the mixture comprises at least two extracellular vesicles that comprise between one and two copies of exogenous extracellular matrix mRNA, and wherein the mixture comprises exosomes, microvesicles, and apoptotic bodies. In some embodiments, the mixture is formulated for injection via an intravenous, intramuscular, or subcutaneous route.

In some aspects, described herein are methods of treating a skin damage in a subject comprising administering at least one extracellular vesicle comprising extracellular matrix mRNA to the subject, thereby treating the skin damage in the subject. In some embodiments, treating the skin damage results in at least a 10% reduction in appearance of wrinkles. In some embodiments, the at least 10% reduction in appearance of wrinkles occurs within 14 days of the administering of the at least one extracellular vesicle comprising extracellular matrix mRNA. In some embodiments, the administering of the at least one extracellular vesicle comprising extracellular matrix mRNA comprises administering the at least one extracellular vesicle comprising extracellular matrix mRNA to the subject via a subcutaneous injection. In some embodiments, subcutaneous injection is performed with a needle with a gauge of at least 14. In some embodiments, the skin damage is caused by aging or sun damage. In some embodiments, the at least one extracellular vesicle comprising extracellular matrix mRNA is administered in at least one dose. In some embodiments, the dose comprises at least 1,000 extracellular vesicles comprising extracellular matrix mRNA. In some embodiments, the at least one extracellular vesicle comprising extracellular matrix mRNA is administered in at least two doses. In some embodiments, administering of the at least one extracellular vesicle comprising extracellular matrix mRNA to the subject comprises administering of the at least one extracellular vesicle comprising VEGF mRNA to a tissue of the subject. In some embodiments, within 72 hours of the administering of the at least one extracellular vesicle comprising extracellular matrix mRNA to the subject, the tissue has a concentration of extracellular matrix protein of at least 6000 μg/ml.

In some aspects, described herein are methods of producing an extracellular vesicle comprising VEGF mRNA comprising: introducing a vector or plasmid encoding VEGF into a donor cell via transfection; incubating the donor cell for a sufficient time for the production of extracellular vesicles encapsulating VEGF mRNA transcribed from the vector or plasmid; and collecting the extracellular vesicles encapsulating the VEGF mRNA transcribed from the vector or plasmid.

In some aspects, described herein are methods of producing an extracellular vesicle comprising extracellular matrix mRNA comprising: introducing a vector or plasmid encoding an extracellular matrix protein into a donor cell via transfection; incubating the donor cell for a sufficient time for the production of extracellular vesicles encapsulating extracellular matrix mRNA transcribed from the vector or plasmid; and collecting the extracellular vesicles encapsulating the extracellular matrix mRNA transcribed from the vector or plasmid.

### BRIEF DESCRIPTION OF THE DRAWINGS

This patent application contains at least one drawing executed in color. Copies of this patent or patent application with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments.

FIG. 1 illustrates Cellular Nanoporation (“CNP,” also referred to herein as cellular nanoelectroporation, or “NEP”) generating large quantities of extracellular vesicles (EVs) loaded with transcribed mRNAs. Schematic representation of CNP generated EVs for targeted nucleic acid delivery. An exemplary CNP system consists of a nanochannel array, with each channel measuring about 500 nm in diameter (top inset).

FIG. 2 illustrates quantification of VEGF mRNA loading levels produced by cellular nanoporation (“NEP”) or bulk electroporation (“BEP”).

FIG. 3 illustrates an in vitro study of cellular nanoporation generated exosomes for VEGF mRNA delivery. FIG. 3A shows representative confocal microscopy images of VEGF staining in human fibroblasts 48 hours after VEGF mRNA loaded EV in vitro delivery (“NEP”). FIG. 3B shows qPCR of human fibroblasts treated for 24 or 48 hours with VEGF mRNA loaded EVs.

FIG. 4 illustrates the in vivo therapeutic efficacy of VEGF mRNA loaded CNP-generated exosomes in an hindlimb ischemia (HLI) model. FIG. 4A shows in vivo imaging showing increased hindlimb perfusion/revascularization following VEGF mRNA loaded EV delivery into HLI model. FIG. 4B shows quantification of average perfusion values of the ischemic footpad to that of the control footpad at various timepoints after VEGF mRNA loaded EV injection. FIG. 4C shows ELISA quantification of VEGF concentration in 0.20 g of ischemic leg tissue 48 hours after VEGF mRNA loaded EV injection. FIG. 4D shows representative confocal microscopy images of VEGF staining in gastrocnemius tissue resected 48 hours after VEGF mRNA loaded EV injection.

FIG. 5 illustrates an in vitro study of CNP generated exosomes for collagen type I alpha I (Col1) mRNA delivery. FIG. 5A shows representative confocal microscopy images of Col1 staining in human fibroblasts 48 hours after Col1 mRNA loaded EV in vitro delivery (“NEP”).

FIG. 5B shows qPCR of human fibroblasts treated for 24 or 48 hours with Col1 mRNA loaded EVs produced by CNP (“NEP”) and bulk electroporation (“BEP”).

FIG. 6 illustrates the in vivo therapeutic efficacy of Col1 mRNA loaded CNP-generated exosomes in a photoaging skin model of aging. FIG. 6A shows in vivo imaging showing improved skin condition (e.g., increased anti-aging) following Col1 mRNA loaded EV delivery into skin photoaging model. FIG. 6B shows in vivo imaging showing improved skin condition (e.g., increased anti-aging) following Col1 mRNA loaded EV delivery into skin photoaging model. FIG. 6C shows representative immunohistochemistry (IHC) staining of skin tissue after Col1 mRNA loaded EV delivery into skin photoaging model. FIG. 6D shows representative confocal microscopy images of Col1 staining in skin tissue after Col1 mRNA loaded EV delivery into skin photoaging model. FIG. 6E shows representative IHC staining (Left) and confocal microscopy images of Col1 staining (Right) of skin tissue after Col1 mRNA loaded EV delivery into skin photoaging model. FIG. 6F shows representative Masson's Trichrome staining of skin tissue after Col1 mRNA loaded EV delivery into skin photoaging model.

FIG. 7 illustrates CNP-generated EVs loaded with COL1A1 mRNA and a successful in-vitro delivery of COL1A1 mRNA-containing EVs. FIG. 7A shows representation of CNP-generated EVs for targeted nucleic acid deliver. FIG. 7B shows a plasmid map of pCMV-COL1A1-GFP. FIG. 7C shows EV number per cell produced by untreated neonatal human dermal fibroblasts (nHDFs) in PBS buffer as control; bulk electroporation (BEP); or cellular nanoporation (CNP) with PBS buffer only (n=3 for each group, *P=0.048 Control vs BEP; **P=0.005 Control vs CNP; ##P=0.002 BEP vs CNP). FIG. 7D shows characterization of exosomes by nanoparticle tracking analysis. FIG. 7E shows western blot results showing differences in the expression levels of EV membrane markers in the control and CNP treated group. FIG. 7F shows number of EVs per cell under different voltage conditions (0, 50, 100, or 150 V). FIG. 7G shows kinetic analyses of CNP-induced EV release at different timepoints (0, 4, 8, 12, 16, 20, and 24 h) (n=3, **P=0.0071 0 V vs 100 V; **P=0.0032 50 V versus 100 V; NS, not significant). FIG. 7H shows RT-qPCR of COL1A1 mRNA revealing that exosomes produced by CNP contained much larger quantities of transcribed mRNAs than exosomes produced by BEP and Control (n=3 for all groups, **P=0.002 Control vs BEP; ***P<0.001 Control vs CNP; ###P<0.001 BEP vs CNP). FIG. 7I shows an electronic gel image of total RNA extracted from supernatant of 2×10⁷ untreated neonatal human dermal fibroblasts (nHDFs) without fetal bovine serum, total RNA collected from 2×10⁷ nHDFs supernatant after CNP platform with COL1A1 plasmid, and 0.4 ng synthesized COL1A1 mRNA. FIG. 7J shows a schematic diagram of in vitro EV-loaded mRNA delivery and expression. FIG. 7K shows proliferation of nHDFs after treatment with different concentrations of CNP EVs at 0 h, 24 h, and 48 h. FIG. 7L shows fluorescence images showing serum-starved nHDFs treated with CNP-generated EVs containing COL1A1-GFP mRNA and protein translated from delivered COL1A1-GFP mRNA. Scale bar, 100 μm. FIG. 7M shows fluorescence intensity of cells treated with COL1A1-EVs (n=3 for all groups, ***P<0.001 Control vs COL1A1 EVs). FIG. 7N shows RT-qPCR shows higher collagen mRNA transcript levels after in vitro delivery of COL1A1 mRNA from EVs (n=3 for all groups, ***P<0.001 Control vs COL1A1 EVs). FIG. 7O shows western blots show elevated COL1A1 protein in treated fibroblasts (n=3 for all groups, **P=0.001 Control vs COL1A1 EVs). FIG. 7P shows fold changes in COL1A1 mRNA in the control and CNP treated groups at 24 h and 48 h (n=3, **P=0.0071 Control vs CNP at 24 h; ***P<0.001 control vs CNP at 48 h). FIG. 7Q shows pro-collagen I collected from supernatant and detected by ELISA (n=3 for all groups, ***P<0.001 Control vs COL1A1 EVs). All data were from three independent experiments and are presented as mean±s.e.m.

FIG. 8 illustrates in vivo kinetics of COL1A1-EVs mRNA delivery and protein formation in murine skin. FIG. 8A shows in situ hybridization of human COL1A1 mRNA by RNAscope after subcutaneous injection of COL1A1 EVs, measured at 0 h, 12 h, 24 h, 48 h, 96 h, 7 days, 10 days, and 14 days. Scale bar, 100 μm. FIG. 8B shows quantification of RNAscope results by average number of brown dots per cells. FIG. 8C shows immunofluorescence over time of COL1A1-EV-derived protein by visualization of co-localized GFP and COL1A1 protein (RFP). Scale bar, 100 μm. FIG. 8D shows fluorescence intensity quantification of COL1A1-GFP protein expression. FIG. 8E shows quantification of GFP+ cells confirms that the COL1A1-EV derived protein grafts decrease in a time-dependent manner over 30 days. All data are from three independent experiments and are presented as mean±s.e.m.

FIG. 9 illustrates COL1A1-EVs mRNA delivery reduced dermal wrinkles in a photoaging UVB-irradiated mouse model. FIG. 9A shows a schematic diagram of UVB-induced skin photoaging mouse model. FIG. 9B shows a schematic representation of 5 low-dose injections of COL1A1 EVs over time. Skin tissue was harvested at 28 days. FIG. 9C shows wrinkle formation was tracked at days 0, 4, 7, 14, 21, and 28 after initial treatment with COL1A1 EVs, unloaded EVs from nHDF cell culture medium, topical 0.05% retinoic acid (RA), or saline (n=4 for all groups). The sham group comprised female nude mice not exposed to UV. Scale bar, 5 cm. FIG. 9D shows total numbers of dorsal-skin wrinkles quantified with custom software (n=4 for all groups, **P=0.003 COL1A1-EVs vs Saline at day 7; ***P<0.001 COL1A1-EVs vs Saline at days 14, 21, and 28; ※P=0.0189 COL1A1-EVs vs Unloaded EVs at days 7; ※※※P<0.001 COL1A1-EVs vs Unloaded EVs at days 14, 21, and 28; †P=0.034 RA vs Saline at day 21; ††P=0.003 RA vs Saline at day 28; #P=0.014 Unloaded EVs vs Saline at day 21; ##P=0.002 Unloaded EVs vs Saline at day 28). FIG. 9E shows quantification of wrinkle area on dorsal skin (n=4 for all groups, *P=0.023 COL1A1-EVs vs Saline at day 7; *P=0.017 COL1A1-EVs vs Saline at day 14; ***P<0.001 COL1A1-EVs vs Saline at days 21 and 28; ※P=0.0205 COL1A1-EVs vs Unloaded EVs at days 7; ※※P=0.0011 COL1A1-EVs vs Unloaded EVs at days 14; ※※※P<0.001 COL1A1-EVs vs Unloaded EVs at days 21, and 28; †P=0.039 RA vs Saline at day 28; #P=0.027 Unloaded EVs vs Saline at day 28). FIG. 9F shows microscopic observations of dorsal skin and skin replicas. Scale bar, 5 cm. FIG. 9G shows mean wrinkle length analyzed on skin replicas (n=4 for all groups, ##P=0.002 Unloaded EVs vs Saline; ***P<0.001 COL1A1-EVs vs Saline; ※※※P<0.001 COL1A1-EVs vs Unloaded EVs). FIG. 9H shows mean wrinkle depth in skin replicas (n=4 for all groups, ***P<0.001 COL1A1-EVs vs Saline; ##P=0.006 Unloaded EVs vs Saline; †P=0.022 RA vs Saline); ※※※P<0.001 COL1A1-EVs vs Unloaded EVs). FIG. 9I shows wrinkles were tracked on days 0, 4, 7, 14, 21, 28, 35, 42, 49, and 56 after the initial treatments (COL1A1-EVs, unloaded EVs, 0.05% retinoic acid [RA], saline); (n=4 for all groups), Scale bar, 5 cm. Female nude mice that were not exposed to UV irradiation comprised the sham group. FIG. 9J shows total wrinkle area (n=4 for all groups, *P=0.012 COL1A1-EVs vs Saline at day 7; ***P<0.001 COL1A1-EVs vs Saline at days 14, 21, 28 and 35; *P=0.015 COL1A1-EVs vs Saline at day 42; ††P=0.008 RA vs Saline at day 14; ††P=0.007 RA vs Saline at days 21 and 35; #P=0.012 Unloaded EVs vs Saline at day 14; #P=0.035 Unloaded EVs vs Saline at day 21; ##P=0.002 Unloaded EVs vs Saline at day 28). FIG. 9K shows numbers of wrinkles on the dorsal skin of the mice. (n=4 for all groups; **P=0.008 COL1A1-EVs vs Saline at day 7; **P=0.004 COL1A1-EVs vs Saline at day 21, **P=0.001 COL1A1-EVs vs Saline at day 35; ***P<0.001 COL1A1-EVs vs Saline at days 14, 28, 42, and 49; †P=0.025 RA vs Saline at day 21; ††P=0.007 RA vs Saline at day 28; #P=0.015 Unloaded EVs vs Saline at day 35; ##P=0.004 Unloaded EVs vs Saline at day 21, ##P=0.002 Unloaded EVs vs Saline at day 28). FIG. 9L shows immunofluorescence staining for GFP and COL1A1 (RFP) protein in sham control group, saline control group, RA treatment group, unloaded-EV treatment group, and COL1A1-EV treatment group; COL1A1-EV-treated mice exhibited GFP+ COL1A1 protein grafts in the dermis and subcutis at 30 days after treatment began. Scale bar, 200 μm. FIG. 9M shows fluorescence intensity of COL1A1 (RFP) for all treatment groups (n=3 for all groups, ***P<0.001 COL1A1-EVs vs Saline; †P=0.022 RA vs Saline; #P=0.038 Unloaded EVs vs Saline; ※※※P<0.001 COL1A1-EVs vs Unloaded EVs). FIG. 9N shows representative Masson trichrome staining of the epidermis, dermis, and subcutis for all mouse groups. Scale bar, 300 μm. FIG. 9O shows quantification of dermal thickness showing increased collagen fibers in the COL1A1-EV group (n=3 for all groups, ***P<0.001 COL1A1-EVs vs Saline; †P=0.022 RA vs Saline; #P=0.018 Unloaded EVs vs Saline; ※※※P<0.001 COL1A1-EVs vs Unloaded EVs).

FIG. 10 illustrates Construction of a novel microneedle delivery platform for improved in vivo EV distribution and retention. FIG. 10A shows a schematic illustration of microneedle fabrication. FIG. 10B shows microscope and scanning electron microscopy images of microneedle (MN) arrays. Scale bar, 500 μm. FIG. 10C shows a schematic diagram of HA MN patch evaluated with a tensile testing machine. FIG. 10D shows the fracture force of HA+EV MN with 10%, 15%, or 20% HA. The dotted line represents the stress intensity of the microneedle inserted into the skin (n=3 for all groups). FIG. 10E shows hematoxylin and eosin (H&E) stained section of mouse skin shows penetration of single MN. Scale bar, 200 μm. FIG. 10F shows time course of HA+EV MN tips pressed into skin; the MNs dissolved within 15 minutes of application. Scale bars, 200 μm. Skin recovery after HA+EV MN treatment shows minimal irritation. Scale bars, 5 cm. FIG. 10G shows skin histology of Dil-labeled EVs shows highly concentrated EVs (red) accumulated in subcutis after dermal injection and well-distributed EVs delivered by MN. Scale bar, 100 μm. FIG. 10H shows schematic diagrams of EV distribution after needle injection. FIG. 10I shows schematic diagrams of EV distribution after MN injection. FIG. 10J shows representative EV distribution analyzed by imageJ software. FIG. 10K shows fluorescence tracking of EVs injected by needle injection. EVs were labeled with Dil. Scale bar, 100 μm. FIG. 10L shows fluorescence tracking of EVs injected by MN injection. EVs were labeled with Dil. Scale bar, 100 μm. FIG. 10M shows cryogenic electron microscopy images. FIG. 10N shows quantification of numbers of broken EVs after injection by syringe needles or and MN (n=3 for all groups; *P=0.0228 Needle injection vs HA-MN). Scale bar, 100 nm. FIG. 10O) shows in vivo fluorescence images of nude mice treated with subcutaneous injection or HA MN patch delivery of Dil-labeled EVs on days 1, 2, 4, 7, 10 and 14 after delivery (n=3 for all groups). FIG. 10P shows quantification of fluorescence intensity over the 14-day treatment period. Data are normalized to the fluorescence intensity at day 1.

FIG. 11 illustrates COL1A1-EVs mRNA delivery via custom microneedle patch improved long term treatment of dermal wrinkles and resulted in long term protein replacement in skin in photoaged mice. FIG. 11A shows a schematic illustration of COL1A1 EVs released by HA microneedles and in vivo transfection patterns. FIG. 11B shows long-term (90-day) observation of 4 treatment groups after a single injection: (1) saline control, (2) COL1A1-EV delivered by 28 G syringe needle, (3) HA MN control, and (4) COL1A1-loaded HA+EV MN (n=4 for all groups). Scale bar, 5 cm. FIG. 11C shows quantification of total numbers of wrinkles (n=4 for all groups, *P=0.014 HA+EV MN vs Saline at day 7; *P=0.038 HA+EV MN vs Saline at day 14; *P=0.012 HA+EV MN vs Saline at day 21; *P=0.039 HA+EV MN vs Saline at day 28; *P=0.031 HA+EV MN vs Saline at day 35; *P=0.03 HA+EV MN vs Saline at day 42; *P=0.022 HA+EV MN vs Saline at day 49; *P=0.031 HA+EV MN vs Saline at day 56; **P=0.008 HA+EV MN vs Saline at day 63; †P=0.047 HA MN vs Saline at day 14; #P=0.030 Needle injection vs Saline at day 7; #P=0.041 Needle injection vs Saline at day 14). FIG. 11D shows quantification of total dorsal skin wrinkle area (n=4 for all groups, *P=0.016 HA+EV MN vs Saline at day 7; *P=0.038 HA+EV MN vs Saline at day 14; *P=0.042 HA+EV MN vs Saline at day 28; *P=0.026 HA+EV MN vs Saline at day 42; *P=0.048 HA+EV MN vs Saline at day 49; #P=0.033 Needle injection vs Saline at day 7; #P=0.031 Needle injection vs Saline at day 14; #P=0.042 Needle injection vs Saline at day 21). FIG. 11E. Microscopic observation of dorsal skin and skin replica at day 30. FIG. 11F shows Microscopic observation of dorsal skin and skin replica at day 60. FIG. 11G shows Microscopic observation of dorsal skin and skin replica at day 90. FIG. 11H shows quantification of mean wrinkle length (n=4 for all groups, ***P<0.001 HA+EV MN vs Saline at day 30 and 60; †††P<0.001 HA MN vs Saline at day 30; ###P<0.001 Needle injection vs Saline at day 30). FIG. 11I shows quantification of wrinkle mean wrinkle depth (n=4 for all groups, ***P<0.001 HA+EV MN vs Saline at days 30 and 60; #P=0.049 Needle injection vs Saline at day 30; HA MN vs Saline not significant at day 30) from skin replicas. FIG. 11J shows immunofluorescence staining of GFP and COL1A1 (RFP) demonstrates GFP+ COL1A1 protein grafts in the skin of mice receiving COL1A1-EVs via 28 G needle injection and via HA+EV MN for up to 30 days after delivery. Scale bar, 200 μm. FIG. 11K shows at day 60, only mice that were treated with HA+EV MN had long-term GFP-positive COL1A1 engraftment. Scale bar, 200 μm. FIG. 11L shows No evidence of GFP-positive collagen protein could be detected in any mice by day 90 after delivery. Scale bar, 200 μm. FIG. 11M shows quantification of GFP and COL1A1 (RFP) co-localized fluorescence signal demonstrates long-term COL1A1-EV derived collagen engraftment in the skin of mice given HA+EV MN vs mice given COL1A1-EV via 28 G needle injection and control groups (n=3 for all groups, ***P<0.001 HA+EV MN vs Needle injection at day 60). FIG. 11N shows IHC and Masson trichrome staining at days 30, 60, and 90. Scale bar, 200 μm. FIG. 11O shows quantification of dermal thickness by Masson trichrome staining (n=3 for all groups, *P=0.017 HA+EV MN vs Saline at day 30; P=0.034 HA+EV MN vs Saline at day 60).

### DETAILED DESCRIPTION

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Overview

This disclosure provides extracellular vesicles that carry exogenous RNA cargo (e.g., messenger RNA (mRNA), extracellular matrix mRNA, collagen mRNA, Col1A mRNA, Col1A1 mRNA) and that can be used to treat a variety of skin conditions and other disorders, often with long-lasting effects. This disclosure also provides extracellular vesicles that contain VEGF mRNA and that can be used to treat disorders such as blood flow disorders. This disclosure also provides needles and microneedles that can be used to deliver the extracellular vesicles to a subject. This disclosure also provides hydrogel needles (e.g., hydrogel microneedles) that comprise extracellular vesicles carrying a variety of cargo (e.g., DNA, RNA, therapeutics) or no exogenous cargo.

The present disclosure also relates to the design and production of one or more extracellular vesicles (e.g., exosomes, microvesicles, apoptotic bodies, or mixtures thereof) that express and/or carry a cargo such as at least one extracellular matrix protein RNA (e.g., extracellular matrix (ECM) mRNA, collagen mRNA, Col1 mRNA, Col1A1 mRNA, ColIV mRNA, elastin mRNA) or vascular endothelial growth factor (VEGF) mRNA (e.g. VEGFA mRNA). In certain particular embodiments, this disclosure provides extracellular vesicles (e.g., exosomes) that contain exogenous collagen mRNA. In certain particular embodiments, this disclosure provides extracellular vesicles (e.g., exosomes) that contain exogenous VEGF mRNA. The extracellular vesicles can be designed to carry a payload such as a therapeutic to be delivered to the targeted cell. In some cases, the therapeutic delivered by the extracellular vesicles can include a therapeutic compound (e.g., a therapeutic polynucleotide, therapeutic DNA, therapeutic RNA, therapeutic mRNA, therapeutic miRNA, therapeutic tRNA, therapeutic rRNA, therapeutic siRNA, therapeutic shRNA, therapeutic SRP RNA, therapeutic tmRNA, therapeutic gRNA, or therapeutic crRNA). In some cases, the therapeutic delivered by the extracellular vesicle can include a therapeutic non-coding polynucleotide (e.g., non-coding RNA, lncRNA, piRNA, snoRNA, snRNAs, exRNA, or scaRNA), therapeutic polypeptide, therapeutic compound, or cancer drug. In some cases, the extracellular vesicles may carry a non-therapeutic compound (e.g., non-therapeutic polynucleotide).

The extracellular vesicles provided herein can be produced by a number of methods and approaches. One approach provided herein involves introducing at least one heterologous polynucleotide such as a vector (e.g., plasmid, DNA) into an extracellular vesicle donor cell, where the at least one heterologous polynucleotide encodes an extracellular matrix RNA (e.g., collagen) or VEGF RNA (e.g., VEGFA). Introduction of the vector (e.g., DNA plasmid) can be via a variety of methods, including transfection, calcium phosphate transfection, electroporation, nanoelectroporation, lipofection, cellular nanoelectroporation, viral delivery, or other methods. In some cases, the extracellular vesicle donor cell is a primary cell (e.g., a primary adherent cell). In some cases, the extracellular vesicle donor cell is from a cell line (e.g., human cell, human fibroblast line, human dermal fibroblast cell line, neonatal human dermal fibroblast line). In some cases, the extracellular vesicle donor cell is not genetically-modified prior to the nanoelectroporation. In some cases, the extracellular vesicle donor cell is genetically-modified prior to the nanoelectroporation. In some cases, this disclosure provides methods of producing large number of exosomes containing a high quantity of mRNA transcripts, even from cells with otherwise low basal secretion of exosomes.

Described herein are methods of treating a skin damage (e.g., caused by sun damage, aging, or other process) comprising administering at least one extracellular vesicle to the subject. The methods provided herein are especially useful for reversing, reducing, or reducing the appearance of fine lines and/or wrinkles. In some cases, the methods provided herein reduce skin damage (e.g., wrinkles, fine lines, etc.) over a period of time, such as at least 30, 60 or 90 days following administration of extracellular vesicles provided herein. In some cases, the methods provided herein can be used for wound healing. In some cases, the extracellular vesicles comprise at least one therapeutic polynucleotide (e.g., therapeutic mRNA, miRNA, etc.). In some cases, the extracellular vesicles comprising therapeutic polynucleotides can be obtained by nanoelectroporating at least one extracellular vesicle donor cell with at least a first vector (e.g. a plasmid), wherein the first vector encodes a therapeutic polypeptides. In some embodiments, the therapeutic polynucleotide comprises an extracellular matrix RNA (e.g., collagen). In some instances, the first vectors can be expressed in the extracellular vesicle donor cells to obtain the therapeutic polynucleotides. In some embodiments, the extracellular vesicles released from the extracellular vesicle donor cells comprise the therapeutic polynucleotides. In some cases, the extracellular vesicles are collected and systematically administered to the subject. In some cases, the extracellular vesicles are collected and locally administered to the subject (e.g., via subcutaneous injection).

Described herein are methods of treating a blood flow disorder (e.g., ischemia) comprising administering at least one extracellular vesicle to the subject. In some cases, the extracellular vesicles comprise at least one therapeutic polynucleotide (e.g., therapeutic mRNA, miRNA, etc.). In some cases, the extracellular vesicles comprising therapeutic polynucleotides can be obtained by nanoelectroporating at least one extracellular vesicle donor cell with at least a first vector (e.g. a plasmid), wherein the first vector encodes a therapeutic polypeptide. In some embodiments, the therapeutic polynucleotide comprises a VEGF RNA (e.g., VEGFA). In some instances, the first vectors can be expressed in the extracellular vesicle donor cells to obtain the therapeutic polynucleotides. In some embodiments, the extracellular vesicles released from the extracellular vesicle donor cells comprise the therapeutic polynucleotides. In some cases, the extracellular vesicles are collected and systemically administered to the subject. In some cases, the extracellular vesicles are collected and locally administered to the subject (e.g., via intramuscular injection).

Also described herein are hydrogel microneedles comprising a plurality of extracellular vesicles. In some cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In some cases, the hydrogel comprises a hyaluronic acid. For example, in some instances, the hydrogel comprises at least 5%, 10%, 15%, or 20% hyaluronic acid.

Also described herein are needles comprising a plurality of extracellular vesicles wherein the extracellular vesicles comprise at least one extracellular matrix mRNA or VEGF mRNA. Also described are syringes comprising a plurality of extracellular vesicles wherein the extracellular vesicles comprise at least one extracellular matrix mRNA or VEGF mRNA.

Further described herein are microneedle devices. In some cases, the microneedle devices comprise a substrate and a plurality of microneedles, wherein the plurality of microneedles protrude from the substrate, and wherein at least one microneedle of the plurality of microneedles comprises at least one EV. Also described herein are methods of manufacturing such microneedle device.

### Extracellular Vesicle Donor Cells

Described herein, in some cases, are extracellular vesicle donor cells that produce extracellular vesicles described herein. The extracellular vesicle donor cell can be any cell that can be genetically modified or manipulated to secrete extracellular vesicles at a level that is higher than the cell's basal level of secretion of extracellular vesicles or to secrete extracellular vesicles containing exogenous nucleic acids (e.g., exogenous collagen RNA or VEGF RNA). As such, a cell with low or negligible basal level of secretion of extracellular vesicles can also be an extracellular vesicle donor cell. In some cases, the extracellular vesicle donor cell can be a nucleated cell. In some cases, the extracellular vesicle donor cell can be an autologous cell. In such cases, the extracellular vesicle donor cell may be obtained from a subject; and then, following modification of the extracellular vesicle donor cell (e.g., introduction of a vector), secreted extracellular vesicles are collected and then administered to the same subject. In some cases, the extracellular vesicle donor cell is an allogeneic cell. In such case, the extracellular vesicle donor cell is a cell obtained from a source that is genetically distinct from the subject who later receives the extracellular vesicles secreted by the extracellular vesicle donor cell. Often, in the case of an allogeneic extracellular vesicle donor cell, the extracellular vesicle donor cell is of the same species, but genetically distinct from the subject who later receives the extracellular vesicles produced and secreted by the extracellular vesicle donor cell.

The extracellular vesicle donor cells can be any type of cell. In some cases, the extracellular vesicle donor cells are eukaryotic cells (e.g., mammalian cells, human cells, non-human mammalian cells, rodent cells, mouse cells, etc.). In some instances, the extracellular vesicle donor cells are cells from a cell line, stem cells, primary cells, or differentiated cells. In some embodiments, the extracellular vesicle donor cells are primary cells. In some instances, the extracellular vesicle donor cells are mouse embryonic fibroblasts (MEF), human embryonic fibroblasts (HEF), human dermal fibroblasts (HDF), dendritic cells, mesenchymal stem cells, bone marrow-derived dendritic cells, bone marrow derived stromal cells, adipose stromal cells, enucleated cells, neural stem cells, immature dendritic cells, or immune cells. In some cases, the extracellular vesicle donor cells are neonatal human primary dermal fibroblasts. In some cases, the neonatal human primary dermal fibroblasts are PCS-201-010 line from ATCC. In some cases, the extracellular vesicle donor cells are primary dermal fibroblasts. In some cases, the primary dermal fibroblasts are PCS-201-012 line from ATCC. The extracellular vesicle donor cells may be adherent cells. In some cases, the extracellular vesicle donor cells are adherent cells. In some cases, the extracellular vesicle donor cells are suspension cells.

In some cases, the extracellular vesicle donor cell comprises at least one heterologous polynucleotide. In some cases, the at least one heterologous polynucleotide is introduced into the extracellular vesicle donor cell by transfection. The at least one heterologous polynucleotide can be transfected into the extracellular vesicle donor cell by any one of the biological, chemical, or physical methods described herein, or by any other biological, chemical, or physical methods. In some instances, the at least one heterologous polynucleotide is transfected into the extracellular vesicle donor cell by electroporation (e.g., nanoelectroporation). In some cases, the electroporation is microchannel electroporation or nanochannel electroporation. In some instances, the at least one heterologous polynucleotide is transfected into the extracellular vesicle donor cell by nanochannel electroporation. In some cases, the extracellular vesicle donor cells comprise genetically modified cells. Examples of genetically modified cells can include induced pluripotent stem cells or cells that are genetically modified by nucleic acid guided nuclease (e.g. CRISPR-Cas). In some cases, the extracellular vesicle donor cells are not genetically-modified. For example, in some cases, the extracellular vesicle donor cells are not genetically-modified prior to electroporation (e.g. nanelectroporation). In some instances, the heterologous polynucleotide transfected into the extracellular vesicle donor cell is integrated into the chromosome of the extracellular vesicle donor cell. In some cases, the heterologous polynucleotide transfected into the extracellular vesicle donor cell is not integrated into the chromosome of the extracellular vesicle donor cell. In some cases, the extracellular vesicle donor cell is stably transfected with the heterologous polynucleotide. In some cases, the extracellular vesicle donor cell is transiently transfected with heterologous polynucleotide. In some cases, the transfected extracellular vesicle donor cell is a cell derived from a cell line. In some instances, the at least one heterologous polynucleotide is a vector (e.g. a plasmid).

In some cases, the extracellular vesicle donor cells can be electroporated by a plurality of vectors to produce and secrete extracellular vesicles. In some cases, the extracellular vesicle donor cells can be nanoelectroporated by a plurality of vectors to produce and secrete the extracellular vesicles. In some cases, the plurality of vectors comprise at least a first vector, at least a second vector, or any additional vector. In some cases, the first vectors and the second vectors can be nanoelectroporated into the extracellular vesicle donor cells at the same time. In some cases, the first vectors and the second vectors can be nanoelectroporated into the extracellular vesicle donor cells at different times. In some cases, the time difference between nanoelectroporating the first vectors and the second vectors can be at least 1 minute, 5 minutes, 10 minutes, 30 minutes, 1 hour, 5 hours, 12 hours, 1 day, 2 days, 5 days, 10 days, 30 days, or longer.

In some cases, the first vectors can encode at least one therapeutic polynucleotide (e.g., ECM or VEGF mRNA). In some cases, the extracellular vesicle donor cells, when nanoelectroporated with the first vectors, can transcribe the first vectors to obtain the therapeutic polynucleotides. In some cases, the extracellular vesicle donor cells produce and secrete the extracellular vesicles or exosomes comprising encapsulation of the therapeutic polynucleotides encoded by the first vectors.

In some cases, the second vectors can encode targeting polypeptides (e.g., cell-specific targeting peptides which can increase the targeting and accumulation of the extracellular vesicles to a targeted cell). In some instances, the extracellular vesicle donor cells, when nanoelectroporated with the second vectors, can translate the second vectors to obtain the targeting polypeptides. In some cases, the extracellular vesicle donor cells can produce extracellular vesicles or exosomes comprising the targeting polypeptides. In some cases, the extracellular vesicle donor cells can secrete and the produced extracellular vesicles or exosomes comprising the targeting polypeptides. In some cases, the extracellular vesicle donor cells can produce and secrete the extracellular vesicles or exosomes comprising the targeting peptide and the therapeutic polynucleotides encoded by the first vectors.

In some cases, the extracellular vesicle donor cells, when nanoelectroporated with the first vectors, can transcribe or translate the first vectors to obtain therapeutic polynucleotides or therapeutic polypeptides. In some cases, the extracellular vesicle donor cells can produce and secrete the extracellular vesicles or exosomes comprising the therapeutic polynucleotides or therapeutic polypeptides encoded by the first vectors. In some cases, the extracellular vesicle donor cells can produce and secrete the extracellular vesicles comprising the targeting peptide and the therapeutic polynucleotides or therapeutic polypeptides encoded by the first vectors. In some instances, the therapeutic polynucleotides and the therapeutic polypeptides can be encapsulated in the same extracellular vesicles or exosomes. In some instances, the therapeutic polynucleotides and the therapeutic polypeptides can be encapsulated in different extracellular vesicles or exosomes.

In some cases, the extracellular vesicle donor cell continuously produces and secretes the extracellular vesicles at a steady or a basal rate. The extracellular vesicle donor cell can be any cell type, including cells that have low basal or negligible rate or production and secretion of the extracellular vesicles. For example, the extracellular vesicle donor cell can be a primary cell or a non-cancerous cell that generally do not secrete, or secrete a low number of, extracellular vesicles.

In some cases, the extracellular vesicle donor cell produces and secretes the extracellular vesicles at a basal rate. In some cases, the extracellular vesicle donor cell can be stimulated to produce and secrete extracellular vesicles at a rate that is higher than the basal rate. For example, the extracellular vesicle donor cell can be stimulated to produce and secrete extracellular vesicles at a rate that is higher than the basal rate by heat shocking the extracellular vesicle donor cell or contacting the extracellular vesicle donor cell with Ca²⁺ ions. In some cases, the extracellular vesicle donor cell can be stimulated to produce and secrete extracellular vesicles at a rate that is higher than the basal rate by activating a stress response signaling pathway such as p53-TSAP6 signaling pathway. In some cases, the extracellular vesicle donor cell can be stimulated to produce and secrete extracellular vesicles at a rate that is higher than the basal rate by electroporating the at least one heterologous polynucleotide into the extracellular vesicle donor cell. In some cases, the extracellular vesicle donor cell can be stimulated to produce and secrete extracellular vesicles at a rate that is higher than the basal rate by microchannel electroporation or nanochannel electroporation the at least one heterologous polynucleotide into the extracellular vesicle donor cell. In some cases, the extracellular vesicle donor cell can be stimulated to produce and secrete extracellular vesicles at a rate that is higher than the basal rate by electroporating (e.g., nanochannel electroporating) the at least one heterologous polynucleotide into the extracellular vesicle donor cell. In some instances, the extracellular vesicle donor cell stimulated by electroporation (e.g., nanochannel electroporation) can produce and secrete the extracellular vesicles at a rate that is at least 0.1 fold, 0.2 fold, 0.3 fold, 0.4 fold, 0.5 fold, 0.6 fold, 0.7 fold, 0.8 fold, 0.9 fold, 2 fold, 5 fold, 10 fold, 50 fold, 100 fold, 500 fold, 1,000 fold, 5,000 fold, 10,000 fold, 50,000 fold, 100,000 fold, or more higher than the basal rate of the extracellular vesicle donor cell producing and secreting the extracellular vesicles. In some cases, the extracellular vesicle donor cell stimulated by nanochannel electroporation can produce and secrete the extracellular vesicles at a rate that is at least 0.1 fold, 0.2 fold, 0.3 fold, 0.4 fold, 0.5 fold, 0.6 fold, 0.7 fold, 0.8 fold, 0.9 fold, 2 folds, 5 folds, 10 folds, 50 folds, 100 folds, 500 folds, 1,000 folds, 5,000 folds, 10,000 fold, 50,000 folds, 100,000 fold, or more higher than the rate of the extracellular vesicle donor cell stimulated by methods other than nanoelectroporation for producing and secreting the extracellular vesicles.

In some cases, a heterologous polynucleotide transfected into the extracellular vesicle donor cell encodes at least one therapeutic described herein. In some cases, the therapeutic is a therapeutic polynucleotide. In some instances, the therapeutic is a therapeutic polypeptide. In some instances, the extracellular vesicle donor cell transfected with at least one heterologous polynucleotide produces and secretes extracellular vesicles comprising the at least one targeting polypeptide. In some instances, the extracellular vesicle donor cell transfected with at least one heterologous polynucleotide produces and secretes extracellular vesicles comprising the at least one therapeutic.

### Extracellular Vesicles

Provided herein, in some cases, are compositions comprising extracellular vesicles and methods of producing extracellular vesicles. In some cases, the extracellular vesicles are any membrane-bound particle (e.g., a vesicle with a lipid bilayer). Often, the extracellular vesicles provided herein are secreted by a cell. In some instances, the extracellular vesicles are membrane-bound particles produced in vitro. In some cases, the extracellular vesicles are produced and secreted by an extracellular vesicle donor cell transfected with at least one heterologous polynucleotide. In some instances, the extracellular vesicle is an exosome, a microvesicle, a retrovirus-like particle, an apoptotic body, an apoptosome, an oncosome, an exopher, an enveloped virus, an exomere, or a very large extracellular vesicle such as a large oncosome. In some cases, the extracellular vesicle is an exosome.

In some cases, the extracellular vesicles can have a diameter about 10 nm to about 50,000 nm. In some cases, the extracellular vesicles can have a diameter about 10 nm to about 20 nm, about 10 nm to about 30 nm, about 10 nm to about 50 nm, about 10 nm to about 100 nm, about 10 nm to about 200 nm, about 10 nm to about 500 nm, about 10 nm to about 1,000 nm, about 10 nm to about 2,000 nm, about 10 nm to about 5,000 nm, about 10 nm to about 10,000 nm, about 10 nm to about 50,000 nm, about 20 nm to about 30 nm, about 20 nm to about 50 nm, about 20 nm to about 100 nm, about 20 nm to about 200 nm, about 20 nm to about 500 nm, about 20 nm to about 1,000 nm, about 20 nm to about 2,000 nm, about 20 nm to about 5,000 nm, about 20 nm to about 10,000 nm, about 20 nm to about 50,000 nm, about 30 nm to about 50 nm, about 30 nm to about 100 nm, about 30 nm to about 200 nm, about 30 nm to about 500 nm, about 30 nm to about 1,000 nm, about 30 nm to about 2,000 nm, about 30 nm to about 5,000 nm, about 30 nm to about 10,000 nm, about 30 nm to about 50,000 nm, about 50 nm to about 100 nm, about 50 nm to about 200 nm, about 50 nm to about 500 nm, about 50 nm to about 1,000 nm, about 50 nm to about 2,000 nm, about 50 nm to about 5,000 nm, about 50 nm to about 10,000 nm, about 50 nm to about 50,000 nm, about 100 nm to about 200 nm, about 100 nm to about 500 nm, about 100 nm to about 1,000 nm, about 100 nm to about 2,000 nm, about 100 nm to about 5,000 nm, about 100 nm to about 10,000 nm, about 100 nm to about 50,000 nm, about 200 nm to about 500 nm, about 200 nm to about 1,000 nm, about 200 nm to about 2,000 nm, about 200 nm to about 5,000 nm, about 200 nm to about 10,000 nm, about 200 nm to about 50,000 nm, about 500 nm to about 1,000 nm, about 500 nm to about 2,000 nm, about 500 nm to about 5,000 nm, about 500 nm to about 10,000 nm, about 500 nm to about 50,000 nm, about 1,000 nm to about 2,000 nm, about 1,000 nm to about 5,000 nm, about 1,000 nm to about 10,000 nm, about 1,000 nm to about 50,000 nm, about 2,000 nm to about 5,000 nm, about 2,000 nm to about 10,000 nm, about 2,000 nm to about 50,000 nm, about 5,000 nm to about 10,000 nm, about 5,000 nm to about 50,000 nm, or about 10,000 nm to about 50,000 nm. In some cases, the extracellular vesicles have a diameter about 10 nm, about 20 nm, about 30 nm, about 50 nm, about 100 nm, about 200 nm, about 500 nm, about 1,000 nm, about 2,000 nm, about 5,000 nm, about 10,000 nm, or about 50,000 nm. In some cases, the extracellular vesicles can have a diameter at least about 10 nm, about 20 nm, about 30 nm, about 50 nm, about 100 nm, about 200 nm, about 500 nm, about 1,000 nm, about 2,000 nm, about 5,000 nm, or about 10,000 nm. In some cases, the extracellular vesicles can have a diameter at most about 20 nm, about 30 nm, about 50 nm, about 100 nm, about 200 nm, about 500 nm, about 1,000 nm, about 2,000 nm, about 5,000 nm, about 10,000 nm, or about 50,000 nm.

Extracellular vesicle surface proteins are generally proteins that are associated with extracellular vesicles. In some cases, the extracellular vesicle surface protein can be expressed by the extracellular vesicle donor cell and integrated and secreted as part of the extracellular vesicle produced and secreted by the extracellular donor cell. In some instances, the extracellular vesicle surface protein comprises at least one an extracellular domain, which can include the N-terminus, the C-terminus, or both the N and C terminus of the extracellular vesicle surface protein. In some cases, the extracellular vesicle surface protein can be encoded by the at least one heterologous polynucleotide or vector described herein. In some cases, the extracellular vesicle surface protein can be a member of the immunoglobulin superfamily. Members of the immunoglobulin superfamily can include antigen receptors, antigen presenting molecules, co-receptors, antigen receptor accessory molecules, co-stimulatory or inhibitory molecules, receptors on natural killer cells, receptors on leukocytes, immunoglobulin-like cell adhesion molecules, cytokine receptors, growth factor receptors, receptor tyrosine kinases, receptor tyrosine phosphatases, immunoglobulin binding receptors, cytoskeletons, or other members. In some cases, the extracellular vesicle surface protein comprising the member of the immunoglobulin superfamily comprises a variable immunoglobulin domain (IgV) or a constant immunoglobulin domain (IgC). In some cases, the extracellular vesicle surface protein comprising the member of the immunoglobulin superfamily comprises an IgV domain. Example of the member of the immunoglobulin superfamily comprising IgV can include cluster of differentiation proteins (e.g. CD2, CD4, CD47, CD80, or CD86), myelin membrane adhesion molecules, junction adhesion molecules (JAM), tyrosine-protein kinase receptors, programmed cell death protein 1 (PD1), or T-cell antigen receptors.

In some cases, the extracellular vesicle comprises components other than therapeutic polynucleotides. In some cases, the extracellular vesicle comprises one or more preservatives. In some cases, the extracellular vesicle comprises one or more stabilizing agents. In some cases, the extracellular vesicle comprises one or more DNAses. In some cases, the extracellular vesicle comprises one or more RNase inhibitors. In some cases, the extracellular vesicle comprises one or more DNAses and/or RNase inhibitors. In some cases, the extracellular vesicle comprises one or more DNAses, DNAse inhibitors, RNAse, and/or RNAse inhibitors.

In some cases, the extracellular vesicle comprising the at least one targeting polypeptide exhibits increased half-life in circulation compared to half-life of an extracellular vesicle without the targeting polypeptide. In some cases, the half-life of the extracellular vesicle comprising the at least one targeting polypeptide is increased by at least 0.1 fold, 0.2 fold, 0.5 fold, 1 fold, 2 fold, 3 fold, 5 fold, 10 fold, 20 fold, 50 fold, 100 fold, 1000 fold, or more compared to half-life of extracellular vesicle without the targeting polypeptide. In some cases, the half-life of the extracellular vesicle comprising the at least one targeting polypeptide is increased by at least 1 minute, 2 minutes, 3 minutes, 4 minutes, 5 minutes, 10 minutes, 15 minutes, 30 minutes, 60 minutes, 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, 30 days, or longer compared to half-life of extracellular vesicle without the targeting polypeptide.

In some cases, the extracellular vesicle comprising the at least one targeting polypeptide exhibits a half-life in circulation of a mammal (e.g., human, rodent, mouse) of at least 30 seconds, at least 1 minute, at least 2 minutes, at least 3 minutes, at least 5 minutes, or at least 10 minutes. In some cases, the extracellular vesicle comprising the targeting exhibits a half-life in the circulation of a mammal of less than 5 hours, less than 2 hours, less than 1 hours, or less than 30 minutes.

In some cases, the targeting polypeptide comprises a heterologous targeting domain. In some instances, heterologous targeting domain is a tumor targeting domain, a tissue-targeting domain, a cell-penetrating peptide, a viral membrane protein, or a combination thereof. The heterologous targeting domain can target a cell-surface marker expressed on the surface of a targeted cell. The cell-surface marker can be any macromolecule or protein expressed on the surface of the targeted cell. Non-limiting examples of the cell-surface marker includes Vascular receptor, Fibronectin receptor, A2B5, CD44, CD24, ESA, SSEA1, CD133, CD34, CD19, CD38, CD26, CD166, or CD90.

In some instances, the accumulation of the extracellular vesicle comprising the at least one targeting polypeptide at the targeted cell expressing the cell-surface marker is higher than accumulation of extracellular vesicle without the at least one targeting polypeptide at the same targeted cell expressing the same cell-surface marker. In some instances, the accumulation of the extracellular vesicle comprising the at least one targeting polypeptide at the targeted cell expressing the cell-surface marker is at least 0.1 fold, 0.2 fold, 0.5 fold, 2 fold, 5 fold, 10 fold, 50 fold, 100 fold, 500 fold, 1,000 fold, 5,000 fold, 10,000 fold, or higher compared to the accumulation of extracellular vesicle without the targeting polypeptide at the same targeted cell expressing the same cell-surface marker.

In some instances, the hepatic and splenic accumulation, e.g. accumulation of the extracellular vesicles at non-targeted cells, of the extracellular vesicles comprising the at least one targeting polypeptide at the targeted cell expressing the cell-surface marker is reduced compared to hepatic and splenic accumulation of extracellular vesicles without the at least one targeting polypeptide at the same targeted cell. In some instances, the hepatic and splenic accumulation of the extracellular vesicles comprising the at least one targeting polypeptide is reduced by at least 0.1 fold, 0.2 fold, 0.5 fold, 2 fold, 5 fold, 10 fold, 50 fold, 100 fold, 500 fold, 1,000 fold, 5,000 fold, or 10,000 fold compared to the hepatic and splenic accumulation of extracellular vesicles without the targeting polypeptide.

In some instances, the targeting polypeptide comprises at least one heterologous targeting domain. In some cases, the at least one heterologous targeting domain is a tumor targeting domain, a tissue-targeting domain, a cell-penetrating peptide, a viral membrane protein, or a combination thereof. In some instances, the at least one heterologous targeting domain is the tumor targeting domain, where the tumor targeting domain targets a cancerous cell. In some instances, the at least one heterologous targeting domain is the tumor targeting domain, where the tumor targeting domain targets a non-cancerous lesion cell.

In some cases, the targeting polypeptide comprises at least one, two, three, four, five, or more heterologous targeting domains. In some instances, the at least two heterologous targeting domains can be identical. In some cases, the at least two heterologous targeting domains can be different.

In some cases, the targeting polypeptide comprises at least one tissue-targeting domain, which targets and directs the extracellular vesicle comprising the targeting polypeptide to a cell of a specific tissue. In some cases, the targeting polypeptide comprises at least two, three, four, five, or more tissue-targeting peptides. In some instances, the at least two tissue-targeting peptides are identical. In some cases, the at least two tissue-targeting peptides are different. In some instances, the tissue-targeting peptide comprises at least 3, 4, 5, 6, 7, 8, 9, 10, 15, 20, 25, 30, 40, 50, or 100 amino acids. Exemplary tissue-targeting domain which targets endothelial or cardiac tissue includes SIGYPLP, LSIPPKA, FQTPPQL, LTPATAI, CNIWGVVLSWIGVFPEC, NTTTH, VHPKQHR(tetramer), CRKRLDRNCCRTLTVRKC, CLWTVGGGC, QPWLEQAYYSTF, YPHIDSLGHWRR, LLADTTHHRPWT, SAHGTSTGVPWP, VPWMEPAYQRFL, TLPWLEESYWRP, HWRR, CSTSMLKAC, DDTRHWG, CARPAR, CKRAVR, CRSTRANPC, CPKTRRVPC, CSGMARTKC, or CRPPR. Exemplary tissue-targeting domain which targets pancreatic tissue includes CRVASVLPC, SWCEPGWCR, LSGTPERSGQAVKVKLKAIP, CHVLWSTRCCVSNPRWKC, or LSALPRT. Exemplary tissue-targeting domain which targets kidney tissue includes CLPVASC, ELRGD(R/M)AX(W/L), GV(K/R)GX3(T/S)RDXR, HITSLLSHTTHREP, or ANTPCGPYTHDCPVKR. Exemplary tissue-targeting domain which targets lung tissue includes CGFELETCCGFECVRQCPERC, QPFMQCLCLIYDASCRNVPPIFNDVYWIAF, VNTANST, CTSGTHPRC, or SGEWVIKEARGWKHW-VFYSCCPTTPYLDITYH. Exemplary tissue-targeting domain which targets intestinal tissue includes YSGKWGW, LETTCASLCYPSYQCSYTMPHPPVVPPHPMTYSCQY, YPRLLTP, CSQSHPRHC, CSKSSDYQC, CKSTHPLSC, CTGKSCLRVG, SFKPSGLPAQSL, or CTANSSAQC. Exemplary tissue-targeting domain which targets brain tissue can include CLSSRLDAC, GHKAKGPRK, HAIYPRH, THRPPMWSPVWP, HLNILSTLWKYRC, CAGALCY, CLEVSRKNC, RPRTRLHTHRNR(D-aa), ACTTPHAWLCG, GLAHSFSDFARDFV, GYRPVHNIRGHWAPG, TGNYKALHPHNG, CRTIGPSVC, CTSTSAPYC, CSYTSSTMC, CMPRLRGC, TPSYDTYAAELR, RLSSVDSDLSGC, CAQK, or SGVYKVAYDWQH. Additional exemplary tissue-targeting domain targeting various tissue includes LMLPRAD (targeting adrenal gland), CSCFRDVCC (targeting retina), CRDVVSVIC (targeting retina), CVALCREACGEGC (targeting skin hypodermal vasculature), GLSGGRS (targeting uterus), WYRGRL (targeting cartilage), CPGPEGAGC (targeting breast vasculature), SMSIARLVSFLEYR (targeting prostate), GPEDTSRAPENQQKTGC (targeting skin Langerhans), CKGGRAKDC (targeting white fat vasculature), CARSKNKDC (targeting wound or damaged tissue), CHAQGSAEC (targeting thymus), LEPRWGFGWWLKLSTHTTESRSMV (targeting ear or cochlea tissue), ACSTEALRHCGGGS (targeting retinal vessel), ASSLNIA (targeting muscle tissue), CSTSMLKAC (targeting ischemia), or CSTSMLKAC (targeting ischemic myocardium).

In some cases, the targeting polypeptide comprises at least two, three, four, five, or more cell-penetrating peptides. In some cases, the targeting polypeptide comprising the cell-penetrating peptide increases the rate of the extracellular vesicle being fused or endocytosed by the targeted cell. In some instances, the at least two cell-penetrating peptides are identical. In some cases, the at least two cell-penetrating peptides are different. In some instances, the cell-penetrating peptide comprises at least 3, 4, 5, 6, 7, 8, 9, 10, 15, 20, 25, 30, 40, 50, or 100 amino acids. Non-limiting example of the cell-penetrating peptide includes DSLKSYWYLQKFSWR, DWLKAFYDKVAEKLKEAF, KSKTEYYNAWAVWERNAP, GNGEQREMAVSRLRDCLDRQA, HTPGNSNKWKHLQENKKGRPRR, DWLKAFYDKVAEKLKEAF, R9GPLGLAGE8, Ac-GAFSWGSLWSGIKNFGSTVKNYG, RLRWR, LGQQQPFPPQQPY, ILGKLLSTAAGLLSNL, TFFYGGSRGKRNNFKTEEY, Ac-LRKLRKRLLRX-Bpg-G, Ac-LRKLRKRLLR, or MVRRFLVTLRIRRACGPPRVRV.

In some cases, the targeting polypeptide comprises at least two, three, four, five, or more viral membrane proteins or fragments thereof. In some cases, the targeting polypeptide comprising the viral membrane protein increases the rate of the extracellular vesicle being fused or endocytosed by the targeted cell. In some instances, the at least two viral membrane proteins are identical. In some cases, the at least two viral membrane proteins are different. In some instances, the viral membrane protein comprises at least 3, 4, 5, 6, 7, 8, 9, 10, 15, 20, 25, 30, 40, 50, or 100 amino acids. Non-limiting example of the viral membrane protein includes hemagglutinin, glycoprotein 41, envelop protein, VSV G, HSV01 gB, ebolavirus glycoprotein, or fusion-associated small transmembrane (FAST) protein.

In some cases, the extracellular vesicle described herein comprises at least one therapeutic. In some cases, the at least one therapeutic is within (e.g. encapsulated) the extracellular vesicle. In some cases, the therapeutic is a therapeutic polynucleotide. In some cases, the therapeutic is a therapeutic polypeptide. In some instances, the therapeutic is a therapeutic compound. In some cases, the therapeutic comprises a therapeutic polynucleotide, therapeutic polypeptide, therapeutic compound, or a combination thereof. In some instances, the extracellular vesicle comprises a plurality of therapeutics, where the plurality of therapeutics comprises therapeutic polynucleotide, therapeutic polypeptide, therapeutic compound, or a combination thereof.

In some cases, the extracellular vesicles described herein comprise at least one targeting polypeptide. In some cases, the targeting polypeptide is a skin targeting polypeptide comprising the skin targeting domain. In some cases, the accumulation of the extracellular vesicles comprising the skin targeting polypeptides comprising the skin targeting domain at the skin is higher compared to accumulation of extracellular vesicles without the skin target polypeptides. In some instances, the accumulation of the extracellular vesicles comprising the skin targeting polypeptides at the skin is at least 2 fold, 5 fold, 10 fold, 50 fold, 100 fold, 200 fold, 500 fold, 1,000 fold, 5,000 fold, or 10,000 fold higher compared to accumulation of extracellular vesicles lacking the skin targeting polypeptide. In some instances, the accumulation of the extracellular vesicles comprising the skin targeting polypeptides at the skin is at least 100 fold higher compared to accumulation of extracellular vesicles lacking the skin targeting polypeptide.

In some cases, the skin targeting polypeptides comprise at least one skin targeting domain. In some cases, the skin targeting domains can be on an N-terminus of the skin targeting polypeptides. In some cases, the skin targeting domains can be on a C-terminus of the skin targeting polypeptides. In some cases, the skin targeting domains can at any peptide location of the skin targeting polypeptides. In some cases, at least two targeting domains can be on the same skin targeting polypeptides. In some cases, the at least two targeting domains on the same skin targeting polypeptides can be the same. In some cases, the at least two targeting domains on the same skin targeting polypeptides can be different. In some instances, the targeting domains comprise at least 3, 4, 5, 6, 7, 8, 9, 10, 15, 20, 25, 30, 40, 50, or 100 amino acids.

In some cases, the extracellular vesicles comprising the extracellular vesicle surface proteins comprise increased half-life in circulation compared to half-life of extracellular vesicles without the extracellular vesicle surface proteins. In some cases, the half-life of the extracellular vesicles increased by the extracellular vesicle surface proteins is at least 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, 30 days, or longer than half-life of extracellular vesicles lacking the extracellular vesicle surface proteins.

In some cases, the extracellular vesicles comprising the extracellular vesicle surface proteins have decreased toxicity compared to the extracellular vesicles lacking the extracellular vesicle surface proteins. In such cases, often the extracellular vesicle surface proteins specifically bind to a target and do not have significant off-target binding. In some cases, the toxicity comprises toxicity to cells that are not targeted by the tumor targeting polypeptides. In some cases, the extracellular vesicles comprising the extracellular vesicle surface proteins have decreased toxicity that is at least is 1 fold, 2 fold, 3 fold, 4 fold, 5 fold, 6 fold, 7 fold, 8 fold, 9 fold, 10 fold, 20 fold, 30 fold, 50 fold, 100 fold, or more decreased compared to the extracellular vesicles lacking the extracellular vesicle surface proteins. In some cases, the decreased toxicity of the extracellular vesicles comprising the extracellular vesicle surface proteins is at least 10%, 20%, 30%, 40%, 50%, 60%, 70%, 80%, 90%, 100%, 200%, 300%, 400%, 500%, or more decreased compared to the extracellular vesicles lacking the extracellular vesicle surface proteins.

In some cases, the extracellular vesicles (e.g., exosomes) are tolerated by the subject following administration of the extracellular vesicles. For example, in some cases, the extracellular vesicles do not induce an immune response, or are not immunogenic.

### Therapeutic Polynucleotides

Described herein, in some cases, are extracellular vesicles comprising at least one therapeutic polynucleotide. In some instances, the at least one therapeutic polynucleotide is encoded by the at least one heterologous polynucleotide or vector transfected into the extracellular vesicle donor cell. In some cases, the at least one therapeutic polynucleotide comprises a peptide sequence that can be translated into a therapeutic polypeptide by the cell targeted and bound by the targeting polypeptide described herein.

In some cases, the extracellular vesicles comprise at least one therapeutic polynucleotide. In some cases, each extracellular vesicle comprises at least 1, 2, 3, 4, 5, 6, 7, 8, 9, 10, 50, 100, 500, 1,000, 5,000, 10,000, 50,000, 100,000, 500,000, 1,000,000 or more copies of the therapeutic polynucleotides. In some cases, each extracellular vesicle comprises at least 1, 2, 3, 4, 5, 6, 7, 8, 9, 10, 50, 100, 500, 1,000, 5,000, 10,000, 50,000, 100,000, 500,000, 1,000,000 or more copies of the therapeutic mRNA described herein. In some instances, the extracellular vesicles comprise at least two therapeutic polynucleotides. In some instances, the extracellular vesicles comprise at least two therapeutic polynucleotides, where the at least two therapeutic polynucleotides are different. In some cases, the at least two different therapeutic polynucleotides encapsulated by the extracellular vesicles comprise different ratio. For example, the ratio between the first and the second of the two different therapeutic polynucleotides can be 1:1,000,000, 1:500,000, 1:100,000, 1:50,000, 1:10,000, 1:5,000, 1:1,000, 1:500, 1:100, 1:50, 1:10, 1:5, 1:4, 1:3, 1:2, or 1:1. In some instances, the extracellular vesicles comprise at least two, three, four, five, six, seven, right, nine, ten or more therapeutic polynucleotides encapsulated in the same extracellular vesicle. In some cases, the extracellular vesicles can be exosomes.

In some cases, the therapeutic polynucleotides comprise mRNA, rRNA, SRP RNA, tRNA, tmRNA, snRNA, snoRNA, gRNA, aRNA, crRNA, lncRNA, miRNA, ncRNA, piRNA, siRNA, and shRNA. In some cases, the therapeutic polynucleotides comprise mRNA. In some cases, the mRNA is fully intact or substantially intact. In some cases, the mRNA encodes a portion of the protein. In some cases, the mRNA comprises at least 50, 100, 200, 500, 1,000, 5,000, 10,000, 50,000, 100,000, 500,000, or 1,000,000 of RNA nucleotides. In some instances, therapeutic polynucleotides comprise DNA. In some instances, therapeutic polynucleotides comprise DNA such as vectors that encode therapeutic polypeptide or RNA therapeutic. The therapeutic polynucleotide can encode therapeutic polypeptide including but not limited to: an extracellular matrix (ECM) protein or peptide or an angiogenic protein or peptide. Examples of the therapeutic polypeptide that can be encoded by the therapeutic polynucleotide (e.g. messenger RNA therapeutic) includes fibrillar collagen (e.g., collagen type I, II, III, V, XI), FACIT collagen (e.g., collagen type IX, XII, XIV), short chain collagen (e.g., collagen type VIII, X), basement membrane collagen (e.g., collagen type IV), other collagens (e.g., collagen type VI, VII, XIII, XV, XVII, XVIIII), elastin (ELN), fibronectin (FN1), laminins (e.g., Laminin-111, Laminin-211, Laminin-121, Laminin-221, Laminin-332/Laminin-3A32, Laminin-3B32, Laminin-311/Lamnin-3A11, Laminin-321/Laminin-3A21, Laminin-411, Lamnin-421, Laminin-511, Laminin-521, Laminin-213, Laminin-423, Laminin-522, Laminin-523) or mixtures thereof, or VEGF (e.g., VEGFA, VEGFB, VEGFC, VEGFD, placental growth factor (PlGF), or mixtures thereof). In some cases, the therapeutic polynucleotide described herein can encode Collagen Type I (Col1). In some cases, the therapeutic polypeptide encoded from the therapeutic polynucleotide described herein can be Collagen Type I (Col1). In some cases, the therapeutic polynucleotide described herein can encode VEGF (VEGFA). In some cases, the therapeutic polypeptide encoded from the therapeutic polynucleotide described herein can be VEGF (VEGFA).

The COL1A1 mRNA is present with a certain copy number per EV with the method of production of EVs disclosed herein. In some cases, the plurality of extracellular vesicles comprises an average of at least one copy of the COL1A1 mRNA per 2000 EVs, per 1000 EVs, per 500 EVs, per 400 EVs, per 300 EVs, per 200 EVs, per 150 EVs, per 100 EVs, per 90 EVs, per 80 EVs, per 70 EVs, 60 EVs, 50 EVs, per 40 EVs, per 30 EVs, per 20 EVs, per 15 EVs, per 10 EVs, per 5 EVs, per 2 EVs, or per 1 EV. In some cases, the plurality of extracellular vesicles comprises an average of at least 1, 2, 5, 10, 50, 100, 200, 300, 500, or 1000 copies of the COL1A1 mRNA per EV. In some cases, comprises an average of at least one copy of the COL1A1 mRNA about per 0.1 to 100 EVs.

The VEGF mRNA is present with a certain copy number per EV with the method of production of EVs disclosed herein. In some cases, the plurality of extracellular vesicles comprises an average of at least one copy of the VEGF mRNA per 2000 EVs, per 1000 EVs, per 500 EVs, per 400 EVs, per 300 EVs, per 200 EVs, per 150 EVs, per 100 EVs, per 90 EVs, per 80 EVs, per 70 EVs, 60 EVs, 50 EVs, per 40 EVs, per 30 EVs, per 20 EVs, per 15 EVs, per 10 EVs, per 5 EVs, per 2 EVs, or per 1 EV. In some cases, the plurality of extracellular vesicles comprises an average of at least 1, 2, 5, 10, 50, 100, 200, 300, 500, or 1000 copies of the VEGF mRNA per EV. In some cases, comprises an average of at least one copy of the VEGF mRNA about per 0.02 to 50 EVs.

In some instances, a copy number of the therapeutic polynucleotide (e.g., RNA therapeutic, mRNA therapeutic) encapsulated in the extracellular vesicles is at least 1, at least 2, at least 3, at least 4, at least 5, at least 6, at least 7, at least 8, at least 9, at least 10, at least 25, at least 50, at least 100, at least 1,000, at least 10,000, at least 100,000, or more copies of the therapeutic polynucleotide per extracellular vesicle. In some instances, a copy number of the therapeutic polynucleotide (e.g., RNA therapeutic, mRNA therapeutic) encapsulated in each extracellular vesicle or exosome described herein is at least 1, at least 2, at least 3, at least 4, at least 5, at least 6, at least 7, at least 8, at least 9, at least 10, at least 25, at least 50, at least 100, at least 1,000, at least 10,000, at least 100,000, or more copies.

In some instances, a copy number of the therapeutic polynucleotide (e.g. RNA therapeutic, mRNA therapeutic) encapsulated in a plurality of extracellular vesicles (e.g., a plurality of EVs secreted by donor cells within a culture vessel) is on average at least 1, at least 2, at least 3, at least 4, at least 5, at least 6, at least 7, at least 8, at least 9, at least 10, at least 25, at least 50, at least 100, at least 1,000, at least 10,000, at least 100,000, or more copies of the therapeutic polynucleotide per extracellular vesicle. In some instances, a copy number of the therapeutic polynucleotide (e.g., RNA therapeutic, mRNA therapeutic) encapsulated in each extracellular vesicle or exosome described herein is, on average, at least 1, at least 2, at least 3, at least 4, at least 5, at least 6, at least 7, at least 8, at least 9, at least 10, at least 25, at least 50, at least 100, at least 1,000, at least 10,000, at least 100,000, or more copies.

In some instances, a copy number of the therapeutic polynucleotide (e.g. RNA therapeutic or messenger RNA therapeutic) encapsulated in the extracellular vesicles produced from extracellular vesicle donor cell transfected (e.g., by microchannel electroporation or nanochannel electroporation) is increased compared to a copy number of the therapeutic polynucleotide encapsulated in the extracellular vesicles produced from extracellular vesicle donor cell transfected by other methods of transfection (e.g. conventional bulk electroporation, gene gun, lipofectamine transfection, etc.) by at least 0.1 fold, 0.2 fold, 0.5 fold, 2 fold, 5 fold, 10 fold, 50 fold, 100 fold, 500 fold, 1,000 fold, 5,000 fold, 10,000 fold, or more. In some instances, a copy number of the therapeutic polynucleotide (e.g. RNA therapeutic or messenger RNA therapeutic) encapsulated in the extracellular vesicles produced from microchannel electroporated or nanochannel electroporated extracellular vesicle donor is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or more compared to a copy number of the therapeutic polynucleotide encapsulated in the extracellular vesicles produced by directly introducing the therapeutic polynucleotide into the extracellular vesicles (e.g., directly transfecting the therapeutic polynucleotide into the extracellular vesicles).

In some instances, the therapeutic polynucleotide (e.g., RNA therapeutic or messenger RNA therapeutic) encapsulated in the extracellular vesicles produced from extracellular vesicle donor cell transfected by microchannel electroporating or nanochannel electroporating is fully or substantially intact, where at least 30%, 40%, 50%, 60%, 70%, 80%, 90%, 95%, 99%, or more of the copies of the encapsulated therapeutic polynucleotide is fully intact or substantially intact. In some cases, a percentage of the fully intact or substantially intact therapeutic polynucleotide (e.g. RNA therapeutic or messenger RNA therapeutic) encapsulated in the extracellular vesicles produced from extracellular vesicle donor cell transfected by microchannel electroporating or nanochannel electroporating is increased compared to a percentage of the fully intact or substantially intact therapeutic polynucleotide (e.g. RNA therapeutic or messenger RNA therapeutic) encapsulated in the extracellular vesicles produced from extracellular vesicle donor cell transfected by other methods of transfection (e.g. conventional bulk electroporation, gene gun, lipofectamine transfection, etc.). In some cases, the number of fully intact or substantially intact therapeutic polynucleotide (e.g. RNA therapeutic or messenger RNA therapeutic) encapsulated in the extracellular vesicles produced from extracellular vesicle donor cell transfected by microchannel electroporating or nanochannel electroporating is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or more compared to the number of the fully intact or substantially intact therapeutic polynucleotide encapsulated in the extracellular vesicles produced from the extracellular vesicle donor cell transfected by other methods of transfection (e.g. conventional bulk electroporation, gene gun, lipofectamine transfection, etc.). In some cases, the number of the fully intact or substantially intact therapeutic polynucleotide (e.g. RNA therapeutic or messenger RNA therapeutic) encapsulated in the extracellular vesicles produced from extracellular vesicle donor cell transfected by microchannel electroporating or nanochannel electroporating is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or more compared to the number of fully intact or substantially intact therapeutic polynucleotide encapsulated in the extracellular vesicles produced from introducing the therapeutic polynucleotide directly into the extracellular vesicles (e.g., directly transfecting the therapeutic polynucleotide into the extracellular vesicles).

In some cases, the therapeutic polynucleotides comprise at least one modified nucleic acid or nucleic acid analog. Exemplary modified nucleic acids include, but are not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain modified nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deazaadenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thiouracil, N4-ethylcytosine, 7-deazaguanine, 7-deaza-8-azaguanine, 5-hydroxycytosine, 2′-deoxyuridine, 2-amino-2′-deoxyadenosine Modified nucleic acids comprising various heterocyclic bases and various sugar moieties (and sugar analogs) are available in the art, and the nucleic acids in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo [2.3-d] pyrimidin-5-yl, 2-amino-4-oxopyrolo [2,3-d] pyrimidin-5-yl, 2-amino-4-oxopyrrolo [2.3-d] pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some cases, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included.

In some cases, modified nucleic acids include 2′,3′-dideoxy-2′,3′-didehydro-nucleosides 5′-substituted DNA and RNA derivatives or 5′-substituted monomers made as the monophosphate with modified bases.

In some cases, modified nucleic acids include modifications at the 5′-position and the 2′-position of the sugar ring (, such as 5′-CH₂-substituted 2′-O-protected nucleosides. In some cases, modified nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃. Modified nucleic acids can include 2′-substituted 5′-CH₂ (or O) modified nucleosides. Modified nucleic acids can include 5′-methylenephosphonate DNA and RNA monomers, and dimers. Modified nucleic acids can include 5′-phosphonate monomers having a 2′-substitution and other modified 5′-phosphonate monomers. Modified nucleic acids can include 5′-modified methylenephosphonate monomers. Modified nucleic acids can include analogs of 5′ or 6′-phosphonate ribonucleosides comprising a hydroxyl group at the 5′ and/or 6′-position. Modified nucleic acids can include 5′-phosphonate deoxyribonucleoside monomers and dimers having a 5′-phosphate group. Modified nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some cases, modified nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain cases, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R₁)(R₂) (R=H, C₁-C₁₂ alkyl or a protecting group); and combinations thereof.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as modified modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙCH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain cases, nucleic acids described herein include one or more bicyclic nucleic acids. In certain such cases, the bicyclic nucleic acid comprises a bridge between the 4′ and the 2′ ribosyl ring atoms. In certain cases, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof; 4′-C(CH₃)(CH₃)—O-2′ and analogs thereof.

In certain cases, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain aspects, inter nucleic acids linkages having a chiral atom can be prepared as a racemic mixture, as separate enantiomers, e.g., alkylphosphonates and phosphorothioates. Modified nucleic acids can contain a single modification. Modified nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some cases, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, cholic acid, a thioether, e.g., hexyl-S-tritylthiol, a thiocholesterol, an aliphatic chain, e.g., dodecandiol or undecyl residues (, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some cases, the at least one modified nucleotide or nucleotide analogue described herein can be resistant toward nucleases such as for example ribonuclease such as RNase H, deoxyribonuclease such as DNase, or exonuclease such as 5′-3′ exonuclease and 3′-5′ exonuclease when compared to natural nucleic acid molecules. In some instances, the at least one modified nucleotide or nucleotide analogue comprises 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase H, deoxyribunuclease such as DNase, or exonuclease such as 5′-3′ exonuclease and 3′-5′ exonuclease. In some instances, 2′-O-methyl modified nucleic acid molecule is nuclease resistance (e.g., RNase H, DNase, 5′-3′ exonuclease or 3′-5′ exonuclease resistance). In some instances, 2′O-methoxyethyl (2′-O-MOE) modified nucleic acid molecule is nuclease resistance (e.g., RNase H, DNase, 5′-3′ exonuclease or 3′-5′ exonuclease resistance). In some instances, 2′-O-aminopropyl modified nucleic acid molecule is nuclease resistance (e.g., RNase H, DNase, 5′-3′ exonuclease or 3′-5′ exonuclease resistance). In some instances, 2′-deoxy modified nucleic acid molecule is nuclease resistance (e.g., RNase H, DNase, 5′-3′ exonuclease or 3′-5′ exonuclease resistance). In some instances, T-deoxy-2′-fluoro modified nucleic acid molecule is nuclease resistance (e.g., RNase H, DNase, 5′-3′ exonuclease or 3′-5′ exonuclease resistance). In some instances, 2′-O-aminopropyl (2′-O-AP) modified nucleic acid molecule is nuclease resistance (e.g., RNase H, DNase, 5′-3′ exonuclease or 3′-5′ exonuclease resistance). In some instances, 2′-O-dimethylaminoethyl (2′-O-DMAOE) modified nucleic acid molecule is nuclease resistance (e.g., RNase H, DNase, 5′-3′ exonuclease or 3′-5′ exonuclease resistance). In some instances, 2′-O-dimethylaminopropyl (2′-O-DMAP) modified nucleic acid molecule is nuclease resistance (e.g., RNase H, DNase, 5′-3′ exonuclease or 3′-5′ exonuclease resistance). In some instances, T-O-dimethylaminoethyloxyethyl (2′-O-DMAEOE) modified nucleic acid molecule is nuclease resistance (e.g., RNase H, DNase, 5′-3′ exonuclease or 3′-5′ exonuclease resistance). In some instances, 2′-O—N-methylacetamido (2′-O-NMA) modified nucleic acid molecule is nuclease resistance (e.g., RNase H, DNase, 5′-3′ exonuclease or 3′-5′ exonuclease resistance). In some instances, LNA modified nucleic acid molecule is nuclease resistance (e.g., RNase H, DNase, 5′-3′ exonuclease or 3′-5′ exonuclease resistance). In some instances, ENA modified nucleic acid molecule is nuclease resistance (e.g., RNase H, DNase, 5′-3′ exonuclease or 3′-5′ exonuclease resistance). In some instances, HNA modified nucleic acid molecule is nuclease resistance (e.g., RNase H, DNase, 5′-3′ exonuclease or 3′-5′ exonuclease resistance). In some instances, morpholinos is nuclease resistance (e.g., RNase H, DNase, 5′-3′ exonuclease or 3′-5′ exonuclease resistance). In some instances, PNA modified nucleic acid molecule is resistant to nucleases (e.g., RNase H, DNase, 5′-3′ exonuclease or 3′-5′ exonuclease resistance). In some instances, methylphosphonate nucleotides modified nucleic acid molecule is nuclease resistance (e.g., RNase H, DNase, 5′-3′ exonuclease or 3′-5′ exonuclease resistance). In some instances, thiolphosphonate nucleotides modified nucleic acid molecule is nuclease resistance (e.g., RNase H, DNase, 5′-3′ exonuclease or 3′-5′ exonuclease resistance). In some instances, nucleic acid molecule comprising 2′-fluoro N3-P5′-phosphoramidites is nuclease resistance (e.g., RNase H, DNase, 5′-3′ exonuclease or 3′-5′ exonuclease resistance). In some instances, the 5′ conjugates described herein inhibit 5′-3′ exonucleolytic cleavage. In some instances, the 3′ conjugates described herein inhibit 3′-5′ exonucleolytic cleavage.

In additional cases, the modified nucleotide or nucleotide analogue described herein is modified to increase its stability. In some embodiment, the nucleic acid molecule is RNA (e.g., mRNA). In some instances, the mRNA can be modified by one or more of the modifications to increase its stability. In some cases, the mRNA can be modified at the 2′ hydroxyl position, such as by 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modification or by a locked or bridged ribose conformation (e.g., LNA or ENA). In some cases, the at least one modified nucleotide or nucleotide analogue is modified by 2′-O-methyl and/or 2′-O-methoxyethyl ribose. In some cases, the at least one modified nucleotide or nucleotide analogue also includes morpholinos, PNAs, HNA, methylphosphonate nucleotides, thiolphosphonate nucleotides, and/or 2′-fluoro N3-P5′-phosphoramidites to increase its stability. In some instances, the at least one modified nucleotide or nucleotide analogue is a chirally pure (or stereo pure) nucleic acid molecule. In some instances, the chirally pure (or stereo pure) nucleic acid molecule is modified to increase its stability.

In some instances, the extracellular vesicle described herein comprises at least one of any one of the therapeutic polypeptides described herein. In some cases, the at least one therapeutic polypeptide is encoded by the at least one heterologous polynucleotide or vector transfected into the extracellular vesicle donor cell.

In some cases, the therapeutic polynucleotides can be translated by the extracellular vesicle donor cells to obtain at least one therapeutic polypeptide. In some cases, the therapeutic polypeptides encoded by the therapeutic polynucleotides can be encapsulated by the extracellular vesicles produced and secreted by the extracellular vesicle donor cells. In some cases, the extracellular vesicles can encapsulate both therapeutic polynucleotides and therapeutic polypeptides encoded by the nanoelectroporated vectors. In some cases, the extracellular vesicles can be exosomes.

In some instances, the extracellular vesicle described herein can comprise at least one therapeutic compound. In some cases, the at least one therapeutic compound is complexed or anchored by any one of the extracellular vesicle surface proteins described herein. In some cases, the at least one therapeutic compound is within the extracellular vesicle. Exemplary therapeutic compounds for use in the compositions and methods described herein include therapeutic compounds which treat skin damage (e.g., due to aging or sun damage) and blood flow disorders (e.g., ischemia).

Described herein, in some aspects, is a plurality of extracellular vesicles comprising an average of at least one copy of exogenous VEGF mRNA. In some embodiments, the exogenous VEGF mRNA is selected from the group consisting of VEGFA, VEGFB, VEGFC, VEGFD, PIGF, and any combination thereof. In some embodiments, the plurality of extracellular vesicles comprises an extracellular vesicle selected from the group consisting of exosome, microvesicle, apoptotic body, or any combination thereof. In some embodiments, the plurality of extracellular vesicles comprises an extracellular vesicle comprising a targeting polypeptide.

In some aspects, described herein are mixtures of extracellular vesicles, wherein the mixture comprises at least two extracellular vesicles that comprise between one and six copies of exogenous VEGF mRNA, and wherein the mixture comprises exosomes, microvesicles, and apoptotic bodies. In some embodiments, the mixture is formulated for injection via an intravenous, intramuscular, or subcutaneous route. In some embodiments, the mixture is formulated for injection via a coronary artery catheter.

In some aspects, described herein are methods of treating a blood flow disorder in a subject comprising administering at least one extracellular vesicle comprising VEGF mRNA to the subject, thereby treating the blood flow disorder. In some embodiments, treating the blood flow disorder results in at least a 5% increase in revascularization. In some embodiments, the at least 5% increase in revascularization occurs within 14 days of the administering of the at least one extracellular vesicle comprising VEGF mRNA. In some embodiments, administering of the at least one extracellular vesicle comprising VEGF mRNA comprises administering the at least one extracellular vesicle comprising VEGF mRNA to the subject via an intravenous, intramuscular or subcutaneous injection or via a coronary artery catheter. In some embodiments, the intravenous, intramuscular or subcutaneous injection is performed with a needle with a gauge of at least 14 gauge. In some embodiments, the blood flow disorder is ischemia. In some embodiments, the at least one extracellular vesicle comprising VEGF mRNA is administered in at least one dose. In some embodiments, the dose comprises at least 1,000 extracellular vesicles comprising VEGF mRNA. In some embodiments, the at least one extracellular vesicle comprising VEGF mRNA is administered in at least two doses. In some embodiments, administering of the at least one extracellular vesicle comprising VEGF mRNA to the subject comprises administering of the at least one extracellular vesicle comprising VEGF mRNA to a tissue of the subject.

In some aspects, described herein is a plurality of extracellular vesicles comprising an average of at least one copy of exogenous extracellular matrix mRNA. In some embodiments, the exogenous extracellular matrix mRNA is selected from the group consisting of collagen, COLT, collagen type I, collagen type II, collagen type III, collagen type V, collagen type XI, collagen type IX, collagen type XII, collagen type XIV, collagen type VIII, collagen type X, collagen type IV, collagen type VI, collagen type VII, collagen type XIII, collagen type XV, collagen type XVII, and collagen type XVIIII. In some embodiments, the plurality of extracellular vesicles comprises an extracellular vesicle selected from the group consisting of exosome, microvesicle, apoptotic body, or any combination thereof. In some embodiments, the plurality of extracellular vesicles comprises an extracellular vesicle comprising a targeting polypeptide.

In some aspects, described herein are mixtures of extracellular vesicles, wherein the mixture comprises at least two extracellular vesicles that comprise between one and two copies of exogenous extracellular matrix mRNA, and wherein the mixture comprises exosomes, microvesicles, and apoptotic bodies. In some embodiments, the mixture is formulated for injection via an intravenous, intramuscular, or subcutaneous route.

In some aspects, described herein are methods of treating a skin damage in a subject comprising administering at least one extracellular vesicle comprising extracellular matrix mRNA to the subject, thereby treating the skin damage in the subject. In some embodiments, treating the skin damage results in at least a 10% reduction in appearance of wrinkles. In some embodiments, the at least 10% reduction in appearance of wrinkles occurs within 14 days of the administering of the at least one extracellular vesicle comprising extracellular matrix mRNA. In some embodiments, the administering of the at least one extracellular vesicle comprising extracellular matrix mRNA comprises administering the at least one extracellular vesicle comprising extracellular matrix mRNA to the subject via a subcutaneous injection. In some embodiments, subcutaneous injection is performed with a needle with a gauge of at least 14. In some embodiments, the skin damage is caused by aging or sun damage. In some embodiments, the at least one extracellular vesicle comprising extracellular matrix mRNA is administered in at least one dose. In some embodiments, the dose comprises at least 1,000 extracellular vesicles comprising extracellular matrix mRNA. In some embodiments, the at least one extracellular vesicle comprising extracellular matrix mRNA is administered in at least two doses. In some embodiments, administering of the at least one extracellular vesicle comprising extracellular matrix mRNA to the subject comprises administering of the at least one extracellular vesicle comprising VEGF mRNA to a tissue of the subject. In some embodiments, within 72 hours of the administering of the at least one extracellular vesicle comprising extracellular matrix mRNA to the subject, the tissue has a concentration of extracellular matrix protein of at least 6000 μg/ml.

In some aspects, described herein are methods of producing an extracellular vesicle comprising VEGF mRNA comprising: introducing a vector or plasmid encoding VEGF into a donor cell via transfection; incubating the donor cell for a sufficient time for the production of extracellular vesicles encapsulating VEGF mRNA transcribed from the vector or plasmid; and collecting the extracellular vesicles encapsulating the VEGF mRNA transcribed from the vector or plasmid.

In some aspects, described herein are methods of producing an extracellular vesicle comprising extracellular matrix mRNA comprising: introducing a vector or plasmid encoding an extracellular matrix protein into a donor cell via transfection; incubating the donor cell for a sufficient time for the production of extracellular vesicles encapsulating extracellular matrix mRNA transcribed from the vector or plasmid; and collecting the extracellular vesicles encapsulating the extracellular matrix mRNA transcribed from the vector or plasmid.

Described herein, in some aspects, is a plurality of extracellular vesicles comprising an exogenous extracellular matrix messenger RNA (mRNA). In some cases, the exogenous extracellular matrix mRNA encodes a collagen. In some cases, the collagen is selected from the group consisting of Collagen type I, Collagen type II, Collagen type III, Collagen type IV, Collagen type V, Collagen type VI, Collagen type VII, Collagen type VIII, Collagen type IX, Collagen type X, Collagen type XI, Collagen type XII, Collagen type XIII, Collagen type XIV, Collagen type XV, Collagen type XVI, Collagen type XVII, Collagen type XVIII, Collagen type XIX, Collagen type XX, Collagen type XXI, Collagen type XXII, Collagen type XXIII, Collagen type XXIV, Collagen type XXV, Collagen type XXVI, Collagen type XXVII, Collagen type XXVIII, or any combination thereof. In some cases, the collagen is collagen type I. In some cases, the collagen is collagen type II. In some cases, the collagen is alpha 1 chain of collagen type I (Col1A1). In some cases, the collagen is Col1A1, and the plurality of extracellular vesicles do not comprise an alpha 2 chain of collagen type I (Col1A2). In some cases, the collagen is Col1A2. In some cases, the exogenous extracellular matrix mRNA is the mRNA encoding pro-alpha1(I) chain.

In some cases, the plurality of extracellular vesicles comprises an average of at least one copy of the exogenous extracellular matrix mRNA per 450 EVs. In some cases, the plurality of extracellular vesicles comprises an average of at least one copy of the exogenous extracellular matrix mRNA per 200 EVs. In some cases, the plurality of extracellular vesicles comprises an average of at least one copy of the exogenous extracellular matrix mRNA per about 0.1 to 100 EVs. In some cases, the plurality of extracellular vesicles comprises an average of at least one copy of the exogenous extracellular matrix mRNA per 2000 EVs, per 1000 EVs, per 500 EVs, per 400 EVs, per 300 EVs, per 200 EVs, per 150 EVs, per 100 EVs, per 90 EVs, per 80 EVs, per 70 EVs, 60 EVs, 50 EVs, per 40 EVs, per 30 EVs, per 20 EVs, per 15 EVs, per 10 EVs, per 5 EVs, per 2 EVs, or per 1 EV. In some cases, the plurality of extracellular vesicles comprise an average of at least 1, 2, 5, 10, 50, 100, 200, 300, 500, or 1000 copies of the exogenous extracellular matrix mRNA per EV. In some cases, the plurality of extracellular vesicles comprise an average of about 1 to 10 copies of the exogenous extracellular matrix mRNA per EV. In some cases, the plurality of extracellular vesicles comprise an average of about 1 copy of the exogenous extracellular matrix mRNA per EV. In some cases, the plurality of extracellular vesicles comprise an average of at least one copy of the exogenous extracellular matrix mRNA per 30 EVs.

In some cases, the plurality of extracellular vesicles are produced by transfecting a cell with a plasmid encoding an extracellular matrix protein. In some cases, the transfecting of the cell is performed by cellular nanoporation. In some cases, the transfecting of the cell is performed by electroporation. In some cases, the cell is a human cell. In some cases, the cell is a fibroblast selected from the group consisting of dermal fibroblast, human fibroblast, adult fibroblast, human adult fibroblast, neonatal fibroblast, neonatal human fibroblast, and neonatal human dermal fibroblast. In some cases, the cell is an adherent cell. In some cases, the cell is a human dermal fibroblast (e.g., neonatal human dermal fibroblast).

In some cases, the plurality of extracellular vesicles comprises an extracellular vesicle selected from the group consisting of exosome, microvesicle, apoptotic body, and any combination thereof. In some cases, the plurality of extracellular vesicles comprises at least one exosome. In some cases, the plurality of extracellular vesicles comprises at least one apoptotic body. In some cases, the plurality of extracellular vesicles comprises at least one microvesicle. In some cases, the plurality of extracellular vesicles comprises a mixture of at least any two of the following: exosome, microvesicle or apoptotic body.

In some cases, the plurality of extracellular vesicles are formulated for injection via an intravenous route, an intramuscular route, a subcutaneous route, or any combination thereof. In some cases, the formulation comprises one or more stabilizing agents and/or one or more preservatives. In some cases, the formulation comprises one or more DNAses and/or RNase inhibitors. In some cases, the formulation comprises one or more DNAses, DNAse inhibitors, RNAse, and/or RNAse inhibitors.

In some cases, the plurality of extracellular vesicles comprises at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles. In some cases, the plurality of extracellular vesicles comprises at least 1×10¹³ extracellular vesicles. In some cases, the plurality of extracellular vesicles comprises at 1×10¹⁴ extracellular vesicles. In some cases, the plurality of extracellular vesicles comprises at least 1×10⁹ extracellular vesicles and at most 1×10²⁰ extracellular vesicles. In some cases, the plurality of extracellular vesicles comprises at most 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹, 1×10²⁰, 1×10²⁵, or 1×10³⁰ extracellular vesicles. In some cases, the plurality of extracellular vesicles comprises at least 1×10⁶ extracellular vesicles and at most 1×10³⁰ extracellular vesicles. In some cases, the plurality of extracellular vesicles comprises at least 1 ng, 10 ng, 20 ng, 40 ng, 50 ng, 100 ng, 500 ng, 1 μg, 10 μg, 20 μg, 30 μg, 40 μg, or 50 μg of the extracellular matrix mRNA. In some cases, the plurality of extracellular vesicles comprises at most 1 ng, 10 ng, 20 ng, 40 ng, 50 ng, 100 ng, 500 ng, 1 μg, 10 μg, 20 μg, 30 μg, 40 μg, or 50 μg of the extracellular matrix mRNA. In some cases, the plurality of extracellular vesicles comprises at least 1 ng and at most 20 μg of the extracellular matrix mRNA. In some cases, the plurality of extracellular vesicles comprises at least 5 ng and at most 30 μg of the extracellular matrix mRNA.

In some cases, the plurality of extracellular vesicles comprises an extracellular vesicle comprising a targeting polypeptide. In some cases, the targeting polypeptide targets dermal cells. In some cases, the targeting polypeptide targets aminopeptidase N, CD26/DPP4, fibroblast activation protein α, or any combination thereof. In some cases, the plurality of extracellular vesicles does not comprise one or more of the following miRNAs: hsa-miR-29c-3p, hsa-miR-29a-3p, hsa-miR-378a-3p, hsa-miR-125b-5p, hsa-miR-23a-3p, hsa-miR-449a, hsa-miR-196a-5p, hsa-miR-744-5p, hsa-miR-223-3p, hsa-miR-23a-3p, hsa-miR-133a-3p, hsa-miR-223-3p, hsa-miR-5011-5p, hsa-miR-325, or hsa-miR-199b-5p.

In some cases, the plurality of extracellular vesicles comprises a size distribution with a peak at 50 nm-200 nm in diameter. In some cases, the plurality of extracellular vesicles comprises a size distribution with a peak at about 100 nm in diameter. In some cases, the plurality of extracellular vesicles comprises a size distribution with a peak at about 75-130 nm in diameter. In some cases, the extracellular vesicles are greater than 20 nm, 30 nm, 50 nm, 75 nm, or 100 nm in diameter. In some cases, at least 90% of the extracellular vesicles are greater than 20 nm, 30 nm, 50 nm, 75 nm, or 100 nm in diameter. In some cases, the extracellular vesicles do not comprise extracellular vesicles less than 50 nm in diameter. In some cases, the extracellular vesicles do not comprise extracellular vesicles less than 30 nm in diameter. In some cases, the extracellular vesicles do not comprise extracellular vesicles less than 20 nm in diameter. In some cases, the extracellular vesicles do not comprise extracellular vesicles less than 10 nm in diameter. In some cases, the extracellular vesicles do not comprise extracellular vesicles less than 5 nm in diameter.

In some cases, the exogenous extracellular matrix mRNA is present at a level that is at least 2-fold higher than a level of the exogenous extracellular matrix mRNA in an identical amount of naturally-occurring extracellular vesicles. In some cases, the exogenous extracellular matrix mRNA is present at a level that is at least 3-fold higher than a level of the exogenous extracellular matrix mRNA in an identical amount of naturally-occurring extracellular vesicles. In some cases, the exogenous extracellular matrix mRNA is present at a level that is at least 1.5 fold, at least 2-fold, at least 3-fold, at least 4-fold, at least 5-fold, at least 6-fold, at least 7-fold, at least 10-fold, at least 15-fold, at least 20-fold, at least 50-fold, at least 75-fold, at least 100-fold, at least 500-fold, at least 1000-fold, at least 1500-fold, or at least 2000-fold higher than a level of the exogenous extracellular matrix mRNA in an identical amount of naturally-occurring extracellular vesicles. In some cases, the exogenous extracellular matrix mRNA is present at a level that is about 3000-fold higher than a level of the exogenous extracellular matrix mRNA in an identical amount of naturally-occurring extracellular vesicles. In some cases, the exogenous extracellular matrix mRNA is present at a level that is about 2000-fold higher than a level of the exogenous extracellular matrix mRNA in an identical amount of naturally-occurring extracellular vesicles. In some cases, the plurality of extracellular vesicles expresses higher levels of at last one of the following markers compared to a comparable number of naturally-occurring extracellular vesicle: CD9, CD63, TSG101, or ARF6.

Described herein, in some aspect, is a vessel comprising the plurality of extracellular vesicles of any one of the preceding claims, wherein the vessel further comprises a cell. In some cases, the cell a comprises a human cell, a human fibroblast cell, a fibroblast cell, a dermal fibroblast, a human fibroblast, an adult fibroblast, a human adult fibroblast, a neonatal fibroblast, a neonatal human fibroblast, a neonatal human dermal fibroblast, or any combination thereof. In some cases, the plurality of extracellular vesicles are present in the vessel at a ratio of at least 1000, 2000, 5000, 10000, or 12000 extracellular vesicles per cell.

Described herein, in some aspects, is a needle comprising the plurality of extracellular vesicles disclosed herein. In some cases, the needle is a microneedle. In some cases, the needle is solid. In some cases, the needle is a hydrogel needle. In some cases, at least 50%, 70%, 75%, 80%, 85%, 90%, 95%, or 99% of the needle comprises hydrogel. In some cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In some cases, the hydrogel comprises hyaluronic acid. In some cases, the hydrogel comprises at least 1%, 5%, 7%, 10%, 12%, 15%, 20%, 25% 50%, 70%, 75%, 80%, 85%, 90%, 95%, or 99% hyaluronic acid. In some cases, the hydrogel comprises at most 1%, 5%, 7%, 10%, 12%, 15%, 20%, 25% 50%, 70%, 75%, 80%, 85%, 90%, 95%, or 99% hyaluronic acid. In some cases, the hydrogel comprises at least 5% hyaluronic acid. In some cases, the hydrogel comprises about 15% hyaluronic acid. In some cases, the hydrogel comprises at least 5% hyaluronic acid and at most 30% hyaluronic acid. In some cases, the hydrogel comprises greater than 10% hyaluronic acid and at most 20% hyaluronic acid. In some cases, the hydrogel comprises greater than 10% hyaluronic acid and at most 15% hyaluronic acid. In some cases, the hydrogel comprises greater than 10% hyaluronic acid and at most 18% hyaluronic acid.

Described herein, in some aspects, is a syringe comprising the plurality of extracellular vesicles disclosed herein. In some cases, the plurality of extracellular vesicles are suspended in hyaluronic acid within the syringe.

Described herein, in some aspects, is a hydrogel microneedle, wherein the hydrogel microneedle comprises a plurality of extracellular vesicles. In some cases, the hydrogel comprises at least 1%, 5%, 7%, 10%1, 12%, 15%, 20%, 25% 50%, 70%, 75%, 80%, 85%, 90%, 95%, or 99% hyaluronic acid. In some cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In some cases, the hydrogel comprises hyaluronic acid. In some cases, the hydrogel comprises at least 5%, 7%, 10%, 15%, or 20% hyaluronic acid. In some cases, the plurality of extracellular vesicles comprises an extracellular vesicle selected from the group consisting of exosome, microvesicle, apoptotic body, and any combination thereof. In some cases, the plurality of extracellular vesicles comprise at least one exosome. In some cases, the plurality of extracellular vesicles comprise at least one apoptotic body. In some cases, the plurality of extracellular vesicles comprise at least one microvesicle. In some cases, the plurality of extracellular vesicles comprises at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles. In some cases, the plurality of extracellular vesicles comprises no great than 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, 1×10³⁰, 1×10³⁰, 1×10⁴⁰, 1×10⁵⁰, 1×10⁶⁰, 1×10⁷⁰, 1×10⁸⁰, 1×10⁹⁰, or 1×10¹⁰⁰ extracellular vesicles. In some cases, the plurality of extracellular vesicles comprises about 1×10⁶ to 1×10³⁰ extracellular vesicles. In some cases, the plurality of extracellular vesicles comprises about 1×10¹⁰ to 1×10¹⁴ extracellular vesicles. In some cases, the plurality of extracellular vesicles comprises about 1×10¹³ to 1×10¹⁴ extracellular vesicles.

In some cases, the hydrogel microneedle has a length less than 2 mm. In some cases, the hydrogel microneedle has a length less than 1 mm. In some cases, the hydrogel microneedle has a length of at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000 μm. In some cases, the hydrogel microneedle has a length no greater than 100, no greater than 200, no greater than 300, no greater than 400, no greater than 500, no greater than 600, no greater than 700, no greater than 800, no greater than 900, no greater than 1000, no greater than 1100, no greater than 1200, no greater than 1300, no greater than 1400, no greater than 1500, no greater than 1600, no greater than 1700, no greater than 1800, no greater than 1900, or no greater than 2000 μm. In other cases, the hydrogel microneedle has a length from 1000 μm to 3000 μm, from 500 μm to 2500 μm, from 1000 μm to 2500 μm, or from 1000 μm to 2000 μm. In specific embodiments, the hydrogel microneedle has a length of about 1000 μm. In specific embodiments, the hydrogel microneedle has a length of about 2000 μm.

In some cases, the hydrogel microneedle has a base with a diameter of no greater than 10000 μm, 9000 μm, 8000 μm, 7000 μm, 6000 μm, 5000 μm, 4000 μm, 3000 μm, 2000 μm, less than 1800 μm, less than 1500 μm, less than 1000 μm, less than 900 μm, less than 800 μm less than 700 μm, less than 600 μm, less than 500 μm, less than 400 μm, less than 300 μm, less than 200 μm, or less than 100 μm. In some cases, the hydrogel microneedle comprises extracellular vesicles loaded with one or more mRNA cargos.

Described herein, in some aspects, is a needle comprising a plurality of extracellular vesicles wherein the extracellular vesicles comprise at least one extracellular matrix messenger RNA (mRNA). In some cases, the extracellular matrix mRNA encodes a collagen. In some cases, the collagen is selected from the group consisting of collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or any combination thereof. In some cases, the collagen is collagen type I. In some cases, the collagen is collagen type II. In some cases, the collagen is alpha 1 chain of collagen type I (Col1A1). In some cases, the collagen is Col1A1, and the plurality of extracellular vesicles do not comprise an alpha 2 chain of collagen type I (Col1A2). In some cases, the collagen is Col1A2. In some cases, the extracellular matrix mRNA is the mRNA encoding pro-alpha 1(I) chain.

In some cases, the plurality of extracellular vesicles comprises an extracellular vesicle selected from the group consisting of exosome, microvesicle, apoptotic body, and any combination thereof. In some cases, the plurality of extracellular vesicles comprise at least one exosome. In some cases, the plurality of extracellular vesicles comprise at least one apoptotic body. In some cases, the plurality of extracellular vesicles comprise at least one microvesicle. In some cases, the plurality of extracellular vesicles comprise a mixture of any two of the following: exosome, microvesicle or apoptotic body.

In some cases, the needle is a microneedle. In some cases, the needle is solid. In some cases, the needle is a hydrogel needle, and optionally wherein at least 50%, 70%, 75%, 80%, 85%, 90%, 95%, or 99% of the needle comprises hydrogel. In some cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In some cases, the hydrogel comprises hyaluronic acid. In some cases, the hydrogel comprises at least 1%, 5%, 7%, 10%, 12%, 15%, 20%, 25% 50%, 70%, 75%, 80%, 85%, 90%, 95%, or 99% hyaluronic acid. In some cases, the hydrogel comprises at least 5% hyaluronic acid. In some cases, the hydrogel comprises about 15% hyaluronic acid. In some cases, the hydrogel comprises at least 5% hyaluronic acid and at most 30% hyaluronic acid. In some cases, the hydrogel comprises greater than 10% hyaluronic acid and at most 20% hyaluronic acid. In some cases, the hydrogel comprises greater than 10% hyaluronic acid and at most 15% hyaluronic acid. In some cases, the hydrogel comprises greater than 10% hyaluronic acid and at most 18% hyaluronic acid.

In some cases, the needle has a length less than 2 mm. In some cases, the needle has a length less than 1 mm. In some cases, the needle has a length of at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000 μm. In some cases, the needle has a length no greater than 100, no greater than 200, no greater than 300, no greater than 400, no greater than 500, no greater than 600, no greater than 700, no greater than 800, no greater than 900, no greater than 1000, no greater than 1100, no greater than 1200, no greater than 1300, no greater than 1400, no greater than 1500, no greater than 1600, no greater than 1700, no greater than 1800, no greater than 1900, or no greater than 2000 μm. In other cases, the needle has a length from 100 μm to 3000 μm, from 500 μm to 2500 μm, from 1000 μm to 2500 μm, or from 1000 μm to 2000 μm. In specific embodiments, the needle has a length of about 1000 μm. In specific embodiments, the needle has a length of about 2000 μm.

In some cases, the needle has a base with a diameter of no greater than 10000 μm, 9000 μm, 8000 μm, 7000 μm, 6000 μm, 5000 μm, 4000 μm, 3000 μm, 2000 μm, less than 1800 μm, less than 1500 μm, less than 1000 μm, less than 900 μm, less than 800 μm less than 700 μm, less than 600 μm, less than 500 μm, less than 400 μm, less than 300 μm, less than 200 μm, or less than 100 μm.

The COL1A1 gene produces a component of type I collagen, called the pro-alpha1(I) chain. This chain combines with another pro-alpha1(I) chain and also with a pro-alpha2(I) chain (produced by the COL1A2 gene) to make a molecule of type I procollagen. These triple-stranded, rope-like procollagen molecules can be processed by enzymes. Once these molecules are processed, they arrange themselves into long, thin fibrils that cross-link to one another in the spaces around cells. The cross-links result in the formation of very strong mature type I collagen fibers. Accordingly, in some cases, the at least one copy of exogenous extracellular matrix mRNA is transcribed and is assembled into a pro-alpha1(I) chain in the cells being exposed to the plurality of extracellular vesicles. In some cases, the at least one copy of exogenous extracellular matrix mRNA is transcribed and is further assembled into a type I procollagen in the cells being exposed to the plurality of extracellular vesicles.

In some cases, the plurality of extracellular vesicles comprise an average of at least 10, 100, 1000, 2000, 3000, 4000, 5000, 6000, 10,000, 15,000, 30,000, 50,000, 80,000, 100,000, 150,000, 300,000, 500,000, 700,000, 1,000,000 copies of the exogenous extracellular matrix mRNA per extracellular vesicle. In some cases, the plurality of extracellular vesicles comprise an average of about 10, 100, 1000, 2000, 3000, 4000, 5000, 6000, 10,000, 15,000, 30,000, 50,000, 80,000, 100,000, 150,000, 300,000, 500,000, 700,000, 1,000,000 copies of the exogenous extracellular matrix mRNA per extracellular vesicle.

In some cases, the extracellular vesicles disclosed herein are exosomes. In other cases, the extracellular vesicles disclosed herein are microvesicles. In other cases, the extracellular vesicles disclosed herein are apoptotic bodies. In other cases, the extracellular vesicles disclosed herein are a combination of the any two or three types mentioned in this paragraph.

In some cases, the plurality of extracellular vesicles comprises an extracellular vesicle comprising a targeting polypeptide as disclosed herein.

Further provided herein are needles comprising the plurality of extracellular vesicles comprising an average of at least one copy of exogenous extracellular matrix mRNA. Further provided herein are needles comprising the plurality of extracellular vesicles comprising an average of at least one copy of endogenous extracellular matrix mRNA. Still further provided herein are needles comprising the plurality of extracellular vesicles comprising an average of at least one copy of endogenous and at least one copy of exogenous extracellular matrix mRNA.

In some cases, the needle is a microneedle. In some cases, the needle comprises hydrogel. In specific cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In specific cases, the hydrogel comprises hyaluronic acid.

Further provided herein are syringes comprising the plurality of extracellular vesicles comprising an average of at least one copy of exogenous extracellular matrix mRNA. Further provided herein are syringes comprising the plurality of extracellular vesicles comprising an average of at least one copy of endogenous extracellular matrix mRNA. Still further provided herein are syringes comprising the plurality of extracellular vesicles comprising an average of at least one copy of endogenous and at least one copy of exogenous extracellular matrix mRNA.

In some cases, the syringe has a gauge of at least 34. In some cases, the syringe has a gauge of less than 10. In some cases, the syringe has a gauge of 34, 33, 32, 31, 30, 29, 28, 27, 26 s, 26, 25 s, 25, 24, 23 s, 23, 22 s, 22, 21, 20, 19, 18, 17, 16, 15, 14, 13, 12, 11, or 10.

In some cases, the plurality of extracellular vesicles in the syringe are suspended in hyaluronic acid.

Further provided herein are mixtures of extracellular vesicles, wherein the mixture comprises at least two extracellular vesicles that comprise one or two copies of exogenous extracellular matrix mRNA. In some cases, the mixture comprises exosomes, microvesicles, apoptotic bodies, or any combination thereof.

In some cases, the mixture disclosed herein is formulated for injection via an intravenous, intramuscular, or subcutaneous route.

Further provided herein are methods of producing the extracellular vesicles comprising extracellular matrix mRNA wherein the producing the extracellular vesicles comprising extracellular matrix mRNA comprises: (a) introducing a vector or plasmid encoding an extracellular matrix protein into a donor cell via transfection; (b) incubating the donor cell for a sufficient time for the production of extracellular vesicles encapsulating extracellular matrix mRNA transcribed from the vector or plasmid; and (c) collecting the extracellular vesicles encapsulating the extracellular matrix mRNA transcribed from the vector or plasmid.

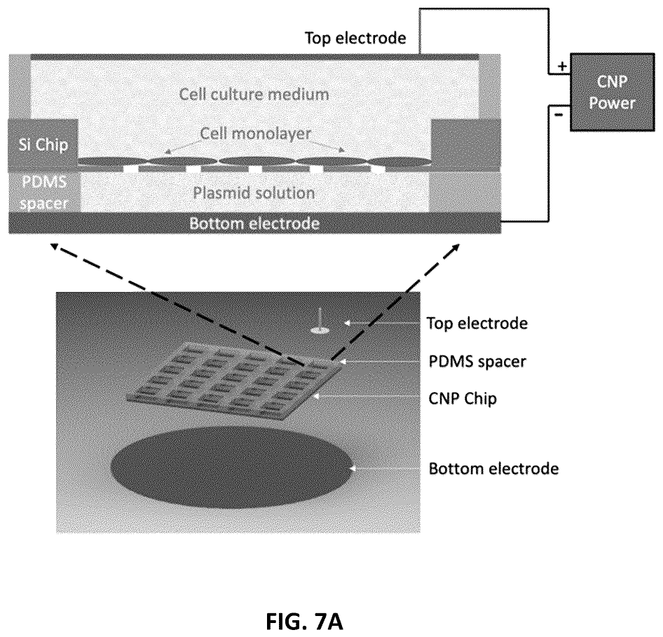

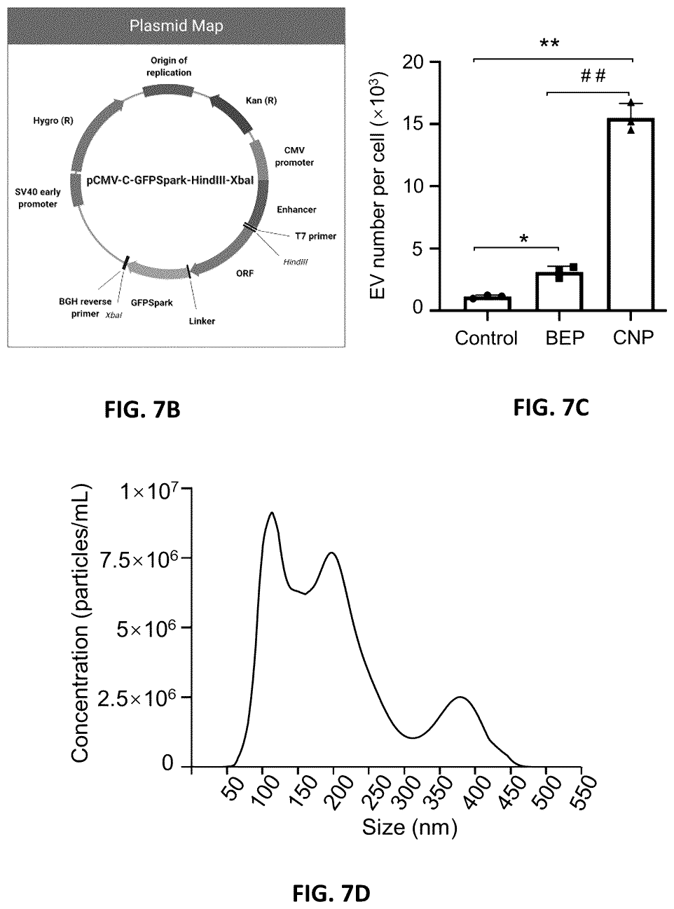

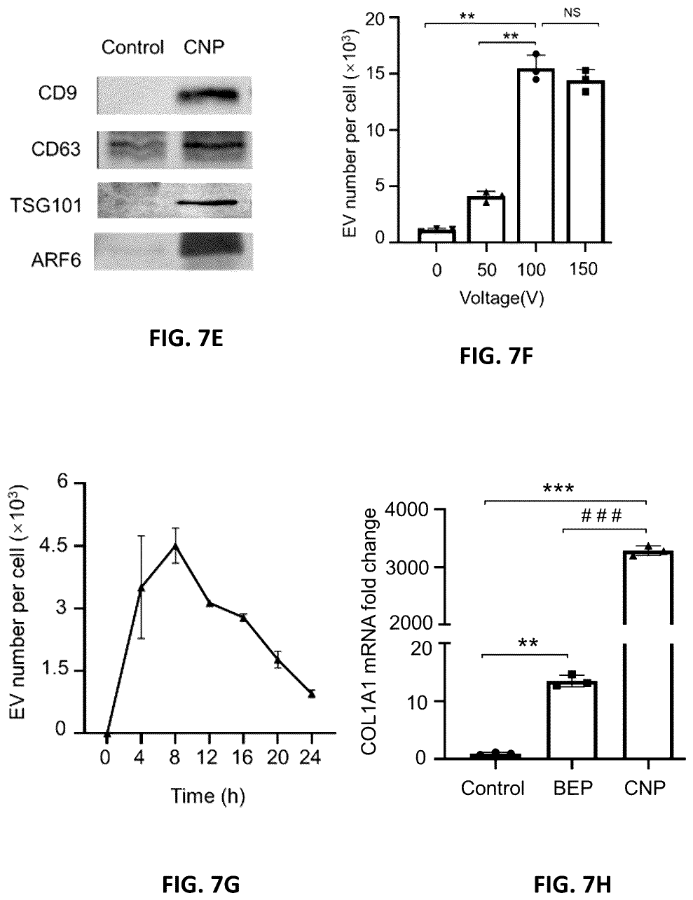

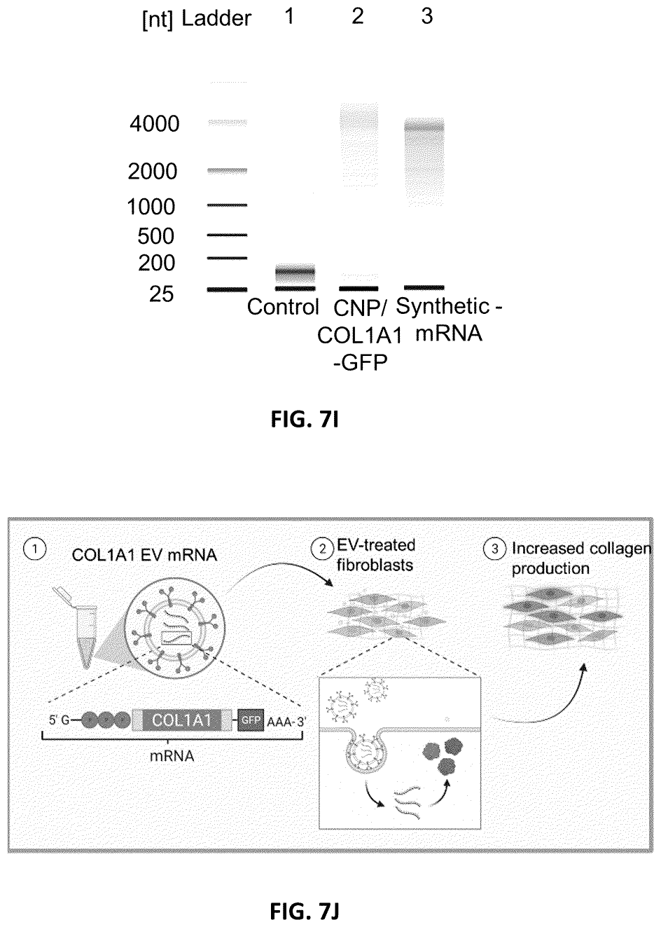

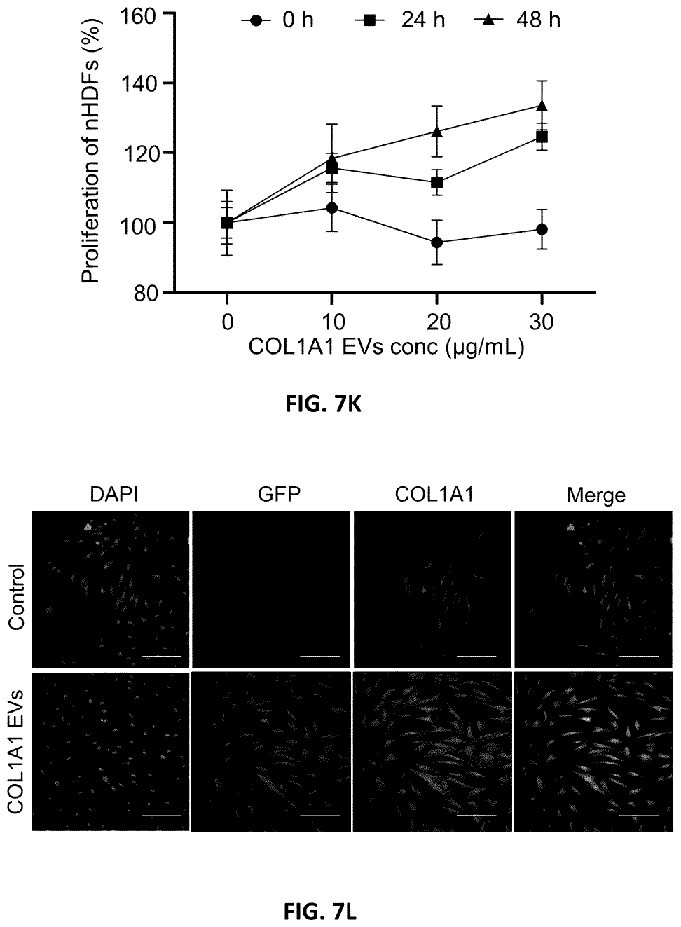

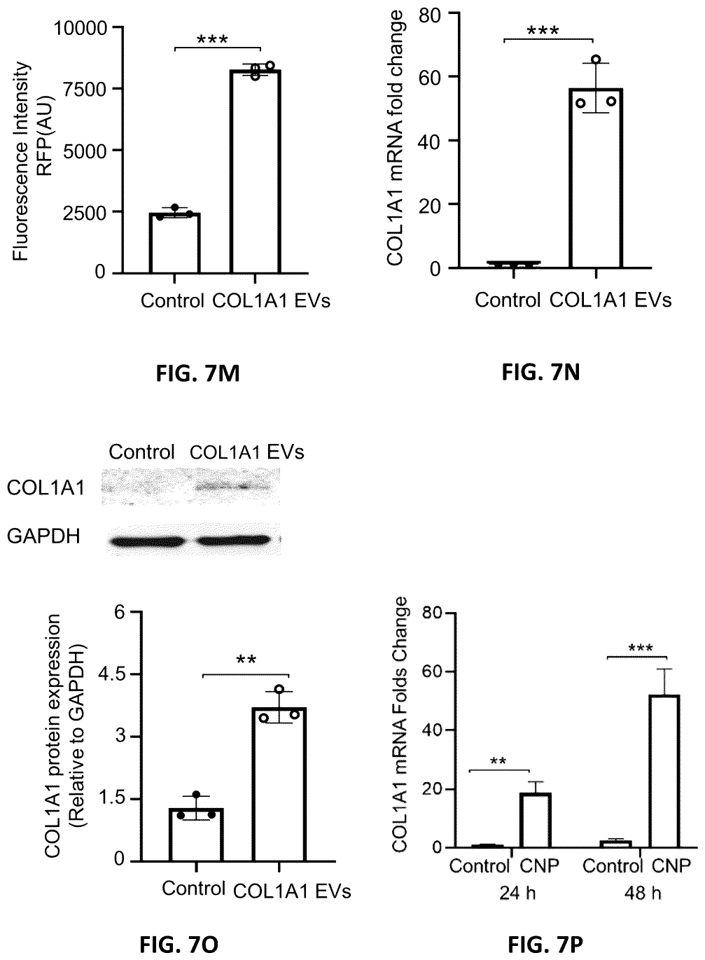

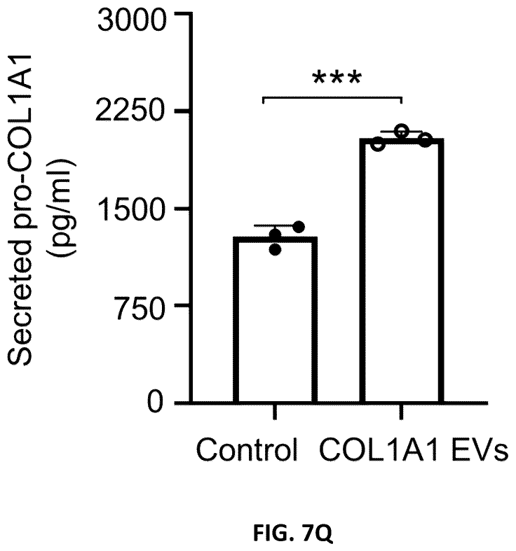

In some cases, in step (a) the transfection is performed with cellular electroporation as shown in FIG. 7A. In some specific cases, in step (a) the transfection comprises seeding a single layer of the donor cells on to the surface of the silicon chip shown in FIG. 7A, and certain voltage of electric field with certain pulses and durations is applied for electroporating the vector or the plasmid into the donor cell. In some further specific cases, the seeding takes overnight incubation as previously described. In some further specific cases, the voltage of the electric field is at least 10V, 50V, 100V, 150V, 200V, 250V, 300V, 500V, or 1000V. In some further specific cases, the voltage of the electric field is about 10V, 50V, 100V, 150V, 200V, 250V, 300V, 500V, or 1000V. In some further specific cases, the voltage of the electric field is at most 500V, 600V, 700V, 800V, 900V, 1000V, 1500V, 2000V, 5000V, or 10,000V. In some further specific cases, the pulses of the electric field is at least 5, 10, 15, 20, 25, 30, 40, 50, or 60. In some further specific cases, the pulses of the electric field is no greater than 5, 10, 15, 20, 25, 30, 40, 50, 60, 70, 80, 90, 100, 110, 120, 130, 140, 150, 160, 170, 180, 190, or 200. In some further specific cases, the pulses of the electric field is about 5, 10, 15, 20, 25, 30, 40, 50, or 60. In some further specific cases, the interval between pulses of the electric field is at least 0.1 s, 0.2 s, 0.3 s, 0.4 s, 0.5 s, 0.6 s, 0.7 s, 0.8 s, 0.9 s, or Is. In some further specific cases, the interval between pulses of the electric field is about 0.1 s, 0.2 s, 0.3 s, 0.4 s, 0.5 s, 0.6 s, 0.7 s, 0.8 s, 0.9 s, or 1 s.

In some cases, in step (c) the collecting the extracellular vesicles occurs about 1, 2, 3, 4, 5, 6, 7, 8, 9, 10, 11, 12, 13, 14, 15, 16, 17, 18, 19, 20, 21, 22, 23, or 24 hr after the transfection. In some cases, in step (c) the collecting the extracellular vesicles occurs at least 1, 2, 3, 4, 5, 6, 7, 8, 9, 10, 11, 12, 13, 14, 15, 16, 17, 18, 19, 20, 21, 22, 23, or 24 hr after the transfection.

Described herein, in some aspect, is a method of manufacturing a microneedle device, wherein the method comprises: (a) mixing extracellular vesicles (EVs) with a first batch of polymerizable solution; and (b) casting the mixture from (a) to a polydimethylsiloxane (PDMS) mold with at least one needle-like shape. In some cases, the mixing in (a) is performed under vacuum.

In some cases, the method further concentrating the EVs in a tip of the at least one needle-like shape of the PDMS mold. In some cases, the concentrating is by maintaining the PDMS mold at a temperature of at most 10° C. In some cases, the concentrating is by maintaining the PDMS mold at about 4° C. In some cases, the concentrating lasts from about 2 to about 6 hours.

In some cases, the method further comprises adding a second batch of the polymerizable solution on top of the PDMS mold. In some cases, the polymerizable solution comprises hydrogel. In some cases, the hydrogel comprises hyaluronic acid, sodium alginate, polylactic acid, polyglycolic acid, and polylactic-glycolic acid, cartilage thioflavin, silk protein, maltose, chitosan, carboxymethyl cellulose, or any combination thereof. In some cases, the hydrogel comprises hyaluronic acid. In some cases, the hydrogel comprises at least 5% hyaluronic acid. In some cases, the hydrogel comprises at least 5% hyaluronic acid. In some cases, the hydrogel comprises about 15% hyaluronic acid. In some cases, the hydrogel comprises at least 5% hyaluronic acid and at most 30% hyaluronic acid. In some cases, the hydrogel comprises greater than 10% hyaluronic acid and at most 20% hyaluronic acid. In some cases, the hydrogel comprises greater than 10% hyaluronic acid and at most 15% hyaluronic acid. In some cases, the hydrogel comprises greater than 10% hyaluronic acid and at most 18% hyaluronic acid.

In some cases, the final ratio between EVs and the polymerizable solution is at least 1:2, 1:3, 1:4, 1:5, 1:10, 1:15, 1:20, 1:25, 1:30, 1:35, 1:40, 1:45, 1:50, 1:55, 1:60, 1:65, 1:70, 1:80, 1:90, or 1:100. In some cases, the final ratio between EVs and the polymerizable solution is no greater than 1:2, 1:3, 1:4, 1:5, 1:10, 1:15, 1:20, 1:25, 1:30, 1:35, 1:40, 1:45, or 1:50. In some cases, the final ratio between EVs and the polymerizable solution is about 1:5, 1:10, 1:15, 1:20, 1:25, 1:30, 1:35, or 1:40.

In some cases, the EVs comprise exogenous extracellular matrix mRNA. In some cases, the exogenous extracellular matrix mRNA comprises collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or any combination thereof. In some cases, the EVs comprise exogenous VEGF mRNA. In some cases, the exogenous VEGF mRNA comprises VEGFA, VEGFB, VEGFC, VEGFD, PIGF, or any combination thereof.

Described herein, in some aspect, is a method of producing a heterodimer or a heterotrimer of collagen type I in a tissue, wherein the method comprises administering to the tissue the plurality of extracellular vesicles disclosed herein. In some cases, the heterodimer or the heterotrimer comprises at least one alpha chain of collagen type I (Col1A1) and at least one alpha chain of collagen type I (Col1A2), and wherein the Col1A1 is exogenously delivered by the plurality of extracellular vesicles disclosed herein. In some cases, the Col1A2 is endogenous to the tissue.

Described herein, in some aspect, is a plurality of extracellular vesicles comprising an average of at least one copy of exogenous VEGF mRNA per 400 extracellular vesicles. In some cases, the exogenous VEGF mRNA is selected from the group consisting of VEGFA, VEGFB, VEGFC, VEGFD, PIGF, and any combination thereof. In some cases, the plurality of extracellular vesicles comprises an extracellular vesicle selected from the group consisting of exosome, microvesicle, apoptotic body, or any combination thereof. In some cases, the plurality of extracellular vesicles comprises an extracellular vesicle comprising a targeting polypeptide.

In some cases, the plurality of extracellular vesicles comprise at least one copy of exogenous VEGF mRNA per at most 1, 5, 10, 15, 20, 25, 30, 40, 45, 50, 60, 70, 80, 90, 100, 150, 200, 250, 300, or 350 extracellular vesicles. In some cases, the plurality of extracellular vesicles comprise at least 1, 2, 5, 10, 20, 25, 30, 35, 30, 50, 60, 70 90 or 100 copies of VEGF mRNA per extracellular vesicle. In some cases, the plurality of extracellular vesicles comprise at least 1, 10, 20, 30, 40, 50, 60, 70, 80, 90, or 100 copies of VEGF mRNA per extracellular vesicle. In some cases, the plurality of extracellular vesicles comprise at most 1, 10, 20, 30, 40, 50, 60, 70, 80, 90, 100, 150, 200, 250, 300, 350, 400, 450, 500, 600, 700, 800, 900, or 1000 copies of VEGF mRNA per extracellular vesicle. In some cases, the plurality of extracellular vesicles comprise at least one copy of exogenous VEGF mRNA per about 0.001 to 100, 0.01 to 100, or 0.01 to 50 extracellular vesicles. In some cases, the plurality of extracellular vesicles comprise at least one copy of exogenous VEGF mRNA per about 0.02 to 50 extracellular vesicles.

In some cases, the plurality of extracellular vesicles comprise at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles. In some cases, the plurality of extracellular vesicles comprise at most 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles.

In some cases, the plurality of extracellular vesicles are formulated for intravenous injection, intramuscular injection, subcutaneous injection, or injection via a coronary artery catheter.

In some cases, the mixture comprises at least two extracellular vesicles that comprise between one and six copies of exogenous VEGF mRNA, and wherein the mixture comprises exosomes, microvesicles, and apoptotic bodies. In some cases, said mixture is formulated for injection via an intravenous, intramuscular, or subcutaneous route. In some cases, said mixture is formulated for injection via a coronary artery catheter. In some cases, VEGF mRNA is present at a level that is at least 2-fold, at least 3-fold, at least 4-fold, at least 5-fold, at least 6-fold, at least 7-fold, at least 10-fold, at least 15-fold, at least 20-fold, at least 50-fold, at least 75-fold, at least 100-fold, at least 500-fold, at least 1000-fold, at least 1500-fold, or at least 2000-fold higher than a level of VEGF mRNA in an identical amount of naturally-occurring extracellular vesicles. In some cases, the plurality of extracellular vesicles comprise at least 10 μg, 50 μg, 100 μg, 200 μg, 300 μg, 400 μg, 500 μg, 600 μg, 700 μg, 800 μg, 900 μg, 1 ng, 100 ng, 500 ng, 1000 ng, 10 μg, 50 μg, 100 μg, 150 μg, or 200 μg of VEGF mRNA. In some cases, the plurality of extracellular vesicles comprise no greater than 10 μg, 50 μg, 100 μg, 200 μg, 300 μg, 400 μg, 500 μg, 600 μg, 700 μg, 800 μg, 900 μg, 1 ng, 100 ng, 500 ng, 1000 ng, 10 μg, 50 μg, 100 μg, 150 μg, 200 μg, 300 μg, 400 μg, 500 μg, 600 μg, 700 μg, 800 μg, 900 μg, or 1000 μg of VEGF mRNA. In some cases, the plurality of extracellular vesicles comprise about 1 ng to 200 μg VEGF mRNA.

Treatment with Extracellular Vesicles

Described herein, in some aspects, is a method of treating a skin condition in a subject comprising administering at least one extracellular matrix mRNA to a subject in need thereof, thereby treating the skin condition. In some cases, the exogenous extracellular matrix mRNA encodes a collagen. In some cases, the collagen is selected from the group consisting of collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or any combination thereof. In some cases, the collagen is collagen type I. In some cases, the collagen is collagen type II. In some cases, the collagen is alpha 1 chain of collagen type I (Col1A1). In some cases, the collagen is Col1A1, but not an alpha 2 chain of collagen type I (Col1A2). In some cases, the collagen is Col1A2. In some cases, the extracellular matrix mRNA is the mRNA encoding pro-alpha1(I) chain.

In some cases, the skin condition is skin damage. In some cases, the skin damage is caused by aging or sun damage. In some cases, the skin condition is a wound.

In some cases, the administering comprises administering at least 1 ng, 10 ng, 20 ng, 40 ng, 50 ng, 100 ng, 500 ng, 1 μg, 10 μg, 20 μg, 30 μg, 40 μg, or 50 μg of the extracellular matrix mRNA to the subject. In some cases, the administering comprises administering no greater than 500 ng, 1 μg, 10 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, or 500 μg of the extracellular matrix mRNA to the subject. In some cases, the administering comprises administering 1 ng-20 μg of the extracellular matrix mRNA to the subject. In some cases, the administering comprises administering 1 ng-10 μg of the extracellular matrix mRNA to the subject. In some cases, the administering comprises administering 10 ng-10 μg, 10 ng-1 μg, 50 ng-1 μg, 100 ng-1 μg, or 500 ng-1 μg of the extracellular matrix mRNA to the subject. In some cases, the administering comprises administering about 50 ng of the extracellular matrix mRNA to the subject.

In some cases, the extracellular matrix mRNA is encapsulated in at least one extracellular vesicle (EV). In some cases, the EV does not comprise one or more of the following miRNAs: hsa-miR-29c-3p, hsa-miR-29a-3p, hsa-miR-378a-3p, hsa-miR-125b-5p, hsa-miR-23a-3p, hsa-miR-449a, hsa-miR-196a-5p, hsa-miR-744-5p, hsa-miR-223-3p, hsa-miR-23a-3p, hsa-miR-133a-3p, hsa-miR-223-3p, hsa-miR-5011-5p, hsa-miR-325, or hsa-miR-199b-5p.

In some cases, the at least one EV is an exosome. In some cases, the at least one EV is an apoptotic body or a microvesicle. In some cases, the at least one EV comprises a mixture of at least two of the following: exosome, microvesicle and/or apoptotic body.

In some cases, the treating the skin damage results in at least a 10% reduction in appearance of wrinkles. In some cases, the at least a 10% reduction in appearance of wrinkles occurs within 7-14 days of the administering of the at least one extracellular vesicle comprising extracellular matrix mRNA to the subject. In some cases, the treating the skin damage results in at least a 20%, 30%, 40%, 50%, 60%, 70%, 80%, 90% reduction in appearance of wrinkles. In some cases, the at least a 20%, 30%, 40%, 50%, 60%, 70%, 80%, 90% reduction in appearance of wrinkles occurs within 7-14 days of the administering of the at least one extracellular vesicle comprising extracellular matrix mRNA to the subject. In some cases, the treating the skin damage results in an at least 30%, 40%, 50%, 60%, 70%, or 80% reduction in total wrinkle number. In some cases, the treating the skin damage results in about 30%, 40%, 50%, 60%, 70%, 80%, 90%, or 95% reduction in total wrinkle number. In some cases, the treating the skin damage results in an at least 10%, 20%, 30%, 40%, 50%, 60%, 70%, 80%, or 90% reduction in total wrinkle area. In some cases, the treating the skin damage results in an at least 70%, 80%, or 90% reduction in total wrinkle area.

In some cases, the treating the skin condition comprises treating skin damage and wherein the treating the skin damage results in a reduction of skin damage that lasts for at least 20 days, 30 days, 40 days, 50 days, 60 days, 70 days, 80 days, 90 days, or 100 days following the treating the skin condition. In some cases, the reduction in skin damage is one or more of the following: reduction in total wrinkle number, reduction in total wrinkle area, reduction in appearance of wrinkles, or any combination thereof.

In some cases, the administering is via a subcutaneous injection. In some cases, the subcutaneous injection is performed at or near a site of the skin damage or the wound. In some cases, the subcutaneous injection is performed with a needle with a gauge of at least 14. In some cases, the subcutaneous injection is performed with a needle with a gauge of 34, 33, 32, 31, 30, 29, 28, 27, 26 s, 26, 25 s, 25, 24, 23 s, 23, 22 s, 22, 21, 20, 19, 18, 17, 16, 15, 14, 13, 12, 11, or 10. In some cases, the subcutaneous injection is performed with a needle with a gauge of 28.

In some cases, the administering comprises administering at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles to the subject in need thereof, and at least a portion of the extracellular vesicles comprise the at least one extracellular matrix mRNA. In some cases, the administering comprises administering at most 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles to the subject in need thereof, and at least a portion of the extracellular vesicles comprise the at least one extracellular matrix mRNA. In some cases, the administering comprises administering about 1×10⁷ to 1×10¹⁷, 1×10⁸ to 1×10¹⁶, 1×10⁹ to 1×10¹⁵ extracellular vesicles to the subject in need thereof, and at least a portion of the extracellular vesicles comprise the at least one extracellular matrix mRNA. In some cases, the administering comprises administering about 1×10¹⁰ to 1×10¹⁴ extracellular vesicles to the subject in need thereof, and at least a portion of the extracellular vesicles comprise the at least one extracellular matrix mRNA.

In some cases, the extracellular vesicles are administered in multiple doses or as a single dose. For example, the extracellular vesicles can be administered to the subject multiple times until a skin condition is treated. In other cases, a single dose is sufficient to achieve a therapeutic effect and thus the subject can receive only a single dose of the extracellular vesicles. In some cases, the single dose is repeated at a later time period in order to maintain the therapeutic effect on the skin condition. In some cases, the multiple dose regimen is repeated at a later time period in order to maintain the therapeutic effect on the skin condition. In some cases, the therapeutic effect wanes over time. For example, the therapeutic effect can wane after at least 30 days, at least 45 days, at least 60 days, at least 75 days, at least 90 days, etc. In such cases, the single dose regimen or multiple dose regimen can be administered to the subject again in order to treat the skin condition. In some cases, the single dose is administered in a single vessel, such as a single needle or syringe. In some cases, the single dose is administered using a microneedle device wherein multiple microneedles of the device deliver the single dose. In some cases, at least one microneedle of a microneedle device contains a dose of extracellular vesicles sufficient to treat the skin condition.

In some cases, the extracellular vesicles are administered to the subject in intervals of at least once a day, once every week, once every 2 weeks, once every 4 weeks, once every 5 weeks, once every 6 weeks, once every 7 weeks, once every 8 weeks, once every 10 weeks, once every 12 weeks, or once every 16 weeks. In some cases, the extracellular vesicles are administered to the subject at most once a day, once every week, once every 2 weeks, once every 4 weeks, once every 5 weeks, once every 6 weeks, once every 7 weeks, once every 8 weeks, once every 10 weeks, once every 12 weeks, or once every 16 weeks. In some cases, the administering is performed a single time and administers at most 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles to the subject. In some cases, the administering is performed a single time and administers at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles to the subject. In some cases, the extracellular vesicles are administered in at least one dose that comprises at least 1,000 extracellular vesicles, wherein at least a portion of the extracellular vesicles comprise the at least one extracellular matrix mRNA. In some cases, the extracellular vesicles are administered in at least one dose that comprises at least 5,000 extracellular vesicles, wherein at least a portion of the extracellular vesicles comprise the at least one extracellular matrix mRNA. In some cases, the extracellular vesicles are administered in at most one dose that comprises at least 1,000 extracellular vesicles, wherein at least a portion of the extracellular vesicles comprise the at least one extracellular matrix mRNA. In some cases, the extracellular vesicles are administered in at most one dose that comprises at least 5,000 extracellular vesicles, wherein at least a portion of the extracellular vesicles comprise the at least one extracellular matrix mRNA. In some cases, the administering comprises administering the at least one extracellular matrix mRNA to a tissue of the subject.

In some cases, the tissue is a subcutis. In some cases, the tissue is a dermis. I In some cases, the tissue is a epidermis. In some cases, the tissue is a gum. In some cases, the tissue is a lip.

In some cases, within 72 hours of the administering of the at least one extracellular vesicle comprising the extracellular matrix mRNA to the subject, the tissue has a concentration of an extracellular matrix protein of at least 6000 μg/ml. In some cases, following the administering of the extracellular matrix mRNA to tissue of the subject, the tissue of the subject has a concentration of an extracellular matrix protein of at least 200, 300, 500, 750, 1000, 2000, 3000, 4000, or 5000 μg/ml. In some cases, following the administering of extracellular vesicles comprising the extracellular matrix mRNA to tissue of the subject, the tissue of the subject has a concentration of an extracellular matrix protein of at least 200, 300, 500, 750, 1000, 2000, 3000, 4000, or 5000 μg/ml. In some cases, within 72 hours of the administering of the at least one extracellular vesicle comprising the extracellular matrix mRNA to the subject, the tissue has a concentration of an extracellular matrix protein of at least 200, 300, 500, 750, 1000, 2000, 3000, 4000, or 5000 μg/ml. In some cases, within about 4 days of the administering of the at least one extracellular vesicle comprising the extracellular matrix mRNA to the subject, the tissue has a peak concentration of an extracellular matrix protein.

In some cases, the administering of the at least one extracellular vesicle comprising the extracellular matrix mRNA to the subject is repeated at least every day, every 3 days, every 5 days, every 7 days, every 10 days, every 20 days, every 30 days, every 40 days, or every 50 days. In some cases, the administering of the at least one extracellular vesicle comprising the extracellular matrix mRNA to the subject is repeated about every 10 days, every 20 days, every 30 days, every 40 days, or every 50 days. In some cases, the administering of the at least one extracellular vesicle comprising the extracellular matrix mRNA to the subject is repeated about every 30 days. In some cases, the administering of the at least one extracellular vesicle comprising the extracellular matrix mRNA to the subject is repeated about every 60 days. In some cases, the administering of the at least one extracellular vesicle comprising the extracellular matrix mRNA to the subject is repeated about every 90 days.

In some cases, the method further comprises producing the extracellular vesicles comprising the at least one extracellular matrix mRNA by: (a) introducing a vector or a plasmid correspond to the extracellular matrix mRNA into a donor cell via transfection; (b) culturing the donor cell for a sufficient amount of time in a culture medium for the production of extracellular vesicles encapsulating the at least one extracellular matrix mRNA transcribed from the vector or the plasmid; and (c) collecting the extracellular vesicles from the culture medium. In some cases, in step (c) the collecting the extracellular vesicles occurs about 8-24 hr after the transfection. In some cases, the extracellular vesicles are present at a ratio of at least 1000, 2000, 5000, 10000, or 12000 extracellular vesicles per donor cell. In some cases, the donor cell is a human cell, a human fibroblast cell, a fibroblast cell, a dermal fibroblast, a human fibroblast, an adult fibroblast, a human adult fibroblast, a neonatal fibroblast, a neonatal human fibroblast, a neonatal human dermal fibroblast, or any combination thereof.

Described herein are methods of treating a disease in a subject by administering a therapeutic effective amount of the composition or pharmaceutical composition comprising the extracellular vesicle described herein. In some cases, the extracellular vesicle comprises the at least one therapeutic described herein. In some cases, the therapeutic comprises a therapeutic polynucleotide, therapeutic polypeptide, a therapeutic compound, or a combination thereof. In some instances, the therapeutic polynucleotide therapeutic polynucleotide comprises an extracellular matrix RNA (e.g., collagen or elastin, or mixtures of collagen and elastin) or a VEGF RNA (e.g., VEGFA, or mixtures of VEGF).

In some cases, the extracellular vesicle comprises the at least one targeting polypeptide and at least one therapeutic described herein. In some cases, the targeting polypeptide comprises a heterologous targeting domain comprising a tumor targeting domain, a tissue-targeting domain, a cell-penetrating peptide, a viral membrane protein, or any combination or fragment thereof. In some instances, the targeting domain respectively binds to a cell-surface marker associated with a diseased cell, where upon binding to the diseased cell the extracellular vesicle delivers the at least one therapeutic to the diseased cell.

In some cases, targeted cell uptake of the therapeutic delivered by the extracellular vesicle comprising the at least one targeting polypeptide is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or higher compared to targeted cell uptake of the therapeutic delivered by an extracellular vesicle without the targeting polypeptide. In some instances, the targeted cell with the increased uptake of the therapeutic delivered by the extracellular vesicle comprising the at least one targeting polypeptide is a cell as part of a tissue (e.g., a skin cell such as a melanocyte).

In some cases, described herein are methods of treating a blood flow disorder by administering the extracellular vesicle comprising therapeutic polynucleotide (and optional targeting polypeptide) described in this instant disclosure to the subject with the blood flow disorder. In some cases, described herein are methods of treating ischemia in the subject with the extracellular vesicle comprising a therapeutic polynucleotide such as a VEGF mRNA. In some cases, the ischemia is selected from the group consisting of: cerebral ischemia, ischemic heart disease, myocardial ischemia, cardiac ischemia, critical limb ischemia, kidney ischemia, acute mesenteric ischemia, bowel ischemia, cyanosis, and gangrene. In some cases, the therapeutic polynucleotide delivered to the ischemic tissue comprises mRNA encoding full length or truncated protein. In some cases, the therapeutic polynucleotide delivered to the ischemic tissue encodes VEGF protein. In some cases, delivery of the therapeutic polynucleotide stimulates revascularization of the ischemic tissue.

In some cases, the methods comprises administering to the subject with a single dose of the extracellular vesicle comprising VEGF mRNA. In some cases, the single dose is at least 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹⁰, 1×10¹¹, 1×10¹², 1×10¹³, 1×10¹⁴, 1×10¹⁵, 1×10¹⁶, 1×10¹⁷, 1×10¹⁸ EVs. In some cases, the single dose is no greater than 1×10¹⁰, 1×10¹¹, 1×10¹², 1×10¹³, 1×10¹⁴, 1×10¹⁵, 1×10¹⁶, 1×10¹⁷, 1×10¹⁸ 1×10¹⁹, 1×10²⁰, 1×10²¹, 1×10²², 1×10²³, 1×10²⁴, 1×10²⁵, 1×10³⁰, 1×10⁴⁰, 1×10⁵⁰, or 1×10⁶⁰ EVs. In some cases, the single dose is about 1×10¹⁰ to 1×10¹⁶ EVs. In some cases, the single dose is at least 10 μg, 50 μg, 100 μg, 200 μg, 300 μg, 400 μg, 500 μg, 600 μg, 700 μg, 800 μg, 900 μg, 1 ng, 100 ng, 500 ng, 1000 ng, 10 μg, 50 μg, 100 μg, 150 μg, or 200 μg of the VEGF mRNA. In some cases, the single dose is no greater than 1 ng, 100 ng, 500 ng, 1000 ng, 10 μg, 50 μg, 100 μg, 150 μg, 200 μg, 300 μg, 400 μg, 500 μg, 600 μg, 700 μg, 800 μg, 900 μg, or 1000 μg of the VEGF mRNA. In some cases, the single dose is about 1 ng to 200 μg of the VEGF mRNA.

In some cases, the methods comprises administering to the subject with multiple doses of the extracellular vesicle comprising VEGF mRNA. In some cases, the multiple doses are administered over a period of time with a frequency of at least once a day, once every week, once every 2 weeks, once every 4 weeks, once every 5 weeks, once every 6 weeks, once every 7 weeks, once every 8 weeks, once every 10 weeks, once every 12 weeks, or once every 16 weeks. In some cases, the multiple doses are administered for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks. In some cases, at least one of the multiple doses is at least 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹⁰, 1×10¹¹, 1×10¹², 1×10¹³, 1×10¹⁴, 1×10¹⁵, 1×10¹⁶, 1×10¹⁷, 1×10¹⁸ EVs. In some cases, at least one of the multiple dose is no greater than 1×10¹⁰, 1×10¹¹, 1×10¹², 1×10¹³, 1×10¹⁴, 1×10¹⁵, 1×10¹⁶, 1×10¹⁷, 1×10¹⁸ 1×10¹⁹, 1×10²⁰, 1×10²¹, 1×10²², 1×10²³, 1×10²⁴, 1×10²⁵, 1×10³⁰, 1×10⁴⁰, 1×10⁵⁰, or 1×10⁶⁰ EVs. In some cases, at least one of the multiple dose is about 1×10¹⁰ to 1×10¹⁶ EVs. In some cases, at least one of the multiple doses is at least 10 μg, 50 μg, 100 μg, 200 μg, 300 μg, 400 μg, 500 μg, 600 μg, 700 μg, 800 μg, 900 μg, 1 ng, 100 ng, 500 ng, 1000 ng, 10 μg, 50 μg, 100 μg, 150 μg, or 200 μg of the VEGF mRNA. In some cases, at least one of the multiple doses is no greater than 1 ng, 100 ng, 500 ng, 1000 ng, 10 μg, 50 μg, 100 μg, 150 μg, 200 μg, 300 μg, 400 μg, 500 μg, 600 μg, 700 μg, 800 μg, 900 μg, or 1000 μg of the VEGF mRNA. In some cases, the single dose is about 1 ng to 200 μg of the VEGF mRNA.

In some cases, described herein are methods of treating skin damage by administering the extracellular vesicle comprising therapeutic polynucleotide (and optional targeting polypeptide) described in this instant disclosure to the subject. In some cases, the described herein are methods of treating sun damage or age-related skin damage in the subject with the extracellular vesicle comprising therapeutic polynucleotide. In some instances, the skin damage includes wrinkling. In some instances, described herein are methods of treating wrinkles with therapeutic polynucleotide delivered to the skin by the extracellular vesicle described in this instant disclosure. In some cases, the therapeutic polynucleotide delivered to the skin comprise mRNA encoding full length or truncated protein. In some cases, the therapeutic polynucleotide delivered to the skin encodes an ECM protein. In some cases, the therapeutic polynucleotide delivered to the skin encodes collagen type I. In some cases, delivery of the therapeutic polynucleotide reduces the appearance of wrinkles in the damaged skin.

In some cases, described herein methods of treating a disease or condition by administering the extracellular vesicle comprising therapeutic polynucleotide (and optional targeting polypeptide) to a subject in need thereof. In some cases, the extracellular vesicle comprising therapeutic polynucleotide (and optional targeting polypeptide) is administered systemically, e.g. via intravenous injection. In some cases, the extracellular vesicle comprising therapeutic polynucleotide (and optional targeting polypeptide) is administered locally, e.g. via intramuscular or subcutaneous injection.

In some cases, the intravenous, intramuscular, or subcutaneous injection is performed with a hypodermic needle. In some cases, the intravenous, intramuscular, or subcutaneous injection is performed with a needle with a gauge of at least 32, 31, 30, 29, 28, 27, 26, 25, 24, 23, 22, 21, 20, 19, 18, 17, 16, 15, or 14. In some cases, the intravenous, intramuscular, or subcutaneous injection is performed with a needle with a gauge of at most 14, 15, 16, 17, 18, 19, 20, 21, 22, 23, 24, 25, 26, 27, 28, 29, 30, 31, or 32. In some cases, the injection is via coronary catheter, such as a coronary artery catheter or a coronary venous catheter. In some cases, the injection is via a large vessel catheter. In some cases, the intravenous, intramuscular, or subcutaneous injection is performed with a needle with a gauge of 28. In some cases, the intravenous, intramuscular, or subcutaneous injection is performed with a needle with a gauge of 14 or above. In some cases, delivery to the heart (such as delivery to the heart to treat myocardial infarction) is achieved via coronary catheter (such as a coronary artery catheter or a coronary venous catheter). In some cases, delivery for treatment of stroke (such as delivery for treatment of ischemic stroke or delivery for treatment of hemorrhagic stroke) is achieved via a catheter in one of the brain vessels or via open heart injection directly into muscle. In some cases, delivery for treating peripheral artery disease is achieved via a catheter into a vessel or direct injection into a muscle. In some cases, delivery for treatment of skin diseases or disorders (such as delivery for skin ulcers) is achieved via subcutaneous injection.

In some cases, the extracellular vesicle comprising therapeutic polynucleotide (and optional targeting polypeptide) is formulated for administration via injection in a solution or a suspension comprising an injection buffer. The injection buffer can comprise saline, Phosphate-Buffered Saline (PBS), and/or 25 mM trehalose PBS. In some cases, the dose is measured by the number of extracellular vesicles administered per dose. In some cases, the dose comprises about 100, 1,000, 10,000, 100,000, 1,000,000, 10,000,000, 100,000,000, 1,000,000,000, 10,000,000,000, 100,000,000,000, 1,000,000,000,000, 10,000,000,000,000, 100,000,000,000,000, 1,000,000,000,000,000, 10,000,000,000,000,000, 100,000,000,000,000,000, 1,000,000,000,000,000,000, 10,000,000,000,000,000,000, 100,000,000,000,000,000,000, or 1,000,000,000,000,000,000,000 extracellular vesicles comprising therapeutic polypeptide per dose. In some cases, the dose comprises between about 100 and 1,000,000,000,000,000,000,000 extracellular vesicles comprising therapeutic polypeptide per dose, or between about 1,000 and 100,000,000,000,000,000,000, or between about 10,000 and 10,000,000,000,000,000,000, or between about 100,000 and 1,000,000,000,000,000,000, or between about 1,000,000 and 1,000,000,000,000,000,000, or between about 10,000,000 and 1,000,000,000,000,000,000, or between about 100,000,000 and 1,000,000,000,000,000,000, or between about 1,000,000,000 and 1,000,000,000,000,000,000, or between about 10,000,000,000 and 1,000,000,000,000,000,000, or between about 100,000,000,000 and 1,000,000,000,000,000,000, or between about 1,000,000,000,000 and 1,000,000,000,000,000,000, or between about 10,000,000,000,000 and 1,000,000,000,000,000,000, or between about 100,000,000,000,000 and 1,000,000,000,000,000,000 extracellular vesicles comprising therapeutic polypeptide per dose. In some cases, the dose is measured by the amount of therapeutic polypeptide administered per dose. In some cases, the dose comprises between about 1 ng and 500 μg of therapeutic polypeptide per dose. In some cases, the dose comprises between about 10 ng and 100 μg of therapeutic polypeptide per dose. In some cases, the dose comprises between about 100 ng and 100 μg, or between about 1 μg and 100 μg, or between about 10 μg and 100 μg of therapeutic polypeptide per dose.

In some cases, described herein methods of treating a disease or condition by administering the extracellular vesicle comprising therapeutic polynucleotide (and optional targeting polypeptide) to a subject in need thereof. In some cases, the extracellular vesicle comprising therapeutic polynucleotide (and optional targeting polypeptide) is administered daily, every day, every alternate day, five days a week, once a week, every other week, two weeks per month, three weeks per month, once a month, twice a month, three times per month, or more. The extracellular vesicle comprising therapeutic polynucleotide (and optional targeting polypeptide) can be administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the subject's status improves, the dose of the extracellular vesicle comprising therapeutic polynucleotide (and optional targeting polypeptide) being administered can be temporarily reduced or temporarily suspended for a certain length of time (a “drug holiday”). In some instances, the length of the drug holiday varies between 2 days and 1 year, including by way of example, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday can be from 10%-100%, including, by way of example, 10%, 15%, 20%, 25%, 30%, 35%, 40%, 45%, 50%, 55%, 60%, 65%, 70%, 75%, 80%, 85%, 90%, 95%, or 100%.

In some cases, an effective amount of the extracellular vesicle comprising therapeutic polynucleotide (and optional targeting polypeptide) can be administered to a subject in need thereof once per week, once every two weeks, once every three weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, once every 26 weeks, once every 27 weeks, or once every 28 weeks.

Once improvement of the subject's disease or condition have occurred, a maintenance dose of extracellular vesicles is administered if necessary. Subsequently, the dosage or the frequency of administration, or both, can be reduced, as a function of the symptoms, to a level at which the improved disease, disorder or condition is retained.

In some cases, the amount of the extracellular vesicle comprising therapeutic polynucleotide (and optional targeting polypeptide) that correspond to such an amount varies depending upon factors such as the severity of the disease or condition, the identity (e.g., weight) of the subject or host in need of treatment, but nevertheless is routinely determined in a manner known in the art according to the particular circumstances surrounding the case, including, e.g., the specific extracellular vesicle being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day. In some cases, the desired dose is administered in a single dose. In some cases, a relatively high dose of extracellular vesicles (e.g., at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles), is administered in a single dose. In some cases, a relatively high dose of exosomes (e.g. 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ exosomes) is administered in a single dose.

In some cases, the dosage can be at least partially determined by occurrence or severity of grade 3 or grade 4 adverse events in the subject. Non-limiting examples of adverse events include hypothermia; shock; bradycardia; ventricular extrasystoles; myocardial ischemia; syncope; hemorrhage; atrial arrhythmia; phlebitis; atrioventricular (AV) block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; acute tubular necrosis; duodenal ulceration; bowel necrosis; myocarditis; supraventricular tachycardia; permanent or transient blindness secondary to optic neuritis; transient ischemic attacks; meningitis; cerebral edema; pericarditis; allergic interstitial nephritis; tracheo-esophageal fistula; malignant hyperthermia; cardiac arrest; myocardial infarction; pulmonary emboli; stroke; liver or renal failure; severe depression leading to suicide; pulmonary edema; respiratory arrest; respiratory failure; leukopenia, thrombocytopenia, increased alanine aminotransferase (ALT), anorexia, arthralgia, back pain, chills, diarrhea, dyslipidemia, fatigue, fever, flu-like symptoms, hypoalbuminemia, increased lipase, injection site reaction, myalgia, nausea, night sweats, pruritis, rash, erythematous rash, maculopapular rash, transaminitis, vomiting, and weakness.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some cases, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

In some cases, an effective amount of the extracellular vesicle comprising therapeutic polynucleotide comprising ECM mRNA (and optional targeting polypeptide) is administered to a subject in at least one dose. In some instances, the extracellular vesicle comprising therapeutic polynucleotide comprising ECM mRNA (and optional targeting polypeptide) is administered to a subject in at least two doses. In some instances, an effective amount of extracellular vesicle comprising therapeutic polynucleotide comprising ECM mRNA (and optional targeting polypeptide) comprises at least 100, 1,000, 10,000, 100,000, 1,000,000, 10,000,000, 100,000,000, 1,000,000,000, 10,000,000,000, 100,000,000,000, 1,000,000,000,000, 10,000,000,000,000, 100,000,000,000,000, 1,000,000,000,000,000, 10,000,000,000,000,000, 100,000,000,000,000,000, 1,000,000,000,000,000,000, 10,000,000,000,000,000,000, 100,000,000,000,000,000,000, or 1,000,000,000,000,000,000,000 extracellular vesicles comprising therapeutic polynucleotide comprising ECM mRNA (and optional targeting polypeptide).

In other cases, an effective amount of the extracellular vesicle comprising therapeutic polynucleotide comprising ECM mRNA (and optional targeting polypeptide) is administered to the subject disclosed herein with at least 3 doses per week, at least 4 doses per week, at least 5 doses per week, at least 6 doses per week, at least 7 doses per week, at least 8 doses per week, at least 9 doses per week, or at least 10 doses per week. In other cases, an effective amount of the extracellular vesicle comprising therapeutic polynucleotide comprising ECM mRNA (and optional targeting polypeptide) is administered to the subject disclosed herein with less than 20 doses per week, less than 19 doses per week, less than 18 doses per week, less than 17 doses per week, less than 16 doses per week, less than 15 doses per week, less than 14 doses per week, less than 13 doses per week, less than 12 doses per week, less than 11 doses per week, or less than 10 doses per week. In other cases, an effective amount of the extracellular vesicle comprising therapeutic polynucleotide comprising ECM mRNA (and optional targeting polypeptide) is administered to the subject disclosed herein with 1 to 10 doses per week, 2-9 doses per week, 3-8 doses per week.

In some cases, an effective amount of the extracellular vesicles comprising therapeutic polynucleotide comprising ECM mRNA (and optional targeting polypeptide) is administered to a subject with one dose, and the effective amount is at least 100, 1,000, 10,000, 100,000, 1,000,000, 10,000,000, 100,000,000, 1,000,000,000, 10,000,000,000, 100,000,000,000, 1,000,000,000,000, 10,000,000,000,000, 100,000,000,000,000, 1,000,000,000,000,000, 10,000,000,000,000,000, 100,000,000,000,000,000, 1,000,000,000,000,000,000, 10,000,000,000,000,000,000, 100,000,000,000,000,000,000, or 1,000,000,000,000,000,000,000 extracellular vesicle. In other cases, an effective amount of the extracellular vesicles comprising therapeutic polynucleotide comprising ECM mRNA (and optional targeting polypeptide) is administered to a subject with a single dose, and the effective amount is at least 0.1 ng, 0.2 ng, 0.3 ng, 0.4 ng, 0.5 ng, 0.6 ng, 0.7 ng, 0.8 ng, 0.9 ng, 1 ng, 10 ng, 20 ng, 30 ng, 40 ng, 50 ng, 60 ng, 70 ng, 80 ng, 90 ng, 100 ng, 200 ng, 300 ng, 400 ng, 500 ng, 600 ng, 700 ng, 800 ng, 900 ng, or 1 μg of the ECM mRNA. In other cases, an effective amount of the extracellular vesicles comprising therapeutic polynucleotide comprising ECM mRNA (and optional targeting polypeptide) is administered to a subject with a single dose, and the effective amount is at most 50 μg, 40 μg, 30 μg, 20 μg, 10 μg, 1 μg, 900 ng, 800 ng, 700 ng, 600 ng, 500 ng, 400 ng, 300 ng, 200 ng, or 100 ng of the ECM mRNA. In other cases, an effective amount of the extracellular vesicles comprising therapeutic polynucleotide comprising ECM mRNA (and optional targeting polypeptide) is administered to a subject with a single dose, and the effective amount is 0.1 ng-50 μg, 0.1 ng-30 μg, 1 ng-20 μg, 1 ng-10 μg, 1 ng-1 μg, 1 ng-500 ng, 1 ng-100 ng, 10 ng-10 μg, 10 ng-1 μg, 10 ng-500 ng, 10 ng-100 ng, 50 ng-10 μg, 50 ng-1 μg, 50 ng-500 ng, or 50 ng-100 ng.

In other cases, an effective amount of the extracellular vesicles comprising therapeutic polynucleotide comprising ECM mRNA (and optional targeting polypeptide) is administered to a subject with multiple doses, and in each dose the effective amount is at least 100, 1,000, 10,000, 100,000, 1,000,000, 10,000,000, 100,000,000, 1,000,000,000, 10,000,000,000, 100,000,000,000, 1,000,000,000,000, 10,000,000,000,000, 100,000,000,000,000, 1,000,000,000,000,000, 10,000,000,000,000,000, 100,000,000,000,000,000, 1,000,000,000,000,000,000, 10,000,000,000,000,000,000, 100,000,000,000,000,000,000, or 1,000,000,000,000,000,000,000 extracellular vesicle. In other cases, an effective amount of the extracellular vesicles comprising therapeutic polynucleotide comprising ECM mRNA (and optional targeting polypeptide) is administered to a subject with multiple doses, and in each dose the effective amount is at least 0.1 ng, 0.2 ng, 0.3 ng, 0.4 ng, 0.5 ng, 0.6 ng, 0.7 ng, 0.8 ng, 0.9 ng, 1 ng, 10 ng, 20 ng, 30 ng, 40 ng, 50 ng, 60 ng, 70 ng, 80 ng, 90 ng, 100 ng, 200 ng, 300 ng, 400 ng, 500 ng, 600 ng, 700 ng, 800 ng, 900 ng, or 1 μg of the ECM mRNA. In other cases, an effective amount of the extracellular vesicles comprising therapeutic polynucleotide comprising ECM mRNA (and optional targeting polypeptide) is administered to a subject with multiple doses, and in each dose the effective amount is at most 50 μg, 40 μg, 30 μg, 20 μg, 10 μg, 1 μg, 900 ng, 800 ng, 700 ng, 600 ng, 500 ng, 400 ng, 300 ng, 200 ng, or 100 ng of the ECM mRNA. In other cases, an effective amount of the extracellular vesicles comprising therapeutic polynucleotide comprising ECM mRNA (and optional targeting polypeptide) is administered to a subject with multiple doses, and in each dose the effective amount is 0.1 ng-50 μg, 0.1 ng-30 μg, 1 ng-20 μg, 1 ng-10 μg, 1 ng-1 μg, 1 ng-500 ng, 1 ng-100 ng, 10 ng-10 μg, 10 ng-1 μg, 10 ng-500 ng, 10 ng-100 ng, 50 ng-10 μg, 50 ng-1 μg, 50 ng-500 ng, or 50 ng-100 ng. In cases where the effective amount of the extracellular vesicles are administered with multiple doses, the interval between the doses is at least 1 day, 5 days, 10 days, 15 days, 20 days, 30 days, 40 days, 50 days, 60 days, 70 days, 80 days, 90 days, 100 days, 110 days, or 120 days.

In some cases, the administering of the at least one extracellular vesicle comprising extracellular matrix mRNA to the subject comprises administering of the at least one extracellular vesicle comprising extracellular matrix mRNA to a tissue of the subject. In some specific cases, the tissue is a hypodermis. In some specific cases, the tissue is a dermis.

In some cases, an effective amount of the extracellular vesicle comprising therapeutic polynucleotide comprising ECM mRNA (and optional targeting polypeptide) is administered to a subject in order to treat skin damage of a subject. In some cases, the effective amount is sufficient to cause at least a 50% reduction in appearance of wrinkles. In some cases, the effective amount is sufficient to cause at least a 10%, 20%, 30%, 40%, or 50% reduction in appearance of wrinkles. In some cases, the effective amount is sufficient to cause at least a 50% reduction in appearance of wrinkles within about 1, 2, 3, 4, or 5 days of administration. In some cases, the effective amount is sufficient to cause at least a 50% reduction in appearance of wrinkles within about 6, 7, 8, 9, or 10 days of administration. In some cases, the effective amount is sufficient to cause at least a 10%, 20%, 30%, 40%, or 50% reduction in the appearance of wrinkles within about 14 days of administration. In some cases, the effective amount is sufficient to cause at least a 10%, 20%, 30%, 40%, or 50% reduction in appearance of wrinkles within about 5 days of administration.

In some cases, the treating the skin damage results in at least 10%, 20%, 30%, 40%, 50%, 60%, 70%, 80%, or 90% reduction in total wrinkle number. In some cases, the treating the skin damage results in about 10%, 20%, 30%, 40%, 50%, 60%, 70%, 80%, or 90% reduction in total wrinkle number. In some cases, the treating the skin damage results in at least 10%, 20%, 30%, 40%, 50%, 60%, 70%, 80%, 90% in total wrinkle area. In some cases, the treating the skin damage results in about 10%, 20%, 30%, 40%, 50%, 60%, 70%, 80%, 90% in total wrinkle area.

In some cases, an effective amount of the extracellular vesicle comprising therapeutic polynucleotide comprising ECM mRNA (and optional targeting polypeptide) is administered to a subject in order to treat skin damage of a subject. In some cases, the effective amount is sufficient to generate a tissue concentration of extracellular matrix protein (e.g., collagen) of at least 6000 μg/ml within about 72 hours of administration.

In some cases, within about 4 days of the administering of the at least one extracellular vesicle comprising extracellular matrix mRNA to the subject, the tissue has a peak concentration of extracellular matrix protein.

In some cases, the administering of the at least one extracellular vesicle comprising extracellular matrix mRNA to the subject is repeated about every day, every other day, every 5 days, every 10 days, every 20 days, every 30 days, every 40 days, every 50 days, every 60 days, every 70 days, every 80 days, every 90 days, or every 100 days. In specific cases, the administering of the at least one extracellular vesicle comprising extracellular matrix mRNA to the subject is repeated about every 30 days.

In some cases, the administering of the at least one extracellular vesicle comprising extracellular matrix mRNA comprises administering the at least one extracellular vesicle comprising extracellular matrix mRNA to the subject via a subcutaneous injection. In some specific cases, the subcutaneous injection is performed at or near a site of the skin damage. In some cases, the subcutaneous injection is performed with a needle with a gauge of about 32, 31, 30, 29, 28, 27, 26, 25, 24, 23, 22, 21, 20, 19, 18, 17, 16, 15, or 14. In some cases, the subcutaneous injection is performed with a needle with a gauge of at least 20, 19, 18, 17, 16, 15, or 14.

In some cases, the skin damage is caused by aging. In other cases, skin damage is caused by sun damage. In other cases, the skin damage is caused by an autoimmune disease. In other cases, the skin damage is caused by burning. In other cases, the skin damage is caused by scars. In other cases, the skin damage is caused by a skin disease. In some specific cases, the skin damage is caused by dermatosparaxis Ehlers-Danlos syndrome. In some specific cases, the skin damage is caused by epidermolysis bullosa. In some specific cases, the skin damage is caused by pseudosyndactyly. In some specific cases, the skin damage is caused by poor wound healing. In some specific cases, the skin damage is caused by digit and limb contractures. In some specific cases, the skin damage is caused by keratodermas. In some specific cases, the skin damage is caused by systemic sclerosis. In some specific cases, the skin damage is caused by scleroderma. In some specific cases, the skin damage is caused by atopic dermatitis. In some specific cases, the skin damage is caused by periodontal disease.

In some cases, the method of treating skin damage of the subject further comprises producing the extracellular vesicles comprising extracellular matrix mRNA wherein the producing the extracellular vesicles comprising extracellular matrix mRNA comprises: (a) introducing a vector or plasmid encoding an extracellular matrix protein into a donor cell via transfection; (b) incubating the donor cell for a sufficient time for the production of extracellular vesicles encapsulating extracellular matrix mRNA transcribed from the vector or plasmid; and (c) collecting the extracellular vesicles encapsulating the extracellular matrix mRNA transcribed from the vector or plasmid.

In some cases, in step (a) the transfection is performed with cellular electroporation as shown in FIG. 7A. In some specific cases, in step (a) the transfection comprises seeding a single layer of the donor cells on to the surface of the silicon chip shown in FIG. 7A, and certain voltage of electric field with certain pulses and durations is applied for electroporating the vector or the plasmid into the donor cell. In some further specific cases, the seeding takes overnight incubation as previously described. In some further specific cases, the voltage of the electric field is at least 10V, 50V, 100V, 150V, 200V, 250V, 300V, 500V, or 1000V. In some further specific cases, the voltage of the electric field is about 10V, 50V, 100V, 150V, 200V, 250V, 300V, 500V, or 1000V. In some further specific cases, the voltage of the electric field is at most 500V, 600V, 700V, 800V, 900V, 1000V, 1500V, 2000V, 5000V, or 10,000V. In some further specific cases, the pulses of the electric field is at least 5, 10, 15, 20, 25, 30, 40, 50, or 60. In some further specific cases, the pulses of the electric field is about 5, 10, 15, 20, 25, 30, 40, 50, or 60. In some further specific cases, the interval between pulses of the electric field is at least 0.1 s, 0.2 s, 0.3 s, 0.4 s, 0.5 s, 0.6 s, 0.7 s, 0.8 s, 0.9 s, or Is. In some further specific cases, the interval between pulses of the electric field is about 0.1 s, 0.2 s, 0.3 s, 0.4 s, 0.5 s, 0.6 s, 0.7 s, 0.8 s, 0.9 s, or Is.

In some cases, in step (c) the collecting the extracellular vesicles occurs about 1, 2, 3, 4, 5, 6, 7, 8, 9, 10, 11, 12, 13, 14, 15, 16, 17, 18, 19, 20, 21, 22, 23, or 24 hr after the transfection. In some cases, in step (c) the collecting the extracellular vesicles occurs at least 1, 2, 3, 4, 5, 6, 7, 8, 9, 10, 11, 12, 13, 14, 15, 16, 17, 18, 19, 20, 21, 22, 23, or 24 hr after the transfection.

In some cases, extracellular vesicle comprising therapeutic polynucleotide comprising ECM mRNA (and optional targeting polypeptide) is administered to a subject in order to treat skin damage (e.g., sun damage or age-related) of a subject via subcutaneous injection.

In some cases, an effective amount of the extracellular vesicle comprising therapeutic polynucleotide comprising VEGF mRNA (and optional targeting polypeptide) is administered to a subject in at least one dose. In some instances, the extracellular vesicle comprising therapeutic polynucleotide comprising VEGF mRNA (and optional targeting polypeptide) is administered to a subject in at least two doses. In some instances, an effective amount of extracellular vesicle comprising therapeutic polynucleotide comprising VEGF mRNA (and optional targeting polypeptide) comprises at least 100, 1,000, 10,000, 100,000, 1,000,000, 10,000,000, 100,000,000, 1,000,000,000, 10,000,000,000, 100,000,000,000, 1,000,000,000,000, 10,000,000,000,000, 100,000,000,000,000, 1,000,000,000,000,000, 10,000,000,000,000,000, 100,000,000,000,000,000, 1,000,000,000,000,000,000, 10,000,000,000,000,000,000, 100,000,000,000,000,000,000, or 1,000,000,000,000,000,000,000 extracellular vesicles comprising therapeutic polynucleotide comprising VEGF mRNA (and optional targeting polypeptide).

In some cases, an effective amount of the extracellular vesicle comprising therapeutic polynucleotide comprising VEGF mRNA (and optional targeting polypeptide) is administered to a subject in order to treat a blood flow disorder of a subject. In some cases, the effective amount is sufficient to cause at least a 50% increase in revascularization and/or neovascularization. In some cases, the effective amount is sufficient to cause at least a 10%, 20%, 30%, 40%, or 50% increase in revascularization and/or neovascularization. In some cases, the effective amount is sufficient to cause at least a 50% increase in revascularization and/or neovascularization within about 1, 2, 3, 4, 5, 6, 7, or 8 days of administration. In some cases, the effective amount is sufficient to cause at least a 5%, 10%, 20%, 30%, 40%, or 50% increase in revascularization and/or neovascularization within about 8 days of administration. In some cases, the effective amount is sufficient to cause at least a 5% increase in revascularization and/or neovascularization within 8 days of administration. In some cases, the effective amount is sufficient to cause between about 5% and 10% increase in revascularization and/or neovascularization within 8 days of administration. In some cases, the effective amount is sufficient to cause between about 5% and 15%, or between about 5% and 20%, or between about 5% and 25%, or between about 5% and 30%, or between about 5% and 40%, or between about 5% and 50%, or between about 5% and 60%, or between about 5% and 70%, or between about 5% and 75% increase in revascularization and/or neovascularization within 8 days of administration. In some cases, the effective amount is sufficient to cause at least a 5% increase in revascularization and/or neovascularization within 14 days of administration. In some cases, the effective amount is sufficient to cause between about 5% and 10% increase in revascularization and/or neovascularization within 14 days of administration. In some cases, the effective amount is sufficient to cause between about 5% and 15%, or between about 5% and 20%, or between about 5% and 25%, or between about 5% and 30%, or between about 5% and 40%, or between about 5% and 50%, or between about 5% and 60%, or between about 5% and 70%, or between about 5% and 75% increase in revascularization and/or neovascularization within 14 days of administration.

In some cases, an effective amount of the extracellular vesicle comprising therapeutic polynucleotide comprising VEGF mRNA (and optional targeting polypeptide) is administered to a subject in order to treat a blood flow disorder of a subject. In some cases, the effective amount is sufficient to generate a tissue concentration of VEGF protein (e.g., VEGFA) of at least 6000 μg/ml within about 72 hours of administration.

In some cases, extracellular vesicle comprising therapeutic polynucleotide comprising VEGF mRNA (and optional targeting polypeptide) is administered to a subject in order to treat a blood flow disorder (e.g., ischemia) of a subject via intravenous or intramuscular injection.

Further provided herein are methods of reducing the risk of pseudosyndactyly, poor wound healing, or digit and limb contracture in a subject in need thereof, and the methods comprise administering to the subject with an effective amount of COL7A1 mRNA loaded EVs. In some cases, the subject is a patient with dystrophic epidermolysis bullosa. In some cases, the patients are pediatric patients. In some cases, the administering is to the hands and/or feet of the subject. In some cases, the effective amount of Col7A1 mRNA is at least 1 ng, 10 ng, 20 ng, 40 ng, 50 ng, 100 ng, 500 ng, 1 μg, 10 μg, 20 μg, 30 μg, 40 μg, or 50 μg of Col7A1 mRNA. In some cases, the effective amount of Col7A1 mRNA is enculturated in at least one extracellular vesicles. In some cases, the administering comprises administering at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles to the subject in need thereof, and at least a portion of the extracellular vesicles comprise the at least one Col7A1 mRNA. In some cases, the at last one extracellular vesicles is injected via the needle, the hydrogel needle, the microneedle, or the microneedle device described herein.

Further provided herein are methods of preventing, alleviating, or treating hair loss in a subject in need thereof, and the methods comprise administering to the subject an effective amount of Col17a1 mRNA. In some cases, the administering is to the affected scalp of the subject. In some cases, the effective amount of Col17a1 mRNA is at least 1 ng, 10 ng, 20 ng, 40 ng, 50 ng, 100 ng, 500 ng, 1 μg, 10 μg, 20 μg, 30 μg, 40 μg, or 50 μg of Col17a1 mRNA. In some cases, the effective amount of Col17a1 mRNA is no greater than 1 ng, 10 ng, 20 ng, 40 ng, 50 ng, 100 ng, 500 ng, 1 μg, 10 μg, 20 μg, 30 μg, 40 μg, 50 μg, 60 μg, 70 μg, 80 μg, 90 μg, 100 μg, 110 μg, 120 μg, 130 μg, 140 μg, or 150 μg of Col17a1 mRNA. In some cases, the effective amount of Col17a1 mRNA is enculturated in at least one extracellular vesicles. In some cases, the administering comprises administering at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10², or 1×10³⁰ extracellular vesicles to the subject in need thereof, and at least a portion of the extracellular vesicles comprise the at least one Col17a1 mRNA. In some cases, the at last one extracellular vesicles is injected via the needle, the hydrogel needle, the microneedle, or the microneedle device described herein.

Further provided herein are methods of preventing, alleviating, or treating palmoplantar keratoderma in a subject in need thereof, and the methods comprise administering to the subject an effective amount of viable keratin mRNA. In some cases, the administering is to the affected skin of the subject.

Further provided herein are methods of preventing, alleviating, or treating keloid scarring in a subject in need thereof, and the methods comprise administering to the subject with an effective amount of an inhibitory agent that reduces TGF-Beta 1, SMAD3, and EPHB2.

Further provided herein are methods of injecting in lips of a subject, and the methods comprise administering to the subject with an effective amount of EVs comprising at least one collagen mRNA. In some cases, the at least one collagen mRNA is collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or any combination thereof. In some cases, effective amount of EVs comprises at least 1 ng, 10 ng, 20 ng, 40 ng, 50 ng, 100 ng, 500 ng, 1 μg, 10 μg, 20 μg, 30 μg, 40 μg, or 50 μg of collagen mRNA. In some cases, effective amount of EVs comprising at least one collagen mRNA is at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles. In some cases, the effective amount of EVs is injected via the needle, the hydrogel needle, the microneedle, or the microneedle device described herein.

Further provided herein are methods of injecting in gums of a subject, and the methods comprise administering to the subject with an effective amount of EVs comprising at least one collagen mRNA. In some cases, the at least one collagen mRNA is collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or any combination thereof. In some cases, the effective amount of EVs comprising at least 1 ng, 10 ng, 20 ng, 40 ng, 50 ng, 100 ng, 500 ng, 1 μg, 10 μg, 20 μg, 30 μg, 40 μg, or 50 μg of collagen mRNA. In some cases, effective amount of EVs comprising at least one collagen mRNA is at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, 1×10¹⁴, 1×10¹⁵, 1×10¹⁶, 1×10²⁰, 1×10²⁵, or 1×10³⁰ extracellular vesicles. In some cases, the effective amount of EVs is injected via the needle, the hydrogel needle, the microneedle, or the microneedle device described herein.

Further provided herein are methods of administering to a subject in need thereof with an effective amount of EVs of any cargos. In some cases, the cargos are microRNAs (miRNAs). In some cases, the cargos are small interfering RNAs (siRNAs). In some cases, the cargos are peptides. In some cases, the cargos are proteins. In some cases, the cargos are inhibitory double stranded RNAs (dsRNAs). In some cases, the cargos are small or short hairpin RNAs (shRNAs). In some cases, the cargos are antisense oligonucleotides (ASOs). In some cases, the cargos are piwi-interacting RNAs (piRNAs). In some cases, the cargos are heterogeneous nuclear RNAs (hnRNAs). In some cases, the cargos are small nuclear RNAs (snRNAs). In some cases, the cargos are enzymatically-prepared siRNAs (esiRNAs). In some cases, the cargos are precursors of any of the above cargos. In some cases, the cargos are any combinations of the above two or more cargos.

Further provided herein are microneedle devices, the microneedle devices comprising a substrate and a plurality of microneedles, wherein the plurality of microneedles protrude from the substrate, and wherein at least one microneedle of the plurality of microneedles comprises at least one extracellular vesicle (EV).

In some cases, the substrate provides a surface where the plurality of microneedles are attached to. In some cases, the microneedle device disclosed herein comprises other components other than the substrate and the plurality of microneedles. In some specific cases, the other components comprise are added to improve skin penetration and depth, as well as drug delivery and/or distribution.

In some cases, the plurality of microneedles comprises one microneedle. In other cases, the plurality of microneedles comprises at least 1, at least 2, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 10000, or at least 100000 microneedles. In some cases, the area of the substrate is at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200 mm². In some cases, the area of the substrate is less than 1000, less than 900, less than 800, less than 700, less than 600, less than 500, less than 400, less than 300, less than 200 mm². In some specific cases, the area of the substrate is about 169 mm².

In some cases, the density of the plurality of microneedles on the substrate is at least 0.1, at least 0.2, at least 0.3, at least 0.4, at least 0.5, at least 0.6, at least 0.7, at least 0.8, at least 0.9, or at least 1 microneedle/mm² of the substrate. In other cases, the density of the plurality of microneedles on the substrate is less than 100, less than 90, less than 80, less than 70, less than 60, less than 50, less than 40, less than 30, less than 20, less than 10, less than 9, less than 8, less than 7, less than 6, less than 5, less than 4, less than 3, less than 2, or less than 1 microneedle/mm² of the substrate. In specific cases, the density of the plurality of microneedles on the substrate is about 0.5 microneedle/mm² of the substrate. In specific cases, the density of the plurality of microneedles on the substrate is about 0.51 microneedle/mm² of the substrate. In specific cases, the density of the plurality of microneedles on the substrate is about 0.52 microneedle/mm² of the substrate. In specific cases, the density of the plurality of microneedles on the substrate is about 0.53 microneedle/mm² of the substrate. In specific cases, the density of the plurality of microneedles on the substrate is about 0.54 microneedle/mm² of the substrate. In specific cases, the density of the plurality of microneedles on the substrate is about 0.55 microneedle/mm² of the substrate. In specific cases, the density of the plurality of microneedles on the substrate is about 0.56 microneedle/mm² of the substrate. In specific cases, the density of the plurality of microneedles on the substrate is about 0.57 microneedle/mm² of the substrate. In specific cases, the density of the plurality of microneedles on the substrate is about 0.58 microneedle/mm² of the substrate. In specific cases, the density of the plurality of microneedles on the substrate is about 0.59 microneedle/mm² of the substrate. In specific cases, the density of the plurality of microneedles on the substrate is about 0.6 microneedle/mm² of the substrate. In specific cases, the density of the plurality of microneedles on the substrate is about 0.61 microneedle/mm² of the substrate. In specific cases, the density of the plurality of microneedles on the substrate is about 0.62 microneedle/mm² of the substrate. In specific cases, the density of the plurality of microneedles on the substrate is about 0.63 microneedle/mm² of the substrate. In specific cases, the density of the plurality of microneedles on the substrate is about 0.64 microneedle/mm² of the substrate. In specific cases, the density of the plurality of microneedles on the substrate is about 0.65 microneedle/mm² of the substrate.

In some cases, the plurality of microneedles are arranged in a single region on the substrate. In other cases, the plurality of microneedles are arranged in multiple regions on the substrate. In some cases, the plurality of microneedles are arranged in linear rows. In some cases, the plurality of microneedles are arranged in circles. In some cases, the plurality of microneedles are arranged in at least 2 rows and at least 2 microneedles in each row. In some cases, the plurality of microneedles are arranged in at least 5 rows and at least 2 microneedles in each row. In some cases, the plurality of microneedles are arranged in at least 10 rows and at least 2 microneedles in each row. In some cases, the plurality of microneedles are arranged in at least 2 rows and at least 5 microneedles in each row. In some cases, the plurality of microneedles are arranged in at least 2 rows and at least 10 microneedles in each row. In some specific embodiments, the plurality of microneedles are arranged in 10 rows and 10 microneedles in each row.

In some cases, the substrate is made of the same material as the plurality of microneedles. In some cases, the substrate is made of hyaluronic acid, sodium alginate, polylactic acid, polyglycolic acid, polylactic-glycolic acid, cartilage thioflavin, silk protein, maltose, chitosan, carboxymethyl cellulose, or any combination thereof. In other cases, the substrate is made of a different material from the plurality of microneedles. In some cases, the substrate is made of polymers (e.g., polycarbonate, polypropylene, or polyethylene) stainless steels, alloys, titanium, or any combination thereof. In some cases, the substrate is at least 100, at least 500, at least 1000, at least 1500, at least 2000, at least 2500, at least 3000, at least 3500, at least 4000, at least 4500, or at least 5000 thick. In some cases, the substrate is near round, near a sector of circle, near square, near rectangle, near triangle, near pentagon, near parallelogram, near trapezoid, or near polygon. In some cases, the substrate has a Young's modulus of less than 10, less than 9, less than 8, less than 7, less than 6, less than 5, less than 4, less than 3, less than 2, less than 1, less than 0.9, less than 0.8, less than 0.7, less than 0.6, less than 0.5, less than 0.4, less than 0.3, less than 0.2, or less than 0.1.

In some cases, each microneedle of the plurality of microneedles has a base that attached to the substrate. In some cases, at least one microneedle of the plurality of microneedles has a point that is capable of piercing a target. In specific cases, the point is capable of piercing stratum corneum of a subject. In specific cases, the point is capable of piercing epidermis of a subject. In specific cases, the point is capable of piercing dermis of a subject.

In some cases, the plurality of microneedles are solid. In some cases, the plurality of microneedles are hollow. In some cases, at least one microneedle of the plurality of microneedles that are hollow has an inner diameter of at least 0.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.9, at least 1, at least 1.5, at least 2, at least 2.5, or at least 3 mm. In some cases, at most one microneedle of the plurality of microneedles that are hollow has an inner diameter of at most 0.05, at most 0.1, at most 0.15, at most 0.2, at most 0.25, at most 0.3, at most 0.35, at most 0.4, at most 0.45, at most 0.5, at most 0.55, at most 0.6, at most 0.65, at most 0.7, at most 0.75, at most 0.8, at most 0.9, at most 1, at most 1.5, at most 2, at most 2.5, at most 3 mm, at most 4 mm, at most 5 mm, at most 6 mm, at most 7 mm, at most 8 mm, at most 9 mm, or at most 10 mm

In some cases, the at least one microneedle of the plurality of microneedles comprises a hydrogel. In specific cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof.

In some cases, at least one microneedle of the plurality of microneedles has a conical shape. In some cases, at least one microneedle of the plurality of microneedles has a funnel shape. In some cases, at least one microneedle of the plurality of microneedles has a pyramidal shape. In some cases, at least one microneedle of the plurality of microneedles has a tapered shape. In some cases, at least one microneedle of the plurality of microneedles has a shape as disclosed in Makvandi et al. Nano-Micro Letters volume 13, Article number: 93 (2021).

The plurality of microneedles provided herein generally have at least one microneedle with a base of a certain length. In some cases, the at least one microneedle of the plurality of microneedles comprises a base with a diameter of less than 2000 μm, less than 1800 μm, less than 1500 μm, less than 1000 μm, less than 900 μm, less than 800 μm less than 700 μm, less than 600 μm, less than 500 μm, less than 400 μm, less than 300 μm, less than 200 μm, or less than 100 μm. In some cases, the at least one microneedle of the plurality of microneedles comprises a base with a diameter of at least 2000 μm, at least 1800 μm, at least 1500 μm, at least 1000 μm, at least 900 μm, at least 800 μm at least 700 μm, at least 600 μm, at least 500 μm, at least 400 μm, at least 300 μm, at least 200 μm, at least 100 μm, at least 10 μm, or at least 1 μm. In some cases, the at least one microneedle of the plurality of microneedles comprises a base with a diameter of from about 100 μm to about 1000 μm, from about 200 μm to about 900 μm, from about 300 μm to about 800 μm, from about 400 μm to about 700 μm, from about 500 μm to about 600 μm. In specific embodiments, the at least one microneedle of the plurality of microneedles comprises a base with a diameter of about 100, 200, 300, 400, 500, 600, 700, 800, 900, or 1000 m. In specific embodiments, the at least one microneedle of the plurality of microneedles comprises a base with a diameter of about 400 μm.

At least one microneedle of the microneedle device has a tip radius of a certain size. In some cases, the tip radius of the at least one microneedle of the microneedle device is no greater than 4 mm, 3.5 mm, 3 mm, 2.5 mm, 2 mm, 1.5 mm, 1 mm, 0.5 mm, 0.4 mm, 0.3 mm, 0.2 mm, or 0.1 mm.

In some cases, the length of the at least one microneedle of the plurality of microneedles is at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000 μm. In some cases, the length of the at least one microneedle of the plurality of microneedles is no greater than 100, no greater than 200, no greater than 300, no greater than 400, no greater than 500, no greater than 600, no greater than 700, no greater than 800, no greater than 900, no greater than 1000, no greater than 1100, no greater than 1200, no greater than 1300, no greater than 1400, no greater than 1500, no greater than 1600, no greater than 1700, no greater than 1800, no greater than 1900, or no greater than 2000 μm. In other cases, the length of the at least one microneedle of the plurality of microneedles is from 100 μm to 3000 μm, from 500 μm to 2500 μm, from 1000 μm to 2500 μm, or from 1000 μm to 2000 μm. In specific embodiments, the length of the at least one microneedle of the plurality of microneedles is about 1000 μm. In specific embodiments, the length of the at least one microneedle of the plurality of microneedles is about 2000 μm.

The length of the at least one microneedle of the plurality of microneedles needs to be adjusted based on the thickness of the target tissue to be penetrated. In some cases, if forehead is the target tissue, the length of the at least one microneedle of the plurality of microneedles is at least 0.25 to 0.5 mm for thin skin, and the length of the at least one microneedle of the plurality of microneedles is at least 0.5 to 0.75 mm for thick skin. In some cases, if between-eyebrow is the target tissue, the length of the at least one microneedle of the plurality of microneedles is at least 0.25 to 0.5 mm for thin skin, and the length of the at least one microneedle of the plurality of microneedles is at least 0.5 to 0.75 mm for thick skin. In some cases, if eye area is the target tissue, the length of the at least one microneedle of the plurality of microneedles is at least 0.25 mm for thin skin, and the length of the at least one microneedle of the plurality of microneedles is at least 0.25 to 0.5 mm for thick skin. In some cases, if nose is the target tissue, the length of the at least one microneedle of the plurality of microneedles is at least 0.25 mm for thin skin, and the length of the at least one microneedle of the plurality of microneedles is at least 0.5 mm for thick skin. In some cases, if cheek bone is the target tissue, the length of the at least one microneedle of the plurality of microneedles is at least 0.5 mm for thin skin, and the length of the at least one microneedle of the plurality of microneedles is at least 0.5 to 1.0 mm for thick skin. In some cases, if lip area is the target tissue, the length of the at least one microneedle of the plurality of microneedles is at least 0.25 mm for thin skin, and the length of the at least one microneedle of the plurality of microneedles is at least 0.25 to 0.75 mm for thick skin. In some cases, if cheek or chin is the target tissue, the length of the at least one microneedle of the plurality of microneedles is at least 0.5 to 1.0 mm for thin skin, and the length of the at least one microneedle of the plurality of microneedles is at least 1.0 to 2.0 mm for thick skin. In some cases, if neck is the target tissue, the length of the at least one microneedle of the plurality of microneedles is at least 0.5 to 1.0 mm for thin skin, and the length of the at least one microneedle of the plurality of microneedles is at least 1.0 to 2.0 mm for thick skin. In some cases, if chest is the target tissue, the length of the at least one microneedle of the plurality of microneedles is at least 0.5 to 1.0 mm for thin skin, and the length of the at least one microneedle of the plurality of microneedles is at least 1.0 to 2.0 mm for thick skin.

In some embodiments, the center-to-center distance between the microneedle of the plurality of microneedles is at least 100, 200, 300, 400, 500, 600, 700 μm. In other embodiments, the center-to-center distance between the microneedle of the plurality of microneedles is about 100 to 2000 μm, 100 to 1500 μm, 100 to 1000 μm, 300 to 2000 μm, 300 to 1500 μm, 300 to 1000 μm, 500 to 2000 μm, 500 to 1500 μm, 500 to 1000 μm. In specific cases, the center-to-center distance between the microneedle of the plurality of microneedles is about 700 μm. In other specific cases, the center-to-center distance between the microneedle of the plurality of microneedles is about 1000 m.

In some cases, the at least one microneedle of the plurality of microneedles comprises a low aspect ratio that results in stronger internal support and less breakage. In some cases, the plurality of microneedles distribute contents in the target tissue evenly.

In some cases, the at least one microneedle of the plurality of microneedles comprises at least 3×10², at least 3×10³, at least 3×10⁴, at least 3×10⁵, at least 3×10⁶, at least 3×10⁷, at least 3×10⁸, at least 3×10⁹, 3×10¹⁰° 3×10¹¹, 3×10¹²° 3×10¹⁵, 3×10²⁰, or 3×10³⁰ EVs. In some cases, the at least one microneedle of the plurality of microneedles comprises about 3×10² to 3×10¹², about 3×10³ to 3×10¹², about 3×10⁴ to 3×10¹², about 3×10⁵ to 3×10¹², about 3×10⁶ to 3×10¹², about 3×10⁷ to 3×10¹², about 3×10⁶ to 3×10¹², about 3×10⁷ to 3×10¹², about 3×10⁸ to 3×10¹², about 3×10⁹ to 3×10¹², about 3×10¹⁰ to 3×10¹², or about 3×10¹¹ to 3×10¹² EVs. In some cases, the at least one microneedle of the plurality of microneedles comprises about 3×10² to 3×10¹¹, about 3×10³ to 3×10¹¹, about 3×10⁴ to 3×10¹¹, about 3×10⁵ to 3×10¹¹, about 3×10⁶ to 3×10¹¹, about 3×10⁷ to 3×10¹¹, about 3×10⁶ to 3×10¹¹, about 3×10⁷ to 3×10¹¹, about 3×10⁸ to 3×10¹¹, about 3×10⁹ to 3×10¹¹, or about 3×10¹⁰ to 3×10¹¹ EVs. In some cases, the at least one microneedle of the plurality of microneedles comprises about 3×10² to 3×10¹⁰, about 3×10³ to 3×10¹⁰, about 3×10⁴ to 3×10¹⁰, about 3×10⁵ to 3×10¹⁰, about 3×10⁶ to 3×10¹⁰, about 3×10⁷ to 3×10¹⁰, about 3×10⁶ to 3×10¹⁰, about 3×10⁷ to 3×10¹⁰, about 3×10⁸ to 3×10¹⁰, or about 3×10⁹ to 3×10¹⁰ EVs. In other cases, the at least one microneedle of the plurality of microneedles comprises about 3×10² to 3×10⁹, about 3×10³ to 3×10⁹, about 3×10⁴ to 3×10⁹, about 3×10⁵ to 3×10⁹, about 3×10⁶ to 3×10⁹, about 3×10⁷ to 3×10⁹, about 3×10⁶ to 3×10⁹, about 3×10⁷ to 3×10⁹, or about 3×10⁸ to 3×10⁹ EVs. In some cases, the at least one microneedle of the plurality of microneedles comprises about 3×10² to 3×10⁸, about 3×10³ to 3×10⁸, about 3×10⁴ to 3×10⁸, about 3×10⁵ to 3×10⁸, about 3×10⁶ to 3×10⁸, about 3×10⁷ to 3×10⁸, about 3×10⁶ to 3×10⁸, or about 3×10⁷ to 3×10⁸ EVs.

In some cases, the EVs in the at least one microneedle of the plurality of microneedles are suspended in hydrogel. In some cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In specific cases, the hydrogel comprises hyaluronic acid.

In some cases, the at least one microneedle of the plurality of microneedles has a breaking strength retention of at most 50%, 40%, 30%, 20%, or 10% in at most 30, 20, 15, 10, or 5 minutes. In some specific cases, the at least one microneedle of the plurality of microneedles has a breaking strength retention of at most 50%, 40%, 30%, 20%, or 10% in about 30, 20, 15, 10, or 5 minutes. In some specific cases, the at least one microneedle of the plurality of microneedles has a breaking strength retention of at most 50%, 40%, 30%, 20%, or 10% in at most 60, 50 or 40 minutes. In some specific cases, the at least one microneedle of the plurality of microneedles has a breaking strength retention of at most 50%, 40%, 30%, 20%, or 10% in about 60, 50 or 40 minutes. In some specific cases, the at least one microneedle of the plurality of microneedles has a breaking strength retention of at most 50%, 40%, 30%, 20%, or 10% in at most 1, 3, 5, 10, 15, 20, or 24 hours. In some specific cases, the at least one microneedle of the plurality of microneedles has a breaking strength retention of at most 50%, 40%, 30%, 20%, or 10% in about 1, 3, 5, 10, 15, 20, or 24 hours. In some specific cases, the at least one microneedle of the plurality of microneedles has a breaking strength retention of at most 50%, 40%, 30%, 20%, or 10% in at most 1, 3, 5, 7, 9, 14, 21, or 30 days. In some specific cases, the at least one microneedle of the plurality of microneedles has a breaking strength retention of at most 50%, 40%, 30%, 20%, or 10% in about 1, 3, 5, 7, 9, 14, 21, or 30 days. In some cases, the at least one microneedle of the plurality of microneedles comprises a type of protein. In some cases, the dissolvable gel comprises a compound from an animal source. In some cases, the at least one microneedle of the plurality of microneedles comprises a synthetic compound. In some cases, the at least one microneedle of the plurality of microneedles comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In some cases, the at least one microneedle of the plurality of microneedles has a breaking strength retention of at most 50%, 40%, 30%, 20%, or 10% when exposed to a tissue. In some specific cases, the at least one microneedle of the plurality of microneedles has a breaking strength retention of at most 50%, 40%, 30%, 20%, or 10% when exposed to epidermis. In some specific cases, the at least one microneedle of the plurality of microneedles has a breaking strength retention of at most 50%, 40%, 30%, 20%, or 10% when exposed to dermis. In some specific cases, the at least one microneedle of the plurality of microneedles has a breaking strength retention of at most 50%, 40%, 30%, 20%, or 10% when exposed to hypodermis.

In some cases, the at least one microneedle of the plurality of microneedles has a breaking strength retention of at most 50%, 40%, 30%, 20%, or 10% when the local temperature is increased by at least 1° C., 2° C., 3° C., 4° C., 5° C., 6° C., 7° C., 8° C., 9° C., or 10° C. In some cases, the at least one microneedle of the plurality of microneedles has a breaking strength retention of at most 50%, 40%, 30%, 20%, or 10% when the local temperature is increased by at most 15° C., 14° C., 13° C., 12° C., 11° C., or 10° C.

In some cases, the EVs in the at least one microneedle of the plurality of microneedles comprise extracellular matrix mRNA. In specific cases, the EVs in the at least one microneedle of the plurality of microneedles comprise exogenous extracellular matrix mRNA. In some cases, the extracellular matrix mRNA or the exogenous extracellular matrix mRNA comprises collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or any combination thereof. In other cases, the EVs in the at least one microneedle of the plurality of microneedles comprise VEGF mRNA. In specific cases, the EVs in the at least one microneedle of the plurality of microneedles comprise exogenous VEGF mRNA. In some cases, the VEGF mRNA or the exogenous VEGF mRNA comprises VEGFA, VEGFB, VEGFC, VEGFD, PIGF, or any combination thereof.

In some cases, the microneedle devices comprise siRNA. In some cases, the microneedle devices comprise miRNA. In some cases, the microneedle devices comprise DNA, plasmid, or other nucleic acids. In some cases, the microneedle devices comprise small molecule drugs with or without EVs. In some cases, the microneedle devices comprise large macromolecules with or without EVs. In some cases, the microneedle devices comprise insulin with or without EVs. In some cases, the microneedle devices comprise a growth hormone with or without EVs. In some cases, the microneedle devices comprise a vaccine or an active ingredient thereof. In some cases, the microneedle devices comprise receptor agonist with or without EVs.

In some cases, the microneedle devices comprise an anesthetic agent with or without EVs. In some cases, the microneedle devices comprise a protein or a peptide with or without EVs.

In some cases, all microneedles of the plurality of microneedles comprise EVs disclosed herein. In other cases, a portion of the plurality of microneedles comprise EVs disclosed herein, and the other portion of the plurality of microneedles are without EVs. In specific cases, a portion of the plurality of microneedles comprise EVs disclosed herein, and the other portion of the plurality of microneedles comprise a different substance without EVs. In some cases, the different substance is, an anesthetic agent, a healing agent, an ingredient for improving skin condition, aloe, or any combination thereof. In specific cases, the anesthetic agent is lidocaine, pramoxine, phenol, prilocaine, benzocaine, dibucaine, methyl salicylate, capsaicin, zinc acetate, tetracaine, hydrocortisone, menthol, or prilocaine. In specific cases, the healing agent is vitamin C, an extract from Aloe vera, an extract from Hippophae rhamnoides (sea buckthorn), an extract from Angelica sinensis, an extract from Catharanthus roseus (Vinca rosea), or green tea extract. In specific cases, the ingredient for improving skin condition is glycerin, coenzyme Q10 (CoQ10), ceramides, cetyl and stearyl alcohol, petrolatum, squalene, or lactic acid.

Further provided herein are methods of manufacturing a microneedle device, wherein the method comprises: (a) mixing extracellular vesicles (EVs) with a first batch of polymerizable solution; and (b) casting the mixture from step (a) to a polydimethylsiloxane (PDMS) mold with at least one needle-like shape. In some cases, the method further comprises a step (c) after step (b), wherein step (c) comprises concentrating the EVs in a tip of the at least one needle-like shape of the PDMS mold. In some specific cases, the concentrating is by maintaining the PDMS mold at a temperature of at most 15° C., at most 14° C., at most 13° C., at most 12° C., at most 11° C., at most 10° C., at most 9° C., at most 8° C., at most 7° C., at most 6° C., at most 5° C., at most 4° C., at most 3° C., at most 2° C., at most 1° C., or at most 0° C. In some specific cases, the concentrating is by maintaining the PDMS mold at a temperature of at least 6° C., at least 5° C., at least 4° C., at least 3° C., at least 2° C., at least 1° C., at least 0° C., at least −1° C., at least −2° C., at least −3° C., at least −4° C., at least −5° C., at least −6° C., at least −7° C., at least −8° C., or at least −10° C. In specific cases, the concentrating is by maintaining the PDMS mold at about 1° C., about 2° C., about 3° C., about 4° C., about 5° C., about 6° C., about 7° C., about 8° C., about 9° C., or about 10° C. In one specific case, the concentrating is by maintaining the PDMS mold at about 4° C. In some cases, the concentration lasts for at least 1, 2, 3, 4, 5, 6, 7, 8, 9, or 10 hours. In some cases, the concentration lasts for no greater than 1, 2, 3, 4, 5, 6, 7, 8, 9, 10, 11, 12, 13, 14, 15, 16, 17, 18, 19, 20, 30, 40, 50, or 60 hours. In some cases, the concentrating lasts from about 1 to about 10, about 2 to about 8, about 2 to about 6, about 2 to about 4, about 3 to about 8, or about 3 to about 6 hours. In some cases, the concentrating lasts for about 1 hour. In some cases, the concentrating lasts for about 2 hours. In some cases, the concentrating lasts for about 3 hours. In some cases, the concentrating lasts for about 4 hours. In some cases, the concentrating lasts for about 5 hours. In some cases, the concentrating lasts for about 6 hours.

In some cases, the method further comprises a step (d) after step (c), wherein step (d) comprises adding a second batch of the polymerizable solution on top of the PDMS mold.

In some cases, the polymerizable solution comprises hydrogel. In specific cases, the hydrogel comprises hyaluronic acid, sodium alginate, polylactic acid, polyglycolic acid, and polylactic-glycolic acid, cartilage thioflavin, silk protein, maltose, chitosan, carboxymethyl cellulose, or any combination thereof. In specific cases, the hydrogel comprises hyaluronic acid. In some cases, the second batch of the polymerizable solution has the same composition with the first batch of the polymerizable solution. In some cases, the second batch of the polymerizable solution has a different composition from the first batch of the polymerizable solution.

The hydrogel disclosed herein comprises a certain percentage of hyaluronic acid, which consists of repeating disaccharide units of D-glucuronic acid and N-acetyl-D-glucosamine, which are linked through alternating β-(1→4) and β-(1→3) glycosidic bonds. In some cases, the hydrogel comprises at least 5%, at least 10%, at least 15%, at least 20%, at least 25%, at least 30%, or at least 35% hyaluronic acid. In some cases, the hydrogel comprises about 5%, about 10%, about 15%, about 20%, about 25%, about 30%, or about 35% hyaluronic acid. In some cases, the second batch of the polymerizable solution has the same percentage of hyaluronic acid with the first batch of the polymerizable solution. In some cases, the second batch of the polymerizable solution has a different percentage of hyaluronic acid from the first batch of the polymerizable solution. In some specific cases, the second batch of the polymerizable solution has a higher percentage of hyaluronic acid from the first batch of the polymerizable solution. In other specific cases, the second batch of the polymerizable solution has a lower percentage of hyaluronic acid from the first batch of the polymerizable solution.

In some cases, the final ratio between EVs and the polymerizable solution is at least 1:2, 1:3, 1:4, 1:5, 1:10, 1:15, 1:20, 1:25, 1:30, 1:35, 1:40, 1:45, 1:50, 1:55, 1:60, 1:65, 1:70, 1:80, 1:90, or 1:100. In some cases, the final ratio between EVs and the polymerizable solution is no greater than 1:2, 1:3, 1:4, 1:5, 1:10, 1:15, 1:20, 1:25, 1:30, 1:35, 1:40, 1:45, or 1:50. In some cases, the final ratio between EVs and the polymerizable solution is about 1:5, 1:10, 1:15, 1:20, 1:25, 1:30, 1:35, 1:40, 1:50, 1:60, 1:70, 1:80, 1:90, or 1:100. In specific cases, the final ratio between EVs and the polymerizable solution is about 1:21, 1:22, 1:23, 1:24, or 1:25.

In some cases, the EVs used in the method of manufacturing a microneedle device comprise extracellular matrix mRNA. In specific cases, the EVs used in the method of manufacturing a microneedle device comprise exogenous extracellular matrix mRNA. In some cases, the extracellular matrix mRNA or the exogenous extracellular matrix mRNA comprises collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or any combination thereof. In other cases, the EVs used in the method of manufacturing a microneedle device comprise VEGF mRNA. In specific cases, the EVs used in the method of manufacturing a microneedle device comprise exogenous VEGF mRNA. In some cases, the VEGF mRNA or the exogenous VEGF mRNA comprises VEGFA, VEGFB, VEGFC, VEGFD, PIGF, or any combination thereof.

Further provided herein are methods of administering extracellular vesicles (EVs) to a tissue of a subject in need thereof, the method comprising applying the microneedle device disclosed herein.

In some embodiments, the applying does not require external pumps. In other embodiments, the applying is performed with an aid of a machine with pre-set pressure. In some embodiments, the applying is performed manually. In some embodiments, the applying is performed manually in certain portion of the tissue and with an aid of a machine with pre-set pressure in other portion of the tissue.

Since the microneedles in the microneedle device disclosed herein dissolve by themselves and release EVs to the tissue, at the end of the administering, the microneedle device is removed. In some cases, the microneedle device is removed after about 5, 10, 15, 20, 25, 30, 35, 40, 45, 50, 55, or 60 minutes. In order to keep the procedure sterile, in some cases, the tissue is disinfected before applying the microneedle device disclosed herein. In other cases, the tissue is disinfected after applying the microneedle device disclosed herein.

In some cases, the tissue is epidermis. In some cases, the tissue is dermis. In some cases, the tissue is hypodermis. In some cases, the subject is a rodent. In other cases, the subject is a mammal. In some specific cases, the subject is a guinea pig. In some specific cases, the subject is a monkey. In some specific cases, the subject is a guinea pig. In some specific cases, the subject is a rabbit. In some specific cases, the subject is a human.

In some cases, the administering is performed one time in high dosage of EVs. In some specific cases, the high dosage of EVs is at least 10¹⁰, 10¹¹, 10¹², 10¹³, 10¹⁴, 10¹⁵, 10²⁰, 10²⁵, 10³⁰, or 1035 EVs. In some cases, the high dosage of EVs is about 10¹⁰, 10¹¹, 10¹², 10¹³, 10¹⁴, 10¹⁵, 10²⁰, 10²⁵, 10³⁰, or 10³⁵ EVs. In other cases, the administering is performed multiple times over a period of time in a low dosage of EVs. In some specific cases, the low dosage of EVs is at most 10, 10², 10³, 10⁴, 10⁵, 10⁶, 10⁷, 10⁸, 10⁹, 10¹⁰, 10¹¹, 10¹², 10¹³, 10¹⁴, or 10¹⁵ EVs. In some specific cases, the administering is performed multiple times over at least 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days. In some specific cases, the administering is performed multiple times over about 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1.5 months, 2 months, 3 months, 4 months, 5 months, or 6 months. In some cases, when the administering is performed multiple times over a period of time, the interval between the doses is at least 1 day, 5 days, 10 days, 15 days, 20 days, 30 days, 40 days, 50 days, 60 days, 70 days, or 80 days.

In some cases, methods of administering EVs with the microneedle device disclosed herein result in less breakage of the EVs than administering the EVs in a conventional microneedle or a syringe. In some cases, methods of administering EVs with the microneedle device disclosed herein result in more even distribution of EVs in the tissue than administering the EVs in a conventional microneedle or a syringe. In some cases, methods of administering EVs with the microneedle device disclosed herein result in less irritation at administration site than administering the EVs in a conventional microneedle or a syringe. In the above cases, the conventional microneedle is a solid or hollow microneedle.

Further provided herein are methods of treating a skin damage in a subject in need thereof, the method comprising applying the microneedle device containing extracellular matrix mRNA disclosed herein. In some cases, the skin damage is caused by aging. In other cases, skin damage is caused by sun damage. In other cases, the skin damage is caused by an autoimmune disease. In other cases, the skin damage is caused by burning. In other cases, the skin damage is caused by scars. In other cases, the skin damage is caused by a skin disease. In some specific cases, the skin damage is caused by dermatosparaxis Ehlers-Danlos syndrome. In some specific cases, the skin damage is caused by epidermolysis bullosa. In some specific cases, the skin damage is caused by pseudosyndactyly. In some specific cases, the skin damage is caused by poor wound healing. In some specific cases, the skin damage is caused by digit and limb contractures. In some specific cases, the skin damage is caused by keratodermas. In some specific cases, the skin damage is caused by systemic sclerosis. In some specific cases, the skin damage is caused by scleroderma. In some specific cases, the skin damage is caused by atopic dermatitis. In some specific cases, the skin damage is caused by periodontal disease.

In some cases, the method of treating the skin damage results in at least a 10% reduction in appearance of wrinkles. In other cases, the method of treating the skin damage results in more collagen fibers. In other cases, the method of treating the skin damage results in a higher dermal thickness.

In some cases, the method of treating the skin damage with the microneedle device disclosed herein results in a prolonged effect for at least 30 days, at least 40 days, at least 50 days, at least 60 days, at least 70 days, at least 80 days, or at least 90 days. In some cases, the method of treating the skin damage with the microneedle device disclosed herein results in a prolonged effect for about 30 days, about 40 days, about 50 days, about 60 days, about 70 days, about 80 days, or about 90 days.

In some cases, the method of treating the skin damage described herein comprises administering to the subject disclosed herein with at least 1 dose per 12 months, at least 1 dose per 11 months, at least 1 dose per 10 months, at least 1 dose per 9 months, at least 1 dose per 8 months, at least 1 dose per 7 months, at least 1 dose per 6 months, at least 1 dose per 5 months, at least 1 dose per 4 months, at least 1 dose per 3 months, or at least 1 dose per 2 months. In other cases, the method of treating the skin damage described herein comprises administering to the subject disclosed herein with at most 1 dose per week, at most 1 dose per 2 weeks, at most 1 dose per 3 weeks, at most 1 dose per 4 weeks, at most 1 dose per month. In other cases, the method of treating the skin damage described herein comprises administering to the subject disclosed herein with about 1 dose per month, about 1 dose per 2 months, or about 1 dose per 3 months.

Further provided herein are methods of reducing the risk of pseudosyndactyly, poor wound healing, or digit and limb contracture in a subject in need thereof, and the methods comprise administering to the subject with the microneedle device described herein containing an effective amount of COL7A1 mRNA loaded EVs. In some cases, the subject is a patient with dystrophic epidermolysis bullosa. In some cases, the administering is to the hands and/or feet of the subject.

Further provided herein are methods of preventing, alleviating, or treating palmoplantar keratoderma in a subject in need thereof, and the methods comprise administering to the subject with an effective amount of viable keratin mRNA. In some cases, the administering comprises using the microneedle device described herein. In some cases, the administering is to the affected skin of the subject.

Further provided herein are methods of preventing, alleviating, or treating keloid scarring in a subject in need thereof, and the methods comprise administering to the subject with an effective amount of an inhibitory agent that reduces TGF-Beta 1, SMAD3, and EPHB2. In some cases, the administering comprises using the microneedle device described herein.

Further provided herein are methods of administering to a subject in need thereof with the microneedle device described herein containing EVs of any cargos. In some cases, the cargos are microRNAs (miRNAs). In some cases, the cargos are small interfering RNAs (siRNAs). In some cases, the cargos are peptides. In some cases, the cargos are proteins. In some cases, the cargos are inhibitory double stranded RNAs (dsRNAs). In some cases, the cargos are small or short hairpin RNAs (shRNAs). In some cases, the cargos are antisense oligonucleotides (ASOs). In some cases, the cargos are piwi-interacting RNAs (piRNAs). In some cases, the cargos are heterogeneous nuclear RNAs (hnRNAs). In some cases, the cargos are small nuclear RNAs (snRNAs). In some cases, the cargos are enzymatically-prepared siRNAs (esiRNAs). In some cases, the cargos are precursors of any of the above cargos. In some cases, the cargos are any combinations of the above two or more cargos.

Further provided herein are methods of treating a blood flow disorder a subject in need thereof, the method comprising applying the microneedle device containing VEGF mRNA disclosed herein. In some specific cases, the blood flow disorder is ischemia.

Described herein, in some aspect, is a method of administering extracellular vesicles (EVs) to a tissue of a subject, the method comprising administering the needle, the syringe, the hydrogel needle, the microneedle, or the microneedle device disclosed herein to the tissue of the subject. In some cases, the method comprises administering the microneedle device disclosed herein to the tissue of the subject. In some cases, the microneedle device is removed after at least 5, 10, 15, 20, 25, or 30 minutes. In some cases, the microneedle device is removed after about 10, 15, 20, or 30 minutes.

In some cases, the EVs comprise at least one mRNA. In some cases, the administering comprises administering at least 1 ng, 10 ng, 50 ng, 100 ng, 1 μg, 10 μg, or 20 μg of the at least one mRNA to the tissue of the subject. In some cases, the administering comprises administering 1 ng-10 μg of the extracellular matrix mRNA to the tissue of the subject. In some cases, the administering comprises administering 1 ng-20 μg of the extracellular matrix mRNA to the tissue of the subject

In some cases, the tissue is subcutis. In some cases, the tissue is dermis. In some cases, the tissue is epidermis. In some cases, the tissue is a gum. In some cases, the tissue is a lip. In some cases, the subject is a mammal. In some cases, the subject is a human. In some cases, the subject is a rodent. In some cases, the subject is a monkey. In some cases, the subject is a rabbit.

In some cases, the administering is performed a single time and administers at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, or 1×10¹⁴ extracellular vesicles. In some cases, the administering is performed a single time and administers at most 1×10⁷, 1×10⁸, 1×10⁹, 1×10¹⁰, 1×10¹¹, 1×10¹², 1×10¹³, 1×10¹⁴, 1×10¹⁵, 1×10¹⁷, 1×10¹⁸, 1×10¹⁹, 1×10²⁰, or 1×10³⁰ extracellular vesicles. In some cases, the administering is performed a single time and administers at most 1×10¹³⁻¹⁴ extracellular vesicles. In some cases, the administering is performed multiple times and administers each time at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, or 1×10¹⁴ extracellular vesicles. In some cases, the administering is performed multiple times and administers each time at most 1×10⁷, 1×10⁸, 1×10⁹, 1×10¹⁰, 1×10¹¹, 1×10¹², 1×10¹³, 1×10¹⁴, 1×10¹⁵, 1×10¹⁷, 1×10¹⁸, 1×10¹⁹, 1×10²⁰, or 1×10³⁰ extracellular vesicles. In some cases, the administering is performed multiple times and administers each time at most 1×10¹³⁻¹⁴ extracellular vesicles. In some cases, the administering is performed multiple times and administers at most 1×10¹³⁻¹⁴ extracellular vesicles within 6-8 weeks. In some cases, the administering is performed multiple times over a period of time and administers at most 1×10⁷, 1×10⁸, 1×10⁹, 1×10¹⁰, 1×10¹¹, 1×10¹², 1×10¹³, 1×10¹⁴, 1×10¹⁵, 1×10¹⁷, 1×10¹⁸, 1×10¹⁹, or 1×10²⁰, 1×10³⁰ extracellular vesicles.

In some cases, the method results in less breakage of the EVs than administering the EVs in a conventional microneedle or a syringe. In some cases, the method results in less irritation at an administration site than administering the EVs in a conventional microneedle or a syringe. In some cases, the method results in more even distribution of EVs in the tissue than administering the EVs in a conventional microneedle or a syringe. In some cases, the conventional microneedle is a solid microneedle or a hollow microneedle.

Described herein, in some aspect, is a method of treating a skin condition in a subject in need thereof, the method comprising applying the needle, the syringe, the hydrogel needle, the microneedle, or the microneedle device disclosed herein to the subject. In some cases, the method comprising administering the microneedle disclosed herein to the subject.

In some cases, the skin condition is skin damage. In some cases, the skin damage is caused by aging or sun damage. In some cases, the skin condition is a wound.

In some cases, the administering comprises administering at least 0.1 ng, 1 ng, 5 ng, 10 ng, 20 ng, 30 ng, 40 ng, 50 ng, 100 ng, 200 ng, 300 ng, 400 ng, 500 ng, 600 ng, 700 ng, 800 ng, 900 ng, 1 μg, or 10 μg of the extracellular matrix mRNA. In some cases, the administering comprises administering no greater 0.1 ng, 1 ng, 5 ng, 10 ng, 20 ng, 30 ng, 40 ng, 50 ng, 100 ng, 200 ng, 300 ng, 400 ng, 500 ng, 600 ng, 700 ng, 800 ng, 900 ng, 1 μg, 10 μg, 20 μg, 30 μg, 40 μg, 50 μg, 60 μg, 70 μg, 80 μg, 90 μg, 100 μg, 200 μg, 300 μg, 400 μg, 500 μg, 600 μg, 700 μg, 800 μg, or 900 μg of the extracellular matrix mRNA. In some cases, the administering comprises administering about 0.1 ng-50 μg, 1 ng-50 μg, 1 ng-30 μg, 1 ng-25 μg of the extracellular matrix mRNA. In some cases, the administering comprises administering about 1 ng-20 μg of the extracellular matrix mRNA.

In some cases, the method results in at least a 10%, 20%, 30%, 40%, 50%, 60%, 70%, 80%, or 90% reduction in appearance of wrinkles. In some cases, the method results in at least 10%, 20%, 30%, 40%, 50%, 60%, 70%, 80%, or 90% more collagen fibers. In some cases, the method results in a higher dermal thickness by at least 10%, 20%, 30%, 40%, 50%, 60%, 70%, 80%, or 90%. In some cases, the method results in a prolonged effect for at least 30 days, at least 40 days, at least 50 days, at least 60 days, at least 70 days, at least 80 days, or at least 90 days.

In some cases, at least 50%, 70%, 75%, 80%, 85%, 90%, 95%, or 99% of the needle, the microneedle, or the microneedle of the microneedle device comprises hydrogel. In some case, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof. In some cases, the hydrogel comprises hyaluronic acid. In some cases, the hydrogel comprises at least 1%, 5%, 7%, 10%, 12%, 15%, 20%, 25% 50%, 70%, 75%, 80%, 85%, 90%, 95%, or 99% hyaluronic acid. In some cases, the hydrogel comprises at least 5% hyaluronic acid. In some cases, the hydrogel comprises about 15% hyaluronic acid. In some cases, the hydrogel comprises at least 5% hyaluronic acid and at most 30% hyaluronic acid. In some cases, the hydrogel comprises greater than 10% hyaluronic acid and at most 20% hyaluronic acid. In some cases, the hydrogel comprises greater than 10% hyaluronic acid and at most 15% hyaluronic acid. In some cases, the hydrogel comprises greater than 10% hyaluronic acid and at most 18% hyaluronic acid.

In some cases, the administering is performed a single time and administers at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, or 1×10¹⁴ extracellular vesicles. In some cases, the administering is performed a single time and administers at most 1×10⁷, 1×10⁸, 1×10⁹, 1×10¹⁰, 1×10¹¹, 1×10¹², 1×10¹³, 1×10¹⁴, 1×10¹⁵, 1×10¹⁷, 1×10¹⁸, 1×10¹⁹, 1×10²⁰, or 1×10³⁰ extracellular vesicles. In some cases, the administering is performed a single time and administers at most 1×10¹³⁻¹⁴ extracellular vesicles. In some cases, the administering is performed multiple times and administers each time at least 1×10⁶, 5×10⁶, 1×10⁷, 5×10⁷, 1×10⁸, 3×10⁸, 5×10⁸, 1×10⁹, 3×10⁹, 1×10¹⁰, 1×10¹¹, 1×10¹², 1×10¹³, or 1×10¹⁴ extracellular vesicles. In some cases, the administering is performed multiple times and administers each time at most 1×10⁷, 1×10⁸, 1×10⁹, 1×10¹⁰, 1×10¹¹, 1×10¹², 1×10¹³, 1×10¹⁴, 1×10¹⁵, 1×10¹⁷, 1×10¹⁸, 1×10¹⁹, 1×10²⁰, or 1×10³⁰ extracellular vesicles. In some cases, the administering is performed multiple times and administers each time at most 1×10¹³-14 extracellular vesicles. In some cases, the administering is performed multiple times and administers at most 1×10¹³⁻¹⁴ extracellular vesicles within 6-8 weeks. In some cases, the administering is performed multiple times over a period of time and administers at most 1×10⁷, 1×10⁸, 1×10⁹, 1×10¹⁰, 1×10¹¹, 1×10¹², 1×10¹³, 1×10¹⁴, 1×10¹⁵, 1×10¹⁷, 1×10¹⁸, 1×10¹⁹, or 1×10²⁰, 1×10³⁰ extracellular vesicles.

Described herein, in some aspect, is a method of treating a blood flow disorder in a subject comprising administering at least one extracellular vesicle comprising VEGF mRNA to the subject, thereby treating the blood flow disorder. In some cases, the treating the blood flow disorder results in at least a 5% increase in revascularization. In some cases, the at least 5% increase in revascularization occurs within 14 days of the administering of the at least one extracellular vesicle comprising VEGF mRNA.

In some cases, the administering of the at least one extracellular vesicle comprising VEGF mRNA comprises administering the at least one extracellular vesicle comprising VEGF mRNA to the subject via an intravenous, intramuscular or subcutaneous injection or via a coronary artery catheter. In some cases, the intravenous, intramuscular or subcutaneous injection is performed with a needle with a gauge of at least 14 gauge.

In some cases, the blood flow disorder is ischemia. In some cases, the at least one extracellular vesicle comprising VEGF mRNA is administered in at least one dose. In some cases, the at least one extracellular vesicle comprising VEGF mRNA comprises at least 1,000 extracellular vesicles comprising VEGF mRNA. In some cases, the at least one extracellular vesicle comprising VEGF mRNA is administered in at least two doses.

In some cases, the administering comprises administering a single time at least 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or 1×10¹⁶ EVs. In some cases, the administering comprises administering a single time no greater than 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶ 1×10¹⁷, 1×10¹⁸, 1×10¹⁹, 1×10²⁰, 1×10²¹, 1×10²², 1×10²³, or 1×10²⁴ EVs. In some cases, the administering comprises administering a single time about 1×10⁵ to 1×10²⁰, 1×10⁶ to 1×10¹⁹, 1×10⁷ to 1×10¹⁸, 1×10⁸ to 1×10¹⁷, or 1×10⁹ to 1×10¹⁶ EVs. In some cases, the administering comprises administering a single time about 1×10¹⁰ to 1×10¹⁶ EVs. In some cases, the administering comprises administering a single time at least 10 μg, 50 μg, 100 μg, 200 μg, 300 μg, 400 μg, 500 μg, 600 μg, 700 μg, 800 μg, 900 μg, 1 ng, 100 ng, 500 ng, 1000 ng, 10 μg, 50 μg, 100 μg, 150 μg, or 200 μg of VEGF mRNA. In some cases, the administering comprises administering a single time no greater than 10 μg, 50 μg, 100 μg, 200 μg, 300 μg, 400 μg, 500 μg, 600 μg, 700 μg, 800 μg, 900 μg, 1 ng, 100 ng, 500 ng, 1000 ng, 10 μg, 50 μg, 100 μg, 150 μg, 200 μg, 300 μg, 400 μg, 500 μg, 600 μg, 700 μg, 800 μg, 900 μg, or 1000 μg of VEGF mRNA.

In some cases, the administering comprises administering multiple times and each time at least 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or 1×10¹⁶ EVs. In some cases, the administering comprises administering multiple times and each time no greater than 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶ 1×10¹⁷, 1×10¹⁸, 1×10¹⁹, 1×10²⁰, 1×10²¹, 1×10²², 1×10²³, or 1×10²⁴ EVs. In some cases, the administering comprises administering multiple times and each time about 1×10⁵ to 1×10²⁰, 1×10⁶ to 1×10¹⁹, 1×10⁷ to 1×10¹⁸, 1×10⁸ to 1×10¹⁷, or 1×10⁹ to 1×10¹⁶ EVs. In some cases, the administering comprises administering multiple times and each time about 1×10¹⁰ to 1×10¹⁶ EVs. In some cases, the administering comprises administering multiple times and each time at least 10 pg, 50 pg, 100 pg, 200 pg, 300 pg, 400 pg, 500 pg, 600 pg, 700 pg, 800 pg, 900 pg, 1 ng, 100 ng, 500 ng, 1000 ng, 10 μg, 50 μg, 100 μg, 150 μg, or 200 μg of VEGF mRNA. In some cases, the administering comprises administering multiple times and each time no greater than 10 pg, 50 pg, 100 pg, 200 pg, 300 pg, 400 pg, 500 pg, 600 pg, 700 pg, 800 pg, 900 pg, 1 ng, 100 ng, 500 ng, 1000 ng, 10 μg, 50 μg, 100 μg, 150 μg, 200 μg, 300 μg, 400 μg, 500 μg, 600 μg, 700 μg, 800 μg, 900 μg, or 1000 μg of VEGF mRNA.

In some cases, wherein administering of the at least one extracellular vesicle comprising VEGF mRNA to the subject comprises administering of the at least one extracellular vesicle comprising VEGF mRNA to a tissue of the subject. In some cases, the administering is performed using microneedles loaded with extracellular vesicles comprising VEGF mRNA.

In some cases, the microneedles comprise hydrogel. In some cases, the hydrogel comprises hyaluronic acid, sodium alginate, polylactic acid, polyglycolic acid, polylactic-glycolic acid, cartilage thioflavin, silk protein, maltose, chitosan, carboxymethyl cellulose, or any combination thereof.

In some cases, the extracellular vesicles are administered to the subject in intervals of at least once a day, once every week, once every 2 weeks, once every 4 weeks, once every 5 weeks, once every 6 weeks, once every 7 weeks, once every 8 weeks, once every 10 weeks, once every 12 weeks, or once every 16 weeks. In some cases, the extracellular vesicles are administered to the subject at most once a day, once every week, once every 2 weeks, once every 4 weeks, once every 5 weeks, once every 6 weeks, once every 7 weeks, once every 8 weeks, once every 10 weeks, once every 12 weeks, or once every 16 weeks.

Described herein, in some aspect, is a method of producing an extracellular vesicle comprising VEGF mRNA comprising: (a) introducing a vector or plasmid encoding VEGF into a donor cell via transfection; (b) incubating the donor cell for a sufficient time for the production of extracellular vesicles encapsulating VEGF mRNA transcribed from the vector or plasmid; and (c) collecting the extracellular vesicles encapsulating the VEGF mRNA transcribed from the vector or plasmid.

Described herein, in some aspect, is a method of treating a blood flow disorder a subject in need thereof, the method comprising administering the extracellular vesicles disclosed herein. In some cases, the blood flow disorder is ischemia.

### Production of Extracellular Vesicles

Described herein, in some aspects, is a method of producing an extracellular vesicle comprising extracellular matrix mRNA comprising: a) introducing a vector or a plasmid correspond to the extracellular matrix mRNA into a donor cell via transfection; (b) culturing the donor cell for a sufficient amount of time in a culture medium for the production of extracellular vesicles encapsulating the at least one extracellular matrix mRNA transcribed from the vector or the plasmid; and (c) collecting the extracellular vesicles from the culture medium. In some cases, in step (c) the collecting the extracellular vesicles occurs about 8-24 hr after the transfection. In some cases, the extracellular vesicles encapsulating the extracellular matrix mRNA transcribed from the vector or plasmid are present at a level of at least 1000, 2000, 5000, 10000, or 12000 extracellular vesicles per donor cell. In some cases, the donor cell is a human cell, a human fibroblast cell, a fibroblast cell, a dermal fibroblast, a human fibroblast, an adult fibroblast, a human adult fibroblast, a neonatal fibroblast, a neonatal human fibroblast, a neonatal human dermal fibroblast, or any combination thereof.

Described herein, in some cases, are methods and systems of producing the extracellular vesicles comprising the therapeutic polypeptide, the therapeutic compound, the cancer drug, the targeting polypeptide, or a combination thereof.

In some cases, the method comprises introducing at least one heterologous polynucleotide into an extracellular vesicle donor cell. In some cases, the at least one heterologous polynucleotide is a vector. In some instances, the at least one heterologous polynucleotide comprises at least therapeutic polynucleotide described herein. In some instances, the at least one heterologous polynucleotide encodes at least one therapeutic polynucleotide described herein. In some instances, the at least one heterologous polynucleotide encodes at least one therapeutic polypeptide described herein. In some instances, the at least one heterologous polynucleotide introduced into the extracellular vesicle donor cells encodes at least one targeting polypeptide described herein. In some cases, the at least one heterologous polynucleotide encodes at least one heterologous targeting domain.

In some instances, at least two heterologous polynucleotides are introduced into an extracellular donor cell, where a first heterologous polynucleotide introduced into the extracellular vesicle donor cell comprises a first vector encoding the at least one therapeutic polynucleotide or the at least one therapeutic polypeptide. In some cases, a second heterologous polynucleotide comprising a second vector encoding at least one targeting polypeptide or tumor targeting polypeptide.

In some cases, the heterologous polynucleotide can be introduced into the cell via the use of expression vectors. In the context of an expression vector, the vector can be readily introduced into the cell described herein by any method in the art. For example, the expression vector can be transferred into the cell by biological, chemical, or physical methods. In some cases, the extracellular vesicle donor cell can be any type of cell described herein. In some cases, the extracellular donor cell can be nucleated cell.

Biological methods for introducing the heterologous polynucleotide of interest into the cell can include the use of DNA or RNA vectors. Viral vectors, and especially retroviral vectors, have become the most widely used method for inserting genes into non-human mammalian cells. Other viral vectors, in some cases, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Leukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV vectors include AAV1, AAV2, AAV4, AAV5, AAV6, AAV7, AAV8, or AAV9 serotype. In some instances, viral vector is a chimeric viral vector, comprising viral portions from two or more viruses. In additional instances, the viral vector is a recombinant viral vector.

Chemical methods for introducing the heterologous polynucleotide into the cell can include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid is associated with a lipid. The nucleic acid associated with a lipid, in some cases, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some cases, they are present in a bilayer structure, as micelles, or with a “collapsed” structure. Alternately, they are simply be interspersed in a solution, possibly forming aggregates that are not uniform in size or shape. Lipids are fatty substances which are, in some cases, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. For example, in some cases, dimyristyl phosphatidylcholine (“DMPC”) is obtained from Sigma, St. Louis, Mo.; in some cases, dicetyl phosphate (“DCP”) is obtained from K & K Laboratories (Plainview, N.Y.); cholesterol (“Choi”), in some cases, is obtained from Calbiochem-Behring; dimyristyl phosphatidylglycerol (“DMPG”) and other lipids are often obtained from Avanti Polar Lipids, Inc. (Birmingham, Ala.). Stock solutions of lipids in chloroform or chloroform/methanol are often stored at about −20° C. Chloroform is used as the solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some cases, assume a micellar structure or merely exist as nonuniform aggregates of lipid molecules. Also contemplated are lipofectamine-nucleic acid complexes.

Physical methods for introducing the heterologous polynucleotide into the cell can include calcium phosphate precipitation, lipofection, particle bombardment, microinjection, gene gun, electroporation, micro-needle array, nano-needle array, sonication, or chemical permeation. Electroporation includes microfluidics electroporation, microchannel electroporation, or nanochannel electroporation. In certain cases, the extracellular vesicle donor cell is transfected with the at least one heterologous polynucleotide by microchannel electroporation or nanochannel electroporation. In some instances, the microchannel electroporation or the nanochannel electroporation comprises use of micropore patterned silicon wafers, nanopore patterned silicon wafers, track etch membranes, ceramic micropore membranes, ceramic nanopore membranes, other porous materials, or a combination thereof. In some instances, the at least one heterologous polynucleotide or the at least one vector is nanoelectroporated into the extracellular vesicle donor cell via a nanochannel located on a biochip.

In some cases, extracellular vesicle donor cells can be grown and attached on a surface of a substrate. In some cases, the substrate comprises a biochip. In some cases, the surface of the substrate comprise metallic material. In some cases, the substrates comprise metallic material.

Non-limiting examples of metallic material include aluminum (Al), indium tin oxide (ITO, In₂O₃:SnO2), chromium (Cr), gallium arsenide (GaAs), gold (Au), molybdenum (Mo), organic residues and photoresist, platinum (Pt), silicon (Si), silicon dioxide (SiO₂), silicon on insulator (SOI), silicon nitride (Si₃N₄) tantalum (Ta), titanium (Ti), titanium nitride (TiN), tungsten (W). In some cases, the metallic material can be treated or etched to create an array or channels. In some cases, the metallic surface can be treated or etched with phosphoric acid (H₃PO₄), acetic acid, nitric acid (HNO₃), water (H₂O), hydrochloric acid (HCl), (HNO₃), ceric ammonium nitrate ((NH₄)₂Ce(NO₃)₆, citric acid (C₆H₈O₇), hydrogen peroxide (H₂O₂), aqua regia, iodine solution, sulfuric acid (H₂SO₄), hydrofluoric acid (HF), potassium hydroxide (KOH), ethylenediamine pyrocatechol (EDP), tetramethylammonium hydroxide (TMAH), buffered oxide, ammonium fluoride (NH₄F), SCl, Cl₂, CCl₄, SiCl₄, BCl₃, SiCl₄, BCl₃, CCl₂F₂, CF₄, O₂, CF₄, SF₆, NF₃, CHF₃, or a combination thereof.

In some cases, the metallic surface can be treated with a gas or plasma to increase hydrophilicity. In some cases, the metallic surface can be treated with a gas or plasma to increase hydrophobicity. Exemplary gas or plasma for increasing hydrophilicity or hydrophobicity of the metallic surface include oxygen, nitrogen, ammonia, argon, chlorine, fluorine, bromine, iodine, astatine, hydrogen, or a combination thereof.

In some cases, the extracellular vesicle donor cells can be grown and attached to a surface of a substrate made of polymers such as polypropylene, polyethylene, polystyrene, ABS, polyamide, polyethylene copolymer, epoxy, polyester, polyvinylchloride, phenolic, polytetrafluoroethylene, polyethylene copolymer, fluorinated ethylene propylene, polyvinylidene, silicone, natural rubber, latex, polyurethane, styrene butadiene rubber, fluorocarbon copolymer elastomer, polyethylene terephthalate, polycarbonate, polyamide, polyaramid, polyaryl ether ketone, polyacetal, polyphenylene oxide, PBT, polysulfone, polyethersulfone, polyarylsulfone, polyphenylene sulfide, polytetrafluoroethylene, beryllium oxide etc. In some cases, the surface made of polymers can be semi-permeable. In some embodiments, pore size of the semi-permeable polymer surface can be between about 0.01 μm to about 10 μm. In some embodiment, pore size of the semi-permeable polymer surface can be between about 0.01 μm to about 0.03 μm, about 0.01 μm to about 0.05 μm, about 0.01 μm to about 0.1 μm, about 0.01 μm to about 0.2 μm, about 0.01 μm to about 0.3 μm, about 0.01 μm to about 0.4 μm, about 0.01 μm to about 0.5 μm, about 0.01 μm to about 1 μm, about 0.01 μm to about 3 μm, about 0.01 μm to about 5 μm, about 0.01 μm to about 10 μm, about 0.03 μm to about 0.05 μm, about 0.03 μm to about 0.1 μm, about 0.03 μm to about 0.2 μm, about 0.03 μm to about 0.3 μm, about 0.03 μm to about 0.4 μm, about 0.03 μm to about 0.5 μm, about 0.03 μm to about 1 μm, about 0.03 μm to about 3 μm, about 0.03 μm to about 5 μm, about 0.03 μm to about 10 μm, about 0.05 μm to about 0.1 μm, about 0.05 μm to about 0.2 μm, about 0.05 μm to about 0.3 μm, about 0.05 μm to about 0.4 μm, about 0.05 μm to about 0.5 μm, about 0.05 μm to about 1 μm, about 0.05 μm to about 3 μm, about 0.05 μm to about 5 μm, about 0.05 μm to about 10 μm, about 0.1 μm to about 0.2 μm, about 0.1 μm to about 0.3 μm, about 0.1 μm to about 0.4 μm, about 0.1 μm to about 0.5 μm, about 0.1 μm to about 1 μm, about 0.1 μm to about 3 μm, about 0.1 μm to about 5 μm, about 0.1 μm to about 10 μm, about 0.2 μm to about 0.3 μm, about 0.2 μm to about 0.4 μm, about 0.2 μm to about 0.5 μm, about 0.2 μm to about 1 μm, about 0.2 μm to about 3 μm, about 0.2 μm to about 5 μm, about 0.2 μm to about 10 μm, about 0.3 μm to about 0.4 μm, about 0.3 μm to about 0.5 μm, about 0.3 μm to about 1 μm, about 0.3 μm to about 3 μm, about 0.3 μm to about 5 μm, about 0.3 μm to about 10 μm, about 0.4 μm to about 0.5 μm, about 0.4 μm to about 1 μm, about 0.4 μm to about 3 μm, about 0.4 μm to about 5 μm, about 0.4 μm to about 10 μm, about 0.5 μm to about 1 μm, about 0.5 μm to about 3 μm, about 0.5 μm to about 5 μm, about 0.5 μm to about 10 μm, about 1 μm to about 3 μm, about 1 μm to about 5 μm, about 1 μm to about 10 μm, about 3 μm to about 5 μm, about 3 μm to about 10 μm, or about 5 μm to about 10 μm. In some embodiment, pore size of the semi-permeable polymer surface can be between about 0.01 μm, about 0.03 μm, about 0.05 μm, about 0.1 μm, about 0.2 μm, about 0.3 μm, about 0.4 μm, about 0.5 μm, about 1 μm, about 3 μm, about 5 μm, or about 10 μm. In some embodiment, pore size of the semi-permeable polymer surface can be between at least about 0.01 μm, about 0.03 μm, about 0.05 μm, about 0.1 μm, about 0.2 μm, about 0.3 μm, about 0.4 μm, about 0.5 μm, about 1 μm, about 3 μm, or about 5 μm. In some embodiment, pore size of the semi-permeable polymer surface can be between at most about 0.03 μm, about 0.05 μm, about 0.1 μm, about 0.2 μm, about 0.3 μm, about 0.4 μm, about 0.5 μm, about 1 μm, about 3 μm, about 5 μm, or about 10 μm.

In some cases, the surface of the polymer can be treated with a gas or plasma to increase hydrophilicity. In some cases, the surface of the polymer can be treated with a gas or plasma to increase hydrophobicity. Exemplary gas or plasma for increasing hydrophilicity or hydrophobicity of the metallic surface include oxygen, nitrogen, ammonia, argon, chlorine, fluorine, bromine, iodine, astatine, hydrogen, or a combination thereof.

### Nanoelectroporation

In some cases, the extracellular vesicle donor cells grown or attached to a metallic or polymer surface can be nanoelectroporated by nanoelectroporation systems as described herein.

In some cases, the extracellular vesicle donor cells to be nanoelectroporated by nanoelectroporation systems described herein can be grown or attached to the metallic or polymer surface such as the biochip described herein. In some cases, the extracellular vesicle donor cells to be nanoelectroporated by nanoelectroporation systems described herein can be grown or attached to the metallic or polymer surface such as the biochip described herein in a monolayer. In some cases, the systems comprise a fluidic chamber with an upper boundary and a lower boundary. The placement of the substrate with the extracellular vesicle donor cells in the fluid chamber create an upper chamber and a lower chamber. In some cases, the systems further comprise at least one nanochannel. In some cases, the nanochannels can be embedded within the substrate.

In some cases, the extracellular vesicle donor cells grown or attached to a metallic or polymer surface and nanoelectroporated with the heterologous polynucleotide described herein can result in high-throughput production of extracellular vesicles (e.g., exosomes). In some cases, such high-throughput production of exosomes can involve use of a plurality (e.g., greater than 1, greater than 2, greater than 3, greater than 5, greater than 10, or additional numbers) biochips (e.g., CNP biochips). In some cases, the CNP biochip comprises a width that is at least 1 cm, 2 cm, 3 cm, 4 cm, 5 cm, 6 cm, 7 cm, 8 cm, 9 cm, 10 cm, 15 cm, 20 cm, 25 cm, 30 cm, 35 cm, 40 cm, 45 cm, 50 cm, 100 cm, or more cm. In some cases, the CNP biochip comprises a length that is at least 1 cm, 2 cm, 3 cm, 4 cm, 5 cm, 6 cm, 7 cm, 8 cm, 9 cm, 10 cm, 15 cm, 20 cm, 25 cm, 30 cm, 35 cm, 40 cm, 45 cm, 50 cm, 100 cm, or more cm. In some cases, the biochip comprises a dimension of 1 cm×1 cm. In some cases, the biochip can comprise exemplary dimensions of 1 cm×2 cm, 1 cm×3 cm, 1 cm×5 cm, 1 cm×10 cm, 2 cm×1 cm, 2 cm×2 cm, 2 cm×3 cm, 2 cm×5 cm, 2 cm×10 cm, 3 cm×1 cm, 3 cm×2 cm, 3 cm×3 cm, 3 cm×5 cm, 3 cm×10 cm, 5 cm×1 cm, 5 cm×2 cm, 5 cm×3 cm, 5 cm×5 cm, 5 cm×10 cm, 10 cm×1 cm, 10 cm×2 cm, 10 cm×3 cm, 10 cm×5 cm, or 10 cm×10 cm.

In some cases, the nanoelectroporation of the extracellular vesicle donor cells can comprise a cycle comprising nanochannel electroporation (CNP) followed by collecting the extracellular vesicles produced and secreted by the nonelectroporated extracellular vesicle donor cells for 1 hour, 2 hours, 3 hours, 4 hours, 5 hours, 6 hours, 12 hours, 24 hours, 36 hours, 48 hours, 3 days, 4 days, 5 days, 6 days, 1 week, or longer period of time for collecting the extracellular vesicles. In some cases, the extracellular vesicle donor cells can produce and secrete extracellular vesicles for 1 cycle, 2 cycles, 3 cycles, 4 cycles, 5 cycles, 6 cycles, 7 cycles, 8 cycles, 9 cycles, 10 cycles, or more cycles of CNP.

In some cases, the secreted extracellular vesicles are biocompatible, measure 40˜150 nm in diameter, and may intrinsically express transmembrane and membrane-anchored proteins. The presence of these proteins may prolong blood circulation, may promote tissue-directed delivery and may facilitate cellular uptake of encapsulated exosomal contents. In some cases, the methods provided herein may involve use of nanoelectroporation without formation of significant aggregates. In some instances, the heterologous polynucleotide or vector introduced into the extracellular vesicle donor cell does not encode a peptide sequence that is incorporated into the extracellular vesicles and binds to target mRNA.

In some cases, the nanochannels comprise the pores of the semi-permeable polymer substrate. In some embodiment, the nanochannels comprise a height from about 0.01 μm to about 500 μm. In some embodiment, the nanochannels comprise a height from about 0.01 μm to about 0.05 μm, about 0.01 μm to about 0.1 μm, about 0.01 μm to about 0.5 μm, about 0.01 μm to about 1 μm, about 0.01 μm to about 2 μm, about 0.01 μm to about 5 μm, about 0.01 μm to about 10 μm, about 0.01 μm to about 20 μm, about 0.01 μm to about 50 μm, about 0.01 μm to about 100 μm, about 0.01 μm to about 500 μm, about 0.05 μm to about 0.1 μm, about 0.05 μm to about 0.5 μm, about 0.05 μm to about 1 μm, about 0.05 μm to about 2 μm, about 0.05 μm to about 5 μm, about 0.05 μm to about 10 μm, about 0.05 μm to about 20 μm, about 0.05 μm to about 50 μm, about 0.05 μm to about 100 μm, about 0.05 μm to about 500 μm, about 0.1 μm to about 0.5 μm, about 0.1 μm to about 1 μm, about 0.1 μm to about 2 μm, about 0.1 μm to about 5 μm, about 0.1 μm to about 10 μm, about 0.1 μm to about 20 μm, about 0.1 μm to about 50 μm, about 0.1 μm to about 100 μm, about 0.1 μm to about 500 μm, about 0.5 μm to about 1 μm, about 0.5 μm to about 2 μm, about 0.5 μm to about 5 μm, about 0.5 μm to about 10 μm, about 0.5 μm to about 20 μm, about 0.5 μm to about 50 μm, about 0.5 μm to about 100 μm, about 0.5 μm to about 500 μm, about 1 μm to about 2 μm, about 1 μm to about 5 μm, about 1 μm to about 10 μm, about 1 μm to about 20 μm, about 1 μm to about 50 μm, about 1 μm to about 100 μm, about 1 μm to about 500 μm, about 2 μm to about 5 μm, about 2 μm to about 10 μm, about 2 μm to about 20 μm, about 2 μm to about 50 μm, about 2 μm to about 100 μm, about 2 μm to about 500 μm, about 5 μm to about 10 μm, about 5 μm to about 20 μm, about 5 μm to about 50 μm, about 5 μm to about 100 μm, about 5 μm to about 500 μm, about 10 μm to about 20 μm, about 10 μm to about 50 μm, about 10 μm to about 100 μm, about 10 μm to about 500 μm, about 20 μm to about 50 μm, about 20 μm to about 100 μm, about 20 μm to about 500 μm, about 50 μm to about 100 μm, about 50 μm to about 500 μm, or about 100 μm to about 500 μm. In some embodiment, the nanochannels comprise a height from about 0.01 μm, about 0.05 μm, about 0.1 μm, about 0.5 μm, about 1 μm, about 2 μm, about 5 μm, about 10 μm, about 20 μm, about 50 μm, about 100 μm, or about 500 μm. In some embodiment, the nanochannels comprise a height from at least about 0.01 μm, about 0.05 μm, about 0.1 μm, about 0.5 μm, about 1 μm, about 2 μm, about 5 μm, about 10 μm, about 20 μm, about 50 μm, or about 100 μm. In some embodiment, the nanochannels comprise a height from at most about 0.05 μm, about 0.1 μm, about 0.5 μm, about 1 μm, about 2 μm, about 5 μm, about 10 μm, about 20 μm, about 50 μm, about 100 μm, or about 500 μm. In some cases, the heights of the nanochannels can be the same. In some cases, the heights of the nanochannels can be different. In some cases, the heights of the nanochannels should be great enough to accelerate the molecules being nanoelectroporated in the high electric field zone (e.g., inside the nanochannel), but also small enough to enable large molecules being nanoelectroporated to squeeze through in a brief electric pulse.

In some embodiment, the nanochannels comprise a diameter from about 0.01 nm to about 10,000 nm. In some embodiment, the nanochannels comprise a diameter from about 0.01 nm to about 0.1 nm, about 0.01 nm to about 0.5 nm, about 0.01 nm to about 1 nm, about 0.01 nm to about 5 nm, about 0.01 nm to about 10 nm, about 0.01 nm to about 50 nm, about 0.01 nm to about 100 nm, about 0.01 nm to about 500 nm, about 0.01 nm to about 1,000 nm, about 0.01 nm to about 5,000 nm, about 0.01 nm to about 10,000 nm, about 0.1 nm to about 0.5 nm, about 0.1 nm to about 1 nm, about 0.1 nm to about 5 nm, about 0.1 nm to about 10 nm, about 0.1 nm to about 50 nm, about 0.1 nm to about 100 nm, about 0.1 nm to about 500 nm, about 0.1 nm to about 1,000 nm, about 0.1 nm to about 5,000 nm, about 0.1 nm to about 10,000 nm, about 0.5 nm to about 1 nm, about 0.5 nm to about 5 nm, about 0.5 nm to about 10 nm, about 0.5 nm to about 50 nm, about 0.5 nm to about 100 nm, about 0.5 nm to about 500 nm, about 0.5 nm to about 1,000 nm, about 0.5 nm to about 5,000 nm, about 0.5 nm to about 10,000 nm, about 1 nm to about 5 nm, about 1 nm to about 10 nm, about 1 nm to about 50 nm, about 1 nm to about 100 nm, about 1 nm to about 500 nm, about 1 nm to about 1,000 nm, about 1 nm to about 5,000 nm, about 1 nm to about 10,000 nm, about 5 nm to about 10 nm, about 5 nm to about 50 nm, about 5 nm to about 100 nm, about 5 nm to about 500 nm, about 5 nm to about 1,000 nm, about 5 nm to about 5,000 nm, about 5 nm to about 10,000 nm, about 10 nm to about 50 nm, about 10 nm to about 100 nm, about 10 nm to about 500 nm, about 10 nm to about 1,000 nm, about 10 nm to about 5,000 nm, about 10 nm to about 10,000 nm, about 50 nm to about 100 nm, about 50 nm to about 500 nm, about 50 nm to about 1,000 nm, about 50 nm to about 5,000 nm, about 50 nm to about 10,000 nm, about 100 nm to about 500 nm, about 100 nm to about 1,000 nm, about 100 nm to about 5,000 nm, about 100 nm to about 10,000 nm, about 500 nm to about 1,000 nm, about 500 nm to about 5,000 nm, about 500 nm to about 10,000 nm, about 1,000 nm to about 5,000 nm, about 1,000 nm to about 10,000 nm, or about 5,000 nm to about 10,000 nm. In some embodiment, the nanochannels comprise a diameter from about 0.01 nm, about 0.1 nm, about 0.5 nm, about 1 nm, about 5 nm, about 10 nm, about 50 nm, about 100 nm, about 500 nm, about 1,000 nm, about 5,000 nm, or about 10,000 nm. In some embodiment, the nanochannels comprise a diameter from at least about 0.01 nm, about 0.1 nm, about 0.5 nm, about 1 nm, about 5 nm, about 10 nm, about 50 nm, about 100 nm, about 500 nm, about 1,000 nm, or about 5,000 nm. In some embodiment, the nanochannels comprise a diameter from at most about 0.1 nm, about 0.5 nm, about 1 nm, about 5 nm, about 10 nm, about 50 nm, about 100 nm, about 500 nm, about 1,000 nm, about 5,000 nm, or about 10,000 nm. In some embodiments, the nanochannels comprise a diameter between about 50 nm to about 3000 nm. In some embodiments, the nanochannels comprise a diameter of 1000 nm or slightly larger. In some cases, the diameters of the nanochannels can be the same. In some cases, the diameters of the nanochannels can be different.

In some cases, the nanochannels can be arranged into a nanochannel array. In some cases, the nanochannels can be arranged into a nanochannel array with spacing between the nanochannels. In some instances, the spacing between the nanochannels can be from about 0.01 μm to about 5,000 μm. In some instances, the spacing between the nanochannels can be from about 0.01 μm to about 0.05 μm, about 0.01 μm to about 0.1 μm, about 0.01 μm to about 0.5 μm, about 0.01 μm to about 1 μm, about 0.01 μm to about 5 μm, about 0.01 μm to about 10 μm, about 0.01 μm to about 50 μm, about 0.01 μm to about 100 μm, about 0.01 μm to about 500 μm, about 0.01 μm to about 1,000 μm, about 0.01 μm to about 5,000 μm, about 0.05 μm to about 0.1 μm, about 0.05 μm to about 0.5 μm, about 0.05 μm to about 1 μm, about 0.05 μm to about 5 μm, about 0.05 μm to about 10 μm, about 0.05 μm to about 50 μm, about 0.05 μm to about 100 μm, about 0.05 μm to about 500 μm, about 0.05 μm to about 1,000 μm, about 0.05 μm to about 5,000 μm, about 0.1 μm to about 0.5 μm, about 0.1 μm to about 1 μm, about 0.1 μm to about 5 μm, about 0.1 μm to about 10 μm, about 0.1 μm to about 50 μm, about 0.1 μm to about 100 μm, about 0.1 μm to about 500 μm, about 0.1 μm to about 1,000 μm, about 0.1 μm to about 5,000 μm, about 0.5 μm to about 1 μm, about 0.5 μm to about 5 μm, about 0.5 μm to about 10 μm, about 0.5 μm to about 50 μm, about 0.5 μm to about 100 μm, about 0.5 μm to about 500 μm, about 0.5 μm to about 1,000 μm, about 0.5 μm to about 5,000 μm, about 1 μm to about 5 μm, about 1 μm to about 10 μm, about 1 μm to about 50 μm, about 1 μm to about 100 μm, about 1 μm to about 500 μm, about 1 μm to about 1,000 μm, about 1 μm to about 5,000 μm, about 5 μm to about 10 μm, about 5 μm to about 50 μm, about 5 μm to about 100 μm, about 5 μm to about 500 μm, about 5 μm to about 1,000 μm, about 5 μm to about 5,000 μm, about 10 μm to about 50 μm, about 10 μm to about 100 μm, about 10 μm to about 500 μm, about 10 μm to about 1,000 μm, about 10 μm to about 5,000 μm, about 50 μm to about 100 μm, about 50 μm to about 500 μm, about 50 μm to about 1,000 μm, about 50 μm to about 5,000 μm, about 100 μm to about 500 μm, about 100 μm to about 1,000 μm, about 100 μm to about 5,000 μm, about 500 μm to about 1,000 μm, about 500 μm to about 5,000 μm, or about 1,000 μm to about 5,000 μm. In some instances, the spacing between the nanochannels can be from about 0.01 μm, about 0.05 μm, about 0.1 μm, about 0.5 μm, about 1 μm, about 5 μm, about 10 μm, about 50 μm, about 100 μm, about 500 μm, about 1,000 μm, or about 5,000 μm. In some instances, the spacing between the nanochannels can be from at least about 0.01 μm, about 0.05 μm, about 0.1 μm, about 0.5 μm, about 1 μm, about 5 μm, about 10 μm, about 50 μm, about 100 μm, about 500 μm, or about 1,000 μm. In some instances, the spacing between the nanochannels can be from at most about 0.05 μm, about 0.1 μm, about 0.5 μm, about 1 μm, about 5 μm, about 10 μm, about 50 μm, about 100 μm, about 500 μm, about 1,000 μm, or about 5,000 μm.

In some cases, the nanoelectroporating systems comprise upper and lower electrode layers for generating an electric field within the fluidic chamber. In some cases, the electric field generated by the electrodes for nanoelectroporation comprises a voltage that is between about 10 V to about 500 V. In some cases, the electric field generated by the electrodes for nanoelectroporation comprises a voltage that is between about 10 V to about 25 V, about 10 V to about 50 V, about 10 V to about 100 V, about 10 V to about 125 V, about 10 V to about 150 V, about 10 V to about 175 V, about 10 V to about 200 V, about 10 V to about 225 V, about 10 V to about 250 V, about 10 V to about 300 V, about 10 V to about 500 V, about 25 V to about 50 V, about 25 V to about 100 V, about 25 V to about 125 V, about 25 V to about 150 V, about 25 V to about 175 V, about 25 V to about 200 V, about 25 V to about 225 V, about 25 V to about 250 V, about 25 V to about 300 V, about 25 V to about 500 V, about 50 V to about 100 V, about 50 V to about 125 V, about 50 V to about 150 V, about 50 V to about 175 V, about 50 V to about 200 V, about 50 V to about 225 V, about 50 V to about 250 V, about 50 V to about 300 V, about 50 V to about 500 V, about 100 V to about 125 V, about 100 V to about 150 V, about 100 V to about 175 V, about 100 V to about 200 V, about 100 V to about 225 V, about 100 V to about 250 V, about 100 V to about 300 V, about 100 V to about 500 V, about 125 V to about 150 V, about 125 V to about 175 V, about 125 V to about 200 V, about 125 V to about 225 V, about 125 V to about 250 V, about 125 V to about 300 V, about 125 V to about 500 V, about 150 V to about 175 V, about 150 V to about 200 V, about 150 V to about 225 V, about 150 V to about 250 V, about 150 V to about 300 V, about 150 V to about 500 V, about 175 V to about 200 V, about 175 V to about 225 V, about 175 V to about 250 V, about 175 V to about 300 V, about 175 V to about 500 V, about 200 V to about 225 V, about 200 V to about 250 V, about 200 V to about 300 V, about 200 V to about 500 V, about 225 V to about 250 V, about 225 V to about 300 V, about 225 V to about 500 V, about 250 V to about 300 V, about 250 V to about 500 V, or about 300 V to about 500 V. In some cases, the electric field generated by the electrodes for nanoelectroporation comprises a voltage that is between about 10 V, about 25 V, about 50 V, about 100 V, about 125 V, about 150 V, about 175 V, about 200 V, about 225 V, about 250 V, about 300 V, or about 500 V. In some cases, the electric field generated by the electrodes for nanoelectroporation comprises a voltage that is between at least about 10 V, about 25 V, about 50 V, about 100 V, about 125 V, about 150 V, about 175 V, about 200 V, about 225 V, about 250 V, or about 300 V. In some cases, the electric field generated by the electrodes for nanoelectroporation comprises a voltage that is between at most about 25 V, about 50 V, about 100 V, about 125 V, about 150 V, about 175 V, about 200 V, about 225 V, about 250 V, about 300 V, or about 500 V.

In some cases, the electric field generated by the electrodes for nanoelectroporation comprises an electric field strength from about 0.1 volt/mm to about 50,000 volt/mm. In some cases, the electric field generated by the electrodes for nanoelectroporation comprises an electric field strength from about 0.1 volt/mm to about 0.5 volt/mm, about 0.1 volt/mm to about 1 volt/mm, about 0.1 volt/mm to about 5 volt/mm, about 0.1 volt/mm to about 10 volt/mm, about 0.1 volt/mm to about 50 volt/mm, about 0.1 volt/mm to about 100 volt/mm, about 0.1 volt/mm to about 500 volt/mm, about 0.1 volt/mm to about 1,000 volt/mm, about 0.1 volt/mm to about 5,000 volt/mm, about 0.1 volt/mm to about 10,000 volt/mm, about 0.1 volt/mm to about 50,000 volt/mm, about 0.5 volt/mm to about 1 volt/mm, about 0.5 volt/mm to about 5 volt/mm, about 0.5 volt/mm to about 10 volt/mm, about 0.5 volt/mm to about 50 volt/mm, about 0.5 volt/mm to about 100 volt/mm, about 0.5 volt/mm to about 500 volt/mm, about 0.5 volt/mm to about 1,000 volt/mm, about 0.5 volt/mm to about 5,000 volt/mm, about 0.5 volt/mm to about 10,000 volt/mm, about 0.5 volt/mm to about 50,000 volt/mm, about 1 volt/mm to about 5 volt/mm, about 1 volt/mm to about 10 volt/mm, about 1 volt/mm to about 50 volt/mm, about 1 volt/mm to about 100 volt/mm, about 1 volt/mm to about 500 volt/mm, about 1 volt/mm to about 1,000 volt/mm, about 1 volt/mm to about 5,000 volt/mm, about 1 volt/mm to about 10,000 volt/mm, about 1 volt/mm to about 50,000 volt/mm, about 5 volt/mm to about 10 volt/mm, about 5 volt/mm to about 50 volt/mm, about 5 volt/mm to about 100 volt/mm, about 5 volt/mm to about 500 volt/mm, about 5 volt/mm to about 1,000 volt/mm, about 5 volt/mm to about 5,000 volt/mm, about 5 volt/mm to about 10,000 volt/mm, about 5 volt/mm to about 50,000 volt/mm, about 10 volt/mm to about 50 volt/mm, about 10 volt/mm to about 100 volt/mm, about 10 volt/mm to about 500 volt/mm, about 10 volt/mm to about 1,000 volt/mm, about 10 volt/mm to about 5,000 volt/mm, about 10 volt/mm to about 10,000 volt/mm, about 10 volt/mm to about 50,000 volt/mm, about 50 volt/mm to about 100 volt/mm, about 50 volt/mm to about 500 volt/mm, about 50 volt/mm to about 1,000 volt/mm, about 50 volt/mm to about 5,000 volt/mm, about 50 volt/mm to about 10,000 volt/mm, about 50 volt/mm to about 50,000 volt/mm, about 100 volt/mm to about 500 volt/mm, about 100 volt/mm to about 1,000 volt/mm, about 100 volt/mm to about 5,000 volt/mm, about 100 volt/mm to about 10,000 volt/mm, about 100 volt/mm to about 50,000 volt/mm, about 500 volt/mm to about 1,000 volt/mm, about 500 volt/mm to about 5,000 volt/mm, about 500 volt/mm to about 10,000 volt/mm, about 500 volt/mm to about 50,000 volt/mm, about 1,000 volt/mm to about 5,000 volt/mm, about 1,000 volt/mm to about 10,000 volt/mm, about 1,000 volt/mm to about 50,000 volt/mm, about 5,000 volt/mm to about 10,000 volt/mm, about 5,000 volt/mm to about 50,000 volt/mm, or about 10,000 volt/mm to about 50,000 volt/mm. In some cases, the electric field generated by the electrodes for nanoelectroporation comprises an electric field strength from about 0.1 volt/mm, about 0.5 volt/mm, about 1 volt/mm, about 5 volt/mm, about 10 volt/mm, about 50 volt/mm, about 100 volt/mm, about 500 volt/mm, about 1,000 volt/mm, about 5,000 volt/mm, about 10,000 volt/mm, or about 50,000 volt/mm. In some cases, the electric field generated by the electrodes for nanoelectroporation comprises an electric field strength from at least about 0.1 volt/mm, about 0.5 volt/mm, about 1 volt/mm, about 5 volt/mm, about 10 volt/mm, about 50 volt/mm, about 100 volt/mm, about 500 volt/mm, about 1,000 volt/mm, about 5,000 volt/mm, or about 10,000 volt/mm. In some cases, the electric field generated by the electrodes for nanoelectroporation comprises an electric field strength from at most about 0.5 volt/mm, about 1 volt/mm, about 5 volt/mm, about 10 volt/mm, about 50 volt/mm, about 100 volt/mm, about 500 volt/mm, about 1,000 volt/mm, about 5,000 volt/mm, about 10,000 volt/mm, or about 50,000 volt/mm.

In some instances, the electric field generated by the electrodes for nanoelectroporation comprises a plurality of pulses with pulse duration from about 0.01 millisecond/pulse to about 5,000 millisecond/pulse. In some instances, the electric field generated by the electrodes for nanoelectroporation comprises a plurality of pulses with pulse duration from about 0.01 millisecond/pulse to about 0.05 millisecond/pulse, about 0.01 millisecond/pulse to about 0.1 millisecond/pulse, about 0.01 millisecond/pulse to about 0.5 millisecond/pulse, about 0.01 millisecond/pulse to about 1 millisecond/pulse, about 0.01 millisecond/pulse to about 5 millisecond/pulse, about 0.01 millisecond/pulse to about 10 millisecond/pulse, about 0.01 millisecond/pulse to about 50 millisecond/pulse, about 0.01 millisecond/pulse to about 100 millisecond/pulse, about 0.01 millisecond/pulse to about 500 millisecond/pulse, about 0.01 millisecond/pulse to about 1,000 millisecond/pulse, about 0.01 millisecond/pulse to about 5,000 millisecond/pulse, about 0.05 millisecond/pulse to about 0.1 millisecond/pulse, about 0.05 millisecond/pulse to about 0.5 millisecond/pulse, about 0.05 millisecond/pulse to about 1 millisecond/pulse, about 0.05 millisecond/pulse to about 5 millisecond/pulse, about 0.05 millisecond/pulse to about 10 millisecond/pulse, about 0.05 millisecond/pulse to about 50 millisecond/pulse, about 0.05 millisecond/pulse to about 100 millisecond/pulse, about 0.05 millisecond/pulse to about 500 millisecond/pulse, about 0.05 millisecond/pulse to about 1,000 millisecond/pulse, about 0.05 millisecond/pulse to about 5,000 millisecond/pulse, about 0.1 millisecond/pulse to about 0.5 millisecond/pulse, about 0.1 millisecond/pulse to about 1 millisecond/pulse, about 0.1 millisecond/pulse to about 5 millisecond/pulse, about 0.1 millisecond/pulse to about 10 millisecond/pulse, about 0.1 millisecond/pulse to about 50 millisecond/pulse, about 0.1 millisecond/pulse to about 100 millisecond/pulse, about 0.1 millisecond/pulse to about 500 millisecond/pulse, about 0.1 millisecond/pulse to about 1,000 millisecond/pulse, about 0.1 millisecond/pulse to about 5,000 millisecond/pulse, about 0.5 millisecond/pulse to about 1 millisecond/pulse, about 0.5 millisecond/pulse to about 5 millisecond/pulse, about 0.5 millisecond/pulse to about 10 millisecond/pulse, about 0.5 millisecond/pulse to about 50 millisecond/pulse, about 0.5 millisecond/pulse to about 100 millisecond/pulse, about 0.5 millisecond/pulse to about 500 millisecond/pulse, about 0.5 millisecond/pulse to about 1,000 millisecond/pulse, about 0.5 millisecond/pulse to about 5,000 millisecond/pulse, about 1 millisecond/pulse to about 5 millisecond/pulse, about 1 millisecond/pulse to about 10 millisecond/pulse, about 1 millisecond/pulse to about 50 millisecond/pulse, about 1 millisecond/pulse to about 100 millisecond/pulse, about 1 millisecond/pulse to about 500 millisecond/pulse, about 1 millisecond/pulse to about 1,000 millisecond/pulse, about 1 millisecond/pulse to about 5,000 millisecond/pulse, about 5 millisecond/pulse to about 10 millisecond/pulse, about 5 millisecond/pulse to about 50 millisecond/pulse, about 5 millisecond/pulse to about 100 millisecond/pulse, about 5 millisecond/pulse to about 500 millisecond/pulse, about 5 millisecond/pulse to about 1,000 millisecond/pulse, about 5 millisecond/pulse to about 5,000 millisecond/pulse, about 10 millisecond/pulse to about 50 millisecond/pulse, about 10 millisecond/pulse to about 100 millisecond/pulse, about 10 millisecond/pulse to about 500 millisecond/pulse, about 10 millisecond/pulse to about 1,000 millisecond/pulse, about 10 millisecond/pulse to about 5,000 millisecond/pulse, about 50 millisecond/pulse to about 100 millisecond/pulse, about 50 millisecond/pulse to about 500 millisecond/pulse, about 50 millisecond/pulse to about 1,000 millisecond/pulse, about 50 millisecond/pulse to about 5,000 millisecond/pulse, about 100 millisecond/pulse to about 500 millisecond/pulse, about 100 millisecond/pulse to about 1,000 millisecond/pulse, about 100 millisecond/pulse to about 5,000 millisecond/pulse, about 500 millisecond/pulse to about 1,000 millisecond/pulse, about 500 millisecond/pulse to about 5,000 millisecond/pulse, or about 1,000 millisecond/pulse to about 5,000 millisecond/pulse. In some instances, the electric field generated by the electrodes for nanoelectroporation comprises a plurality of pulses with pulse duration from about 0.01 millisecond/pulse, about 0.05 millisecond/pulse, about 0.1 millisecond/pulse, about 0.5 millisecond/pulse, about 1 millisecond/pulse, about 5 millisecond/pulse, about 10 millisecond/pulse, about 50 millisecond/pulse, about 100 millisecond/pulse, about 500 millisecond/pulse, about 1,000 millisecond/pulse, or about 5,000 millisecond/pulse. In some instances, the electric field generated by the electrodes for nanoelectroporation comprises a plurality of pulses with pulse duration from at least about 0.01 millisecond/pulse, about 0.05 millisecond/pulse, about 0.1 millisecond/pulse, about 0.5 millisecond/pulse, about 1 millisecond/pulse, about 5 millisecond/pulse, about 10 millisecond/pulse, about 50 millisecond/pulse, about 100 millisecond/pulse, about 500 millisecond/pulse, or about 1,000 millisecond/pulse. In some instances, the electric field generated by the electrodes for nanoelectroporation comprises a plurality of pulses with pulse duration from at most about 0.05 millisecond/pulse, about 0.1 millisecond/pulse, about 0.5 millisecond/pulse, about 1 millisecond/pulse, about 5 millisecond/pulse, about 10 millisecond/pulse, about 50 millisecond/pulse, about 100 millisecond/pulse, about 500 millisecond/pulse, about 1,000 millisecond/pulse, or about 5,000 millisecond/pulse. In some cases, the nanoelectroporation comprises 1 pulse, 2 pulses, 3 pulses, 4 pulses, 5 pulses, 6 pulses, 7 pulses, 8 pulses, 9 pulses, 10 pulses, 11 pulses, 12 pulses, 13 pulses, 14 pulses, 15 pulses, 16 pulses, 17 pulses, 18 pulses, 19 pulses, 20 pulses or more.

In some cases, the extracellular vesicles produced and secreted by the extracellular vesicle donor cells are collected and purified from a cell culture medium by centrifugation or ultracentrifugation, which may allow the extracellular vesicles to be purified from other cellular debris or molecules based on the density of the extracellular vesicles.

In some cases, the methods and systems of producing the extracellular vesicles comprising the therapeutic polynucleotides (and optional targeting polypeptide) comprise loading the nanochannels with the plurality of vectors to be nanoelectroporated into the cells. In some cases, molecules other than vectors (e.g., proteins, biomolecules, compounds, etc.) can be loaded into the nanochannels to be nanoelectroporated into the cells. In some cases, the electric field generated by the upper and the lower electrodes accelerate the vectors into the cells. In some cases, the electric field generated for nanoelectroporation creates pores in the cells of the membrane to allow the nanoelectroporation of the vectors. In some cases, the pores in the membrane of the extracellular vesicle donor cells can be formed at a focal point, e.g. exit of the nanochannel where the electric field directly contacts the cell membrane.

In some cases, a nanoelectroporated extracellular vesicle donor cell can produce and secrete at least 10%, 50%, 1 fold, 5 fold, 10 fold, 50 fold, 100 fold, 500 fold, 1000 fold, 5000 fold, or more extracellular vesicles than an extracellular vesicle donor cell transfected by non-nanoelectroporation (e.g. conventional bulk electroporation, gene gun, lipofectamine transfection, etc.) In some cases, an nanoelectroporated extracellular vesicle donor cell can produce and secrete a number of extracellular vesicles that is increased by at least 10%, 50%, 1 fold, 5 fold, 10 fold, 50 fold, 100 fold, 500 fold, 1000 fold, 5000 fold, or more compared to a number of extracellular vesicles produced and secreted by an extracellular vesicle donor cell stimulated by non-nanoelectroporation (e.g. conventional bulk electroporation, gene gun, lipofectamine transfection, global cellular stress response, starvation, hypoxia, and heat treatment, etc.)

In some cases, the extracellular vesicle donor cell can produce and secrete more extracellular vesicles, when the extracellular vesicle donor cell is cultured and nanoelectroporated at an increased temperature. For example, the extracellular vesicle donor cell is cultured and nanoelectroporated at 37° C. produces and secretes more extracellular vesicles than the extracellular vesicle donor cell cultured and nanoelectroporated at 4° C. In some cases, the extracellular vesicle donor cell produces and secretes at least 10%, 50%, 1 fold, 5 fold, 10 fold, 50 fold, 100 fold, 1000 fold, or more extracellular vesicles for each 1° C. increased over 4° C. during the culturing and nanoelectroporating of the extracellular vesicle donor cell.

In some cases, the extracellular vesicle donor cell can produce and secrete more extracellular vesicles, when the extracellular vesicle donor cell is cultured in a buffer comprising Ca²⁺. For example, after nanoelectroporation an extracellular vesicle donor cell cultured in a buffer comprising 500 nM Ca²⁺ produces and secretes more extracellular vesicles compared to if the extracellular vesicle donor cell is cultured in a buffer comprising no Ca²⁺ after nanoelectroporation. In some cases, the extracellular vesicle donor cell produces and secretes at least 10%, 50%, 1 fold, 5 fold, 10 fold, 50 fold, 100 fold, 1000 fold, or more extracellular vesicles when cultured in a buffer comprising increased concentration of Ca²⁺ after nanoelectroporation compared to if the extracellular vesicle donor cell is cultured in a buffer comprising no Ca²⁺ after nanoelectroporation. Example of the increased concentration of Ca²⁺ in the buffer includes 10 nM, 50 nM, 100 nM, 150 nM, 200 nM, 250 nM, 300 nM, 400 nM, 500 nM, 600 nM, 7000 nM, 800 nM, 900 nM, 1000 nM, 1100 nM, 1200 nM, 1300 nM, 1400 nM, 1500 nM, 2000 nM, 2500 nM, 3000 nM, 5000 nM, 10000 nM, or higher concentration of Ca²⁺.

In some cases, the extracellular vesicle donor cell can produce and secrete more extracellular vesicles, when the extracellular vesicle donor cell is transfected with the at least one heterologous polynucleotide comprising a vector encoding 6-kbp Achaete-Scute Complex Like-1 (Ascl1), 7-kbp Pou Domain Class 3 Transcription factor 2 (Pou3f2 or Brn2), and 9-kbp Myelin Transcription Factor 1 Like (Myt1l). By transfecting the extracellular vesicle donor cell with the 6-kbp Achaete-Scute Complex Like-1 (Ascl1), 7-kbp Pou Domain Class 3 Transcription factor 2 (Pou3f2 or Brn2), and 9-kbp Myelin Transcription Factor 1 Like (Myt1l), the number of extracellular vesicles produced and secreted by the extracellular vesicle donor cell stimulated by nanoelectroporation can be increased by at least 10%, 50%, 1 fold, 5 fold, 10 fold, 50 fold, 100 fold, 1000 fold, or more folds compared to the number of extracellular vesicles produced and secreted by nanoelectroporating the extracellular vesicle donor cell without being transfected with the 6-kbp Achaete-Scute Complex Like-1 (Ascl1), 7-kbp Pou Domain Class 3 Transcription factor 2 (Pou3f2 or Brn2), and 9-kbp Myelin Transcription Factor 1 Like (Myt1l).

In some instances, extracellular vesicles produced and secreted by nanoelectroporated extracellular vesicle donor cells comprise at least 50%, 1 fold, 2 fold, 5 fold, 100 fold, 500 fold, 1000 fold, or more therapeutic polynucleotides compared to extracellular vesicles produced and secreted by extracellular vesicle donor cells transfected by non-nanoelectroporation. In some cases, the therapeutic polynucleotides encapsulated by the extracellular vesicles produced and secreted by nanoelectroporated extracellular vesicle donor cells are at least 10%, 20%, 30%, 40%, 50%, 60%, 70%, 80%, 90%, 95%, 99%, or more likely to be intact for encoding therapeutic polypeptides than therapeutic polynucleotides encapsulated by the extracellular vesicles produced and secreted by extracellular vesicle donor cells transfected by non-nanoelectroporation.

In some cases, a microchannel-electroporated or nanochannel-electroporated extracellular vesicle donor cell produces and secretes an increased percentage of extracellular vesicles comprising at least one copy of the therapeutic polynucleotide compared to a percentage of extracellular vesicles comprising at least one copy of the therapeutic polynucleotide produced and secreted by an extracellular vesicle donor cell transfected by other methods of transfection (e.g. conventional bulk electroporation, gene gun, lipofectamine transfection, etc.). In some cases, the percentage of extracellular vesicles comprising at least one copy of the therapeutic polynucleotide produced and secreted by microchannel electroporated or nanochannel electroporated extracellular vesicle donor cell is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or more compared to the percentage of extracellular vesicles comprising at least one copy of the therapeutic polynucleotide produced and secreted by extracellular vesicle donor cell transfected by other methods of transfection. In some cases, the percentage of extracellular vesicles comprising at least one copy of the therapeutic polynucleotide can be determined by measuring the number of extracellular vesicles comprising the at least one copy of the therapeutic polynucleotide produced and secreted by extracellular vesicle donor cells over a span of 1 minute, 10 minutes, 30 minutes, 1 hour, 2 hours, 3 hours, 4 hours, 5 hours, 6 hours, 12 hours, 24 hours, 36 hours, 48 hours, 72 hours, 4 days, 5 days, 6 days, 1 week, 2 weeks, 3 weeks, 1 month, 2 moths, or a longer span of time. In some cases, microchannel electroporated or nanochannel electroporated extracellular vesicle donor cell produces and secretes an increased number of extracellular vesicles comprising at least one copy of the therapeutic polynucleotide (e.g., therapeutic mRNA, therapeutic miRNA) compared to a number of extracellular vesicles comprising at least one copy of the therapeutic polynucleotide produced and secreted by extracellular vesicle donor cell transfected by other methods of transfection (e.g. conventional bulk electroporation, gene gun, lipofectamine transfection, etc.). In some cases, the microchannel electroporated or nanochannel electroporated extracellular vesicle donor cell produces and secretes an increased number of extracellular vesicles comprising at least one copy of the therapeutic polynucleotide is increased by at least 0.1 fold, 0.2 fold, 0.5 fold, 2 fold, 5 fold, 10 fold, 50 fold, 100 fold, 500 fold, 1,000 fold, 5,000 fold, 10,000 fold, or more compared to extracellular vesicles produced and secreted by extracellular vesicle donor cell transfected by other methods of transfection (e.g., conventional bulk electroporation, gene gun, lipofectamine transfection, etc.). In some cases, microchannel electroporated or nanochannel electroporated extracellular vesicle donor cell produces and secretes an increased number of extracellular vesicles, where at least 1 out of 500 extracellular vesicles, at least 1 out of 200 extracellular vesicles, at least 1 out of 100 extracellular vesicles, at least 1 out of 50 extracellular vesicles, at least 1 out of 25 extracellular vesicles, or at least 1 out of 10 extracellular vesicles comprise at least 1 copy of therapeutic polynucleotide (e.g., therapeutic mRNA, therapeutic miRNA).

### Pharmaceutical Compositions

In some cases, the extracellular vesicles can be formulated into pharmaceutical composition. In some cases, the pharmaceutical composition comprising the extracellular vesicles or exosomes can be administered to a subject by multiple administration routes, including but not limited to, parenteral, oral, buccal, rectal, sublingual, or transdermal administration routes. In some cases, parenteral administration comprises intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration. In some cases, the pharmaceutical composition and formulations described herein are administered to a subject by intravenous, subcutaneous, and intramuscular administration. In some cases, the pharmaceutical composition and formulations described herein are administered to a subject by intravenous administration. In some cases, the pharmaceutical composition and formulations described herein are administered to a subject by administration. In some cases, the pharmaceutical composition and formulations described herein are administered to a subject by intramuscular administration.

### Kits/Article of Manufacture

Disclosed herein, in certain aspects, are kits and articles of manufacture for use with one or more methods and compositions described herein. Also described herein are systems of manufacturing the extracellular vesicles or exosomes. In some cases, the systems comprise methods to nanoelectroporate extracellular vesicle donor cells to stimulate the production of extracellular vesicles or exosomes comprising the therapeutic polynucleotides.

In some cases, the kits can include a carrier, package, or container that is compartmentalized to receive one or more containers such as vials, tubes, and the like, each of the container(s) comprising one of the separate elements to be used in the methods described herein. Suitable containers include, for example, bottles, vials, syringes, and test tubes. In some cases, the containers can be formed from a variety of materials such as glass or plastic. kit typically includes labels listing contents and/or instructions for use, and package inserts with instructions for use. A set of instructions may also be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain cases, the extracellular vesicles comprising the therapeutic polynucleotides can be presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, the extracellular vesicles comprising the therapeutic polynucleotides containing a compound provided herein formulated in a compatible pharmaceutical carrier are also prepared, placed in an appropriate container, and labeled for treatment of an indicated condition.

In some cases, the kits comprise articles of manufacture that are useful for developing adoptive therapies and methods of treatment described herein. In some cases, kits comprise at least one extracellular vesicle comprising the therapeutic polynucleotides or components to manufacture the at least one extracellular vesicles comprising the therapeutic polynucleotides. In some cases, kits comprise at least one exosome comprising the therapeutic polynucleotides or components to manufacture the at least one exosome comprising the therapeutic polynucleotides.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular and not limited to sequential steps. Accordingly, terms such as “first” and “second” do not necessarily imply priority, order of importance, or order of acts.

As used herein, the term “about” when referring to a number or a numerical range means that the number or numerical range referred to is an approximation within experimental variability (or within statistical experimental error), and the number or numerical range may vary from, for example, from 1% to 10% of the stated number or numerical range. Unless otherwise indicated by context, the term “about” refers to +10% of a stated number or value.

The term “approximately” means within an acceptable error range for the particular value as determined by one of ordinary skill in the art, which will depend in part on how the value is measured or determined, e.g., the limitations of the measurement system. For example, “approximately” can mean within 1 or more than 1 standard deviation, per the practice in the given value. Where particular values are described in the application and claims, unless otherwise stated the term “approximately” should be assumed to mean an acceptable error range for the particular value.

The terms “increased”, “increasing”, or “increase” are used herein to generally mean an increase by a statically significant amount. In some cases, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased”, “decreasing”, or “decrease” are used herein generally to mean a decrease by a statistically significant amount. In some cases, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “patient” or “subject” are used interchangeably herein, and encompass mammals. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

As used herein, a “cell” generally refers to a biological cell. A cell is the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), or a cell from a mammal (e.g., a pig, a cow, a goat, a sheep, a rodent, a rat, a mouse, a non-human primate, a human, etc.). Sometimes a cell is not originating from a natural organism (e.g. a cell is a synthetically made, sometimes termed an artificial cell). In some cases, the cell is a primary cell. In some cases, the cell is derived from a cell line.

The term “nucleotide,” as used herein, generally refers to a base-sugar-phosphate combination. A nucleotide comprises a synthetic nucleotide. A nucleotide comprises a synthetic nucleotide analog. Nucleotides is monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP.

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. In some cases, a polynucleotide is exogenous (e.g. a heterologous polynucleotide). In some cases, a polynucleotide is endogenous to a cell. In some cases, a polynucleotide can exist in a cell-free environment. In some cases, a polynucleotide is a gene or fragment thereof. In some cases, a polynucleotide is DNA. In some cases, a polynucleotide is RNA. A polynucleotide can have any three-dimensional structure, and can perform any function, known or unknown. In some cases, a polynucleotide comprises one or more analogs (e.g. altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), non-coding RNA, ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. In some cases, the sequence of nucleotides is interrupted by non-nucleotide components.

“Fully intact” and “substantially intact” refer to a nucleic acid described herein having a nucleic acid sequence that can be transcribed and/or translated into a therapeutic polypeptide described herein. Fully intact nucleic acid refers to full-length nucleic acid sequence, which is not partially degraded or fragmented. For example, a fully intact nucleic acid can be a messenger RNA that can be translated into a full-length protein such as any one of the therapeutic polypeptides described herein. In general, a fully intact or substantially intact messenger RNA is capable of being translated into a polypeptide. Generally, messenger RNA comprises a 5′ cap which may assist with binding to a ribosome and a poly (A) tail, which may be useful for translation. The term “substantially intact” refers to a nucleic acid sequence that can be partially degraded or fragmented but still can be transcribed and/or translated into any one of the therapeutic polypeptides described herein. For example, a substantially intact nucleic acid can be a partially degraded or fragmented messenger RNA that can be translated into any one of the therapeutic polypeptides described herein.

As used herein, the terms “polypeptide”, “peptide”, and “protein” can be used interchangeably herein in reference to a polymer of amino acid residues. A polypeptide can refer to a full-length polypeptide as translated from a coding open reading frame, or as processed to its mature form. A polypeptide can refer to a degradation fragment or a processing fragment of a protein that nonetheless uniquely or identifiably maps to a particular protein. A polypeptide can be a single linear polymer chain of amino acids bonded together by peptide bonds between the carboxyl and amino groups of adjacent amino acid residues. A polypeptide can be modified, for example, by the addition of carbohydrate, phosphorylation, etc. A polypeptide can be a heterologous polypeptide.

As used herein, the terms “fragment” or equivalent terms can refer to a locus of a protein that has less than the full length of the protein and optionally maintains the function of the protein. “Percent identity” and “% identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO:Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN, FASTA, gapped BLAST, BLASTP, BLASTN, or GCG.

As used herein, the term “in vivo” is used to describe an event that takes place in a subject's body.

As used herein, the term “ex vivo” is used to describe an event that takes place outside of a subject's body. An “ex vivo” assay cannot be performed directly on a subject. Rather, it is performed upon a sample separate from a subject, such as a biological sample obtained from the subject. Ex vivo is used to describe an event occurring in an intact cell or other type of biological sample outside a subject's body.

As used herein, the term “in vitro” is used to describe an event that takes place contained in a container for holding a laboratory reagent such that it is separated from the living biological source organism from which the material is obtained. In vitro assays can encompass cell-based assays in which live or dead cells or other biological materials are employed. In vitro assays can also encompass a cell-free assay in which no intact cells are employed.

“Treating” or “treatment” can refer to a therapeutic treatment, a cosmetic treatment, and/or prophylactic or preventative measures, wherein the object is to prevent or slow down (lessen) a targeted condition (e.g., pathologic condition) or a disorder, often by preventing, reducing, or lessening one or more symptoms of the condition or disorder. Those in need of treatment include those already with the disorder, as well as those prone to have the disorder, or those in whom the disorder is to be prevented. As used herein, a condition or disorder includes a condition or disorder associated with aging. A therapeutic benefit can refer to eradication or amelioration of symptoms or of an underlying disorder being treated. Also, a therapeutic benefit is achieved with the eradication or amelioration of one or more of the physiological symptoms associated with the underlying disorder such that an improvement is observed in the subject, notwithstanding that the subject can still be afflicted with the underlying disorder. A prophylactic effect can include delaying, preventing, or eliminating the appearance of a disease or condition, delaying or eliminating the onset of symptoms of a disease or condition, slowing, halting, or reversing the progression of a disease or condition, or any combination thereof. A prophylactic benefit, a subject at risk of developing a particular disease, or to a subject reporting one or more of the physiological symptoms of a disease can undergo treatment, even though a diagnosis of this disease cannot have been made.

The term “effective amount” and “therapeutically effective amount,” as used interchangeably herein, generally refer to the quantity of a pharmaceutical composition, for example a pharmaceutical composition comprising a composition described herein, that is sufficient to result in a desired activity upon administration to a subject in need thereof. Within the context of the present disclosure, the term “therapeutically effective” refers to that quantity of a pharmaceutical composition that can be sufficient to delay the manifestation, arrest the progression, relieve or alleviate at least one symptom of a disorder treated by the methods of the present disclosure.

The terms “therapeutic extracellular vesicle,” “therapeutic EV,” “therapeutic exosome,” “therapeutic microvesicle,” “therapeutic apoptotic body,” and the like, as used herein, generally refer to an extracellular vesicle (or, where applicable, an exosome, microvesicle, or apoptotic body) that can be used to treat a condition or disorder. Depending on the context in which the terms are used, they may, at times, refer to an extracellular vesicle (or, exosome, microvesicle, or apoptotic body) that can be used to treat a condition or disorder associated with aging.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component is “pharmaceutically acceptable” in the sense of being compatible with the other ingredients of a pharmaceutical formulation. It can also be suitable for use in contact with the tissue or organ of humans and non-human mammals without excessive toxicity, irritation, allergic response, immunogenicity, or other problems or complications, commensurate with a reasonable benefit/risk ratio.

The term “pharmaceutical composition” refers to the systems or a mixture of the systems or compositions comprising each component of the systems disclosed herein with other chemical components, such as diluents or carriers. The pharmaceutical composition can facilitate administration of the systems or components of the systems to the subject. Multiple techniques of administering a compound exist in the art including, but not limited to, oral, injection, aerosol, parenteral, and topical administration.

The terms “transfection” or “transfected” generally refers to introduction of a nucleic acid construct into a cell by non-viral or viral-based methods. In some cases, the nucleic acid molecules are gene sequences encoding complete proteins or functional portions thereof. In some cases, the nucleic acid molecules are non-coding sequences. In some cases, the transfection methods are utilized for introducing nucleic acid molecules into a cell for generating a transgenic animal. Such techniques can include pronuclear microinjection, retrovirus mediated gene transfer into germ lines, gene targeting into embryonic stem cells, electroporation of embryos, sperm mediated gene transfer, and in vitro transformation of somatic cells, such as cumulus or mammary cells, or adult, fetal, or embryonic stem cells, followed by nuclear transplantation.

“Nanoelectroporation” or “nanochannel electroporation” refers to transfecting a cell with at least one heterologous polynucleotide such as a vector by loading the at least one heterologous polynucleotide into a nanochannel and accelerating the at least on heterologous polynucleotide into the cell with by generating an electric field. The cell to be transfected is situated at an opening of the nanochannel, where the electric field of the nanoelectroporation creates pores in the cell membrane to allow the at least one heterologous polynucleotide to be introduced into the cell. “nanochannel” as used herein, generally refers to a channel with an opening of a size in the scale of nanometers or micrometers.

A “plasmid,” as used herein, generally refers to a non-viral expression vector, e.g., a nucleic acid molecule that encodes for genes and/or regulatory elements necessary for the expression of genes. The term “vector,” as used herein, generally refers to a nucleic acid molecule capable transferring or transporting a payload nucleic acid molecule. The payload nucleic acid molecule can be generally linked to, e.g., inserted into, the vector nucleic acid molecule. A vector can include sequences that direct autonomous replication in a cell, or can include sequences sufficient to allow integration into host cell gene (e.g., host cell DNA). Examples of a vector can include, but are not limited to, plasmids (e.g., DNA plasmids or RNA plasmids), transposons, cosmids, bacterial artificial chromosomes, and viral vectors. A “viral vector,” as used herein, generally refers to a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to Gamma-retroviral, Alpha-retroviral, Foamy viral, lentiviral, adenoviral, or adeno-associated viral vectors. A vector of any of the aspects of the present disclosure can comprise exogenous, endogenous, or heterologous control sequences such as promoters and/or enhancers.

Whenever the term “at least,” “greater than,” “greater than or equal to”, or a similar phrase precedes the first numerical value in a series of two or more numerical values, the term “at least,” “greater than,” “greater than or equal to” or similar phrase applies to each of the numerical values in that series of numerical values. For example, “at least 1, 2, or 3” is equivalent to “at least 1, at least 2, and/or at least 3.”

Whenever the term “no more than,” “less than,” “less than or equal to,” “no greater than,” “at most” or a similar phrase, precedes the first numerical value in a series of two or more numerical values, the term “no more than,” “less than,” “less than or equal to,” “no greater than,” “at most,” or similar phrase applies to each of the numerical values in that series of numerical values. For example, “less than 3, 2, or 1” is equivalent to “less than 3, less than 2, and/or less than 1.”

As used here, the term “exogenous RNA” generally refers to RNA that is artificially introduced into a cell or extracellular vesicle. For example, an extracellular vesicle comprising exogenous RNA can refer to an extracellular vesicle produced from a cell after an exogenous vector (e.g., exogenous DNA, a DNA plasmid, or other exogenous vector) encoding the exogenous mRNA is introduced into the cell, such as by transfection of the cell (e.g., by electroporation, nanoporation, cellular nanoporation, calcium phosphate transfection, lipofection, or the like) or transduction of the cell (e.g., by viral transduction). The term “exogenous RNA” can also refer to other methods of introducing exogenous mRNA into an extracellular vesicle or cell, including by direct or bulk transfection of the extracellular vesicle or cell. Similarly, as used here, the term “exogenous mRNA” generally refers to mRNA that is artificially introduced into a cell or extracellular vesicle. For example, an extracellular vesicle comprising a certain type of exogenous mRNA (e.g., exogenous extracellular matrix mRNA, exogenous VEGF mRNA, etc.) can refer to an extracellular vesicle produced from a cell after an exogenous vector (e.g., exogenous DNA, a DNA plasmid, or other exogenous vector) encoding the exogenous mRNA is introduced into the cell, such as by transfection of the cell (e.g., by electroporation, nanoporation, cellular nanoporation, calcium phosphate transfection, lipofection, or the like) or transduction of the cell (e.g., by viral transduction). The term “exogenous mRNA” can also refer to other methods of introducing exogenous mRNA into an extracellular vesicle or cell, including by direct or bulk transfection of the extracellular vesicle or cell. The term “exogenous” can be similarly interpreted when modifying other types of nucleic acids such as siRNA, miRNA, or DNA.

As used here, the term “gauge” generally refers to a set combination of inner diameter and outer diameter of the penetration portion of the needle, the hydrogel needle, the microneedle, or the microneedle device described herein.

As used herein, the term “microneedle” generally refers to a needle with at least one dimension, such as length, width or diameter that is less than 15 mm.

### EXAMPLES

The following illustrative examples are representative of embodiments of the compositions, systems, and methods described herein and are not meant to be limiting in any way.

### Example 1. Cellular Nanoporation (CNP) Generated Extracellular Vesicles

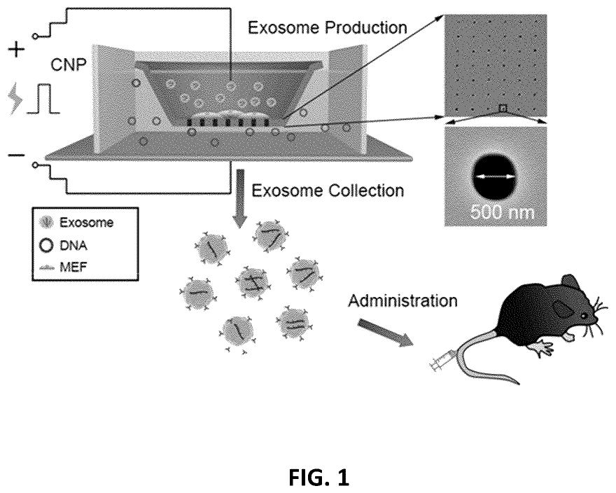

A cellular nanoporation (CNP) biochip, CNP system, and CNP method to stimulate cells to produce and release exosomes containing nucleotide sequences of interest including mRNA, microRNA and shRNA is used, as described herein. The system and method allowed a monolayer of source cells such as mouse embryonic fibroblasts (MEFs) and dendritic cells (DCs) to be cultured over the chip surface, which contained an array of nanochannels (FIG. 1). The nanochannels (˜500 nm in diameter) enabled the passage of transient electrical pulses to shuttle DNA plasmids from buffer into the attached cells (FIG. 1).

### Example 2. In Vitro Delivery of VEGF mRNA Extracellular Vesicles

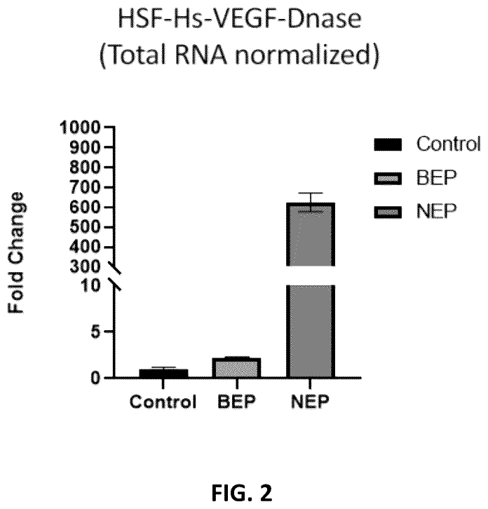

VEGF were first delivered into Human Dermal Fibroblasts (HDFs) using cellular nanoporation (CNP) and bulk electroporation (BEP) to generate exosomes containing VEGF mRNA. To compare the amount of VEGF mRNA within both CNP and BEP prepared exosomes, qtr.-PCR was used. Although both CNP and BEP produced exosomes containing VEGF mRNA, the concentrations of VEGF mRNA within CNP exosomes was much higher. Unlike BEP, CNP efficiently produced exosomes containing large mRNA, in which CNP-secreted exosomes contained >100 times more VEGF mRNA than exosomes from BEP (FIG. 2).

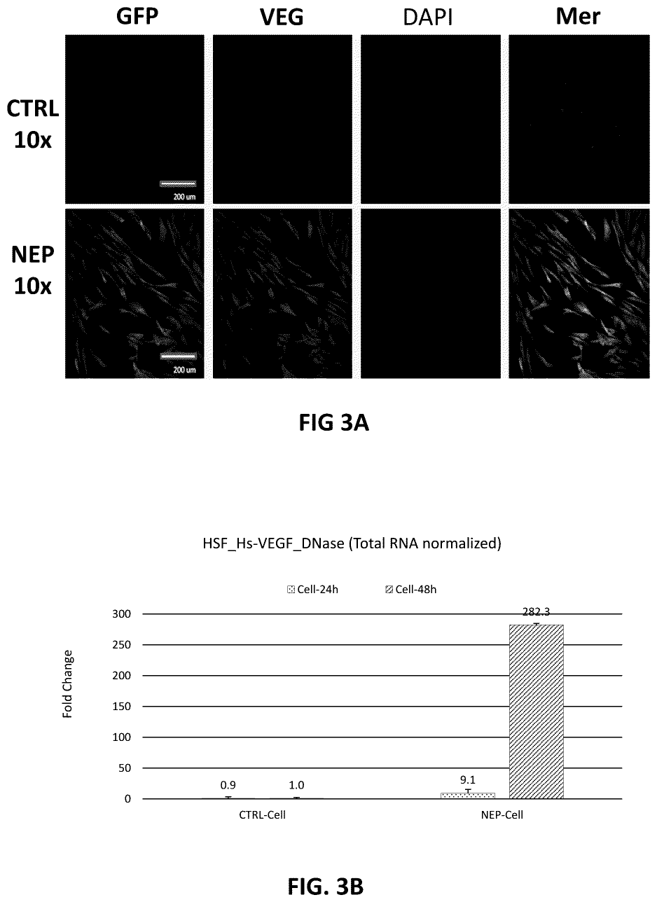

To investigate the delivery potential of VEGF mRNA EVs, 10,000 human dermal fibroblast cells were incubated with ranges from 1 μg to 1 ng of GFP-labelled VEGF mRNA (in EVs) or control EVs for 24 or 48 hours. Translation of VEGF protein was significantly upregulated after 48 hours (FIG. 3A) in VEGF mRNA EV-treated cells compared to control EV-treated cells. Qtr.-PCR at 24 hours and 48 hours after EV exposure confirmed significant delivery of VEGF mRNA to cells by 48 hours (FIG. 3B).

### Example 3. In Vivo Therapeutic Efficacy of VEGF mRNA EVs in Preclinical Models of Ischemia

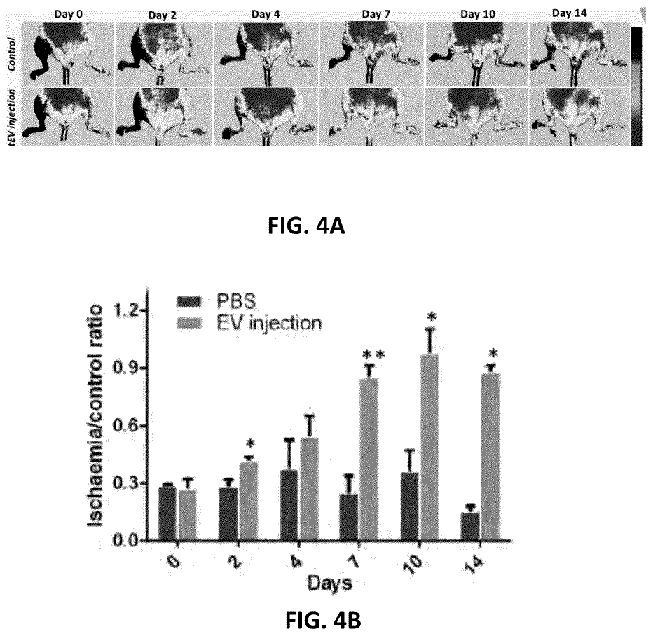

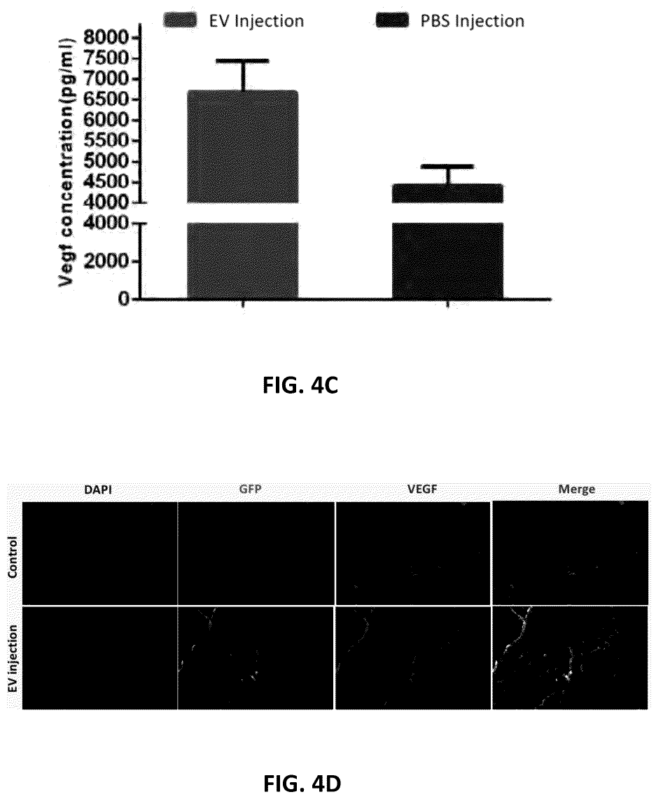

To assess the therapeutic potential of VEGF mRNA EVs in hindlimb ischemia (HLI) models, VEGF mRNA EVs were intramuscularly injected into HLI mice. Compared to PBS injection, VEGF mRNA EVs exhibited significantly improved revascularization over time (FIG. 4A). Quantification of average perfusion values of the ischemic footpad to that of the control footpad revealed significantly better perfusion starting as early as two days after EV delivery, which nearly total reperfusion occurring at 7 days after EV delivery (FIG. 4B). To further evaluate the in vivo delivery of VEGF mRNA EVs within the ischemic tissue, 0.20 g of ischemic leg tissue was collected 48 hours after VEGF mRNA loaded EV injection and analyzed for VEGF protein expression using ELISA. Tissue treated with VEGF mRNA EVs exhibited increased concentrations of VEGF protein compared to PBS control-treated tissue, indicating that VEGF mRNA delivery resulted in translation of VEGF protein as early as two days after EV delivery (FIG. 4C). Immunofluorescent staining results further confirmed that VEGF mRNA EV treatment increased VEGF expression by 48 hours after EV delivery (FIG. 4D).

### Example 4. In Vitro Delivery of Col1 mRNA Extracellular Vesicles

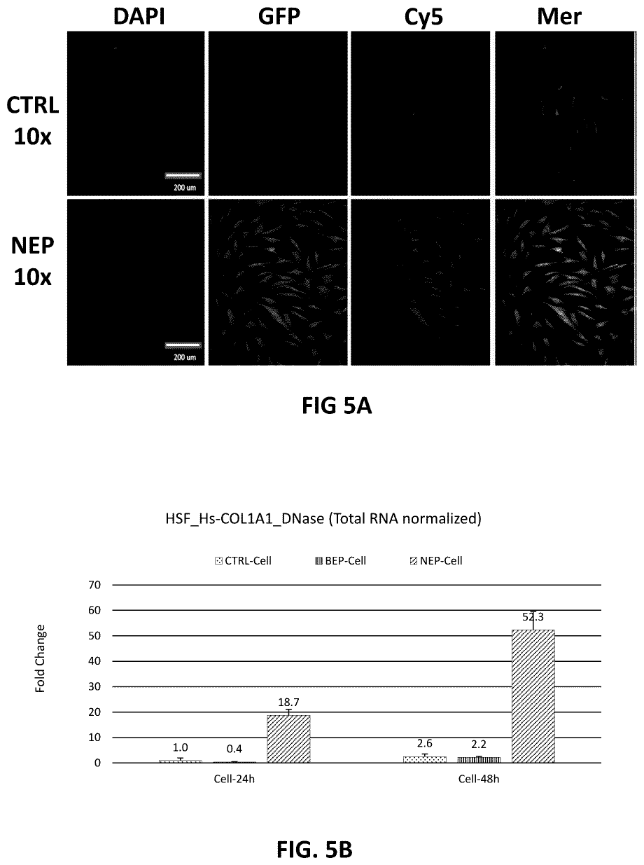

To investigate the delivery potential of Col1 mRNA EVs, 10,000 human dermal fibroblast cells were incubated with ranges from 1 μg to 1 ng of GFP-labelled Col1 mRNA (contained in CNP-produced EVs), Col1 mRNA BEP-produced exosomes, or control EVs for 24 or 48 hours. Translation of COL1 protein was significantly upregulated after 48 hours (FIG. 5A) in Col1 mRNA EV-treated cells compared to control EV-treated cells. RT-PCR at 24 hours and 48 hours after EV exposure confirmed significant delivery of Col1 mRNA to cells by Col1 mRNA CNP-produced EVs at 24 or 48 hours compared to both BEP and control EVs (FIG. 5B and Table 1).

TABLE 1
qPCR of human fibroblasts treated for 24 h/48 h with Col1a mRNA loaded EVs.

| | | ΔCr = Ct | | | | Total | Fold on | Total | |
|---|---|---|---|---|---|---|---|---|---|
| | Cr | (Col1) − Ct | | GAPDH | Cr Fold | RNA | total | RNA | EP |
| Sample | Mean | (GAPDH) | ΔΔCr | normalized | Change | (ng/uL) | RNA | normalized | Conditions |
| CTR-Cell_24 h | 28.79 | −8.349192 | 0 | 1 | 1 | 3.1 | 1 | 1 | No treatment |
| Ct (GAPDH) | 37.14 | | | | | | | | |
| BEP-Cell_24 h | 28.21 | −8.924286 | −0.58 | 1.48977 | 1.49 | 0.9 | 0.2647 | 0.39435 | 250 v-20 ms-1pulse |
| Ct (GAPDH) | 37.14 | | | | | | | | |
| NEP-Cell_24 h | 25.78 | −11.36102 | −3.01 | 8.06588 | 8.066 | 7.9 | 2.3235 | 18.7413 | 100 v-10 ms-10pulse |
| Ct (GAPDH) | 34.17 | | | | | | | | |
| CTR-Cell_48 h | 28.25 | −8.55369 | −0.2 | 1.15229 | 1.451 | 6 | 1.7647 | 2.56104 | No treatment |
| Ct (GAPDH) | 36.81 | | | | | | | | |
| BEP-Cell_48 h | 27.2 | −9.299859 | −0.95 | 1.93277 | 3.012 | 2.5 | 0.7353 | 2.21466 | 250 v-20 ms-1pulse |
| Ct (GAPDH) | 36.5 | | | | | | | | |
| NEP-Cell_48 h | 25.81 | −11.33076 | −2.98 | 7.89844 | 7.898 | 22.5 | 6.6176 | 52.2691 | 100 v-10 ms-10pulse |
| Ct (GAPDH) | 37.14 | | | | | | | | |

### Example 5. In Vivo Therapeutic Efficacy of Col1 mRNA EVs in Preclinical Models of Photoaging

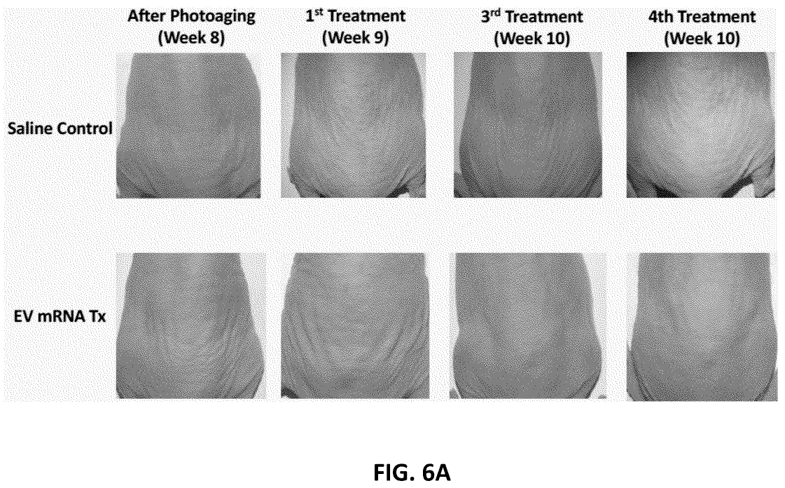

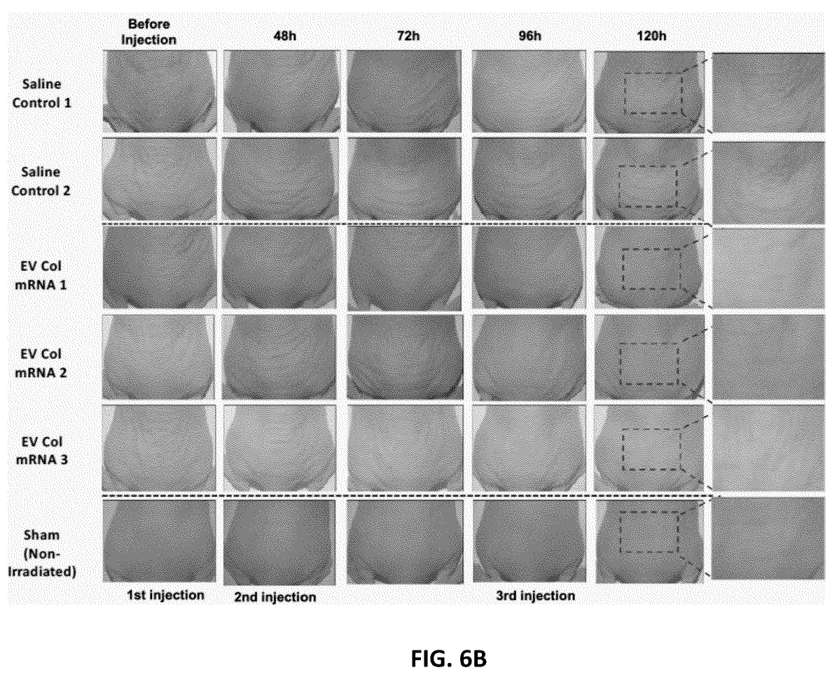

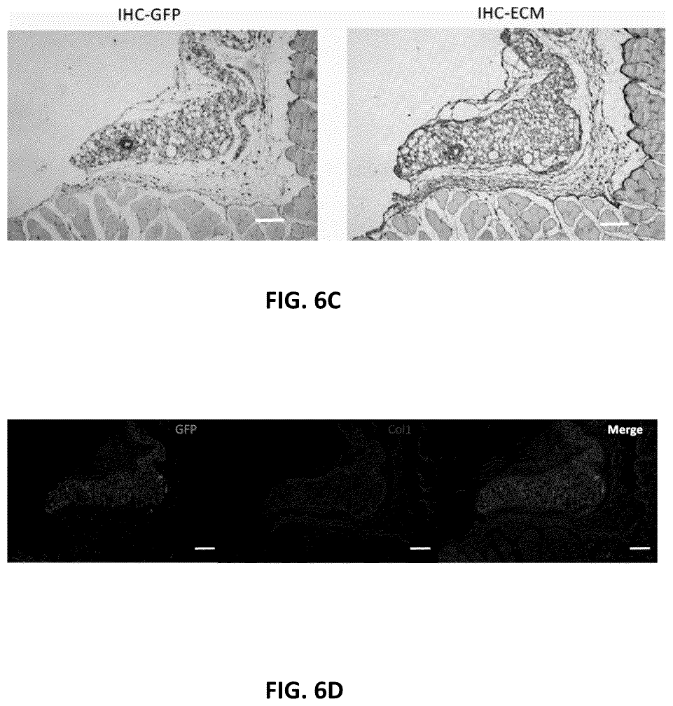

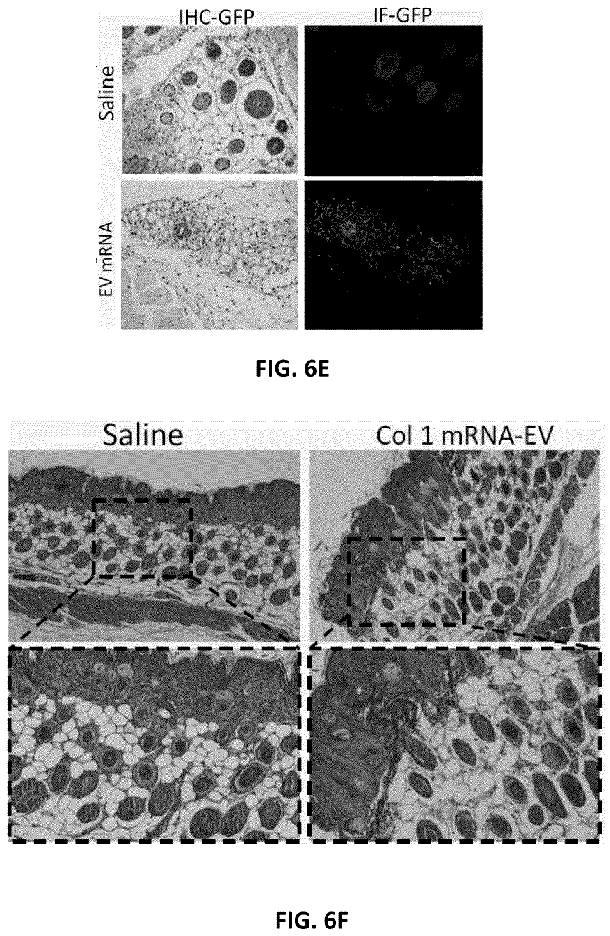

To assess the therapeutic potential of Col1 mRNA EVs in skin damage models, VEGF mRNA EVs were subcutaneously injected into mice using 28 gauge needles loaded with 1e11 EVs containing Col1 mRNA. In one example, mice received four doses of Col1 mRNA EVs after photoaging treatment (FIG. 6A). In another example, mice received three doses of Col1 mRNA EVs—a first dose, a second dose 48 hours after the first injection, and a third dose at 96 hours (FIG. 6B). Compared to saline injection, Col1 mRNA EVs exhibited significantly improved skin condition (e.g., reduced appearance of wrinkles) over time (FIGS. 6A-6B), with significant differences observed within about a week of the first injection. Immunohistochemical (FIGS. 6C and 6E) and immunofluorescent (FIG. 6D-6E) staining results confirmed that Col1 mRNA EV treatment increased collagen expression after EV delivery. Masson Trichrome staining results further confirmed that Col1 mRNA EV treatment increased collagen expression after EV delivery.

### Example 6. Methods and Systems of Synthesizing Therapeutic Extracellular Vesicles

Described herein are exemplary methods and systems for producing extracellular vesicles or exosomes for encapsulating therapeutic polynucleotides. The extracellular vesicles and exosomes produced by the instant methods and systems can be suitable to be formulated into a pharmaceutical composition for therapeutic uses.

### Conventional Cell Culture for EV Production

Human Dermal Fibroblast Normal (HDFn) cells can be obtained from ATCC (ATCC® PCS-201-010™), and used to generate the cell clones for therapeutic EV (tEV) production at GMP facility. For conventional cell culture using tissue culture flasks (Fisherbrand, Cat #FB012937), HDFn cells are maintained in Dulbecco's minimal essential medium (DMEM) supplemented with 10% fetal bovine serum (FBS), 100 U/mL penicillin/streptomycin and incubated at 37 C in 5% CO₂.

Clean nanopore electroporation (NEP) chips in bleach bath, followed by DI water rinse and N2 blow dry. Make surface treatment of the NEP chips using UV/Ozone Cleaner at the Support room in GMP facility. (optional. can be used for new PDMS spacer bonding)

Transfer cleaned NEP chips inside biosafety cabinets and HDFn cells are seeded on NEP chips with 80,000-100,000 cells per chip. After cell seeding, transfer the NEP chips to incubator and culture the cells in the NEP chips overnight.

Next day, check the cell growth condition under microscope and make sure the cells attach and spread well and reach >70% confluency on NEP chip surfaces. Replace the cell culture media in NEP chips with serum free media (DMEM only) before NEP treatment.

For reproducible NEP treatment, the concentration of target plasmid can be determined by Spectrophotometer (NanoDrop 2000c from Thermo Scientific) at EV validation (QC) room. The cells are treated by NEP using an electroporation system (Gene Pulser Xcell from Bio-Rad) with consistent electroporation parameters.

For EV harvests after NEP treatment for bench scale (<20 L), the cells are incubated for 24 hours in serum free media. For the pilot scale (>20 L), the cells are incubated for 24 hours in adherent cell bioreactor such as iCELLis 500+ system.

Cell culture conditioned media (CCM) are collected 24 hours after NEP treatment, and cell cultures are trypsinized and analyzed for cell viability with trypan blue staining. For accurate cell viability assays, the MTT assay can be used by absorbance detection using a microplate reader (Synergy™ HTX Multi-Mode from BioTek).

The processed NEP chips are cleaned in bleach bath, followed by DI water rinse and N2 blow dry. NEP chips are now ready for next batch of EV production run.

Centrifuge the CCM at 200×g for 5 minutes to remove cells and debris. Centrifuge the cell removed CCM at 2000×g for 30 minutes to remove cell debris. Transfer the cell debris removed CCM to a new tube without disturbing the pellet.

CCM is stored at 4 C for measurement of EV number and size distribution using NTA/DLS equipment as well as EV isolation. If CCM is stored for more than 48 hours at 4 C, the CCM is frozen and stored at −80 C for downstream use.

Before release the CCM to EV isolation room, send sampling specimen to EV validation. The NanoSight NS300 utilize Nanoparticle tracking analysis (NTA) to characterize nanoparticles from 10 nm-2000 nm in solution at EV validation. Compare NTA results with DLS and qNano measurement for further analysis of size based EV number quantification at EV validation.

If necessary, other tests such as Sterility test can be conducted for pre-validation of EVs. If all of test are done and passed criteria, process the EV isolation with the pass specimen.

cGMP Compatible EV Isolation by TFF System

Tangential flow filtration (TFF) can be used in EV isolation to remove non-EV components from collected CCM and for subsequent concentration. For EV isolation of CCM using a TFF system, filter the CCM sample with a 0.8 μm filter. In small-scale EV isolation from less than 50 mL of CCM, set up a TFF system, which consists of a Masterflex L/S® Digital Drive pump (Cole-Parmer, EW-77921-65) with 25-cm length tubing, a 50 mL Conical Centrifuge Tube, Female and Male Luer Thread Style Barbs, Stopcocks and a 500 kDa TFF-hollow fiber filter (Repligen, C02-S500-05-N). Screw the 50 mL Conical Centrifuge Tube into the cap of 50 mL Conical Centrifuge Tube in the TFF system. Add the filtered CCM sample to the 50 mL Conical Centrifuge Tube.

Start the Digital Drive pump and then the media is gently circulated through TFF-hollow fiber filter by automatic pumping with the pump. The pump is set at a speed of 35 mL/min.

The sample is first filtrated and concentrated until 5 mL of CCM (hold up volume) left in the Conical Centrifuge Tube. Stop the pump then open the PBS Stopcocks to add the washing buffer into the CCM sample. Restart the pump at 35 mL/min for diafiltration process for 2 hours to completely remove protein contaminants. Stop the pump. Close the PBS Stopcocks. Restart the pump at 35 mL/min to concentrate the sample until zero volume left in the Conical Centrifuge Tube. Stop the pump. Recover the purified CCM sample in 2 mL.

After NTA measurement of TFF-purified EV containing solution, if EV concentration from NTA data is less than 1e11/mL, the samples are concentrated using Amicon Ultra-4 Centrifugal Filter Unit with 10 KDa (from Millipore, Cat #UFC801096D).

Transfer the required volume (1-4 mL) of the purified EV solution to a Centrifugal Filter Unit. Centrifuge the TFF-processed CCM sample at 3000×g for 30 minutes at 4° C. using Eppendorf 5804 (15 mL) benchtop centrifuge to make a final volume of 80 uL.

The TFF-processed CCM sample is stored at 4 C for measurement of EV number and size distribution as well as EV validation.

For the larger-scale EV isolation from more than 500 mL of CCM, use a benchtop automatic TFF system of KRONOS from Solaris Biotech Solution. Measure NTA with 70 mL (hold up volume) of TFF-processed EV solution, if EV concentration from NTA data is less than 1e11/mL, the samples are concentrated using Amicon Ultra-15 Centrifugal Filter Unit with 10 KDa (from Millipore, Cat #UFC901096). Transfer the required volume (4-15 mL) of the purified EV solution to a Centrifugal Filter Unit. Centrifuge the TFF-processed CCM sample at 3000×g for 30 minutes at 4° C. using Eppendorf 5804 (50 mL) benchtop centrifuge to make a final volume of 250 uL. If TFF-processed CCM is stored for more than 6 hours at 4 C, the CCM is frozen and stored at −80 C for downstream use.

For clinical pilot scale (>20 L), Single-use TFF system can be developed with CS 1000 System from Sartorius AG.

cGMP Compatible EV Isolation by SEC Column

Size-exclusion chromatography (SEC) column has been employed to isolate EV from non-EV components from collected CCM. For EV isolation of CCM using a SEC column, concentrate CCM to higher EV number per mL (>1e11/mL).

In small-scale EV isolation from less than 0.5 mL of condensed CCM, place a SEC column in a holder and qEV column was washed with 20 mL PBS before use. With the bottom cap, pipette out the buffer above the top filter. Load 0.5 ml of condensed CCM and immediately remove the bottom cap to start collecting 0.5 mL fractions. Add more buffer as the last of the samples just enters the column, but add no more than 2 mL above the top filter. Collect 30 sequential fractions each of 0.5 mL if needed. If using qEV35 column, combine the fractions 8 to 10 into EV solution. If using qEV70 column, combine the fractions 9 to 11 into EV solution.

Each fraction or combined fractions were concentrated using Amicon Ultra-4 device with 10 kDa MWCO. Centrifuge the SEC-processed CCM sample at 3000×g for 30 minutes at 4° C. using Eppendorf 5804 (15 mL) benchtop centrifuge to make a final volume of 80 uL. For the medium-scale EV isolation, qEV100-35 column can be used with 100 mL of collected CCM or CCM concentrated by Amicon Ultra-15. For high end EV purification, combine TFF and SEC system together.

The TFF-processed CCM sample can be further purified by SEC column for high end downstream application. For the high-end SEC, qEV10-35 (SP7 w/ AFC) column can be used with Automatic Fraction Collector (AFC). For clinical pilot scale (>20 L), Single-use Liquid Chromatography can be developed with ÅKTA ready single-use system (customized ready packed column) from Cytiva (Formerly part of GE Healthcare).

### Example 7. CNP Generated Large Quantities of EVs Loaded with COL1A1 mRNA and Delivered Successfully In Vitro

Collagen I is the predominant collagen in the dermal extracellular matrix and partially encoded by the collagen I alpha I (COL1A1) gene. To generate EVs loaded with human COL1A1 mRNA, a cellular nanoporation (CNP) technique was employed that involved plating a monolayer of neonatal human dermal fibroblasts (nHDFs) on a nanopore surface and nano-transfecting the cells with a COL1A1-GFP plasmid (see FIG. 7A and FIG. 7B). Briefly, Neonatal human Primary dermal fibroblast (nHDF, PCS-201-010) were purchased from ATCC. nHDF were cultured in DMEM (Thermo Fisher Scientific) containing 10% heat-inactivated fetal bovine serum (FBS; catalogue no.: 10099141C Thermo Fisher Scientific) at 37° C. in humidified conditions equilibrated with 5% CO2. For CNP, a single layer of nHDF was seeded on a 1 cm×1 cm 3D CNP silicon chip surface for overnight incubation as previously described. Human COL1A1 cDNA (NM_000088.3) plasmid with GFP tag was purchased from Sino Biological (CAT #HG11776-ACG). Plasmids pre-loaded in PBS buffer were injected into individual cells via nanochannels using a 100V electric field with 10 pulses at 10 ms per pulse with a 0.1 s interval. Various electroporation conditions were tested to determine optimal conditions. BEP (Gene Pulser Xcell, Bio-Rad), using a 250V electric field with 20 ms, 1 pulse. pCMV-COL1A1-GFP plasmids at a concentration of 500 ng ml-1 in PBS for transfection.

EVs were isolated from culture medium the day after transfection. Briefly, cells were cultured in DMEM medium containing serum. The cell culture medium containing serum was removed when conduct CNP. Cells were then washed with PBS 3 times and cultured in serum-free cell culture medium for 24 h after CNP. EVs were collected from cell culture supernatants. In brief, the cell culture media (CCM) was centrifuge at 200×g for 5 minutes to remove cells and debris after with 2000×g for 30 minutes. Amicon Ultra-4 Centrifugal Filter Unit, 10 kDa (Millipore, Cat #801024) was used to concentrate the CCM. EVs sample was purified using total exosome isolation reagent (Invitrogen, Cat #4478360). EV particle size and number was measured by NanoSight NS300 (Malvern, UK). The RNA yield and size distribution were analyzed using an Agilent 2100 Bioanalyzer with an RNA 6000 Pico kit (Agilent Technologies, Foster City, CA, USA).

CNP treated cells were found to have a 10-fold higher EV numbers per cell as compared with cells treated with standard bulk electroporation (BEP) methods or untreated nHDFs in culture (see FIG. 7C). The EVs produced by each method were physically homogenous, with a size distribution peaking at about 100 nm in diameter as determined by nanoparticle tracking analysis (NTA) for the CNP-produced EVs (see FIG. 7D).

Western blot experiments showed that expression of exosome (CD9, CD63, TSG101) and microvesicle (ARF6) biomarkers in the CNP-treated group were significantly higher than in the untreated group, confirming the increase in secreted EVs (see FIG. 7E). Briefly, exosomes were lysed in RIPA buffer (50 mM Tris-HCl pH 8, 150 mM NaCl, 1% Triton X-100, 0.1% sodium deoxycholate and 0.1% SDS) containing a protease inhibitor cocktail (Complete, Roche). All sample proteins were transferred to polyvinylidene fluoride (PVDF) (MerckMillipore NO:IPVH00010) membranes and electrophoresed at 300 mA for 90 minutes. Membranes were blocked with BSA for 1 hour, and the appropriate concentration of primary antibody (1:1000) was added for overnight incubation at 4° C. Membranes were cut to the appropriate size and incubated with primary antibody, and incubated with the appropriate concentration of secondary antibody (1:15,000) for 1 hour at room temperature in the dark. The protein bands in the membrane were visualized by a UVP Bio Imaging System. The following antibodies were used: anti-CD63 antibody (Abcam, ab68418), anti-CD9 antibody (Cell signaling, cat #134035), anti-TSG101 (Abcam, ab125011), and anti-Arf6 (Abcam, ab77581). Kinetic analyses further showed that voltage optimized EV (see FIG. 7F where the maximum number of EVs was generated at a voltage of 100V) release peaked at 8 h after CNP induction, with continued secretion noted over the next 24 h when the supernatant was collected every 4 hours (see FIG. 7G). Finally, RT-qPCR showed that CNP-secreted EVs contained more than 200 times the COL1A1 mRNA than BEP-secreted EVs and 3000-fold higher COL1A1 mRNA than EVs secreted from non-transfected cells (see FIG. 7H). Briefly, the expression of human COL1A1 mRNA in EVs was measured using RT-qPCR following the manufacturer's recommended protocol. In brief, total RNA from purified EVs was obtained by RNA purification Mini kit (Norgen Biotek, Cat #55000) and DNA removal kit (Norgen Biotek, Cat #25720). SuperScript™ III First-Strand Synthesis System (Invitrogen) was used to synthesis the first-strand cDNA with random hexamers as primers. The expression of genes was measured using TB Green® Premix Ex Taq™ II (Takara, Cat #RR820). The primer sequences used were as follows: COL1A1(human), forward: 5′-CCTGGAAAGAATGGAGATGA-3′ and reverse: 5′-ACCATCCAAACCACTGAAAC-3′; Gapdh (human), forward: 5′-CAGCCTCAAGATCATCAGCA-3′ and reverse: 5′-AGAGGCAGGGATGATGTTCT-3′ Bioanalyzer assessment of gel agarose demonstrated large amounts of full length transcribed COL1A1 mRNA at about 4000 nucleotides (see FIG. 7I).

To assess the therapeutical potential of COL1A1 mRNA-containing EVs (COL1A1-EVs) in vitro, COL1A1-EVs were incubated with fibroblasts for 48 h (see FIG. 7J). Proliferation of fibroblasts was observed to increase with COL1A1-EV treatment in a dose-dependent manner (see FIG. 7K). Briefly, 60,000 nHDF cells were serum starved overnight by removing the serum from culture media. After 24 h, nHDF cells were treated with 10 ul different concentration of EVs (0, 10, 20, 30 μg/mL). Proliferation was performed using a Cell Counting Kit-8 (ab228554, abeam). After treated with EVs, CCK-8 reagent was added to each well at 0, 24, 48 hours. Absorbance at 460 nm was read and recorded using BioTek (BioTek Instruments, Winooski, VT, USA). After treatment, elevated COL1A1 protein expression was observed in fibroblasts treated with COL1A1-EVs, as evidenced by increased co-localization of GFP with COL1A1 under immunofluorescence microscopy. In contrast, COL1A1 levels were substantially lower without co-localization of GFP in the non-treated group (see FIG. 7L and FIG. 7M). Briefly, tissue sections were fixed with 4% paraformaldehyde for 20 min at room temperature and washed 3 times with PBS (Vetec) for 5 min each, Then the tissue was transferred to 0.2% Triton X-100 for 15 minutes (permeabilization), followed by blocking with BSA for 40 minutes and the addition of primary antibody (ab34710 and ab6556, Abcam) for blocking overnight at 4° C. Finally, the secondary antibody (ab6939 and ab6717, Abcam) was added and placed at room temperature for 60 minutes. After washing with PBS, DAPI (ThermoFisher) was added for nuclear staining and then mounted for observation. In EV cellular update assay, EVs were collect from CNP and incubated with 60,000 nHDF cells in a 24-well plate at 37° C. for 4 h before treatment. After incubation, cells were rinsed three times with cold PBS and fixed in 4% paraformaldehyde solution. Cell fluorescence intensity was analyzed using ImageJ (NIH).

Successful mRNA delivery into recipient cells was further confirmed by elevated collagen mRNA expression and collagen protein levels measured via qPCR and western blot analysis, respectively, in fibroblasts treated with COL1A1-EVs (see FIG. 7N, FIG. 7O and FIG. 7P). Briefly, after the tissue of interest was dissected (on ice), 300 μL of ice-cold lysis buffer per 5 mg of tissue was added and the mixture homogenized with an electric homogenizer, with an additional 300-600 μL of lysis buffer added during homogenization. After the homogenization, the mixture was agitated for 2 h at 4° C. and then centrifuged at 16,000×g for 20 min at 4° C. The supernatant was then collected in fresh tubes on ice, and the pellet was discarded. EVs at 24 and 48 h were collected for quantitative real-time PCR, and results showed an increase in COL1A1 mRNA that reached a maximum at 48 h. Moreover, pro-collagen I, a precursor of COL1 protein, was significantly increased following COL1A1-EV treatment, further indicating improved COL1A1 protein synthesis by the EV-treated fibroblasts compared with untreated fibroblasts (see FIG. 7Q). Briefly, cell culture medium was collected from EV treated cells. Human type I procollagen concentration was measured by ELISA (ab210966, Abcam, Cambridge, UK). Absorbance at 450 nm was measured using BioTek (BioTek Instruments, Winooski, VT, USA).

Furthermore, in order to investigate if the exogenously delivered Col1A1 form collagen with the endogenous Col1A2 in the recipient cells, co-staining of IHC was performed. Co-localization of the signal from Col1A1 and Col1A2 was observed, while some other exogenously delivered Col1A1 signals did not co-localize with Col1A2.

Previously, most studies focusing on nucleic-acid delivery had encapsulated small molecules in the 10-20 nucleotides range such as miRNAs and siRNAs as the payload, whereas larger nucleic acids such as mRNAs were seldom evaluated owing to the difficulty in loading them into EVs. However, the above findings demonstrated that CNP was able to load high copy number of COL1A1 mRNA (more than 4,000 nucleotides) into EVs, a feat that cannot be achieved by post-insertion loading methods. It has also been shown that functional COL1A1 mRNA could be encapsulated within EVs by CNP and that this COL1A1-EV delivery system significantly increased COL1A1 protein expression in vitro.

### Example 8. In Vivo Kinetics of COL1A1 mRNA Expression and Protein Translation Following COL1A1-EV Delivery

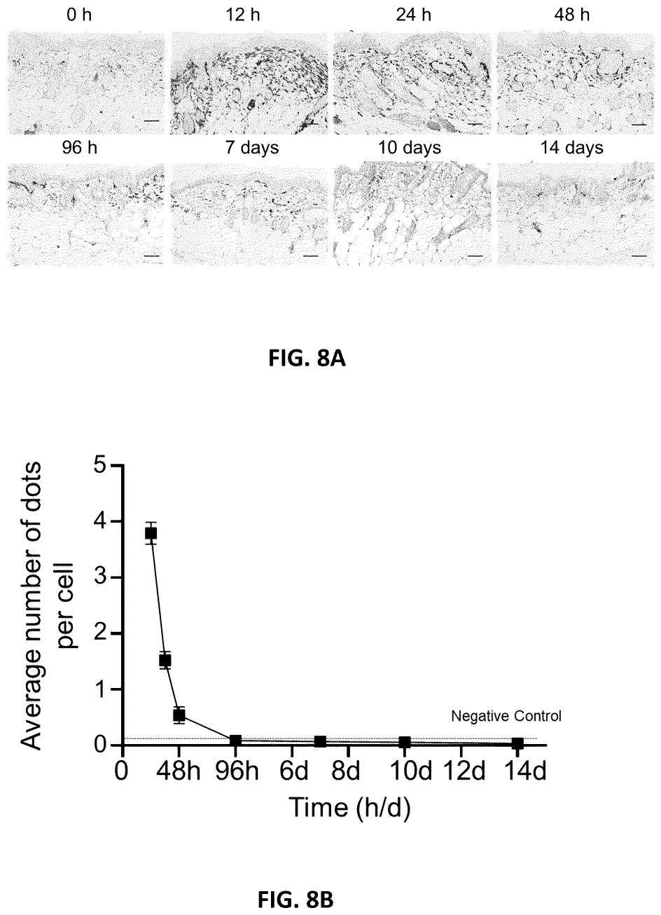

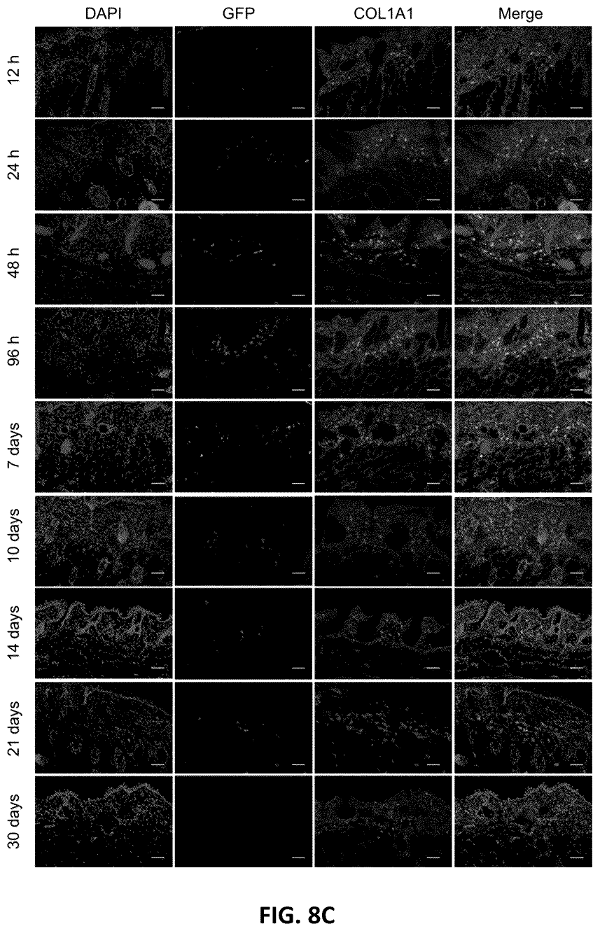

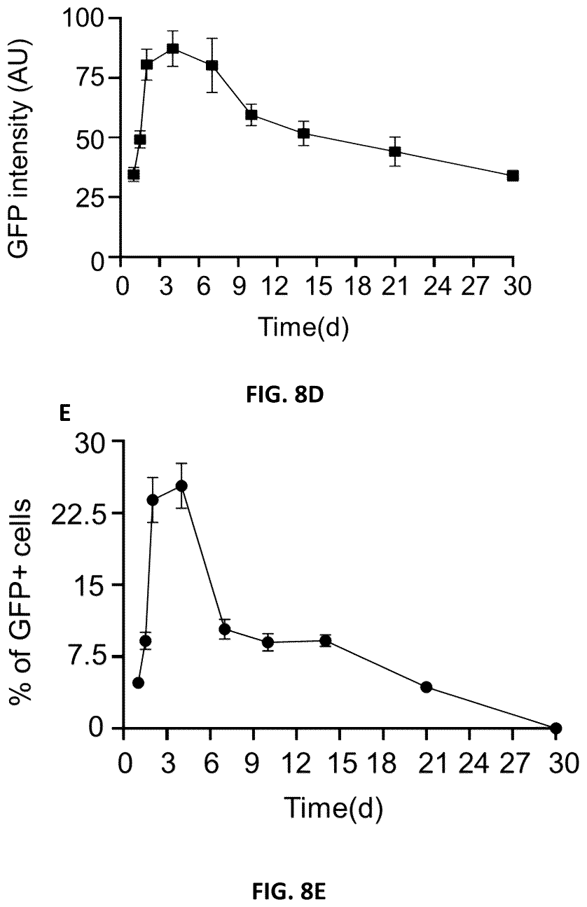

To understand the kinetics of EV-mediated COL1A1 mRNA delivery and protein formation in vivo, COL1A1-EVs were delivered subcutaneously into the dermis of mice via an insulin needle syringe. The mice were sacrificed over the next 14 days for histological analysis by RNAscope for COL1A1 mRNA quantification. COL1A1 mRNA was found to be significantly elevated in local skin tissue at 12 h after delivery, with notable decreases at 24 h and 48 h observed after injection, and a return to baseline COL1A1 mRNA levels by 96 h (see FIG. 8A and FIG. 8B). Briefly, 10-12 week nude mice were anesthetized with isoflurane, and the dorsal skin region was injected with 50 uL 3×10⁸ CNP COL1A1 mRNA EVs. At predefined time points (0, 12, 24, 48, 96 hr, 7 d, 10 d, 14 d) following the skin injection, the skin was excised and put in 4% formaldehyde for fixation. Subsequently, the slices were embedded in paraffin and further sectioned into 4-um slices. RNAscope automated in situ hybridization assay for detection of human COL1A1 mRNA was performed, and all in situ hybridization reagents were ACD products (Advanced Cell Diagnostics, Newark, CA, USA) using the HybEZ™ II Hybridization System (ACD) to perform. In brief, target retrieval was performed at 95° C. for 15 min using Leica Epitope Retrieval Buffer 2 followed by protease treatment at 42° C. for 15 min. The probe (RNAscope® Probe-Hs-COL1A1 cat. 401891; ACD) was hybridized for 2 hr at 40° C. followed by RNA scope amplification, and RNAscope® 2.5 HD Assay—BROWN kit was used for visualization of staining. RNAscope 2.5 LS probe-Rn-Ppib used as negative controls.

In vivo translation of COL1A1 mRNA into protein was assessed by immunofluorescence microscopy of explanted tissue over a 30-day period. GFP⁺ COL1A1⁺ immunostained grafts were observed as early as 12 h after injection with peak fluorescence at 4 days after delivery (see FIG. 8C, FIG. 8D, and FIG. 8E). GFP⁺ COL1A1⁺ protein grafts were observed to decrease in a time dependent manner from day 4 to day 30 after injection as observed by immunofluorescence microscopy with the majority of COL1A1 EV-derived protein turning over by day 30. These findings suggest that COL1A1-EV delivery resulted in a 3- to 4-day pulse of COL1A1 mRNA expression in recipient tissues with protein expression peaking early after mRNA turnover, and decreasing to near baseline levels by day 30 after injection.

### Example 9. In Vivo Therapeutic Efficacy of COL1A1-EVs in Collagen Depletion Skin Photoaging Model

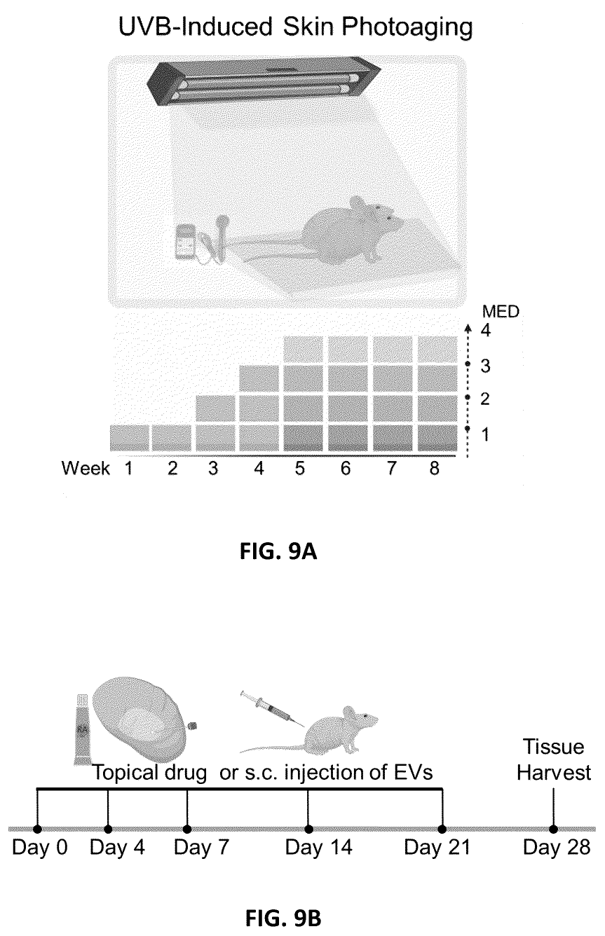

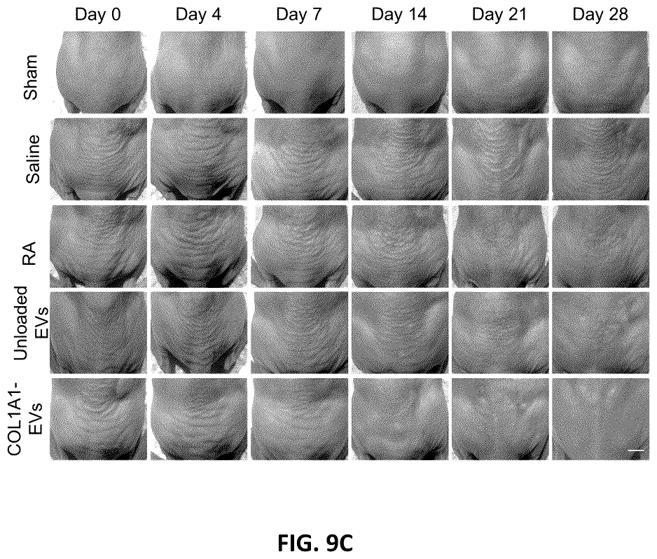

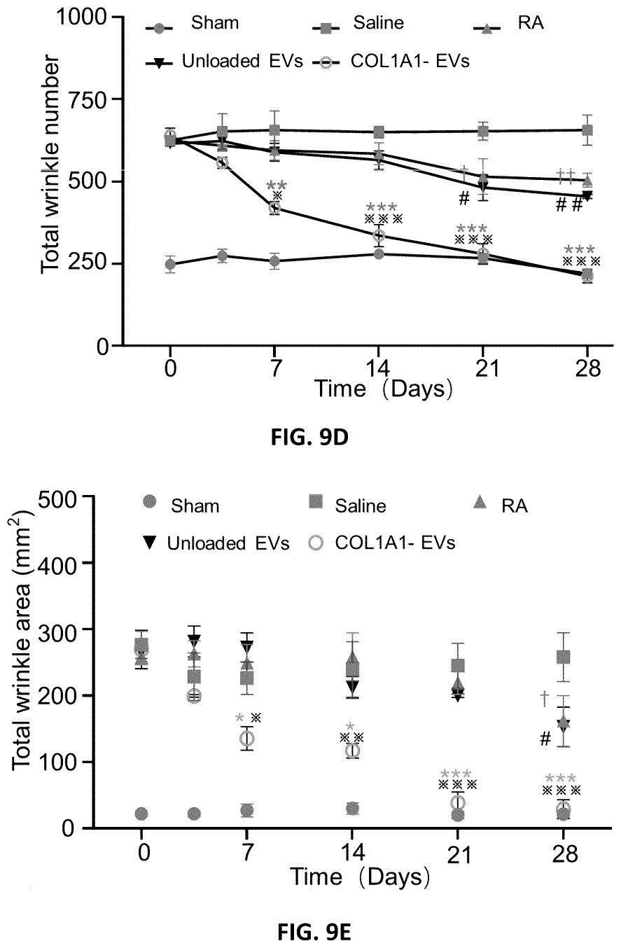

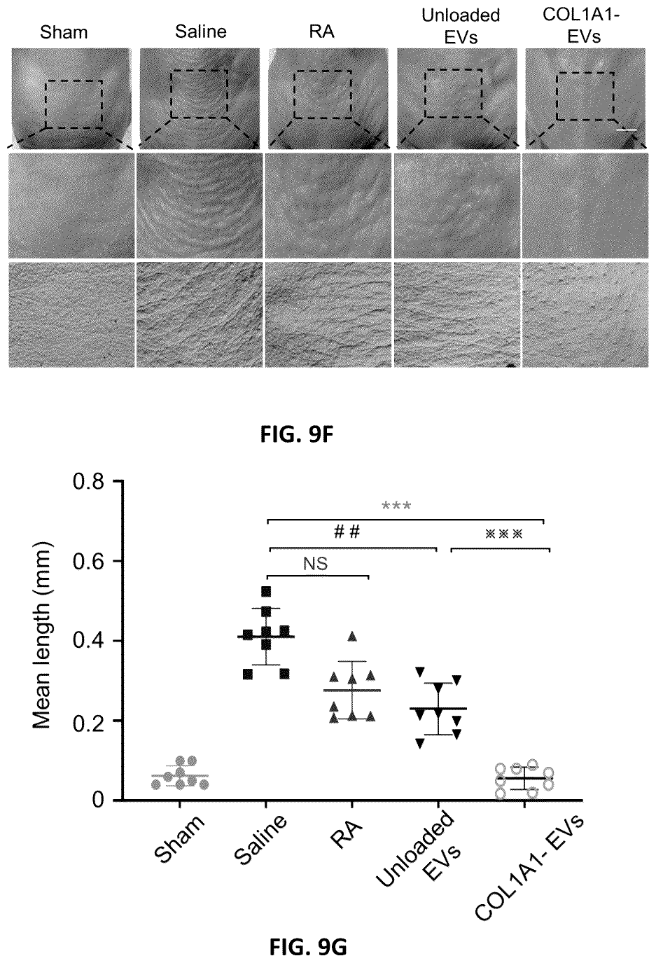

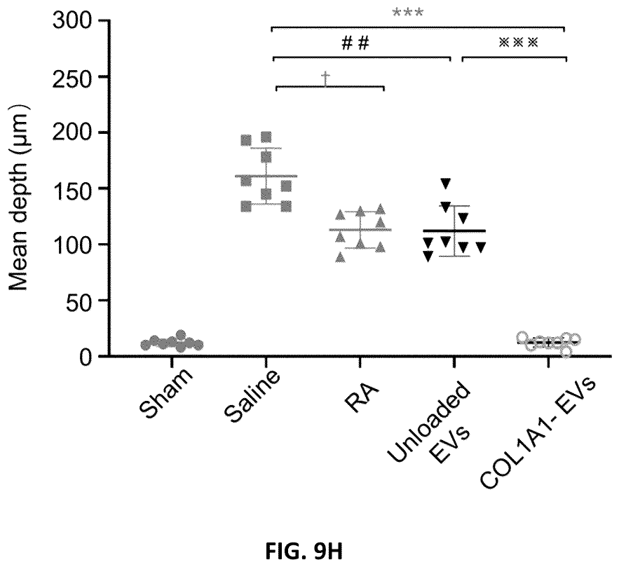

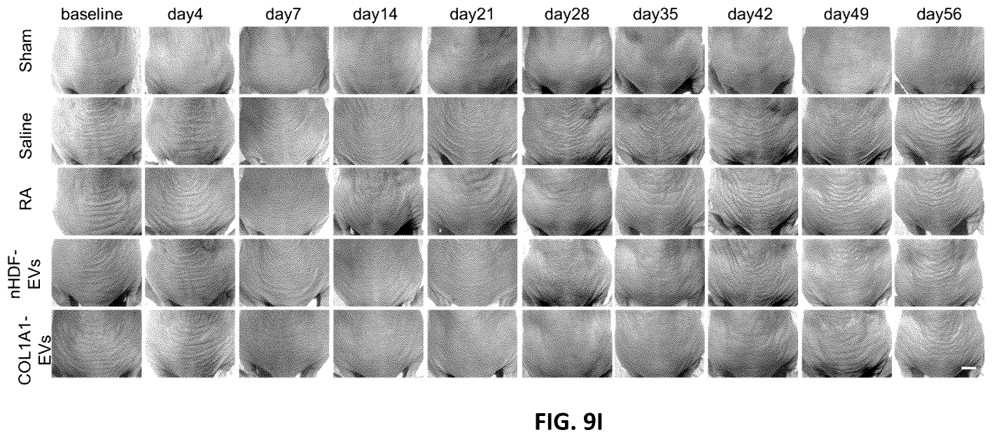

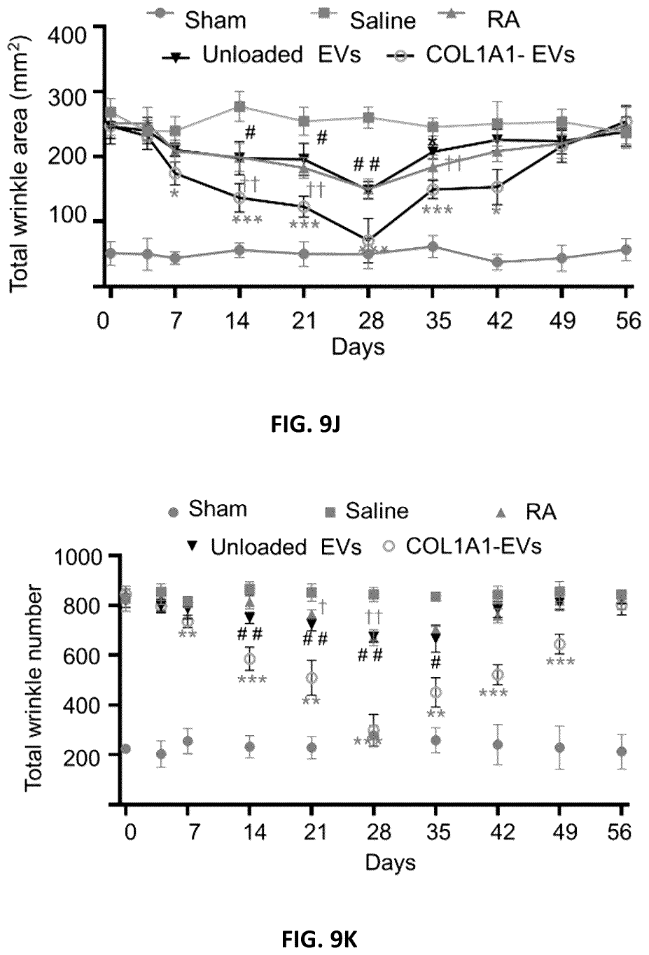

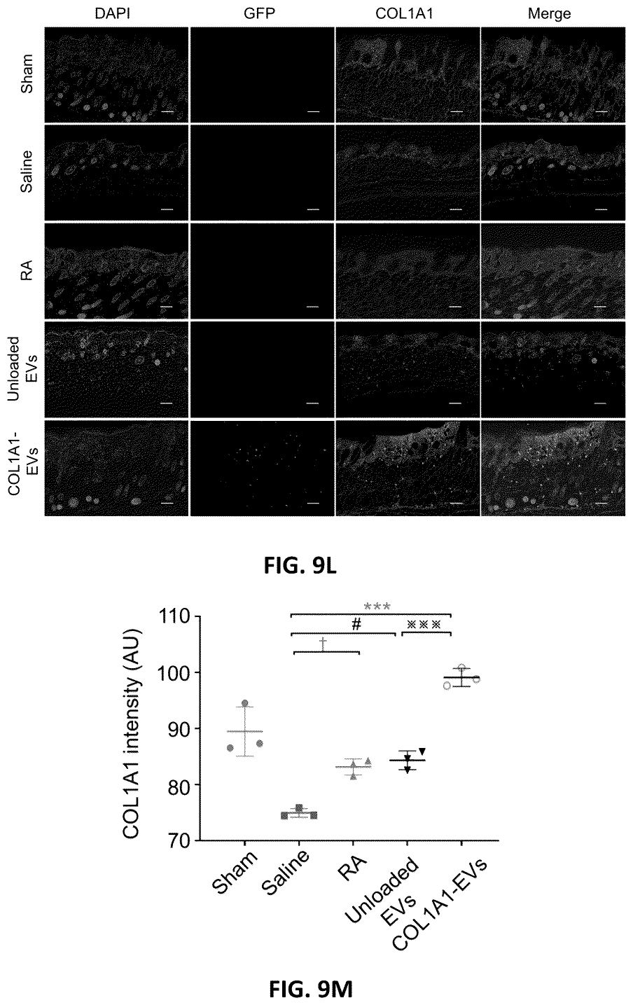

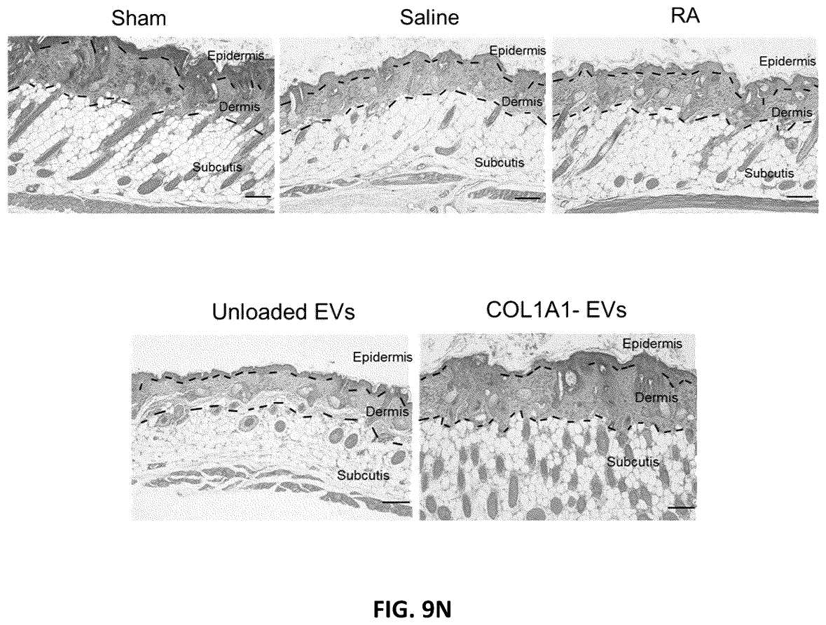

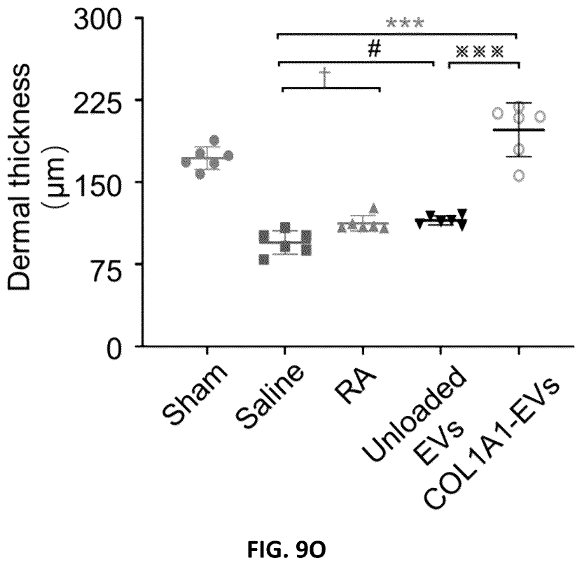

The therapeutic potential of COL1A1-EVs to replace dermal collagen was assessed in a photoaging model involving UV skin irradiation (see FIG. 9A). Athymic hairless mice were irradiated with UVB (311 nm) over an 8-week period, resulting in the formation of dermal wrinkles secondary to collagen depletion. Briefly, all animal work was approved by the Institutional Animal Care and Use Committee (IACUC) of Shenzhen Bay Laboratory or partner laboratories. To create the skin photoaging model, female nude mice (10-12 weeks old) were subjected to UVB irradiation of the dorsal skin every other day for 8 weeks as follows. Mice were anesthetized with 1.5% isoflurane, and then UV irradiation was delivered with UV lamp (Philips, Somerset, NJ, USA): emission spectrum 311 nm, positioned 30 cm above the dorsal skin of the mice every other day, for 8 weeks. The UV irradiation intensity, represented as the minimal erythemal dose (MED), was set at 1 MED during the first 2 weeks (60 mJ/cm²) and was elevated to 2 MED (120 mJ/cm²) in the third week, to 3 MED (180 mJ/cm²) in the fourth week, and to 4 MED (240 mJ/cm²) during the fifth to eighth weeks of the experiment. The total irradiated UVB volume was approximately 80 MED. For the syringe-based treatment of photoaged skin, nude mice, after the 8-week irradiation period described above, were assigned to 1 of 5 treatment groups (4 mice each): (a) UVB irradiation+50 μL saline; (b) UVB irradiation+0.05% retinoic acid; (c) UVB irradiation+50 μL nHDF-EVs delivered with a 32 G Hamilton syringe; (d) UVB irradiation+50 μL (3.78×108 particles diluted in saline) nHDF CNP COL1A1-GFP-EVs delivered with a 32 G Hamilton syringe; and (e) no UVB exposure (sham). Skin treatments took place on days 0, 4, 7, 14, and 21. The whole back skin was divided into three parts to be analyzed. Wrinkle formation was tracked at days 0, 4, 7, 14, 21, and 28 after initiation of treatment, and all animals were sacrificed at day 28 for histology and skin plaster replica analyses (see FIG. 9B).

Microscopic photography of dermal skin wrinkles over the treatment period demonstrated modest decreases in wrinkle number and area by day 28 for the unloaded EVs and retinoic acid groups relative to the saline control group (see FIG. 9C). In contrast, photoaged mice treated with subcutaneous COL1A1-EVs exhibited a reduction in wrinkle number and area beginning on day 7 after treatment initiation with a significant reduction from day 14 onward to levels similar to those observed in unirradiated sham controls (see FIG. 9D and FIG. 9E). Then skin replica was performed at the end of the treatment on the back skin of mice. A SILFLO silicone replica and ring locator were brought from Clinical & Derm, LLC (Dallas, TX, USA). Replicas of the back (dorsal) skin of the mice were obtained at the end of the treatment period. The replicas were analyzed by stereomicroscopy (Olympus SZX7), and corresponding images were analyzed by ImageJ (NIH). Skin plaster replicas of the dorsal skin taken at day 28 after treatment initiation confirmed the effectiveness of COL1A1-EVs for treating photoaged skin as compared with saline control, unloaded EV controls, and retinoic acid (see FIG. 9F, FIG. 9G, and FIG. 9H). After skin plaster assessment at day 28, a subset of animals were retained for 4 additional weeks to monitor wrinkle reduction duration. Dermal wrinkles were seen to reappear as early as 1 week later, beginning at day 35 after treatment initiation, and by day 56 dermal wrinkles were statistically indistinguishable from pre-treatment levels. (see FIG. 9I, FIG. 9J, and FIG. 9K). Specifically, the results indicated a gradual decrease in skin wrinkle area of the COL1A1-EV group beginning on day 7 and continuing through day 28 compared with the saline control (***P<0.001). Dorsal wrinkles in the COL1A1-EVs cohort reappeared starting at the 49th day after treatment. Quantification of wrinkles in the COL1A1-EVs cohort indicated recovery from the 56th day. However, the photoaged mice receiving COL1A1-EVs showed a significant effect from day 14 onwards to similar levels in unirradiated sham controls compared with control unloaded EV- and topical RA-treated mice.

To evaluate dermal collagen engraftment from COL1A1 EV delivery, skin was excised from all groups at day 28 after treatment initiation and evaluated by immunofluorescence microscopy, as well as Masson trichrome staining. Histological analysis revealed that COL1A1 protein expression was significantly restored after COL1A1 EV treatment relative to other groups (see FIG. 9L and FIG. 9M). Masson trichrome staining confirmed higher levels of collagen staining and dermal thickness in mice given COL1A1-EVs than in mice given saline control, retinoic acid, or unloaded EVs (see FIG. 9N and FIG. 9O).

### Example 10. Design of a Novel Microneedle (MN) EV mRNA Delivery System for Improved Protein Engraftment

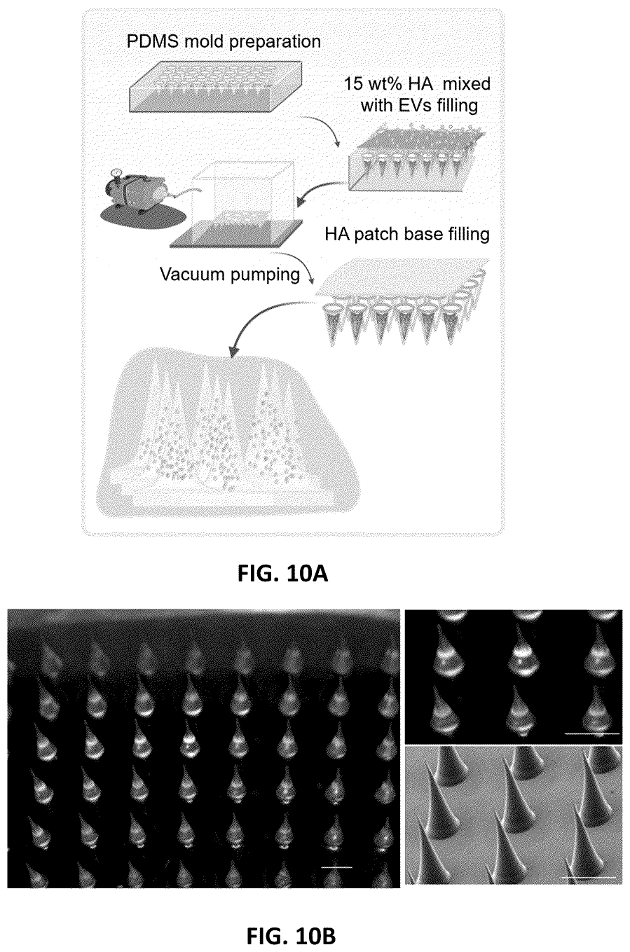

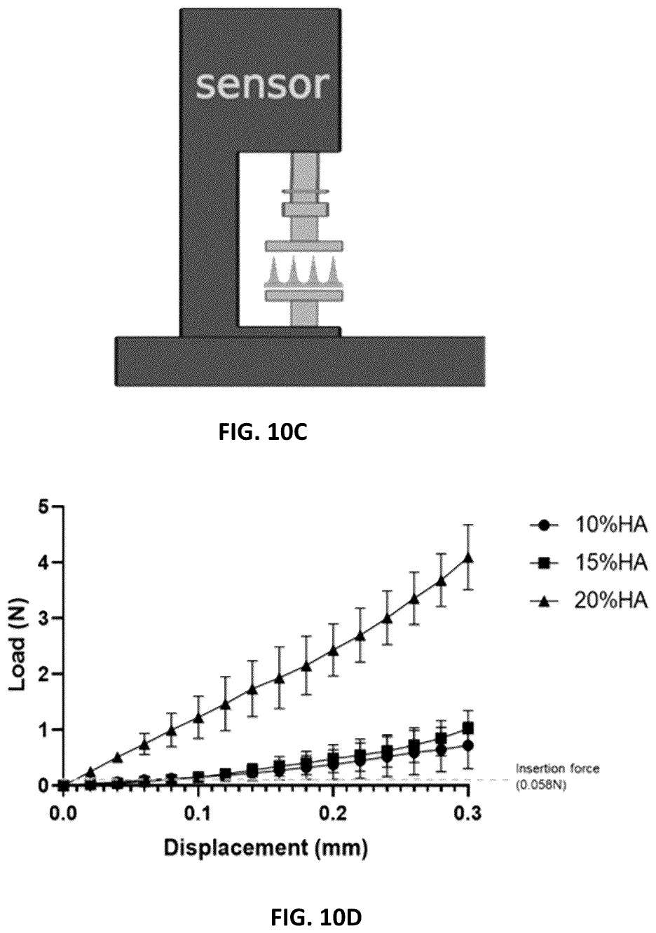

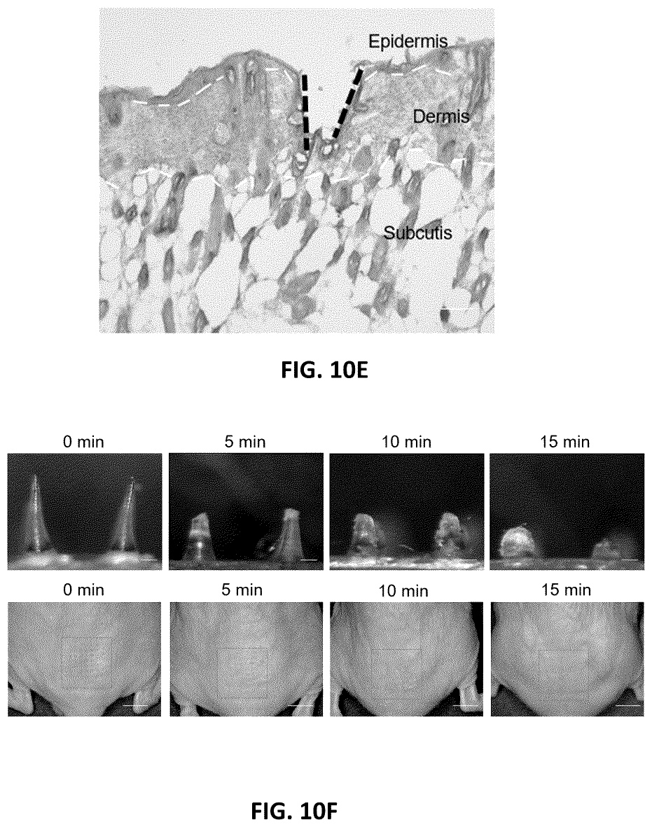

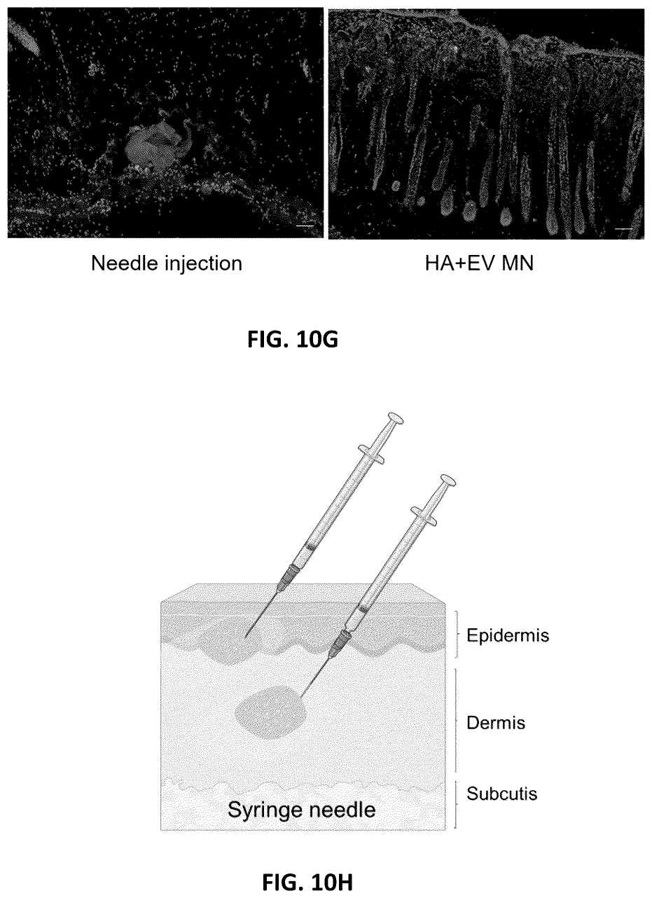

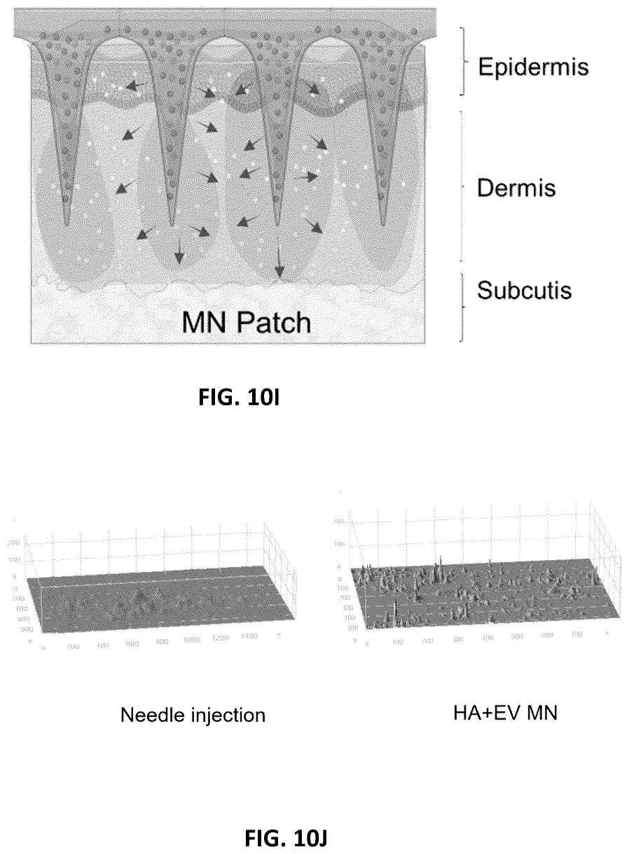

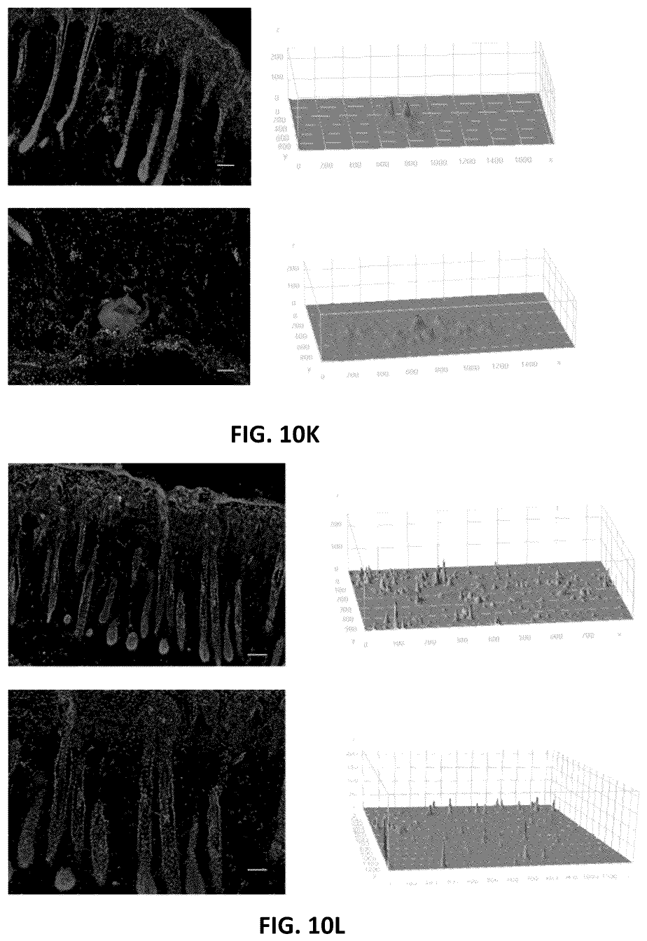

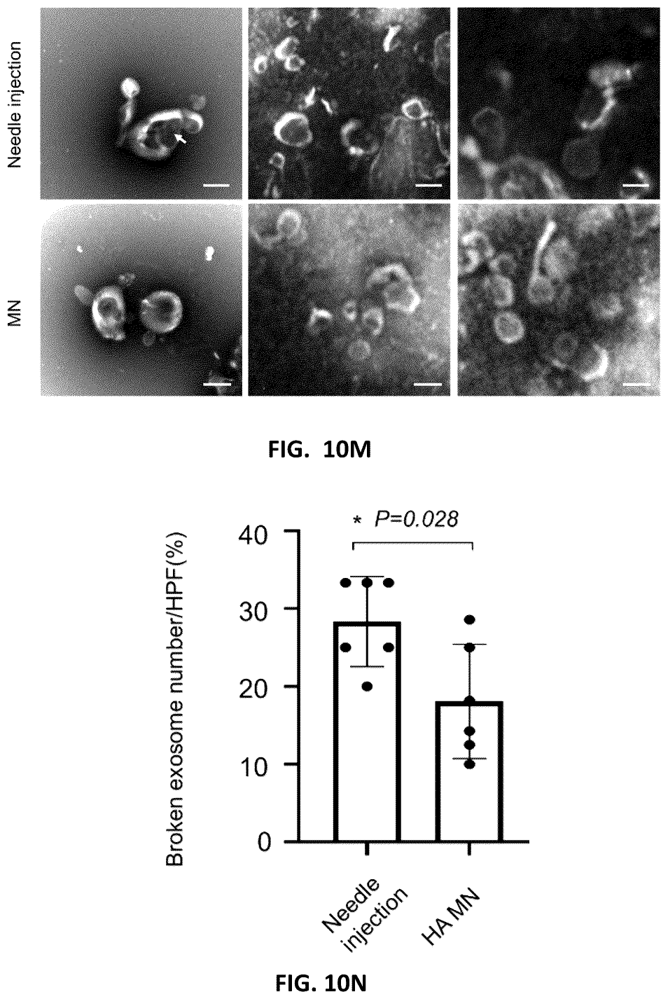

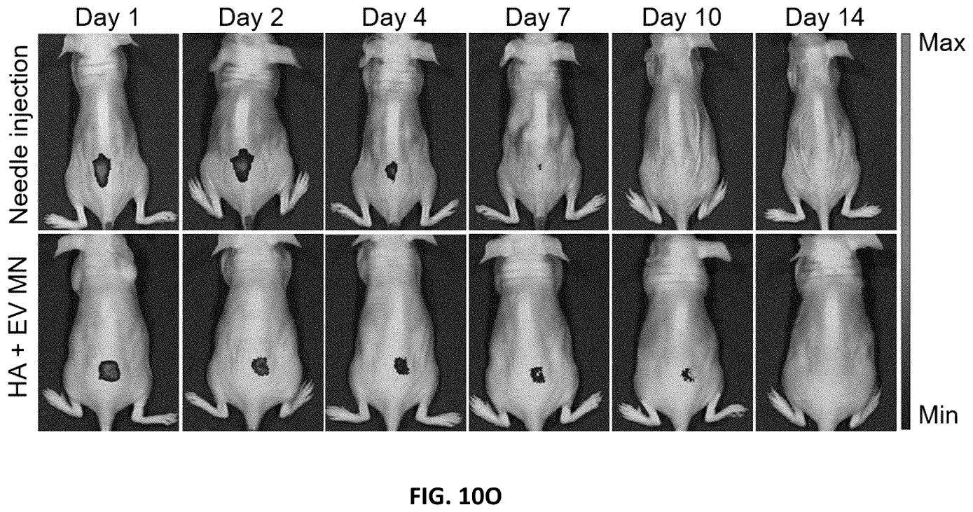

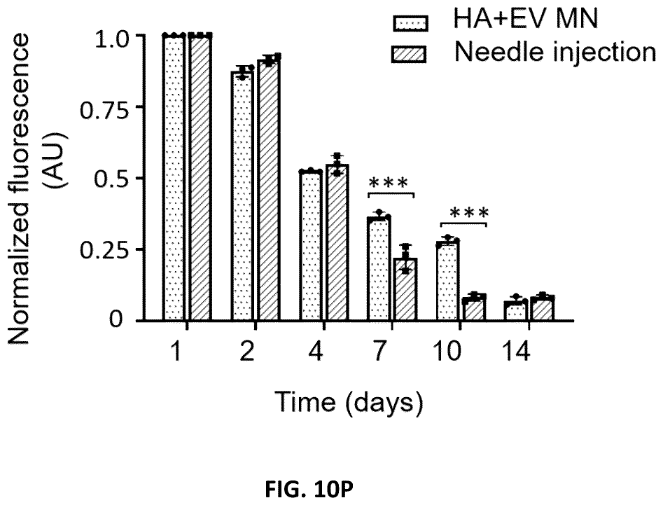

To improve the duration of protein replacement and effectiveness of collagen engraftment, a novel hyaluronic acid microneedle formulation (HA+EV MN) was designed to improve EV-mediated mRNA tissue delivery. These needle cavities were arranged in a 10×10 array with 700 um tip-tip spacing. For the preparation of the MN patch, first, ddH2O was used to dissolve hyaluronic acid powder (Low MW Hyaluronate 10000 Da-0.2 million Da) which allowed for polymerization. 150 ul of 15 wt % HA solution was mixed with 50 ul EVs and kept under vacuum for 30 minutes. The mixture was cast into the tips of a poly dimethylsiloxane (PDMS) mold. To concentrate EVs at needle tips, molds were kept at 4° C. for 4 h. Finally, 1 mL of 15 wt % HA solution was loaded on the micro mold and left to dry. After drying, the MN patch was detached from the silicone mold for further use. (see FIG. 10A). Each needle of HA+EV MN patch was molded in a conical shape, with a circular diameter of 400 μm at their base and a height of 1000 μm (see FIG. 10B). The mechanical strength of patches with 10%, 15%, or 20% HA loaded with EVs was evaluated with a tensile testing machine (see FIG. 10C). MN were placed on a flat metal plate and the liquid nitrogen was used to cool the probe and metal plate. A 5 mm diameter flat-tipped stainless steel cylindrical probe (at a constant speed of 0.5 mm min-1) was applied with a vertical force to the MN tip. The mechanical strength of the MN were found to increase with the concentration of HA. The load fracture force of 15% HA+EV MN was confirmed to be higher than the minimum average force needed for skin penetration (0.058N), and was shown to have less broken microneedles than 10% HA (see FIG. 10D).

Hematoxylin and eosin (H&E) staining confirmed that MNs penetrated through the stratum corneum into the dermis (516±76 μm) (see FIG. 10E). Briefly, Skin specimens of nude mice were fixed in 4% paraformaldehyde solution for 48 hours and embedded in paraffin, and sectioned into 5 μm thick sections. For H&E staining, the sample was de-waxed, hydrated, stained with hematoxylin and eosin dyes, and dehydrated. For EV delivery, HA+EV MN patches were pressed into the dorsal skin of mice, and the MN base was removed after 15 minutes. During this period, the MNs dissolved completely, with no visible skin irritation or marking at the site of administration (see FIG. 10F). To compare the tissue distribution of EVs delivered via MN with that of EVs delivered by insulin syringe, EVs were labelled with DiI (1,1′-dioctadecyl-3,3,3′,3′-tetramethylindocarbocyanine perchlorate; Beyotime, C1036) and administered subcutaneously under the dorsal skin of recipient mice, with subsequent histological assessment by immunofluorescence microscopy (see FIG. 10G, FIG. 10H, and FIG. 10I). Tissue distribution analysis revealed that syringe needle injection resulted in uneven delivery of EVs with clumping in specific areas of the dermis and subcutis, whereas EVs delivered by MN were better dispersed in dermis and subcutis. Specifically, dil-labeled EVs were injected subcutaneously either by syringe needle (see FIG. 10H) or by the MN patch (see FIG. 10I). The subcutaneous distribution of EVs delivered by the HA MN was more uniform than that delivered by the syringe needle (see FIG. 10K and FIG. 10L). To further explore any potential damage to EVs caused by shear stress of the syringe needle, the same number of EVs were taken and injected onto blank cell culture plates with PBS with either a syringe or an HA MN. Cryogenic electron microscopy showed that the MN condition better preserved the morphology and structure of the EVs (see FIG. 10M and FIG. 10N), mainly because of the shear force produced by the flow rate of the liquid in the syringe needle condition. Briefly, collected EVs were suspended in tubes and incubated with paraformaldehyde, and then loaded onto a pre-prepared copper grid and incubated to allow adsorption. Next, uranyl acetate was added to copper mesh (a heavy metal stain that binds uranyl ions to membrane proteins and lipids). Finally, the grids were washed with water and dried to remove excess stains for TEM imaging.

To assess whether the improved tissue distribution and EV membrane protection resulted in improved EV engraftment, serial in-vivo fluorescence imaging of DiI labelled EVs injected subcutaneously in mice was employed over a 14 day period. Fluorescence imaging confirmed similar DiI EV signal between syringe delivered EVs and MN delivered EVs for the first 4 days after injection. However, the HA+EV MN group had significantly higher fluorescence signal at day 7 (36.7+1.5% HA+EV MN vs 30+4.3% Needle, P<0.001) and at day 10 after injection (28.0+1.5% HA+EV MN vs 8.4+1.3% Needle, P<0.001), suggesting that use of the MN patch improved long-term EV retainment of EVs in tissue (see FIG. 10O) and FIG. 10P). Briefly, MN-treated mice were imaged with an in vivo imaging system (IVIS, Spectrum, Perkin Elmer) on days 0, 1, 3, 7 and 14. The parameters were set as follows: the exposure time was 15 s, excitation 570 nm, emission 680 nm, 2F/stop, and 13.6-cm field of view in the specified RFP imaging times. Quantitative analysis of RFP fluorescence intensity was performed by measuring the average radiation efficiency (photon s-1 cm-2 sr-1 μW-1) in a region of interest (ROI). Data were normalized to fluorescence intensity on day 0.

### Example 11. Therapeutic Efficacy of HA+EV MN Delivery System for In Vivo Protein Replacement of Collagen

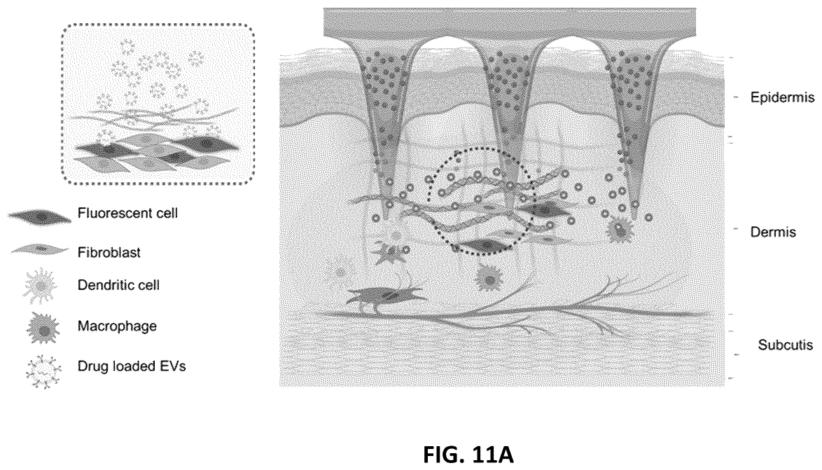

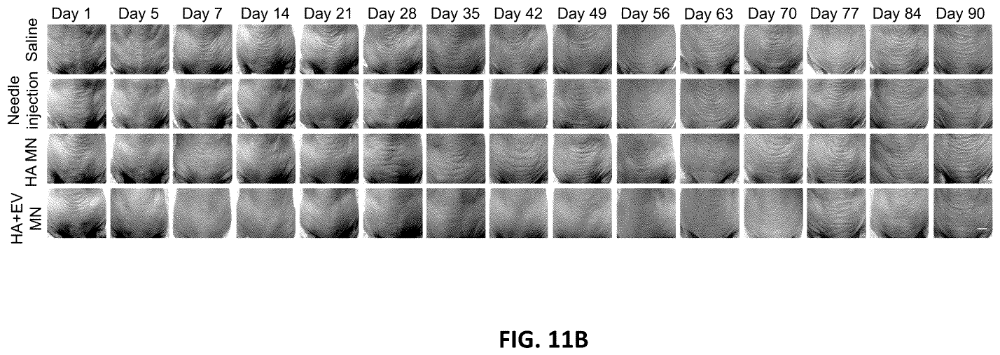

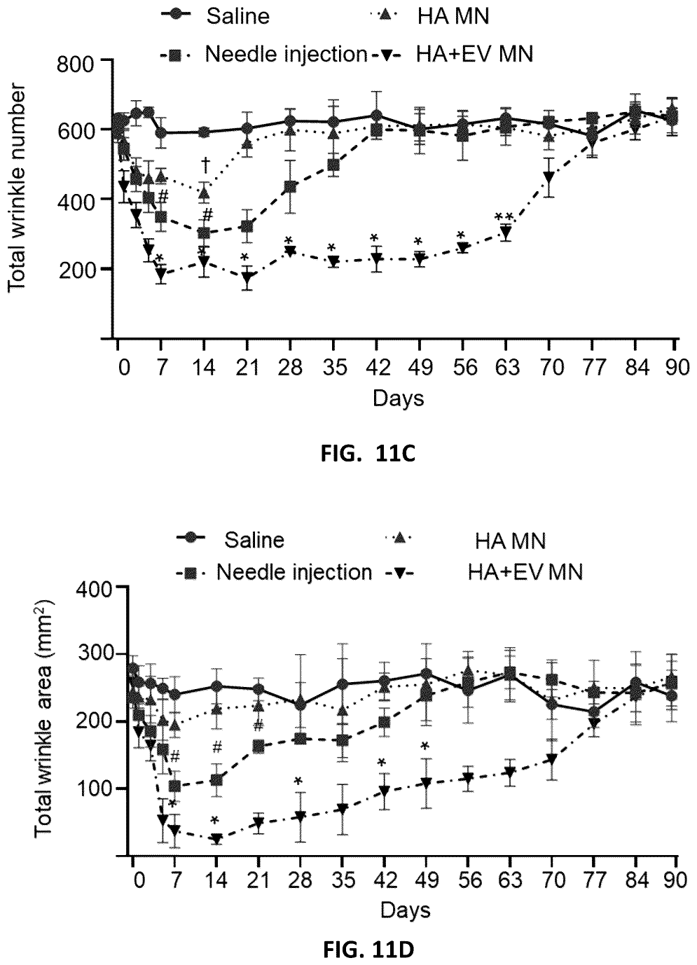

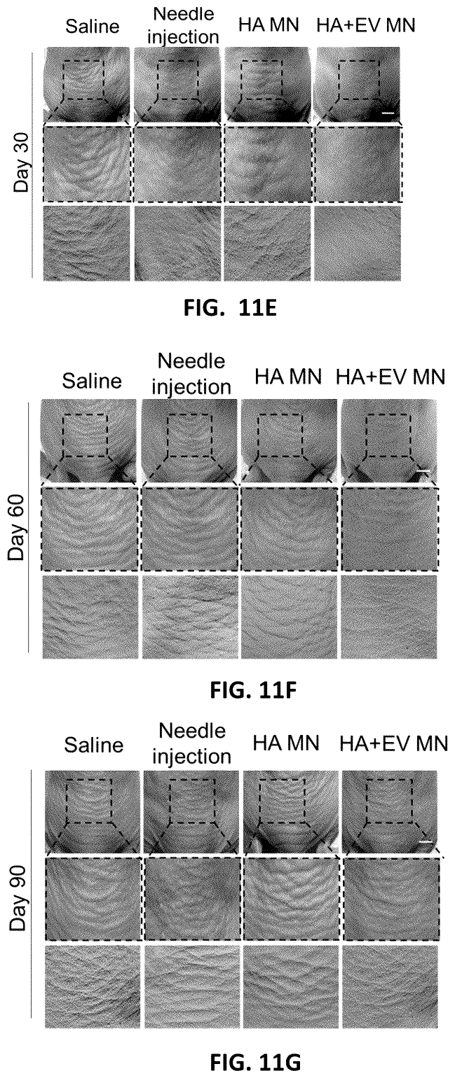

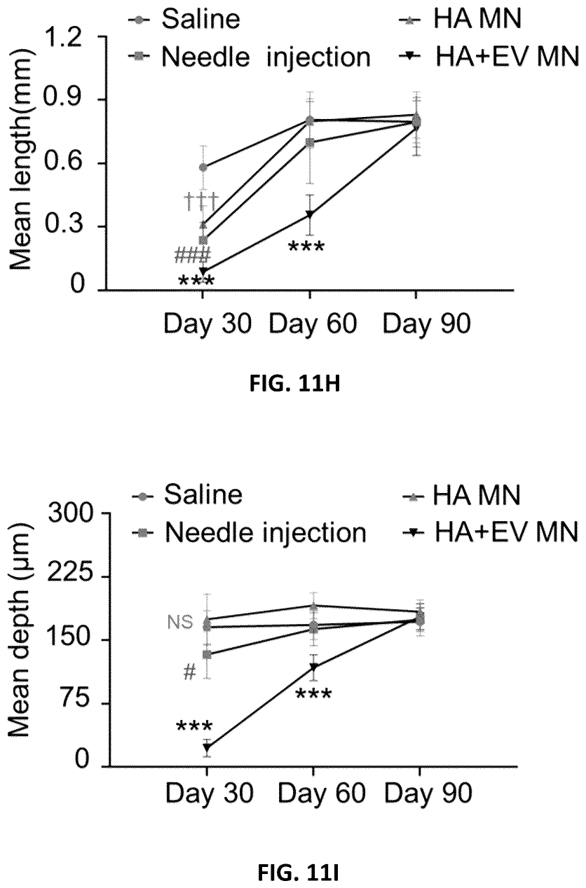

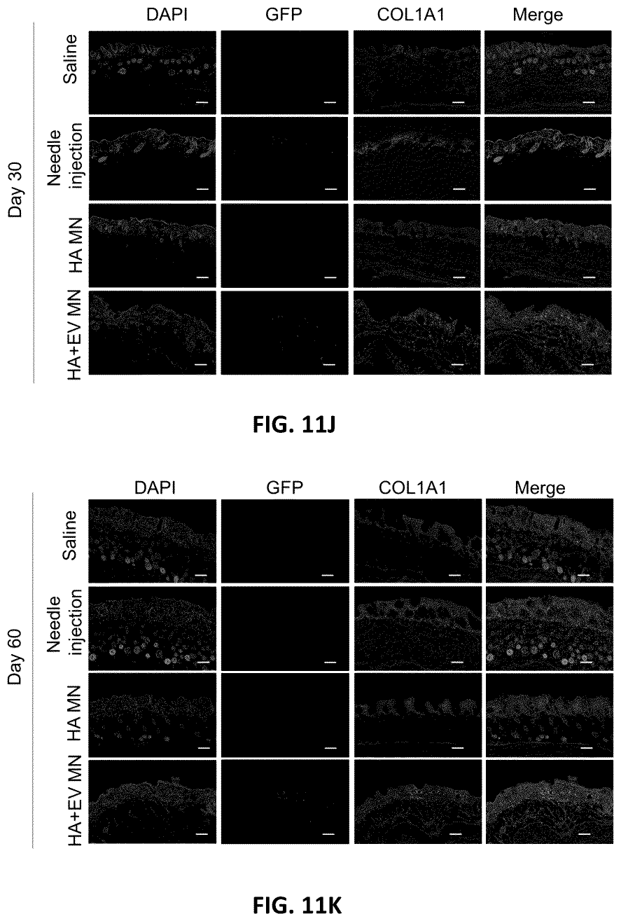

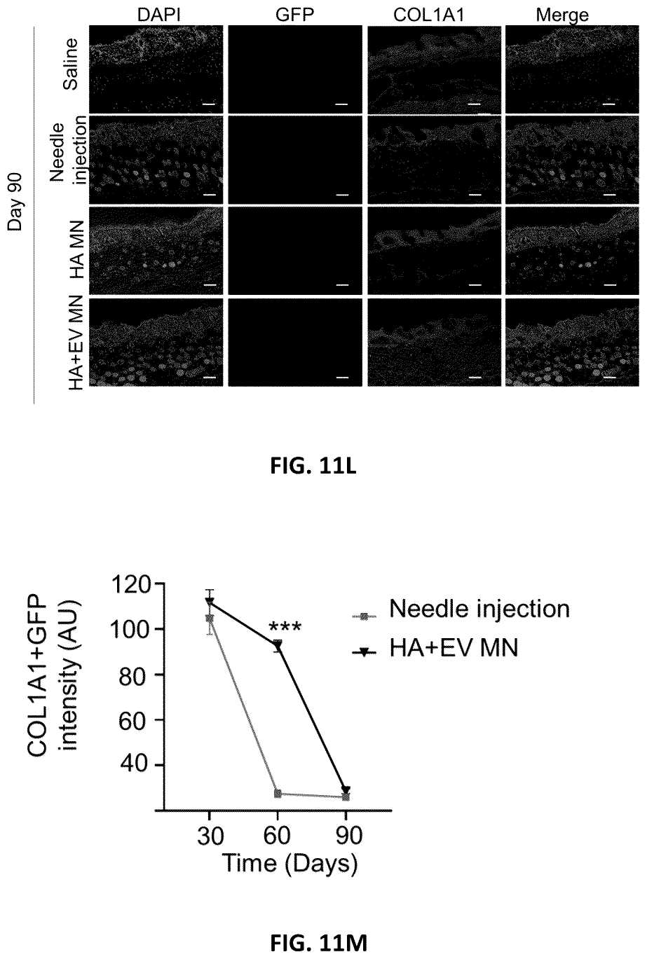

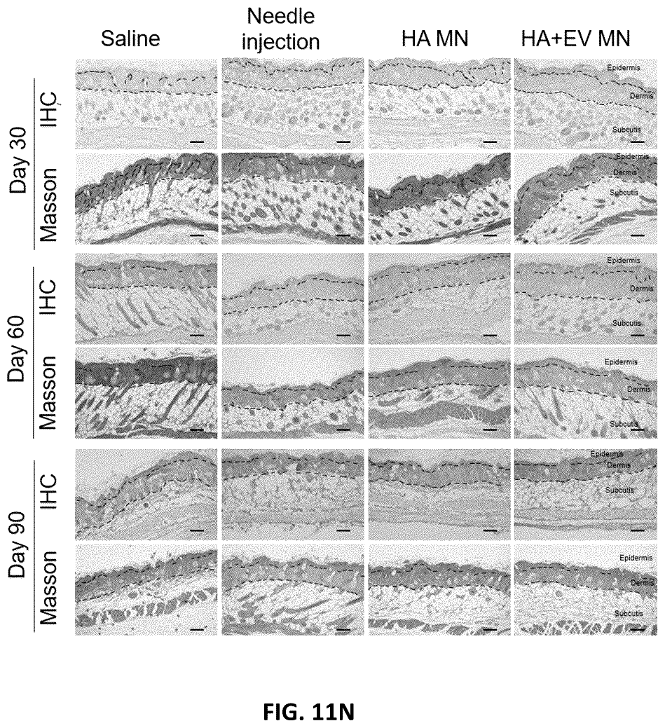

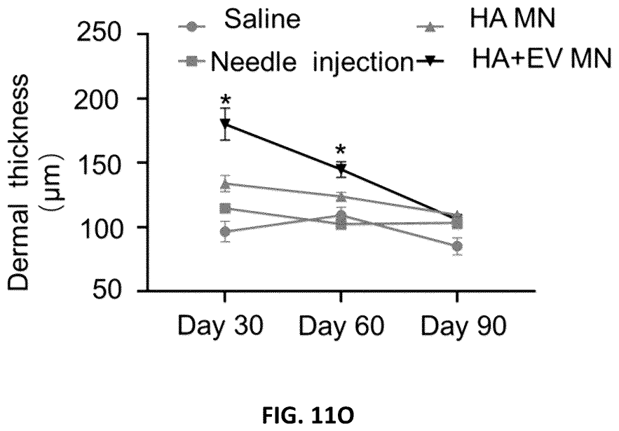

Whether use of the custom HA+EV MN patch disclosed herein for delivering COL1A1 mRNA EV delivery could result in improved in vivo protein replacement in the skin of photoaged mice (see FIG. 11A) was investigated. Athymic mice were again subjected to 8 weeks UV irradiation (see FIG. 9A) and assigned to 1 of 4 treatment groups (4 mice each): (a) UVB irradiation+50 μL saline; (b) UVB irradiation+50 μL (about 10¹⁰ particles, diluted in saline) nHDF CNP COL1A1-GFP mRNA EVs delivered by 32 G Hamilton syringe; (c) UVB irradiation+HA MN patch; and (d) UVB irradiation+15% HA mixed with CNP COL1A1-GFP mRNA EVs. All mice were given a single dose injection of 50 ng COL1A1 mRNA (or an equivalent volume for control groups) at day 0 of the treatment timeline.

After injection, all mice were monitored via microscopic photography of dorsal skin wrinkles for up to day 90 (see FIG. 11B). Relative to syringe needle injection, which reduced wrinkle formation for up to 35 days before a return to pre-treatment baseline, delivery of COL1A1 mRNA by HA+EV MN was found to substantially reduce wrinkle area and number for up to 70 days before a return to baseline levels (see FIG. 11C and FIG. 11D). To further confirm these findings, a subset of mice from each group were sacrificed at day 30, 60, or 90 for skin replica plaster assessment of the of the dorsal skin and histology (see FIG. 11E, FIG. 11F, and FIG. 11G). Both needle injection and HA+EV MN treatments reduced the wrinkle length and depth up to day 30 post treatment (see FIG. 11H and FIG. 11I). However, only mice treated with HA+EV MN showed significantly long-lasting reductions in wrinkle length and depth for up to 60 days.

Histologic analysis of skin samples taken at day 30, 60, and 90 of the study confirmed the lasting engraftment of GFP+ COL1A1 protein engraftment in the dermis and subcutis of mice, in both the needle injection group and the HA+EV MN group, at day 30 (see FIG. 11J). However, at day 60, only the mice that received HA+EV MN showed GFP+ COL1A1 engraftment in the dermis and subcutis (see FIG. 11K). Immunofluorescence microscopy of skin samples taken on day 90 revealed no GFP collagen grafts in any of the treatment groups (see FIG. 11L and FIG. 11M), supporting the wrinkle microscopy findings which demonstrated effective treatment at days 30-60, and a return to pre-treatment baseline from days 70-90.

Immunohistochemical (IHC) staining of COL1A1 protein and Masson trichrome staining of skin tissues confirmed that the amount of collagen in the dermis correlated with immunofluorescence microscopy findings and that among all cohorts, the HA+EV MN group had the most abundant collagen fibers and dermal thickness up to day 60 of the study (see FIG. 11N and FIG. 11O). Briefly, the tissue sections were fixed in 4% paraformaldehyde for 20 min, washed 3 times with PBS (pH 7.4) for 5 minutes each and then transferred to a retrieval box containing ethylenediaminetetraacetic acid (EDTA) (pH 9.0) antigen retrieval solution for antigen retrieval in a microwave oven. Subsequently, the sections were incubated in 3% hydrogen peroxide solution at room temperature for 25 min in the dark. After washing with PBS, the tissue was evenly covered with 3% BSA or 10% normal rabbit serum. After blocking at room temperature for 30 min, primary antibody (ab34710 and ab6556, Abcam) was added and incubated overnight at 4° C. Then, a secondary antibody (HRP-labeled) corresponding to the primary antibody was added to cover the tissue sections and incubated at room temperature for 50 min. DAB color development: The slides were placed in PBS (PH7.4) and washed 3 times with shaking on a destaining shaker, 5 minutes each time. After the slices were slightly dried, the freshly prepared DAB color developing solution was added dropwise in the circle. Developing time was controlled under the microscope. Hematoxylin, hematoxylin differentiation solution, and hematoxylin blue-returning solution were sequentially added for nuclei counterstaining. Finally, the glass slides were placed in anhydrous ethanol and xylene for dehydration and sealing. For Masson's trichrome staining, paraffin-embedded skin specimens were stained and dehydrated with Bouin solution, Weigert iron hematoxylin working solution, phosphomolybdenum-phosphotungstic acid solution and aniline blue solution, respectively. The dorsal skin thickness of the mice was measured with calipers on the day of sacrifice.

Taken together, these findings indicated that encapsulation and delivery of COL1A1 mRNA via a novel HA+EV MN system prolonged collagen protein replacement in photoaged skin for more than twice the duration as syringe needle delivery. Notably, EVs not loaded with COL1A1 mRNA were also able to achieve a modest decrease in winkle number.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A plurality of extracellular vesicles comprising an exogenous extracellular matrix messenger RNA (mRNA), wherein the exogenous extracellular matrix mRNA encodes a collagen, and wherein the plurality of extracellular vesicles does not comprise an alpha 2 chain of collagen type I (Col1A2).

2. (canceled)

3. The plurality of extracellular vesicles of claim 1, wherein the collagen is selected from the group consisting of collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, collagen type XX, collagen type XXI, collagen type XXII, collagen type XXIII, collagen type XXIV, collagen type XXV, collagen type XXVI, collagen type XXVII, collagen type XXVIII, or any combination thereof.

4. The plurality of extracellular vesicles of claim 1, wherein the collagen is collagen type I.

5. The plurality of extracellular vesicles of claim 1, wherein the collagen is collagen type II.

6. The plurality of extracellular vesicles of claim 1, wherein the collagen is alpha 1 chain of collagen type I (Col1A1) or pro-alpha1(I) chain.

7. (canceled)

8. The plurality of extracellular vesicles of claim 1, wherein the collagen is Col1A1.

9. The plurality of extracellular vesicles of op claim 1, wherein the plurality of extracellular vesicles comprises an average of at least one copy of the exogenous extracellular matrix mRNA per 450 EVs.

10. (canceled)

11. The plurality of extracellular vesicles of claim 1, wherein the plurality of extracellular vesicles is produced by transfecting a cell with a plasmid encoding an extracellular matrix protein.

12. The plurality of extracellular vesicles of claim 11, wherein the transfecting of the cell is performed by cellular nanoporation.

13. The plurality of extracellular vesicles of claim 11, wherein the cell is a human cell.

14. The plurality of extracellular vesicles of claim 11, wherein the cell is a fibroblast selected from the group consisting of dermal fibroblast, human fibroblast, adult fibroblast, human adult fibroblast, neonatal fibroblast, neonatal human fibroblast, and neonatal human dermal fibroblast.

15. The plurality of extracellular vesicles of claim 1, wherein the plurality of extracellular vesicles comprises an extracellular vesicle selected from the group consisting of exosome, microvesicle, apoptotic body, and any combination thereof.

16. The plurality of extracellular vesicles of claim 1, wherein the plurality of extracellular vesicles comprises at least one exosome.

17. (canceled)

18. The plurality of extracellular vesicles of claim 1, wherein the plurality of extracellular vesicles is formulated for injection via an intravenous route, an intramuscular route, a subcutaneous route, or any combination thereof.

19. The plurality of extracellular vesicles of claim 1, wherein the plurality of extracellular vesicles comprises at least 5×10⁶ extracellular vesicles.

20. The plurality of extracellular vesicles of claim 1, wherein the plurality of extracellular vesicles comprises at least 10 ng, of the extracellular matrix mRNA.

21. The plurality of extracellular vesicles of claim 1, wherein the plurality of extracellular vesicles comprises a size distribution with a peak at 50 nm-200 nm in diameter.

22. (canceled)

23. The plurality of extracellular vesicles of claim 1, wherein the exogenous extracellular matrix mRNA is present at a level that is at least 2-fold higher than a level of the exogenous extracellular matrix mRNA in an identical amount of naturally-occurring extracellular vesicles.

24. The plurality of extracellular vesicles of claim 1, wherein the plurality of extracellular vesicles expresses higher levels of at last one of the following markers compared to a comparable number of naturally-occurring extracellular vesicle: CD9, CD63, TSG101, or ARF6.

25.-48. (canceled)

49. A method of treating a skin condition in a subject comprising administering at least one extracellular matrix mRNA to a subject in need thereof, thereby treating the skin condition.

50. The method of claim 49, wherein the exogenous extracellular matrix mRNA encodes a collagen, and wherein the plurality of extracellular vesicles does not comprise an alpha 2 chain of collagen type I (Col1A2).

51.-55. (canceled)

56. The method of claim 49, wherein the skin condition is skin damage or a wound.

57.-58. (canceled)

59. The method of claim 49, wherein the EV is an exosome, an apoptotic body, or a microvesicle.

60.-77. (canceled)

78. A method of producing an extracellular vesicle comprising extracellular matrix mRNA comprising:
(a) introducing a vector or a plasmid correspond to the extracellular matrix mRNA into a donor cell via transfection;
(b) culturing the donor cell for a sufficient amount of time in a culture medium for the production of extracellular vesicles encapsulating the extracellular matrix mRNA transcribed from the vector or the plasmid; and
(c) collecting the extracellular vesicles from the culture medium.

79. A microneedle device, the microneedle device comprising a substrate and a plurality of microneedles, wherein the plurality of microneedles protrude from the substrate, and wherein at least one microneedle of the plurality of microneedles comprises at least one extracellular vesicle (EV).

80.-108. (canceled)

109. A plurality of extracellular vesicles comprising an exogenous extracellular matrix messenger RNA (mRNA), wherein the plurality of extracellular vesicles comprises an average of at least one copy of the exogenous extracellular matrix mRNA per 450 EVs.
